(19) United States
(12) Patent Application Publication
(10) Pub. No.: US 2004/0116684 A1
(43) Pub. Date: Jun. 17, 2004

(54) Ancestral viruses and vaccines

(76) Inventors: Allen Rodrigo, (US); Howard A. Ross, (US); James I. Mullins, Seattle, WA (US)

Correspondence Address: FISH & RICHARDSON PC, 225 FRANKLIN ST, BOSTON, MA, 02110, US

(21) Appl. No.: 10/441,949

(22) Filed: May 19, 2003

Related U.S. Application Data
(63) Continuation-in-part of application No. PCT/US01/05288, filed on Feb. 16, 2001.
(60) Provisional application No. 60/183,659, filed on Feb. 18, 2000.

(51) Int. Cl. C07H 21/04; C12N 7/00

(52) U.S. Cl. 536/23.72; 435/235.1

(57) ABSTRACT

The present invention is directed to ancestral viral nucleic acid and amino acid sequences, methods for producing such sequences and uses thereof, including prophylactic and diagnostic uses.

### CROSS-REFERENCE TO RELATED APPLICATIONS

This application is a continuation-in-part application of and claims priority to International PCT Application Serial No. PCT/US01/05288, filed on Feb. 16, 2001, which claims the benefit of U.S. Provisional Application Serial No. 60/183,659, filed on Feb. 18, 2000, the entire contents of which are herein incorporated by reference including figures and tables.

### BACKGROUND

The use of nonhuman species as sources of organs for human transplantation, i.e., xenotransplantation, is a potential solution to the shortage of human organs and tissues for transplantation. Advances in the biology of interspecies transplantation have been accompanied by concerns that recipients of xenografts are at risk for infection by organisms transferred with the xenograft. The risk of viral infection in transplantation is heightened by the presence of factors commonly associated with viral activation, e.g., immune suppression, graft-versus host disease, graft rejection, viral coinfection, and cytotoxic therapies.

Pigs are among the most likely source species for xenografts for clinical use in humans. Donor animals can be raised in specific pathogen free conditions such that they are free of many pathogens liable to be transmitted from a swine graft to a human recipient. However, particular pathogens, such as endogenous retroviruses, present a greater banier to transplantation biologists. Porcine Endogenous Retrovirus-A (PERV-A) and Porcine Endogenous Retrovirus-B (PERV-B) are two classes of endogenous porcine retroviruses that are widely distributed in different pig breeds and can be present in as many as 50 copies in the genome of a given pig breed (Le Tissier, et al. Nature 389:681-682, 1997). A third porcine endogenous retrovirus, PERV-C, has also been described. Germline transmission of these viruses as well as their presence in multiple copies presents a major challenge to removal by breeding or rearing in pathogen-free conditions. Other approaches may be necessary to protect transplant recipients from infection by donor pathogens.

### SUMMARY

The present invention provides compositions and methods for determining ancestral viral gene sequences and viral ancestor protein sequences. In one aspect, computational methods are provided that can be used to determine an ancestral viral sequence for highly diverse viruses. In particular, methods are provided that can be used to determine an ancestral viral sequence for a virus that can be transmitted as a result of transplantation across a species barrier (e.g., to a xenograft recipient, e.g., to a human recipient of a non-human graft, such as a non-human primate or porcine graft). These computational methods use samples of viruses (e.g., viruses endogenous or common to a donor species) to determine an ancestral viral nucleic acid or amino acid sequence by maximum likelihood phylogeny analysis. The ancestral viral sequence can be, for example, an endogenous retrovirus ancestral sequence (e.g., a mammalian endogenous retroviral ancestral sequence, e.g., a porcine endogenous retroviral ancestral sequence). In other embodiments, the ancestral viral gene sequence is of Porcine Endogenous Retrovirus (PERV) subtype A, B, or C. PERV-A, PERV-B, and PERV-C are three classes of mammalian type C retroviruses found in pigs. Each class, or subtype of PERV has a distinct env gene (Takeuchi Y, et al. J Virol 72(12):9986-91, 1998).

Typically, the endogenous retroviral sequence is an env nucleic acid or amino acid sequence. In other embodiments, the ancestral viral sequence is of viruses other than endogenous retroviruses.

The ancestral viral nucleic acid sequence is more closely related, on average, to a nucleic acid sequence of any given circulating or germline transmitted virus than to any other variant. In some embodiments, the ancestral viral gene sequence has at least 70% identity with the sequence set forth in SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, or SEQ ID NO:41, but does not have 100% identity with any circulating (e.g., potentially replication-competent, transmissible) viral variant.

The ancestral viral sequence can encode an ancestor protein of SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:43, SEQ ID NO:44, or SEQ ID NO:45, or a fragment thereof.

In one aspect, the present invention provides an ancestral sequence for the env gene of an endogenous retrovirus, e.g., a mammalian endogenous retrovirus, e.g., PERV-A, PERV-B, or PERV-C. The determined ancestral viral sequence is, on average, more closely related to any potentially replication-competent and transmissible virus than to any other variant. The env ancestral gene sequence encodes an open reading frame that is approximately 630-680 amino acids in length.

An isolated viral ancestor protein or fragment thereof is also provided. The ancestor protein is from a virus that can be transmitted as a result of transplantation across a species barrier. For example, the ancestor protein can be from an endogenous retrovirus. The endogenous retrovirus can be a mammalian endogenous retrovirus, e.g., a porcine endogenous retrovirus. The isolated ancestor protein can be, for example, the contiguous sequence of PERV, subtype A, env ancestor protein, PERV, subtype B, env ancestor protein or PERV, subtype C, env ancestor protein. The present invention also provides computational methods for determining other ancestral viral sequences. The computational methods can be extended, for example, to determine an ancestral viral sequence for other endogenous retroviruses, and for other diverse viruses common to species from which donation organs are derived. The computational methods can also be extended to determine an ancestral viral sequence for all known and newly emerging highly diverse virus. In other embodiments, the ancestral viral sequence is determined for the genes other than the env gene of endogenous retroviruses. For example, the ancestral viral sequence can be determined for gag or pol genes.

The present invention also provides an expression construct including a transcriptional promoter; a nucleic acid encoding an ancestor protein; and a transcriptional terminator. The nucleic acid can encode, for example, a viral ancestor protein (e.g., an endogenous retroviral ancestor protein, e.g., a PERV ancestor protein. The nucleic acid can be, for example, a PERV env nucleic acid sequence (e.g., SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, or SEQ ID NO:41). In one embodiment, the nucleic acid sequence is optimized for expression in a host cell (e.g., a PERV nucleic acid sequence that is optimized for expression in a human cell, e.g., SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, or SEQ ID NO:66). The nucleic acid can encode, for example, an ancestor protein of an endogenous retrovirus (e.g., PERV, e.g., SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:43, SEQ ID NO:44, or SEQ ID NO:45).

The promoter can be a heterologous promoter, such as the cytomegalovirus promoter. The expression construct can be expressed in prokaryotic or eukaryotic cells. Suitable cells include, for example, mammalian cells, human cells, porcine cells, Escherichia coli cells, and Saccharoinyces cerevisiae cells. In one embodiment, the expression construct has the nucleic acid sequence operably linked to a Semliki Forest Virus replicon, wherein the resulting recombinant replicon is operably linked to a cytomegalovirus promoter.

In another aspect, compositions are provided for inducing an immune response in a recipient mammal, the compositions include a viral ancestor protein or an immunogenic fragment of an ancestor protein, wherein the viral ancestor protein is from a virus of a donor species. For example, the viral ancestor protein can be derived from an endogenous retrovirus, a hepatitis virus, influenza virus, or a herpesvirus of a donor species. The composition can be used as a vaccine, such as a vaccine to protect against infection of a human xenograft recipient by a highly diverse virus (e.g., a PERV). The choice of virus from which to derive the ancestor protein can depend on the host/donor species combination. For example, for inducing an immune response in a human recipient of a porcine graft, ancestor proteins from porcine viruses such as porcine endogenous retroviruses can be used. For inducing an immune response in a human recipient of a non-human primate organ, e.g., baboon, ancestor proteins from simian foamy virus and/or baboon endogenous virus can be used. The composition can include ancestor proteins of one or more subtypes, e.g., ancestor proteins of PERV subtype A, B, and C.

In another aspect, isolated antibodies are provided that bind specifically to a viral ancestor protein and that bind specifically to a plurality of circulating descendant viral ancestor proteins. The ancestor protein can be from a virus that can be transmitted as a result of transplantation across a species barrier (e.g., to a xenograft recipient). The ancestor protein can be from an endogenous retrovirus, e.g., a mammalian endogenous retrovirus, e.g., a porcine endogenous retrovirus. The antibody can be a monoclonal antibody or antigen binding fragment thereof. In one embodiment, the antibody is a humanized monoclonal antibody. Other suitable antibodies or antigen binding fragments thereof can be a single chain antibody, a single heavy chain antibody, an antigen binding F(ab′)₂ fragment, an antigen binding Fab′ fragment, an antigen binding Fab fragment, or an antigen binding Fv fragment.

In addition to determining ancestral viral sequences, the present invention also provides methods for preparing and testing immunogenic compositions based on an ancestral viral sequence. In specific embodiments, immunogenic compositions (based on an ancestral viral sequence) are prepared and administered to a mammal, employing an appropriate model, such as, for example, a mouse model or primate model. Immunogenic compositions can be prepared using an isolated ancestral viral gene sequence, or polypeptide sequence, or a portion thereof.

In another aspect, a method of preparing an ancestral amino acid sequence (e.g., an endogenous retroviral ancestral sequence) is provided. The method can include, for example:

(a) selecting sequences of a virus (e.g., a replication-competent endogenous retrovirus);

(b) determining an ancestral sequence by maximum likelihood phylogeny analysis that is a most recent common ancestor of the given viral sequences, the ancestral viral sequence representative of the evolutionary center of an evolutionary tree of the given viral sequences; and

(c) synthesizing a viral sequence that is not 100% identical to any of the given viral sequences but whose deduced amino acid sequence is at least 70% identical to any of them.

In one embodiment of the method, the virus is an endogenous retrovirus, e.g. a porcine endogenous retrovirus (e.g., PERV subtype A, PERV subtype B, or PERV subtype C). The method can further include testing fragments in an assay for immunogenicity. The maximum likelihood phylogeny analysis can include coalescent likelihood analysis.

In another aspect, a method for inducing an immune response to a donor virus in a transplant recipient or a potential transplant recipient is provided. The method includes administering to the recipient or potential recipient an immunologically effective amount of a composition comprising a donor virus ancestor protein or an antigenic fragment thereof. The method can further include repeating the administering of the composition to the recipient one or more times. The composition can include at least two ancestor proteins or fragments thereof. The recipient can be a human recipient. The composition can be administered prior to, simultaneously with, and/or after transplantation of a donor organ. The donor virus ancestor protein can be, for example an endogenous retrovirus ancestor protein, e.g., an endogenous retrovirus env ancestor protein. The ancestor protein can include at least 10 contiguous amino acids of a sequence set forth in one of the following: SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

In yet another aspect, a method for protecting a host from infection by a donor virus is provided. The method can include, for example: administering to the host an immunologically effective amount of a composition comprising a donor virus ancestor protein or an antigenic fragment thereof. The method can further include repeating the administering of the composition to the host one or more times. The composition can include at least two ancestor proteins or fragments thereof. The host can be a human host. The composition can be administered prior to, simultaneously with, and/or after transplantation of a donor organ. The donor virus ancestor protein can be, for example an endogenous retrovirus ancestor protein, e.g., an endogenous retrovirus env ancestor protein. The ancestor protein can include at least 10 contiguous amino acids of a sequence set forth in one of the following: SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

In still another aspect, another method for inducing an immune response to a donor virus in a transplant recipient or a potential transplant recipient is provided. The method can include administering to the transplant recipient or potential transplant recipient a composition comprising a nucleic acid encoding a donor virus ancestor protein or an antigenic fragment thereof. The method can further include administering a compound comprising the donor virus ancestor protein or an antigenic fragment thereof. The transplant recipient or potential transplant recipient can be a human recipient.

In another aspect, a method for making a vaccine is provided. The method can include, for example: expressing a nucleic acid encoding a virus (e.g., an endogenous retrovirus) ancestor protein in a host cell; and isolating a preparation comprising the ancestor protein from the host cell. In one embodiment, the endogenous retrovirus ancestor protein is a mammalian endogenous retrovirus ancestor protein, e.g., a porcine endogenous retrovirus ancestor protein, e.g., an ancestor protein of PERV subtype A, B, or C. The PERV ancestor protein can be a PERV env ancestor protein. The PERV ancestor protein can include at least 10 contiguous amino acids of a sequence set forth in one of the following: SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

In another aspect, a kit is provided. The kit can include, for example, a composition comprising an endogenous retroviral ancestor protein or an antigenic fragment of an endogenous retroviral ancestor protein, and instructions for administering the composition to a transplant recipient or a potential transplant recipient. In another embodiment, a kit comprises a composition comprising a nucleic acid encoding an endogenous retroviral ancestor protein or an antigenic fragment of an endogenous retroviral ancestor protein, and instructions for administering the composition to a transplant recipient or a potential transplant recipient.

In another aspect, a method for detecting infection with an endogenous retrovirus is provided. The method can include, for example, providing a sample comprising nucleic acid molecules present in a biological sample obtained from a subject; contacting a sample with a probe, wherein the probe is an ancestral nucleic acid sequence of an endogenous retrovirus, and determining if the sample comprises a nucleic acid molecule that hybridizes to the probe.

In another aspect, the invention features a method for performing xenotransplantation in a subject. The method can include administering to a subject a composition comprising an ancestor protein or an antigenic fragment thereof, e.g., an ancestor protein described herein, and transplanting in the subject an organ from a different species. In one embodiment, the subject is a human subject. In one embodiment, the organ is from a porcine species.

### DESCRIPTION OF THE DRAWINGS

FIG. 1 shows a phylogenetic classification of HIV-1. The circled nodes approximate the ancestral state of the HIV-1 main group (Group M) and the main group clades A-G, J, AGI and AG.

FIG. 2 shows the phylogenetic relationship of HIV-1 subtype B and the placement of the determined subtype B ancestral node on that tree. The phylogenetic relationship of HIV-1 subtype D is shown as an outgroup.

FIG. 3 shows an ancestral viral sequence reconstruction of the most recent common ancestor using maximum likelihood reconstruction for an SIV inoculum up to three years after infection into macaques. The consensus sequence and the most recent common ancestor sequence were found to differ 1.5% in nucleotide sequence.

FIG. 4 provides an example of the development of a digital vaccine using an ancestral viral sequence.

FIG. 5 shows a comparison of a “most parsimonious reconstruction” methodology and a “maximum likelihood reconstruction methodology.”

FIG. 6 shows another comparison of the “most parsimonious reconstruction” methodology and the “maximum likelihood reconstruction methodology.”

FIG. 7 illustrates a map of the pJW4304 SV40EBV vector.

FIG. 8 shows the phylogenetic relationship of HIV-1 subtype C and the placement of the determined subtype C ancestral node on that tree.

FIG. 9 shows the phylogenetic trees estimated from the input sequences of the PERV env gene viewed as amino acids (Tree A, left) or nucleotides (Tree N, right). The trees have been rooted for presentation purposes only.

FIG. 10 shows a summary of the reconstructed ancestral sequences for the PERV env gene. The differences among the sequences are illustrated by the calculation of a neighbor-joining (NJ) tree using distances estimated with the general time reversible model of evolution. The naming convention for sequence names is described in the text. The tree was rooted arbitrarily for presentation purposes only.

The details of one or more embodiments of the invention are set forth in the accompanying drawings and the description below. Other features, objects, and advantages of the invention will be apparent from the description and drawings, and from the claims.

Like reference symbols in the various drawings indicate like elements.

### DETAILED DESCRIPTION

Prior to setting forth the invention in more detail, it may be helpful to a further understanding thereof to set forth definitions of certain terms as used hereinafter.

Definitions

Unless defined otherwise, all technical and scientific terms used herein have the same meaning as commonly understood by one of ordinary skill in the art to which this invention pertains. Although any methods and materials similar to those described herein can be used in the practice or testing of the present invention, only exemplary methods and materials are described. For purposes of the present invention, the following terms are defined below.

In the context of the present invention, an “ancestral sequence” refers to a determined founder sequence, typically one that is more closely related, on average, to any given variant than to any other variant. An “ancestral viral sequence” refers to a determined founder sequence, typically one that is more closely related, on average, to any given circulating virus than to any other variant. An “ancestral viral sequence” is determined through application of maximum likelihood phylogenetic analysis (as more fully described herein) using the nucleic acid and/or amino acid sequences of circulating viruses. An “ancestor virus” is a virus comprising the “ancestral viral sequence.” An “ancestor protein” is a protein, polypeptide or peptide having an amino acid ancestral viral sequence.

The term “circulating virus” refers to virus found in an infected individual.

The term “endogenous retrovirus” refers to a retrovirus that can be found as a provirus in the genome of an organism. Endogenous retroviruses are inherited in a Mendelian fashion, and can also spread by infection.

The term “variant” refers to a virus, gene or gene product that differs in sequence from other viruses, genes or gene products by one or more nucleotide or amino acids.

The terms “immunological” or “immune response” refer to the development of a beneficial humoral (i.e., antibody mediated) and/or a cellular (i.e., mediated by antigen-specific T-cells or their secretion products) response directed against an HIV peptide in a recipient subject. Such a response can be, in particular, an active response induced by the administration of an immunogen. A cellular immune response is elicited by the presentation of epitopes in association with Class I or Class II MHC molecules to activate antigen-specific CD4⁺ T helper cells (i.e., Helper T lymphocytes) and/or CD8⁺ cytotoxic T cells. The presence of a cell-mediated immunological response can be determined by, for example, proliferation assays of CD4⁺ T cells (i.e., measuring the HTL (Helper T lymphocyte) response) or by CTL (cytotoxic T lymphocyte) assays (see, e.g., Burke et al., J. Inf. Dis. 170:1110-19 (1994); Tigges et al., J. Immunol. 156:3901-10 (1996)). The relative contributions of humoral and cellular responses to the protective or therapeutic effect of an immunogen can be distinguished by separately isolating IgG and T-cells from an immunized syngeneic animal and measuring protective or therapeutic effects in a second subject. For example, the effector cells can be deleted and the resulting response analyzed (see, e.g., Schmitz et al., Science 283:857-60 (1999); Jin et al., J Exp. Med. 189:991-98 (1999)).

“Antibody” refers to a polypeptide substantially encoded by an immunoglobulin gene or immunoglobulin genes, or fragments thereof, that specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain has a variable region of about 100 to 110 or more amino acids primarily responsible for antigen recognition. The terms variable light chain (VL) and variable heavy chain (VH) refer to these light and heavy chains, respectively.

Antibodies exist, for example, as intact immunoglobulins or as a number of well characterized antigen-binding fragments produced by digestion with various peptidases. For example, pepsin digests an antibody below the disulfide linkages in the hinge region to produce an F(ab′)₂ fragment, a dimer of Fab which itself is a light chain joined to VH-CH1 by a disulfide bond. The F(ab′)₂ fragment can be reduced under mild conditions to break the disulfide linkage in the hinge region, thereby converting the F(ab′)₂ dimer into an Fab′ monomer. The Fab′ monomer is essentially an Fab with part of the hinge region (see, Fundamental Immunology, Third Edition, W. E. Paul (ed.), Raven Press, N.Y. (1993)). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments, such as a single chain antibody, an antigen binding F(ab′)₂ fragment, an antigen binding Fab′ fragment, an antigen binding Fab fragment, an antigen binding Fv fragment, a single heavy chain or a chimeric antibody. Such antibodies can be produced by the modification of whole antibodies or synthesized de novo using recombinant DNA methodologies.

The term “biological sample” refers to any tissue or liquid sample having genomic or viral DNA or other nucleic acids (e.g., mRNA, viral RNA, etc.) or proteins. “Biological sample” further includes fluids, such as serum and plasma, that contain cell-free virus, and also includes both normal healthy cells and cells suspected of HIV infection.

The term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (see, e.g., Batzer et al., Nucleic Acid Res. 19:5081 (1991); Ohtsuka et al., J. Biol. Chem. 260:2605-08 (1985); Rossolini et al., Mol. Cell. Probes 8:91-98 (1994)). Nucleic acids also include fragments of at least 10 contiguous nucleotides (e.g., a hybridizable portion); in other embodiments, the nucleic acids comprise at least 25 nucleotides, 50 nucleotides, 100 nucleotides, 150 nucleotides, 200 nucleotides, or even up to 250 nucleotides or more. The term “nucleic acid” is used interchangeably with gene, cDNA, and mRNA encoded by a gene.

As used herein a “nucleic acid probe” is defined as a nucleic acid capable of binding to a target nucleic acid (e.g., an HIV-1 nucleic acid) of complementary sequence through one or more types of chemical bonds, usually through complementary base pairing, such as by hydrogen bond formation. As used herein, a probe may include natural (e., A, G, C, or T) or modified bases (e., 7-deazaguanosine, inosine, etc.). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence, at levels that depend upon the stringency of the hybridization conditions.

Nucleic acid probes can be DNA or RNA fragments. DNA fragments can be prepared, for example, by digesting plasmid DNA, by use of PCR, or by chemical synthesis, such as by the phosphoramidite method described by Beaucage and Carruthers (Tetrahedron Lett. 22:1859-62 (1981)), or by the triester method according to Matteucci et al. (J. Am. Chem. Soc. 103:3185 (1981)). A double stranded fragment can then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double stranded nucleic acid.

A “labeled nucleic acid probe” is a nucleic acid probe that is bound, either covalently, through a linker, or through ionic, van der Waals or hydrogen bonds, to a label such that the presence of the probe can be detected by detecting the presence of the label bound to the probe.

The term “operably linked” refers to functional linkage between a nucleic acid expression control sequence (such as a promoter, signal sequence, or any of an array of transcription factor binding sites) and a second nucleic acid sequence, wherein the expression control sequence affects transcription and/or translation of the nucleic acid corresponding to the second sequence.

“Amplification primers” are nucleic acids, typically oligonucleotides, comprising either natural or analog nucleotides that can serve as the basis for the amplification of a selected nucleic acid sequence. They include, for example, both polymerase chain reaction primers and ligase chain reaction oligonucleotides.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The terms “amino acid” or “amino acid residue”, as used herein, refer to naturally occurring L-amino acids or to D-amino acids as described further below. The commonly used one- and three-letter abbreviations for amino acids are used herein (see, e.g., Alberts et al., Molecular Biology of the Cell, Garland Publishing, Inc., New York (3d ed. 1994); Creighton, Proteins, W. H. Freeman and Company (1984)).

A “conservative substitution,” when describing a protein, refers to a change in the amino acid composition of the protein that is less likely to substantially alter the protein's activity. Thus, “conservatively modified variations” of a particular amino acid sequence refers to amino acid substitutions of those amino acids that are less likely to be critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing amino acids that are often functionally similar are well known in the art (see, e.g., Creighton, Proteins, W. H. Freeman and Company (1984)). In addition, individual substitutions, deletions or additions which alter, add or delete a single amino acid or a small percentage of amino acids in an encoded sequence are also “conservatively modified variations.”

The terms “identical” or “percent identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region, as measured using one of the following sequence comparison algorithms or by manual alignment and visual inspection. Such sequences are then said to be “substantially identical.” This definition also refers to the complement of a test sequence. Optionally, the identity exists over a region that is at least about 30 amino acids or nucleotides in length, typically over a region that is 50, 75 or 150 amino acids or nucleotides. In one embodiment, the sequences are substantially identical over the entire length of the coding regions.

The terms “similarity,” or “percent similarity,” in the context of two or more polypeptide sequences, refer to two or more sequences or subsequences that have a specified percentage of amino acid residues that are either the same or similar as defined in the conservative amino acid substitutions defined above (i.e., at least 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Such sequences are then said to be “substantially similar.” Optionally, this identity exists over a region that is at least about 25 amino acids in length, or more preferably over a region that is at least about 50, 75 or 100 amino acids in length.

For sequence comparison, typically one sequence acts as a reference sequence to which test sequences are compared. When using a sequence comparison algorithm, test and reference sequences are typically input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981)), by the homology alignment algorithm of Needleman and Wunsch (J. Mol. Biol. 48:443 (1970)), by the search for identity method of Pearson and Lipman (Proc. Natl. Acad. Sci. USA 85:2444 (1988)), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally Ausubel et al., Current Protocols in Molecular Biology, John Wiley and Sons, New York (1996)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (J. Mol. Evol. 35:351-60 (1987)). The method used is similar to the CLUSTAL method described by Higgins and Sharp (Gene 73:237-44 (1988); CABIOS 5:151-53 (1989)). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al. (J. Mol. Biol. 215:403-10 (1990)). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin and Altschul, Proc. Natl. Acad. Sci. USA 90:5873-87 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is typically between about 0.35 and about 0.1. Another indication that two nucleic acids are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization experiments, such as Southern and northern hybridizations, are sequence-dependent, and are different under different environmental parameters. Longer sequences hybridize specifically at higher temperatures. An extensive guide to the hybridization of nucleic acids is found in Tijssen, Laboratory Techniques in Biochemistry and Molecular Biology—Hybridization with Nucleic Acid Probes, part I, chapter 2 “Overview of principles of hybridization and the strategy of nucleic acid probe assays,” Elsevier, N.Y. (1993). Generally, highly stringent hybridization and wash conditions are selected to be about 5° C. lower than the thermal melting point (Tₘ) for the specific sequence at a defined ionic strength and pH. Typically, under “stringent conditions,” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide in 4-6×SSC or SSPE at 42° C., or 65-68° C. in aqueous solution containing 4-6×SSC or SSPE. An example of highly stringent wash conditions is 0.15 M NaCl at 72° C. for about 15 minutes. An example of stringent wash conditions is a 0.2×SSC wash at 65° C. for 15 minutes. (ee generally Sambrook et al., Molecular Cloning, A Laboratory Manual, 2nd ed., Cold Spring Harbor Publish., Cold Spring Harbor, N.Y. (1989)). Often, a high stringency wash is preceded by a low stringency wash to remove background probe signal. An example of medium stringency wash for a duplex of, for example, more than 100 nucleotides, is 1×SSC at 45° C. for 15 minutes. An example of low stringency wash for a duplex of, for example, more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code.

A further indication that two nucleic acids or polypeptides are substantially identical is that the polypeptide encoded by the first nucleic acid is immunologically cross reactive with, or specifically binds to, antibodies raised against the polypeptide encoded by the second nucleic acid. Thus, a polypeptide is typically substantially identical to a second polypeptide, for example, where the two peptides differ only by conservative substitutions.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for the particular protein. For example, antibodies raised to the protein with the amino acid sequence encoded by any of the nucleic acids of the invention can be selected to obtain antibodies specifically immunoreactive with that protein and not with other proteins except for polymorphic variants. A variety of immunoassay formats can be used to select antibodies specifically immunoreactive with a particular protein. For example, solid-phase ELISA immunoassays, Western blots, or immunohistochemistry are routinely used to select monoclonal antibodies specifically immunoreactive with a protein (see, e.g., Harlow and Lane, Antibodies, A Laboratory Manual, Cold Spring Harbor Publications, N.Y. (1988), for a description of immunoassay formats and conditions that can be used to determine specific immunoreactivity). Typically, a specific or selective reaction will be at least twice background signal or noise and more typically more than 10 to 100 times background.

The term “immunogenic composition” refers to a composition that elicits an immune response which produces antibodies or cell-mediated immune responses against a specific immunogen. Immunogenic compositions can be prepared as injectables, as liquid solutions, suspensions, emulsions, and the like. The term “antigenic composition” refers to a composition that can be recognized by a host immune system. For example, an antigenic composition contains epitopes that can be recognized by humoral (e.g., antibody) and/or cellular (e.g., T lymphocytes) components of a host immune system.

The term “vaccine” refers to an immunogenic composition for in vivo administration to a host, which may be a primate, particularly a human host, to confer protection against disease, particularly a viral disease.

The term “isolated” refers to a virus, nucleic acid or polypeptide that has been removed from its natural cellular environment. An isolated virus, nucleic acid or polypeptide is typically at least partially purified from cellular nucleic acids, polypeptides and other constituents.

In the context of the present invention, a “Coalescent Event” refers to the joining of two lineages on a genealogy at the point of their most recent common ancestor.

A “Coalescent Interval” describes the time between coalescent events. The expected time for each coalescent interval is exponentially distributed with mean E [_{tnyn}−1]=2N/n(n−1) generations for n<<N.

Phylogenetic Determination of Ancestral Sequences

In one aspect, computational methods are provided for determining ancestral sequences. Such methods can be used, for example, to determine ancestral sequences for viruses. These computational methods are typically used to determine an ancestral sequence of a virus that exists as a highly diverse viral population. For example, some highly diverse viruses (including FIV, HIV-1, HIV-2, Hepatitis C, endogenous retroviruses such as PERV and the like) do not appear to evolve through a succession of variants, where one prototypical strain is replaced by successive uniform strains. Instead, an evolutionary tree of viral sequences can form a “star-burst pattern,” with most of the variants approximately equidistant from the center of the star-burst. This star-burst pattern indicates that multiple, diverse circulating strains evolve from a common ancestor. The computational methods can be used to determine ancestral sequences for such highly diverse viruses, such as, for example, FIV, HIV-1, HIV-2, Hepatitis C, endogenous retroviruses and other viruses.

Methods for determining ancestral sequences are typically based on the nucleic acid sequences of circulating viruses. As a viral nucleic acid sequence is replicated, it acquires base changes due to errors in the replication process. For example, as some nucleic acid sequences are replicated, thymine (T) might bind to a guanine (G) rather than its normal complement, cytosine (C). Most of these base changes (or mutations) are not reproduced in subsequent replication events, but a certain proportion of mutations are passed down to the descendant sequences. With more replication cycles, nucleic acid sequences acquire more mutations. If a nucleic acid sequence bearing one or more mutations gives rise to two separate lineages, then the resulting two lineages will share the same parental nucleic acid sequence, and have the same parental mutation(s). If the “histories” of these lineages are traced backwards, they will have a common branch point, at which the two lineages arose from a common ancestor. Similarly, if the histories of presently circulating viral nucleic acid sequences are traced backwards, the branching points in these histories also correspond to points, designated as nodes, at which a single ancestor gave rise to the descendant lineages.

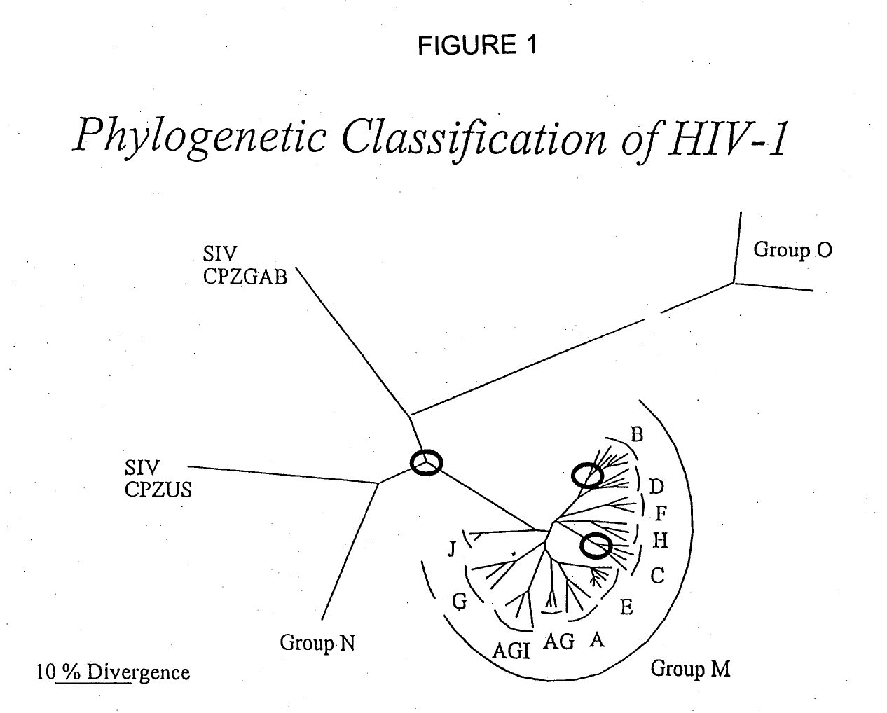

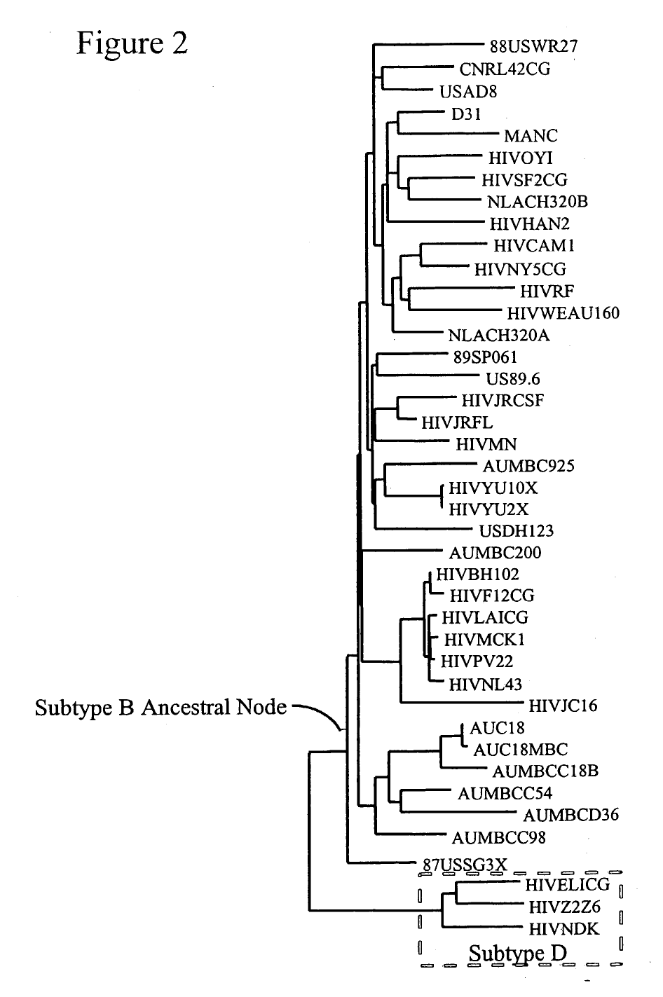

The present computational methods are based on the principle of maximum likelihood and use samples of nucleic acid sequences of circulating viruses. The sequences of the viruses in the samples typically share a common feature, such as being from the same viral strain, subtype or group. A phylogeny is constructed by using a model of evolution that specifies the probabilities of nucleotide substitutions in the replicating viral nucleic acids. At positions in the sequences where the nucleotides differ (i.e., at the site of a mutation), the methodology assigns one of the nucleotides to the node (i.e., the branch point of the lineages) such that the probability of obtaining the observed viral sequences is maximized. The assignment of nucleotides to the nodes is based on the predicted phylogeny or phylogenies. For each data set, several sequences from a different viral strain, subtype or group are used as an outgroup to root the sequences of interest. A model of sequence substitutions and then a maximum likelihood phylogeny are determined for each data set (e.g., subtype and outgroup). The maximum likelihood phylogeny is the one that has the highest probability of giving the observed nucleic acid sequences in the samples. The sequence at the base node of the maximum likelihood phylogeny is referred to as the ancestral sequence (or most recent common ancestor). (See, e.g., FIGS. 1 and 2). This ancestral sequence is thus approximately equidistant from the different sequences within the samples.

Maximum likelihood phylogeny uses samples of the sequences of circulating virus or endogenous virus. The sequences of circulating and endogenous viruses can be determined, for example, by extracting nucleic acids from blood, tissues or other biological samples of virally infected persons and sequencing the viral nucleic acids. (See, e.g., Sambrook et al., Molecular Cloning, A Laboratory Manual, 2nd ed., Cold Spring Harbor Publish., Cold Spring Harbor, N.Y. (1989); Kriegler, Gene Transfer and Expression: A Laboratory Manual, W. H. Freeman, N.Y. (1990); Ausubel et al., supra.) In one embodiment, extracted viral nucleic acids can be amplified by polymerase chain reaction, and then DNA sequenced. Samples of circulating virus can be obtained from stored biological samples and/or prospectively from samples of circulating or endogenous virus (e.g., sampling HIV-1 subtype C in India versus Ethiopia). Viral sequences can also be identified from databases (e.g., GenBank and Los Alamos sequence databases).

Once samples of circulating viruses are collected (typically about 20 to about 50 samples), the nucleic acid sequences for one or more genes are analyzed using the computational methods according to the present invention. In one method, for any given site in the sequence, the nucleotides at all nodes on a tree are assigned. The configuration of the nucleotides for all nodes that maximizes the probability of obtaining the observed sequences of circulating viruses is determined. With this method, the joint likelihood of the states across all nodes is maximized.

A second method is to choose, for a given nucleotide site and a given node on the tree, the nucleotide that maximizes the probability of obtaining the observed sequences of circulating viruses, allowing for all possible assignments of nucleotides at the other nodes on the tree. This second method maximizes the marginal likelihood of a particular assignment. For these methods, the reconstruction of the ancestral sequence (i.e., ancestral state) need not result in only a single determined sequence, however. It is possible to choose a number of ancestral sequences, ranked in order of their likelihood.

With HIV populations, a second layer of modeling can be added to the maximum likelihood phylogenetic analysis, in particular the layer is added to the model of evolution that is employed in the analysis. This second layer is based on coalescent likelihood analysis. The coalescent is a mathematical description of a genealogy of sequences, taking account of the processes that act on the population. If these processes are known with some certainty, the use of the coalescent can be used to assign prior probabilities to each type of tree. Taken together with the likelihood of the tree, the posterior probability can be determined that a determined phylogenetic tree is correct given the data. Once a tree is chosen, the ancestral states are determined, as described above. Thus, coalescent likelihood analysis can also be applied to determine the sequence of an ancestral viral sequence (e.g, a founder, or Most Recent Common Ancestor (MRCA), sequence).

In a typical embodiment, maximum likelihood phylogeny analysis is applied to determine an ancestor sequence (e.g., an ancestral viral sequence). Typically, between 20 and 50 nucleic acid sequence samples are used that have a common feature, such as a viral strain, subtype or group (e.g., samples encompassing a worldwide diversity of the same subtype). Additional sequences from other viruses (e.g., another strain, subtype, or group) are obtained and used as an outgroup to root the viral sequences being analyzed. The samples of viral sequences are determined from presently circulating or endogenous viruses, identified from the database (e.g., GenBank and Los Alamos sequence databases), or from similar sources of sequence information. The sequences are aligned using CLUSTALW (Thompson et al., Nucleic Acids Res. 22:4673-80 (1994), the disclosure of which is incorporated by reference herein) and these alignments are refined using GDE (Smith et al., CABIOS 10:671-75 (1994) the disclosure of which is incorporated by reference herein). The amino acid sequences are also translated from the nucleic acid sequences. Gaps are manipulated so that they are inserted between codons. This alignment (alignment I) is modified for phylogenetic analysis so that regions that can not be unambiguously aligned are removed (Learn et al., J. Virol. 70:5720-30 (1996), the disclosure of which is incorporated by reference herein) resulting in alignment II.

An appropriate evolutionary model for phylogeny and ancestral state reconstructions for these sequences (alignment II) is selected using the Akaike Information Criterion (AIC) (Akaike, IEEE Trans. Autom. Contr. 19:716-23 (1974); which is incorporated by reference herein) as implemented in Modeltest 3.0 (Posada and Crandall, Bioinformatics 14:817-8 (1998), which is incorporated by reference herein). For example, for the analysis for the subtype C ancestral sequence the optimal model is equal rates for both classes of transitions and different rates for all four classes of transversions, with invariable sites and a Γ distribution of site-to-site rate variability of variable sites (referred to as a TVM+I+G model). The parameters of the model in this case can be, for example, equilibrium nucleotide frequencies: ƒ_{A}=0.3576, ƒ_{C}=0.1829, ƒ_{G}=0.2314, ƒ_{T}=0.2290; proportion of invariable sites=0.2447; shape parameter (α) of the Γ distribution=0.7623; rate matrix (R) matrix values: R_{A→C}=1.7502, R_{A→G}=R_{C→T}=4.1332, R_{A→T}=0.6825, R_{C→G}=0.6549, R_{G→T}=1.

Evolutionary trees for the sequences (alignment II) are inferred using maximum likelihood estimation (MLE) methods as implemented in PAUP* version 4.0b (Swofford, PAUP 4.0: Phylogenetic Analysis Using Parsimony (And Other Methods); Sinauer Associates, Inc. (2000) the disclosure of which is incorporated by reference herein). For example, for HIV-1 subtype C sequences, ten different subtree-pruning-regrafting (SPR) heuristic searches can be performed, each using a different random addition order. The ancestral viral nucleotide sequence is determined to be the sequence at the basal node using the phylogeny, the sequences from the databases (alignment II), and the TVM+I+G model above using marginal likelihood estimation (see below).

The methods described above use sequences which have been aligned as codons, but which are then reconstructed as nucleotides. Methods that reconstruct the ancestral sequences as codons, using a 64 codon×64 codon rate matrix of possible substitutions, (rather than a 4 base×4 base rate matrix, as is used for nucleotides) can also be used. In these methods, the matrix is constrained so that substitution from an amino acid codon to a stop codon has near zero probability.

In some cases, the determined sequence may not include ancestral sequence for portions of variable regions (e.g., variable regions V1, V2, V4 and V5 for HIV-1-C), and or some short regions may not be unambiguously aligned. The following procedure can optionally be used to predict amino acid sequences for the complete sequence, including the highly variable regions (such as those deleted from alignment I). The determined ancestral sequence is visually aligned to alignment I and translated using GDE (Smith et al., supra). Since the highly variable regions can be deleted as complete codons, the translational reading frame can be preserved and codons can be maintained. The ancestral amino acid sequence for the regions deleted from alignment II can be predicted visually and refined using a parsimony-based sequence reconstruction for these sites using the computer program MacClade, version 3.08a (Maddison and Maddison. MacClade—Analysis of Phylogeny and Character Evolution—Version 3. Sinauer Associates, Inc. (1992)).

The ancestral amino acid sequence is optionally optimized for expression in a particular cell type. Amino acid sequences can converted to a DNA sequence optimized for expression in certain cell types (e.g., human cells, or porcine cells) using, for example, the BACKTRANSLATE program of the Wisconsin Sequence Analysis Package (GCG), version 10 and a human gene codon table from the Codon Usage Database (http://www.kazusa.orjp/codon/cgi-bin/showcodon.cgi?species=Homo+sapiens+[gbpri]), both incorporated by reference herein.

The optimized sequences encode the same amino acid sequence for the gene of interest (e.g., the env gene) as the non-optimized ancestral sequence. A synthetic virus having the optimized sequence may not be fully functional due to the disruption of auxiliary genes in different reading frames the presence of RNA secondary structural feature (e.g., the Rev responsive element (RRE) of HIV-1), and the like. The optimization process may affect the coding region of the auxiliary genes (e.g., vpu, tat and rev genes of HIV-1), and may disrupt RNA secondary structure. Thus, the ancestral sequences can be semi-optimized. A semi-optimized sequence has the optimized sequence for portions of the sequence that do not span other features, where the non-optimized ancestral sequence is used instead. For example, for HIV-1 ancestral sequences, the optimized ancestral sequence is used for portions of the sequence that do not span the vpu, tat, rev and RRE regions, while the “non-optimized” ancestral sequence is used for the portions of the sequence that overlap the vpu, tat, rev and RRE regions.

Phylogenetic Determination of HIV Ancestral Viral Sequences

Ancestral viral sequences can be determined for any gene or genes from HIV type 1 (HIV-1), HIV type 2 (HIV-2), or other HIV viruses, including, for example, for an HIV-1 subtype, for an HIV-2 subtype, for other HIV subtypes, for an emerging HIV subtype, and for HIV variants, such as widely dispersed or geographically isolated variants. For example, an ancestral viral gene sequence can be determined for env and gag genes of HIV-1, such as for HIV-1 subtypes A, B, C, D, E, F, G, H, J, AG, AGI, and for groups M, N, O, or for HIV-2 viruses or HIV-2 subtypes A or B. In specific embodiments, ancestral viral sequences are determined for env genes of HIV-1 subtypes B and/or C, or for gag genes from subtypes B and/or C. In other embodiments, the ancestral viral sequence is determined for other HIV genes or polypeptides, such as nef, pol, or other auxiliary genes or polypeptides.

Nucleic acid sequences of a selected HIV-1 or HIV-2 gene from presently and/or formerly circulating viruses can be identified from existing databases (e.g., from GenBank or Los Alamos sequence databases). The sequence of circulating viruses can also be determined by recombinant DNA methodologies. (See, e.g., Sambrook et al., Molecular Cloning, A Laboratory Manual, 2nd ed., Cold Spring Harbor Publish., Cold Spring Harbor, N.Y. (1989); Kriegler, Gene Transfer and Expression: A Laboratory Manual, W. H. Freeman, N.Y. (1990); Ausubel et al., supra.) For each data set, several sequences from a different viral strain, subtype or group are used as an outgroup to root the sequences of interest. A model of sequence substitutions and then a maximum likelihood phylogeny is determined for each data set (e.g., subtype and outgroup). The ancestral viral sequence is determined as the sequence at the basal node of the variant sequences (see, e.g., FIGS. 1 and 2). This ancestral viral sequence is thus approximately equidistant from the different sequences within the subtype.

In one embodiment, an ancestral HIV-1 group M, subtype B, env sequence was determined using 41 distinct isolates. (The determined nucleic acid and amino acid sequences are depicted in Tables 1 and 2 (SEQ ID NO:1 and SEQ ID NO:2), respectively). Referring to FIG. 2, 38 subtype B sequences and 3 subtype D (outgroup) sequences were used to root the subtype B sequences. The subtype B sequences were from nine countries, representing a broad sample of subtype B diversity: Australia, 8 sequences; China, 1 sequence; France, 5 sequences; Gabon, 1 sequence; Germany, 2 sequences; Great Britain, 2 sequences; the Netherlands, 2 sequences; Spain, 1 sequence; U.S.A., 15 sequences. The determined ancestor protein is 884 amino acids in length. The distances between this ancestral viral sequence and circulating strains used to determine it were on average 12.3% (range: 8.0-21.0%) while the available specimens were 17.3% different from each other (range: 13.3-23.2%). The ancestor sequence is therefore, on average, more closely related to any given circulating virus than to any other variant. When compared with other subtype B strains, the ancestral sequence is most similar to USAD8 (Theodore et al., AIDS Res. Human Retrovir. 12:191-94 (1996)), with an identity of 94.6% at the amino acid level.

Surprisingly, the determined ancestral viral sequence of the HIV-1 subtype B env gene encodes a wide variety of immunologically active peptides when processed for antigen presentation. Nearly all known subtype B CTL epitope consensus amino acids (387/390; 99.23%) are represented in the determined ancestral viral sequence for the subtype B, gp160 sequence. In contrast, most other variants of HIV-1 subtype B have below 95% epitope sequence conservation (although this is a not a necessary feature of ancestral viral sequences, but is a consequence of the rapid expansion of HIV-1). Thus, an immunogenic composition to this subtype B ancestor protein will elicit broad neutralizing antibody against HIV-1 isolates of the same subtype. An immunogenic composition to this subtype B ancestor protein will also elicit a broad cellular response mediated by antigen-specific T-cells.

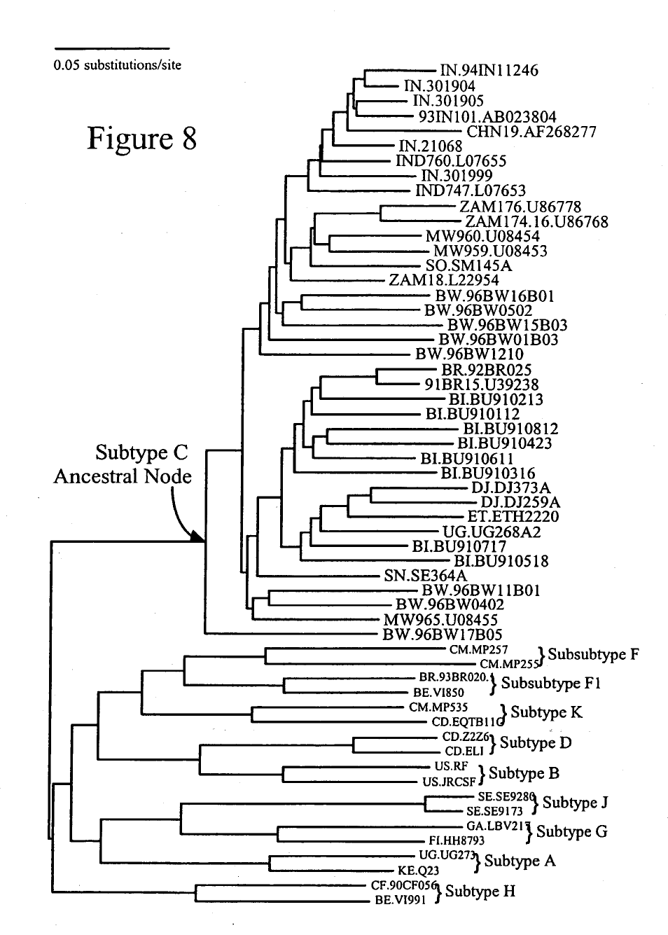

In another embodiment, similar computational methods were used to determine the ancestral viral sequence of the HIV-1 subtype C env gene sequence. HIV-1 subtype C is widespread in developing countries. Subtype C is the most common subtype worldwide, responsible for an estimated 30% of HIV-1 infections, and a major component of epidemics in Africa, India and China. The ancestral viral sequence for HIV-1 group M, subtype C, env gene was determined using 57 distinct isolates (39 subtype C sequences and 18 outgroup sequences (two from each of the other group M subtypes); FIG. 8). The determined amino acid sequence is depicted in Table 4 (SEQ ID NO:4). The determined nucleic acid sequence, optimized for expression in human cells, is depicted in Table 3 (SEQ ID NO:3).

The subtype C sequences were from twelve African and Asian countries, representing a broad sample of subtype C diversity worldwide: Botswana, 8 sequences; Brazil, 2 sequences; Burundi, 8 sequences; Peoples Republic of China, 1 sequence; Djibouti, 2 sequences; Ethiopia, 1 sequence; India, 8 sequences; Malawi, 3 sequences; Senegal, 1 sequence; Somalia, 1 sequence; Uganda, 1 sequence; and Zambia, 3 sequences. The determined ancestor protein is 853 amino acids in length. The distances between this ancestral viral sequence and circulating strains used to determine it were on average 11.7% (range: 9.3-14.3%) while the available specimens were on average 16.6% different from each other (range: 7.1-21.7%). The ancestor protein sequence is therefore, on average, more closely related to any given circulating virus than to any other variant. When compared with other subtype C strains, the ancestral sequence is most similar to MW965 (Gao et al., J Virol. 70:1651-67 (1996)), with an identity of 89.5% at the amino acid level.

Surprisingly, the determined ancestral viral sequence encodes a wide variety of immunologically active peptides when processed for antigen presentation. Nearly all known subtype C CTL epitope consensus sequences (389/396; 98.23%) are represented in the determined ancestral viral sequence for the subtype C, gp160 sequence. In contrast, typical variants of HIV-1 subtype C (those used to determine the ancestral sequence) have less than 95.19% epitope sequence conservation (average 90.36%, range 64.56-95.19%). Thus, a vaccine to this subtype C ancestral viral sequence will elicit broad neutralizing antibody against HIV-1 isolates of the same subtype. An immunogenic composition to this subtype C ancestor protein will also elicit a broad cellular response mediated by antigen-specific T-cells.

Optimized and semi-optimized sequences for an HIV ancestral sequence are also provided. Ancestral viral sequences can be optimized for expression in particular host cells. While the optimized ancestral sequence encodes the same amino acid sequence for a gene as the non-optimized sequence, the optimized sequence may not be fully functional in a synthetic virus due to the disruption of auxiliary genes in different reading frames, disruption of the RNA secondary structure, and the like. For example, optimization of the HIV-1 env sequence can disrupt the auxiliary genes for vpu, tat and/or rev, and/or the RNA secondary structure Rev responsive element (RRE). Semi-optimized sequences are prepared by using optimized sequences for portions of the sequence that do not span other genes, RNA secondary structure, and the like. For portions of the sequence that overlap such features, the “non-optimized” ancestral sequence is used (e.g., for regions overlapping vpu, tat, rev and/or RRE). In specific embodiments, semi-optimized ancestral viral sequences for HIV-1 subtypes B and C are provided. (See Tables 5 (SEQ ID NO: 5) and 6 (SEQ ID NO:6).)

In other embodiments, ancestral viral sequences are determined for widely circulating variants or geographically-restricted variants. For example, samples can be collected of an HIV-1 subtype which is widely spread (e.g., present in many countries or in regions without obvious geographic boundaries). Similarly, samples can be collected of an HIV-1 subtype which is geographically restricted (e.g., to a country, regions or other physically defined area). The sequences of the genes (e.g., gag or env) in the samples are determined by recombinant DNA methods (see, e.g., Sambrook et al., supra; Kriegler, supra; Ausubel et al., supra), or from information in databases. Typically, the number of samples will range from about 20 to about 50, depending on their current availability and the time the virus has been circulating in the region of interest (e.g., the longer the time the virus has been circulating, the greater the diversity and the greater the information to be gleaned from the samples). The ancestral viral sequence, either nucleic acid or amino acid, is then determined using the computational methods described herein.

Phylogenetic Determination of Endogenous Retroviral Ancestral Sequences

Ancestral viral sequences can be determined for any gene or genes from endogenous retroviruses, including, for example, PERV. For example, an ancestral viral gene sequence can be determined for env and gag genes of PERV, such as for PERV subtypes A, B, and C.

Nucleic acid sequences of a selected PERV gene can be identified from existing databases (e.g., from GenBank or Los Alamos sequence databases). The sequence of additional viruses can also be determined by recombinant DNA methodologies. (See, e.g., Sambrook et al., Molecular Cloning, A Laboratory Manual, 2nd ed., Cold Spring Harbor Publish., Cold Spring Harbor, N.Y. (1989); Kriegler, Gene Transfer and Expression: A Laboratory Manual, W. H. Freeman, N.Y. (1990); Ausubel et al., supra.). For each data set, several sequences from a different viral strain, subtype or group are used as an outgroup to root the sequences of interest. A model of sequence substitutions and then a maximum likelihood phylogeny is determined for each data set (e.g., subtype and outgroup). The ancestral viral sequence is determined as the sequence at the basal node of the variant sequences. This ancestral viral sequence is thus approximately equidistant from the different sequences within the subtype.

In one embodiment, an ancestral PERV subtype A env sequence was determined using 17 distinct isolates. (The determined nucleic acid and amino acid sequences are depicted in Tables 7 and 8, respectively). The determined nucleic acid sequences, optimized for expression in human cells, are depicted in Table 9.

In other embodiments, similar computational methods were used to determine the ancestral viral sequence of the PERV subtypes B and C env gene sequences. The ancestral viral sequence for the PERV subtype B env gene was determined using 15 distinct isolates. The ancestral viral sequence for the PERV subtype C env gene was determined using 4 distinct isolates. The determined amino acid sequences are depicted in Table 8. The determined nucleic acid sequences, optimized for expression in human cells, are depicted in Table 9.

Optimized and semi-optimized sequences for a PERV ancestral sequence are also provided. Ancestral viral sequences can be optimized for expression in particular host cells. While the optimized ancestral sequence encodes the same amino acid sequence for a gene as the non-optimized sequence, the optimized sequence may not be fully functional in a synthetic virus due to the disruption of auxiliary genes in different reading frames, disruption of the RNA secondary structure, and the like. For example, optimization of the PERV env sequence can disrupt auxiliary genes. Semi-optimized sequences are prepared by using optimized sequences for portions of the sequence that do not span other genes, RNA secondary structure, and the like. For portions of the sequence that overlap such features, the “non-optimized” ancestral sequence is used.

Nucleic Acids Encoding Ancestral Viral Sequences

Once an ancestral viral sequence is determined by the methods described herein, recombinant DNA methods can be used to prepare nucleic acids encoding the ancestral viral sequence of interest. Suitable methods include, but are not limited to: (1) modifying an existing viral strain most similar to the ancestor viral sequence; (2) synthesizing a nucleic acid encoding the ancestral viral sequence by joining shorter oligonucleotides (e.g., 160-200 nucleotides in length); or (3) a combination of these methods (e.g., by modifying an existing sequence using fragments with very high similarity to the ancestral viral sequence, while synthesizing de novo more divergent sequences).

The nucleic acid sequences can be produced and manipulated using routine techniques. (See, e.g., Sambrook et al supra; Kriegler, supra; Ausubel et al., supra.) Unless otherwise stated, all enzymes are used in accordance with the manufacturer's instructions.

In a typical embodiment, a nucleic acid encoding the ancestral viral sequence is synthesized by joining long oligonucleotides. By synthesizing a nucleic acid de novo, desired features are easily incorporated into the gene. Such features include, but are not limited to, the incorporation of convenient restriction sites to enable further manipulation of the nucleic acid sequence, optimization of the codon frequencies (e.g., human codon frequencies) to greatly enhance in vivo expression levels, which can favor the immunogenicity of the polypeptide sequence, and the like. Long oligonucleotides can be synthesized with a very low error rate using the solid-phase method. Long oligonucleotides designed with a 20-25 nucleotide complementary sequence at both 5′ and 3′ ends can be joined using DNA polymerase, DNA ligase, and the like. If necessary, the sequence of the synthesized nucleic acid can be verified by DNA sequence analysis.

Oligonucleotides that are not commercially available can be chemically synthesized. Suitable methods include, for example, the solid phase phosphoramidite triester method first described by Beaucage and Caruthers (Tetrahedron Letts 22(20):1859-62 (1981)), and the use of an automated synthesizer (see, e.g., Needham Van Devanter et al., Nucleic Acids Res. 12:6159-68 (1984)). Purification of oligonucleotides is, for example, by native acrylamide gel electrophoresis or by anion-exchange HPLC, as described in Pearson and Reanier (J. Chrom. 255:137-49 (1983)).

The sequence of the nucleic acids can be verified, for example, using the chemical degradation method of Maxam et al. (Methods in Enzymology 65:499-560 (1980)), or the chain termination method for sequencing double stranded templates (see, e.g., Wallace et al., Gene 16:21-26 (1981)). Southern blot hybridization techniques can be carried out according to Southern et al. (J. Mol. Biol. 98:503 (1975)), Sambrook et al. (supra), or Ausubel et al. (supra).

Expression of Ancestral Viral Sequences

The nucleic acids encoding ancestral viral sequences can be inserted into an appropriate expression vector (i.e., a vector which contains the necessary elements for the transcription and translation of the inserted polypeptide-coding sequence). A variety of host-vector systems can be utilized to express the polypeptide-coding sequence(s). These include, for example, mammalian cell systems infected with virus (e.g., vaccinia virus, adenovirus, sindbis virus, Venezuelan equine encephalitis (VEE) virus, and the like), insect cell systems infected with virus (e.g., baculovirus), microorganisms such as yeast containing yeast vectors, or bacteria transformed with bacteriophage DNA, plasmid DNA, or cosmid DNA. The expression elements of vectors vary in their strengths and specificities. Depending on the host-vector system utilized, any one of a number of suitable transcription and translation elements can be used. In specific embodiments, the ancestral viral sequence is expressed in human cells, other mammalian cells, yeast or bacteria. In yet another embodiment, a fragment of an ancestral viral sequence comprising an immunologically active region of the sequence is expressed.

Any suitable method can be used for insertion of nucleic acids encoding ancestral viral sequences into an expression vector. Suitable expression vectors typically include appropriate transcriptional and translational control signals. Suitable methods include in vitro recombinant DNA and synthetic techniques and in vivo recombination techniques (genetic recombination). Expression of nucleic acid sequences can be regulated by a second nucleic acid sequence so that the encoded nucleic acid is expressed in a host transformed with the recombinant DNA molecule. For example, expression of an ancestral viral sequence can be controlled by any suitable promoter/enhancer element known in the art. Suitable promoters include, for example, the SV40 early promoter region (Benoist and Chambon, Nature 290:304-10 (1981)), the promoter contained in the 3′ long terminal repeat of Rous sarcoma virus (Yamamoto et al., Cell 22:787-97 (1980)), the herpes thymidine kinase promoter (Wagner et al., Proc. Natl. Acad. Sci. USA 78:1441-45 (1981)), the Cytomegalovirus promoter, the translational elongation factor EF-1α promoter, the regulatory sequences of the metallothionein gene (Brinster et al., Nature 296:39-42 (1982)), prokaryotic promoters such as, for example, the β-lactamase promoter (Villa-Komaroff et al., Proc. Natl. Acad. Sci. USA 75:3727-31 (1978)) or the tac promoter (deBoer et al., Proc. Natl. Acad. Sci. USA 80:21-25 (1983)), plant expression vectors including the cauliflower mosaic virus 35S RNA promoter (Gardner et al., Nucl. Acids Res. 9:2871-88 (1981)), and the promoter of the photosynthetic enzyme ribulose biphosphate carboxylase (Herrera-Estrella et al., Nature 310:115-20 (1984)), promoter elements from yeast or other fungi such as the GAL7 and GAL4 promoters, the ADH (alcohol dehydrogenase) promoter, the PGK (phosphoglycerol kinase) promoter, the alkaline phosphatase promoter, and the like.

Other exemplary mammalian promoters include, for example, the following animal transcriptional control regions, which exhibit tissue specificity: the elastase I gene control region which is active in pancreatic acinar cells (Swift et al., Cell 38:639-46 (1984); Ornitz et al., Cold Spring Harbor Symp. Quant. Biol. 50:399-409 (1986); MacDonald, Hepatology 7(1 Suppl.):42S-51S (1987); the insulin gene control region which is active in pancreatic beta cells (Hanahan, Nature 315:115-22 (1985)), the immunoglobulin gene control region which is active in lymphoid cells (Grosschedl et al., Cell 38:647-58 (1984); Adams et al., Nature 318:533-38 (1985); Alexander et al., Mol. Cell. Biol. 7:1436-44 (1987)), the mouse mammary tumor virus control region which is active in testicular, breast, lymphoid and mast cells (Leder et al., Cell 45:485-95 (1986)), the albumin gene control region which is active in liver (Pinkert et al., Genes Dev. 1:268-76 (1987)), the alpha-fetoprotein gene control region which is active in liver (Krumlauf et al., Mol. Cell. Biol. 5:1639-48 (1985); Hammer et al., Science 235:53-58 (1987); the alpha 1-antitrypsin gene control region which is active in the liver (Kelsey et al., Genes and Devel. 1:161-71 (1987)); the beta-globin gene control region which is active in myeloid cells (Magram et al., Nature 315:338-40 (1985); Kollias et al., Cell 46:89-94 (1986); the myelin basic protein gene control region which is active in oligodendrocyte cells in the brain (Readhead et al., Cell 48:703-12 (1987)); the myosin light chain-2 gene control region which is active in skeletal muscle (Shani, Nature 314:283-86 (1985)); and the gonadotropic releasing hormone gene control region which is active in the hypothalamus (Mason et al., Science 234:1372-78 (1986)).

In a specific embodiment, a vector is used that comprises a promoter operably linked to the ancestral viral sequence encoding nucleic acid, one or more origins of replication, and, optionally, one or more selectable markers (e.g., an antibiotic resistance gene). Suitable selectable markers include, for example, those conferring resistance to ampicillin, tetracycline, neomycin, G418, and the like. An expression construct can be made, for example, by subcloning a nucleic acid encoding an ancestral viral sequence into a restriction site of the pRSECT expression vector. Such a construct allows for the expression of the ancestral viral sequence under the control of the T7 promoter with a histidine amino terminal flag sequence for affinity purification of the expressed polypeptide.

In an exemplary embodiment, a high efficiency expression system can be used which employs a high-efficiency DNA transfer vector (the pJW4304 SV40/EBV vector) with a very high efficiency RNA/protein expression component (e.g., from the Semliki Forest Virus) to achieve maximal protein expression, as further discussed infra. pJW4304 SV40/EBV was prepared from pJW4303, which is described by Robinson et al. (Ann. New York Acad. Sci. 27:209-11 (1995)) and Yasutomi et al. (J. Virol. 70:678-81 (1996)).

Expression vector/host systems expressing an ancestral viral sequences can be identified by general approaches well known to the skilled artisan, including: (a) nucleic acid hybridization, (b) the presence or absence of “marker” gene function, (c) expression of inserted sequences; or (d) screening transformed cells by standard recombinant DNA methods. In the first approach, the presence of an ancestral viral sequence nucleic acid inserted in host cells can be detected by nucleic acid hybridization using probes comprising sequences that are homologous to an inserted nucleic acid. In the second approach, the expression vector/host system can be identified and selected based upon the presence or absence of certain “marker” gene functions (e.g., thymidine kinase activity, resistance to antibiotics, transformation phenotype, occlusion body formation in baculovirus, and the like) caused by the insertion of a vector containing the desired nucleic acids. For example, if the nucleic acid is inserted within the marker gene sequence of the vector, recombinants containing the ancestral viral sequence can be identified by the absence of the marker gene function.

In the third approach, expression vector/host systems can be identified by assaying for the ancestral viral sequence polypeptide expressed by the recombinant host organism. Such assays can be based, for example, on the physical or functional properties of the ancestral viral sequence polypeptide in in vitro assay systems (e.g., binding by antibody). In the fourth approach, expression vector/host cells can be identified by screening transformed host cells by known recombinant DNA methods.

Once a suitable expression vector host system and growth conditions are established, methods that are known in the art can be used to propagate it. In addition, host cells can be chosen that modulate the expression of the inserted nucleic acid sequences, or that modify or process the gene product in the specific fashion desired. Expression from certain promoters can be elevated in the presence of certain inducers; thus, expression of the ancestral viral sequence can be controlled. Furthermore, different host cells having characteristic and specific mechanisms for the translational and post-translational processing and modification (e.g., glycosylation or phosphorylation) of polypeptides can be used. Appropriate cell lines or host systems can be chosen to ensure the desired modification and processing of the expressed polypeptide. For example, expression in a bacterial system can be used to produce an unglycosylated polypeptide.

Ancestor Proteins

The invention further relates to ancestor proteins based on a determined ancestral viral sequence. Such ancestor proteins include, for example, full-length protein, polypeptides, fragments, derivatives and analogs thereof. In one aspect, the invention provides amino acid sequences of ancestor proteins (see, e.g., Tables 2, 4, and 8; SEQ ID NO:2; SEQ ID NO:4, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:43, SEQ ID NO:44, or SEQ ID NO:45). In some embodiments, the ancestor protein is functionally active. Ancestor proteins, fragments, derivatives and analogs typically have the desired immunogenicity or antigenicity and can be used, for example, in immunoassays, for immunization, in vaccines, and the like. A specific embodiment relates to an ancestor protein, fragment, derivative or analog that can be bound by an antibody. Such ancestor proteins, fragments, derivatives or analogs can be tested for the desired immunogenicity by procedures known in the art. (See e.g., Harlow and Lane, supra).

In another aspect, a polypeptide is provided which consists of or comprises a fragment that has at least 8-10 contiguous amino acids of the ancestor protein. In other embodiments, the fragment comprises at least 20 or 50 contiguous amino acids of the ancestor protein. In other embodiments, the fragments are not larger than 35, 100 or 200 amino acids.

Ancestor protein derivatives and analogs can be produced by various methods known in the art. The manipulations which result in their production can occur at the gene or protein level. For example, a nucleic acid encoding an ancestor protein can be modified by any of numerous strategies known in the art (see, e.g., Sambrook et al., supra), such as by making conservative substitutions, deletions, insertions, and the like. The nucleic acid sequence can be cleaved at appropriate sites with restriction endonuclease(s), followed by further enzymatic modification, if desired, isolated, and ligated in vitro. In the production of nucleic acids encoding a fragment, derivative or analog of an ancestor protein, the modified nucleic acid typically remains in the proper translational reading frame, so that the reading frame is not interrupted by translational stop signals or other signals that interfere with the synthesis of the fragment, derivative or analog. The ancestral viral sequence nucleic acid can also be mutated in vitro or in vivo to create and/or destroy translation, initiation and/or termination sequences. The ancestral viral sequence-encoding nucleic acid can also be mutated to create variations in coding regions and/or to form new restriction endonuclease sites or destroy preexisting ones and to facilitate further in vitro modification. Any technique for mutagenesis known in the art can be used, including but not limited to chemical mutagenesis, in vitro site-directed mutagenesis, and the like.

Manipulations of the ancestral viral sequence can also be made at the protein level. Included within the scope of the invention are ancestor protein fragments, derivatives or analogs that are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide); enzymatic cleavage (e.g., by trypsin, chymotrypsin, papain, V8 protease, and the like); modification by, for example, NaBH₄ acetylation, formylation, oxidation and reduction; metabolic synthesis in the presence of tunicamycin; and the like.

In addition, fragments, derivatives and analogs of ancestor proteins can be chemically synthesized. For example, a peptide corresponding to a portion, or fragment, of an ancestor protein, which comprises a desired domain, can be synthesized by use of chemical synthetic methods using, for example, an automated peptide synthesizer. (See also Hunkapiller et al., Nature 310:105-11 (1984); Stewart and Young, Solid Phase Peptide Synthesis, 2nd ed., Pierce Chemical Co., Rockford, Ill., (1984).) Furthermore, if desired, nonclassical amino acids or chemical amino acid analogs can be introduced as a substitution or addition into the polypeptide sequence. Non-classical amino acids include, but are not limited to, the D-isomers of the common amino acids, α-amino isobutyric acid, 4-aminobutyric acid, 2-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and other amino acid analogs. Furthermore, the amino acid can be D (dextrorotary) or L (levorotary).

The ancestor protein, fragment, derivative or analog can also be a chimeric, or fusion, protein comprising an ancestor protein, fragment, derivative or analog thereof (typically consisting of at least a domain or motif of the ancestor protein, or at least 10 contiguous amino acids of the ancestor protein) joined at its amino- or carboxy-terminus via a peptide bond to an amino acid sequence of a different protein. In one embodiment, such a chimeric protein is produced by recombinant expression of nucleic acid encoding the chimeric protein. The chimeric nucleic acid can be made by ligating the appropriate nucleic acid sequences to each other in the proper reading frame and expressing the chimeric product by methods commonly known in the art. Alternatively, the chimeric protein can be made by protein synthetic techniques (e.g. by use of an automated peptide synthesizer).

Ancestor protein can be isolated and purified by standard methods including chromatography (e.g., ion exchange, affinity, sizing column chromatography, high pressure liquid chromatography), centrifugation, differential solubility, or by any other standard technique for the purification of proteins.

Antibodies to Ancestor Proteins, Fragments, Derivatives and Analogs

Ancestor proteins (including fragments, derivatives, and analogs thereof) can be used as an immunogen to generate antibodies which immunospecifically bind such ancestor proteins and to circulating variants. Such antibodies include but are not limited to polyclonal antibodies, monoclonal antibodies, chimeric antibodies, single chain antibodies, antigen binding antibody fragments (e.g., Fab, Fab′, F(ab′)₂, Fv, or hypervariable regions), and an Fab expression library. In some embodiments, polyclonal and/or monoclonal antibodies to an ancestor protein are produced. In other embodiments, antibodies to a domain of an ancestor protein are produced. In yet other embodiments, fragments of an ancestor protein that are identified as immunogenic (e.g., hydrophilic) are used as immunogens for antibody production.

Various procedures known in the art can be used for the production of polyclonal antibodies. For the production of such antibodies, various host animals (including, but not limited to, rabbits, mice, rats, sheep, goats, camels, and the like) can be immunized by injection with the ancestor protein, fragment, derivative or analog. Various adjuvants can be used to increase the immunological response, depending on the host species including, but not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward an ancestor protein, fragment, derivative, or analog thereof, any technique that provides for the production of antibody molecules by continuous cell lines in culture can be used. Such techniques include, for example, the hybridoma technique originally developed by Kohler and Milstein (see, e.g., Nature 256:495-97 (1975)), the trioma technique (see, e.g., Hagiwara and Yuasa, Hum. Antibodies Hybridomas. 4:15-19 (1993); Hering et al., Biomed. Biochim. Acta 47:211-16 (1988)), the human B-cell hybridoma technique (see e.g., Kozbor et al., Immunology Today 4:72 (1983)), and the EBV-hybridoma technique to produce human monoclonal antibodies (see, e.g., Cole et al., In: Monoclonal Antibodies and Cancer Therapy, Alan R. Liss, Inc., pp. 77-96 (1985)). Human antibodies can be used and can be obtained by using human hybridomas (see, e.g., Cote et al., Proc. Natl. Acad. Sci. USA 80:2026-30 (1983)) or by transforming human B cells with EBV virus in vitro (see, e.g., Cole et al., supra).

Further to the invention, “chimeric” or “humanized” antibodies (see, e.g., Morrison et al., Proc. Natl. Acad. Sci. USA 81:6851-55 (1984); Neuberger et al.,Nature 312:604-08 (1984); Takeda et al., Nature 314:452-54 (1985)) can be prepared. Such chimeric antibodies are typically prepared by splicing the non-human genes for an antibody molecule specific for ancestor protein together with genes from a human antibody molecule of appropriate biological activity. It can be desirable to transfer the antigen binding regions (e.g., Fab′, F(ab′)₂, Fab, Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; International Patent Publications WO 87/02671 and WO 90/00616; and European Patent Publication EP 239 400 (the disclosures of which are incorporated by reference herein). Alternatively, a human monoclonal antibody or portions thereof can be identified by first screening a human B-cell cDNA library for DNA molecules that encode antibodies that specifically bind to an ancestor protein according to the method generally set forth by Huse et al. (Science 246:1275-81 (1989)). The DNA molecule can then be cloned and amplified to obtain sequences that encode the antibody (or binding domain) of the desired specificity. Phage display technology offers another technique for selecting antibodies that bind to ancestor proteins, fragments, derivatives or analogs thereof. (See, e.g., International Patent Publications WO 91/17271 and WO 92/01047; Huse et al., supra.)

According to another aspect of the invention, techniques described for the production of single chain antibodies (see, e.g., U.S. Pat. Nos. 4,946,778 and 5,969,108) can be adapted to produce single chain antibodies. An additional aspect of the invention utilizes the techniques described for the construction of a Fab expression library (See, e.g., Huse et al., supra) to allow rapid and easy identification of monoclonal Fab fragments with the desired specificity for ancestor proteins, fragments, derivatives, or analogs thereof.

Antibody that contains the idiotype of the molecule can be generated by known techniques. For example, such fragments include but are not limited to, the F(ab′)₂ fragment which can be produced by pepsin digestion of the antibody molecule, the Fab′ fragments which can be generated by reducing the disulfide bridges of the F(ab′)₂ fragment, the Fab fragments which can be generated by treating the antibody molecule with papain and a reducing agent, and Fv fragments. Recombinant Fv fragments can also be produced in eukaryotic cells using, for example, the methods described in U.S. Pat. No. 5,965,405.

In the production of antibodies, screening for the desired antibody can be accomplished by techniques known in the art (e.g., ELISA (enzyme-linked immunosorbent assay)). In one example, antibodies that recognize a specific domain of an ancestor protein can be used to assay generated hybridomas for a product which binds to polypeptide containing that domain. Antibodies specific to a domain of an ancestor protein are also provided.

Antibodies against ancestor proteins (including fragments, derivatives and analogs) can be used for passive antibody treatment, according to methods known in the art. Antibodies can be introduced into an individual to prevent or treat viral infection. Typically, such antibody therapy is practiced as an adjuvant to the vaccination protocols. The antibodies can be produced as described supra and can be polyclonal or monoclonal antibodies and administered intravenously, enterally (e.g., as an enteric coated tablet form), by aerosol, orally, transdermally, transmucosally, intrapleurally, intrathecally, or by other suitable routes.

Immunogenic Compositions and Vaccines

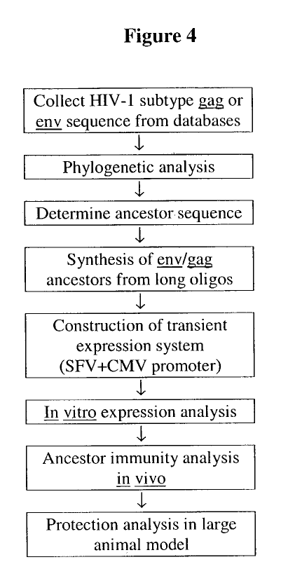

The present invention also provides immunogenic compositions, such as vaccines. An example of the development of a vaccine (“digital vaccine”) using the sequences of the invention is illustrated in FIG. 4. The present invention also provides a new way to produce vaccines, using HIV ancestral viral sequences or PERV ancestral viral gene sequences (e.g., HIV env or gag genes or polypeptides; or PERV env genes or polypeptides). Such ancestral viral sequences typically correspond to the structure of a real biological entity—the founding virus (i.e., “the viral Eve”).

Formulations

Immunogenic compositions and vaccines that contain an immunogenically effective amount of one or more ancestral viral protein sequences, or fragments, derivatives, or analogs thereof, are provided. Immunogenic epitopes in an ancestral protein sequence can be identified according to methods known in the art, and proteins, fragments, derivatives, or analogs containing those epitopes can be delivered by various means, in a vaccine composition. Suitable compositions can include, for example, lipopeptides (e.g., Vitiello et al., J. Clin. Invest. 95:341 (1995)), peptide compositions encapsulated in poly(DL-lactide-co-glycolide) (“PLG”) microspheres (se, e.g., Eldridge et al., Molec. Immunol. 28:287-94 (1991); Alonso et al., Vaccine 12:299-306 (1994); Jones et al., Vaccine 13:675-81 (1995)), peptide compositions contained in immune stimulating complexes (ISCOMS) (see, e.g., Takahashi et al., Nature 344:873-75 (1990); Hu et al., Clin. Exp. Immunol. 113:235-43 (1998)), multiple antigen peptide systems (MAPs) (see, e.g., Tam, Proc. Natl. Acad. Sci. U.S.A. 85:5409-13 (1988); Tam, J. Immunol. Methods 196:17-32 (1996)), viral delivery vectors (see, e.g., Perkus et al., In: Concepts in vaccine development, Kaufmann (ed.), p. 379 (1996)), particles of viral or synthetic origin (see, e.g., Kofler et al., J. Immunol. Methods. 192:25-35 (1996); Eldridge et al., Sem. Hematol. 30:16 (1993); Falo et al., Nature Med. 7:649 (1995)), adjuvants (see e.g., Warren et al., Annu. Rev. Immunol. 4:369 (1986); Gupta et al., Vaccine 11:293 (1993)), liposomes (see, e.g., Reddy et al., J. Immunol. 148:1585 (1992); Rock, Immunol. Today 17:131 (1996)), or naked or particle absorbed CDNA (see, e.g., Shiver et al., In: Concepts in vaccine development, Kaufmann (ed.), p. 423 (1996)). Toxin-targeted delivery technologies, also known as receptor-mediated targeting, such as those of Avant Immunotherapeutics, Inc. (Needham, Mass.) can also be used.

Furthermore, useful carriers that can be used with immunogenic compositions and vaccines of the invention are well known in the art, and include, for example, thyroglobulin, albumins such as human serum albumin, tetanus toxoid, polyamino acids such as poly L-lysine, poly L-glutamic acid, influenza, hepatitis B virus core protein, and the like. The compositions and vaccines can contain a physiologically tolerable (i.e., acceptable) diluent such as water, or saline, typically phosphate buffered saline. The compositions and vaccines also typically include an adjuvant. Adjuvants such as incomplete Freund's adjuvant, aluminum phosphate, aluminum hydroxide, or alum are examples of materials well known in the art. Additionally, as disclosed herein, CTL responses can be primed by conjugating ancestor proteins (or fragments, derivative or analogs thereof) to lipids, such as tripalmitoyl-S-glycerylcysteinyl-seryl-serine (P₃CSS).

As disclosed in greater detail herein, upon immunization with a composition or vaccine containing an ancestor viral sequence protein composition in accordance with the invention, via injection, aerosol, oral, transdermal, transmucosal, intrapleural, intrathecal, or other suitable routes, the immune system of the host responds to the composition or vaccine by producing large amounts of CTL's, HTL's and/or antibodies specific for the desired antigen. Consequently, the host typically becomes at least partially immune to later infection, or at least partially resistant to developing an ongoing chronic infection, or derives at least some therapeutic benefit.

For therapeutic or prophylactic immunization, ancestor proteins (including fragments, derivatives and analogs) can also be expressed by viral or bacterial vectors. Examples of expression vectors include attenuated viral hosts, such as vaccinia or fowlpox. In one embodiment, this approach involves the use of vaccinia virus, for example, as a vector to express nucleotide sequences that encode the polypeptide. Upon introduction into an acutely or chronically infected host, or into a non-infected host, the recombinant vaccinia virus expresses the immunogenic protein, and thereby elicits a host CTL, HTL and/or antibody response. Vaccinia vectors and methods useful in immunization protocols are described in, for example, U.S. Pat. No. 4,722,848, the disclosure of which is incorporated by reference herein. A wide variety of other vectors useful for therapeutic administration or immunization of the peptides of the invention, for example, adeno and adeno-associated virus vectors, retroviral vectors, Salmonella typhimurium vectors, detoxified anthrax toxin vectors, Alphavirus, and the like, can also be used, as will be apparent to those skilled in the art from the description herein. Alphavirus vectors that can be used include, for example, Sindbis and Venezuelan equine encephalitis (VEE) virus. (See, e.g., Coppola et al., J. Gen. Virol. 76:635-41 (1995); Caley et al., Vaccine 17:3124-35 (1999); Loktev et al., J. Biotechnol. 44:129-37 (1996).)

Polynucleotides (e.g., DNA or RNA) encoding one or more ancestral proteins (including fragments, derivative or analogs) can also be administered to a patient. This approach is described in, for example, Wolff et al., (Science 247:1465 (1990)), in U.S. Pat. Nos. 5,580,859; 5,589,466; 5,804,566; 5,739,118; 5,736,524; 5,679,647; and WO 98/04720; and in more detail below. Examples of DNA-based delivery technologies include “naked DNA”, facilitated (bupivicaine, polymer, or peptide-mediated) delivery, cationic lipid complexes, particle-mediated (“gene gun”), or pressure-mediated delivery (see, e.g., U.S. Pat. No. 5,922,687).

The direct injection of naked plasmid DNA encoding a protein antigen as a means of vaccination is, among several delivery and expression systems that have been developed in the last decade (e.g., for HIV vaccines), one that has attracted much attention. In mouse models, as well as in large animal models, both humoral and cellular immune responses are readily induced, resulting in protective immunity against challenge infections in some instances. A Semliki Forest Virus (SFV) replicon can also be used, for example, in the context of naked DNA immunization. SFV belongs to the Alphavirus family wherein the genome consists of a single stranded RNA of positive polarity encoding its own replicase. By replacing the SFV structural genes with the gene of interest, expression levels as high as 25% of the total cell protein are obtained. Another advantage of this alphavirus over plasmid vectors is its non-persistence: the antigen of interest is expressed at high levels but for a short period (typically <72 hours). In contrast, plasmid vectors generally induce synthesis of the antigen of interest over extended time periods, risking chromosomal integration of foreign DNA and cell transformation. Furthermore, antigen persistence or repeated inoculations of small amounts of antigen has been shown experimentally to induce tolerance. Prolonged antigen synthesis, therefore, can theoretically result in unresponsiveness rather than immunity.

Ancestor proteins, fragments, derivative, and analogs can also be introduced into a subject in vivo or ex vivo. For example, ancestral viral sequences can be transferred into defined cell populations. Suitable methods for gene transfer include, for example:

1) Direct gene transfer. (See, e., Wolff et al., Science 247:1465-68 (1990)).

2) Liposome-mediated DNA transfer. (See, e.g., Caplen et al., Nature Med. 3:39-46 (1995); Crystal, Nature Med. 1:15-17 (1995); Gao and Huang, Biochem. Biophys. Res. Comm. 179:280-85 (1991).)

3) Retrovirus-mediated DNA transfer. (See, e.g., Kay et al., Science 262:117-19 (1993); Anderson, Science 256:808-13 (1992).) Retroviruses from which the retroviral plasmid vectors can be derived include lentiviruses. They further include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, Myeloproliferative Sarcoma Virus, and mammary tumor virus. In one embodiment, the retroviral plasmid vector is derived from Moloney Murine Leukemia Virus. Examples illustrating the use of retroviral vectors in gene therapy further include the following: Clowes et al. (J. Clin. Invest. 93:644-51 (1994)); Kiem et al. (Blood 83:1467-73 (1994)); Salmons and Gunzberg (Human Gene Therapy 4:129-41 (1993)); and Grossman and Wilson (Curr. Opin. in Genetics and Devel. 3:110-14 (1993)).

4) DNA Virus-mediated DNA transfer. Such DNA viruses include adenoviruses (e.g., Ad-2 or Ad-5 based vectors), herpes viruses (typically herpes simplex virus based vectors), and parvoviruses (e.g., “defective” or non-autonomous parvovirus based vectors, or adeno-associated virus based vectors, such as AAV-2 based vectors). (See, e.g., Ali et al., Gene Therapy 1:367-84 (1994); U.S. Pat. Nos. 4,797,368 and 5,139,941, the disclosures of which are incorporated herein by reference.) Adenoviruses have the advantage that they have a broad host range, can infect quiescent or terminally differentiated cells, such as neurons or hepatocytes, and appear essentially non-oncogenic. Adenoviruses do not appear to integrate into the host genome. Because they exist extrachromosomally, the risk of insertional mutagenesis is greatly reduced. Adeno-associated viruses exhibit similar advantages as adenoviral-based vectors. However, AAVs exhibit site-specific integration on human chromosome 19.

Kozarsky and Wilson (Current Opinion in Genetics and Development 3:499-503 (1993)) present a review of adenovirus-based gene therapy. Bout et al. (Human Gene Therapy 5:3-10 (1994)) demonstrated the use of adenovirus vectors to transfer genes to the respiratory epithelia of rhesus monkeys. Herman et al. (Human Gene Therapy 10:1239-49 (1999)) describe the intraprostatic injection of a replication-deficient adenovirus containing the herpes simplex thymidine kinase gene into human prostate, followed by intravenous administration of the prodrug ganciclovir in a phase I clinical trial. Other instances of the use of adenoviruses in gene therapy can be found in Rosenfeld et al. (Science 252:431-34 (1991)); Rosenfeld et al. (Cell 68:143-55 (1992)); Mastrangeli et al. (J. Clin. Invest. 91:225-34 (1993)); Thompson (Oncol. Res. 11:1-8 (1999)).

The choice of a particular vector system for transferring the ancestral viral sequence of interest will depend on a variety of factors. One important factor is the nature of the target cell population. Although retroviral vectors have been extensively studied and used in a number of gene therapy applications, these vectors are generally unsuited for infecting non-dividing cells. In addition, retroviruses have the potential for oncogenicity. However, recent developments in the field of lentiviral vectors may circumvent some of these limitations. (See Naldini et al., Science 272:263-67 (1996).)

The skilled artisan will appreciate that any suitable expression vector containing nucleic acid encoding an ancestor protein, or fragment, derivative or analog thereof can be used in accordance with the present invention. Techniques for constructing such a vector are known. (See, e.g., Anderson, Nature 392:25-30 (1998); Verma, Nature 389:239-42 (1998).) Introduction of the vector to the target site can be accomplished using known techniques.

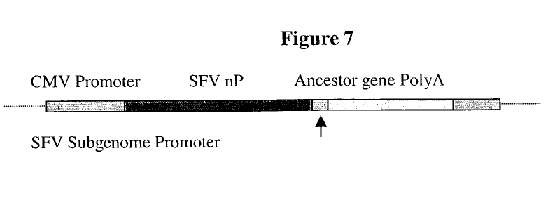

In another one embodiment, a novel expression system employing a high-efficiency DNA transfer vector (the pJW4304 SV40/EBV vector (pJW4304 SV40/EBV was prepared from pJW4303, which is described by Robinson et al., Ann. New York Acad. Sci. 27:209-11 (1995) and Yasutomi et al., J. Virol. 70:678-81 (1996)) with a very high efficiency RNA/protein expression system (the Semliki Forest Virus) is used to achieve maximal protein expression in vaccinated hosts with a safe and inexpensive vaccine. SFV CDNA is placed, for example, under the control of a cytomegalovirus (CMV) promoter (see FIG. 7). Unlike conventional DNA vectors, the CMV promoter does not directly drive the expression of the antigen encoding nucleic acids. Instead, it directs the synthesis of recombinant SFV replicon RNA transcript. Translation of this RNA molecule produces the SFV replicase complex, which catalyzes cytoplasmic self-amplification of the recombinant RNA, and eventual high-level production of the actual antigen-encoding mRNA. Following vector delivery, the transfected host cell dies within a few days. In the context of the present invention, env and/or gag genes are typically cloned into this vector. In vitro experiments using Northern blot, Western blot, SDS-PAGE, immunoprecipitation assay, and CD4 binding assays can be performed, as described infra, to determine the efficiency of this system by assessing protein expression level, protein characteristics, duration of expression, and cytopathic effects of the vector.

In some embodiments, ancestor protein (or a fragment, derivative or analog thereof) is administered to a subject in need thereof. The dosage for an initial therapeutic immunization generally occurs in a unit dosage range where the lower value is about 1, 5, 50, 500, or 1,000 μg and the higher value is about 10,000; 20,000; 30,000; or 50,000 μg. Dosage values for a human typically range from about 500 μg to about 50,000 μg per 70 kilogram patient. Boosting dosages of between about 1.0 μg to about 50,000 μg of polypeptide pursuant to a boosting regimen over weeks to months can be administered depending upon the patient's response and condition as determined by measuring the antibody levels or specific activity of CTL and HTL obtained from the patient's blood.

A human unit dose form of the protein or nucleic acid composition is typically included in a pharmaceutical composition that comprises a human unit dose of an acceptable carrier, typically an aqueous carrier, and is administered in a volume of fluid that is known by those of skill in the art to be used for administration of such compositions to humans (see, e.g., Remington “Pharmaceutical Sciences”, 17 Ed., Gennaro (ed.), Mack Publishing Co., Easton, Pa. (1985)).

The ancestor proteins and nucleic acids can also be administered via liposomes, which serve to target the peptides to a particular tissue, such as lymphoid tissue, or to target selectively to infected cells, as well as to increase the half-life of the composition. Liposomes include emulsions, foams, micelles, insoluble monolayers, liquid crystals, phospholipid dispersions, lamellar layers and the like. In these preparations, the protein or nucleic acid to be delivered is incorporated as part of a liposome, alone or in conjunction with a molecule that binds to a receptor prevalent among lymphoid cells, such as monoclonal antibodies that bind to the CD45 antigen, or with other therapeutic or immunogenic compositions. Thus, liposomes either filled or decorated with a desired protein or nucleic acid can be directed to the site of lymphoid cells, where the liposomes then deliver the protein compositions to the cells. Liposomes for use in accordance with the invention are formed from standard vesicle-forming lipids, which generally include neutral and negatively charged phospholipids and a sterol, such as cholesterol. The selection of lipids is generally guided by consideration of, for example, liposome size, acid lability and stability of the liposomes in the blood stream. A variety of methods are available for preparing liposomes, as described in, for example, Szoka et al., Ann. Rev. Biophys. Bioeng. 9:467 (1980), and U.S. Pat. Nos. 4,235,871; 4,501,728; 4,837,028; and 5,019,369.

For targeting cells of the immune system, a ligand to be incorporated into the liposome can include, for example, antibodies or fragments thereof specific for cell surface determinants of the desired immune system cells. A liposome suspension containing a protein or nucleic acid can be administered, for example, intravenously, locally, topically, etc., in a dose which varies according to, inter alia, the manner of administration, the protein or nucleic acid being delivered, and the like.

For solid compositions, conventional nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, the ancestor proteins or nucleic acids, and typically at a concentration of 25%-75%.

For aerosol administration, the immunogenic proteins or nucleic acids are typically in finely divided form along with a surfactant and propellant. Suitable percentages of peptides are about 0.01% to about 20% by weight, typically about 1% to about 10%. The surfactant is, of course, nontoxic, and typically soluble in the propellant. Representative of such agents are the esters or partial esters of fatty acids containing from 6 to 22 carbon atoms, such as caproic, octanoic, lauric, palmitic, stearic, linoleic, linolenic, stearic and oleic acids with an aliphatic polyhydric alcohol or its cyclic anhydride. Mixed esters, such as mixed or natural glycerides can be employed. The surfactant can constitute about 0.1% to about 20% by weight of the composition, typically 0.25-5%. The balance of the composition is ordinarily propellant. A carrier can also be included, as desired, as with, for example, lecithin for intranasal delivery.

Immune Responses Elicited by the Ancestral Viral Sequences

Ancestor proteins (including fragments, derivative and analogs) can be used as a vaccine, as described supra. Such vaccines, referred to as a “digital vaccine”, are typically screened for those that elicit neutralizing antibody and/or viral (e.g., HIV or PERV) specific CTLs against a larger fraction of circulating strains than a vaccine comprising a protein antigen encoded by any sequences of existing viruses or by consensus sequences. Such a digital vaccine will typically provide protection when challenged by the same subtype of virus (e.g., HIV-1 virus, PERV) as the subtype from which the ancestral viral sequence was derived.

The invention also provides methods to analyze the function of ancestral viral gene sequences. For example, in one embodiment, the HIV gp 160 ancestor viral gene sequence is analyzed by assays for functions, such as, for example, CD4 binding, co-receptor binding, receptor specificity (e.g., binding to the CCR5 receptor), protein structure, and the ability to cause cell fusion. Although the ancestor sequences can result in a viable virus, such a viable virus is not necessary for obtaining a successful vaccine. For example, a gp160 ancestor not correctly folded can be more immunogenic by exposing epitopes that are normally buried to the immune system. Further, although the ancestor viral sequence can be successfully used as a vaccine, such a sequence need not include alternate open reading frames that encode proteins such a tat or rev, when used as an immunogen (e.g., a vaccine).

Accordingly, in one aspect, mice are immunized with an ancestor protein and tested for humoral and cellular immune responses. Typically, 5-10 mice are intradermally or intramuscularly injected with a plasmid containing a gag and/or env gene encoding an ancestral viral sequence in, for example, 50 μl volume. Two control groups are typically used to interpret the results. One control group is injected with the same vector containing the gag or env gene from a standard laboratory strain (e.g., HIV-1-IIIB). A second control group is injected with same vector without any insert. Antibody titration against gag or env protein is performed using standard immunoassays (e.g., ELISA), as described infra. The neutralizing antibody is analyzed by subtype-specific laboratory HIV-1 strains, such as for example pNL4-3 (HIV-1-IIIB), as well as primary isolates from HIV-1 infected individuals. The ability of an ancestor viral sequence protein-elicited neutralizing antibody to neutralize a broad primary isolates is one factor indicative of an immunogenic or vaccine composition. Similar studies can be performed in large animals, such as non-human animals (e.g., macaques) or in humans.

Immunoassays for Titrating the Ancestor Protein-elicited Antibodies

There are a variety of assays known to those of ordinary skill in the art for detecting antibodies in a sample (see, e.g., Harlow and Lane, supra). In general, the presence or absence of antibodies in a subject immunized with an ancestor protein vaccine can be determined by (a) contacting a biological sample obtained from the immunized subject with one or more ancestor proteins (including fragments, derivatives or analogs thereof); (b) detecting in the sample a level of antibody that binds to the ancestor protein(s); and (c) comparing the level of antibody with a predetermined cut-off value.

In a typical embodiment, the assay involves the use of an ancestor protein (including fragment, derivative or analog) immobilized on a solid support to bind to and remove the antibody from the sample. The bound antibody can then be detected using a detection reagent that contains a reporter group. Suitable detection reagents include antibodies that bind to the antibody/ancestor protein complex and free protein labeled with a reporter group (e.g., in a semi-competitive assay). Alternatively, a competitive assay can be utilized, in which an antibody that binds to the ancestor protein of interest is labeled with a reporter group and allowed to bind to the immobilized antigen after incubation of the antigen with the sample. The extent to which components of the sample inhibit the binding of the labeled antibody to the ancestor protein of interest is indicative of the reactivity of the sample with the immobilized ancestor protein.

The solid support can be any solid material known to those of ordinary skill in the art to which the antigen may be attached. For example, the solid support can be a test well in a microtiter plate or a nitrocellulose or other suitable membrane. Alternatively, the support can be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681, the disclosure of which is incorporated by reference herein.

The ancestor proteins can be bound to the solid support using a variety of techniques known to those of ordinary skill in the art, which are amply described in the patent and scientific literature. In the context of the present invention, the term “bound” refers to both non-covalent association, such as adsorption, and covalent attachment (see, e.g., Pierce Immunotechnology Catalog and Handbook, at A12-A13 (1991)).

In certain embodiments, the assay is an enzyme-linked immunosorbent assay (ELISA). This assay can be performed by first contacting an ancestor protein that has been immobilized on a solid support, commonly the well of a microtiter plate, with the sample, such that antibodies present within the sample that recognize the ancestor protein of interest are allowed to bind to the immobilized protein. Unbound sample is then removed from the immobilized ancestor protein and a detection reagent capable of binding to the immobilized antibody-protein complex is added. The amount of detection reagent that remains bound to the solid support is then determined using a method appropriate for the specific detection reagent.

More specifically, once the ancestor protein is immobilized on the support as described above, the remaining protein binding sites on the support are typically blocked. Any suitable blocking agent known to those of ordinary skill in the art, such as bovine serum albumin or TWEEN™ 2O (Sigma Chemical Co., St. Louis, Mo.), can be employed. The immobilized ancestor protein is then incubated with the sample, and the antibody is allowed to bind to the protein. The sample can be diluted with a suitable diluent, such as phosphate-buffered saline (PBS) prior to incubation. In general, an appropriate contact time (i.e., incubation time) is a period of time that is sufficient to detect the presence of antibody within a biological sample of an immunized subject. Those of ordinary skill in the art will recognize that the time necessary to achieve equilibrium can be readily determined by assaying the level of binding that occurs over a period of time. At room temperature, an incubation time of about 30 minutes is generally sufficient.

Unbound sample can then be removed by washing the solid support with an appropriate buffer, such as PBS containing 0.1% TWEEN™ 20. Detection reagent can then be added to the solid support. An appropriate detection reagent is any compound that binds to the immobilized antibody-protein complex and that can be detected by any of a variety of means known to those in the art. Typically, the detection reagent contains a binding agent (such as, for example, Protein A, Protein G, immunoglobulin, lectin or free antigen) conjugated to a reporter group. Suitable reporter groups include enzymes (such as horseradish peroxidase or alkaline phosphatase), substrates, cofactors, inhibitors, dyes, radionuclides, luminescent groups, fluorescent groups, and biotin. The conjugation of a binding agent to the reporter group can be achieved using standard methods known to those of ordinary skill in the art. Common binding agents, pre-conjugated to a variety of reporter groups, can be purchased from many commercial sources (e.g., Zymed Laboratories, San Francisco, Calif., and Pierce, Rockford, Ill.).

The detection reagent is then incubated with the immobilized antibody-protein complex for an amount of time sufficient to detect the bound antibody. An appropriate amount of time can generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods can be used to detect dyes, luminescent groups and fluorescent groups. Biotin can be detected using avidin, coupled to a different reporter group (commonly a radioactive or fluorescent group or an enzyme). Enzyme reporter groups can generally be detected by the addition of substrate (generally for a specific period of time), followed by spectroscopic or other analysis of the reaction products.

To determine the presence or absence of anti-ancestor protein antibodies in the sample, the signal detected from the reporter group that remains bound to the solid support is generally compared to a signal that corresponds to a predetermined cut-off value. In one embodiment, the cut-off value is the average mean signal obtained when the immobilized ancestor protein is incubated with samples from non-immunized subject.

In a related embodiment, the assay is performed in a rapid flow-through or strip test format, wherein the ancestor protein is immobilized on a membrane, such as, for example, nitrocellulose, nylon, PVDF, and the like. In the flow-through test, antibodies within the sample bind to the immobilized polypeptide as the sample passes through the membrane. A detection reagent (e.g., protein A-colloidal gold) then binds to the antibody-protein complex as the solution containing the detection reagent flows through the membrane. The detection of bound detection reagent can then be performed as described above. In the strip test format, one end of the membrane to which the ancestor protein is bound is immersed in a solution containing the sample. The sample migrates along the membrane through a region containing the detection reagent and to the area of immobilized ancestor protein. The concentration of the detection reagent at the protein indicates the presence of anti-ancestor protein antibodies in the sample. Typically, the concentration of detection reagent at that site generates a pattern, such as a line, that can be read visually. The absence of such a pattern indicates a negative result. In general, the amount of protein immobilized on the membrane is selected to generate a visually discernible pattern when the biological sample contains a level of antibodies that would be sufficient to generate a positive signal (e.g., in an ELISA) as discussed supra. Typically, the amount of protein immobilized on the membrane ranges from about 25 ng to about 1 μg, and more typically from about 50 ng to about 500 ng. Such tests can typically be performed with a very small amount (e.g., one drop) of subject serum or blood.

Cytotoxic T-lymphocyte Assay

Another factor in treating and detecting an infection such as an infection transmitted from a xenograft or HIV-1 infection is the cellular immune response, in particular the cellular immune response involving the CD8⁺ cytotoxic T lymphocytes (CTL's). A cytotoxic T lymphocyte assay can be used to monitor the cellular immune response following sub-genomic immunization with an ancestral viral sequence against homologous and heterologous HIV strains, as above using standard methods (see, e.g., Burke et al., supra; Tigges et al., supra).

Conventional assays utilized to detect T cell responses include, for example, proliferation assays, lymphokine secretion assays, direct cytotoxicity assays, limiting dilution assays, and the like. For example, antigen-presenting cells that have been incubated with an ancestor protein can be assayed for the ability to induce CTL responses in responder cell populations. Antigen-presenting cells can be cells such as peripheral blood mononuclear cells or dendritic cells. Alternatively, mutant non-human mammalian cell lines that are deficient in their ability to load class I molecules with internally processed peptides and that have been transfected with the appropriate human class I gene, can be used to test the capacity of an ancestor peptide of interest to induce in vitro primary CTL responses.

Peripheral blood mononuclear cells (PBMCs) can be used as the responder cell source of CTL precursors. The appropriate antigen-presenting cells are incubated with the ancestor protein, after which the protein-loaded antigen-presenting cells are incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

Another suitable method allows direct quantification of antigen-specific T cells by staining with Fluorescein-labeled HLA tetrameric complexes (Altman et al., Proc. Natl. Acad. Sci. USA 90:10330 (1993); Altman et al., Science 274:94 (1996)). Other relatively recent technical developments include staining for intracellular lymphokines, and interferon release assays or ELISPOT assays. Tetramer staining, intracellular lymphokine staining and ELISPOT assays are typically at least 10-fold more sensitive than more conventional assays (Lalvani et al., J. Exp. Med. 186:859 (1997); Dunbar et al., Curr. Biol. 8:413 (1998); Murali-Krishna et al., Immunity 8:177 (1998)).

Diagnosis

The present invention also provides methods for diagnosing viral (e.g., HIV, PERV) infection and/or AIDS, using the ancestor viral sequences described herein. Diagnosing viral (e.g., HIV, PERV) infection and/or AIDS can be carried out using a variety of standard methods well known to those of skill in the art. Such methods include, but are not limited to, immunoassays, as described supra, and recombinant DNA methods to detect the presence of nucleic acid sequences. The presence of a viral gene sequence can be detected, for example, by Polymerase Chain Reaction (PCR) using specific primers designed using the sequence, or a portion thereof, set forth in Tables 1 or 3, using standard techniques (see, e.g., Innis et al., PCR Protocols A Guide to Methods and Application (1990); U.S. Pat. Nos. 4,683,202; 4,683,195; and 4,889,818; Gyllensten et al., Proc. Natl. Acad. Sci. USA 85:7652-56 (1988); Ochman et al., Genetics 120:621-23 (1988); Loh et al., Science 243:217-20 (1989)). Alternatively, a viral gene sequence can be detected in a biological sample using hybridization methods with a nucleic acid probe having at least 70% identity to the sequence set forth in Tables 1 or 3, according to methods well known to those of skill in the art (see, e.g., Sambrook et al., supra).

### EXAMPLES

### Example 1

### Determination of Ancestral Viral Sequences

Sequences representing genes of a HIV-1 subtype C were selected from the GenBank and Los Alamos sequence databases. 39 subtype C sequences were used. 18 outgroup sequences (two from each of the other group M subtypes (FIG. 8) were used as an outgroup to root the subtype C sequences. The sequences were aligned using CLUSTALW (Thompson et al., Nucleic Acids Res. 22:4673-80 (1994)), the alignments were refined using GDE (Smith et al., CABIOS 10:671-5 (1994)), and amino acid sequences translated from them. Gaps were manipulated so that they were inserted between codons. This alignment (alignment I) was modified for phylogenetic analysis so that regions that could not be unambiguously aligned were removed (Learn et al., J. Virol. 70:5720-30 (1996)) resulting in alignment II.

An appropriate evolutionary model for phylogeny and ancestral state reconstructions for these sequences (alignment II) was selected using the Akaike Information Criterion (AIC) (Akaike, IEEE Trans. Autom. Contr. 19:716-23 (1974)) as implemented in Modeltest 3.0 (Posada and Crandall, Bioinformatics 14: 817-8 (1998)). For the analysis for the subtype C ancestral sequence the optimal model is equal rates for both classes of transitions and different rates for all four classes of transversions, with invariable sites and a F distribution of site-to-site rate variability of variable sites (referred to as a TVM+I+G model). The parameters of the model in this case were: equilibrium nucleotide frequencies: ƒ_{A}=0.3576, ƒ_{C}=0.1829, ƒ_{G}=0.2314, ƒ_{T}=0.2290; proportion of invariable sites=0.2447; shape parameter (α) of the Γ distribution=0.7623; rate matrix (R) matrix values: R_{A→C}=1.7502, R_{A→G}=R_{C→T}=4.1332, R_{A→T}=0.6825, R_{C→G}=0.6549, R_{G→T}=1.

Evolutionary trees for the sequences (alignment II) were inferred using maximum likelihood estimation (MLE) methods as implemented in PAUP* version 4.0b (Swofford, PAUP 4.0: Phylogenetic Analysis Using Parsimony (And Other Methods). Sinauer Associates, Inc. (2000)). Specifically for the subtype C sequences, ten different subtree-pruning-regrafting (SPR) heuristic searches were performed each using a different random addition order. All ten searches found the same MLE phylogeny (LnL=−33585.74). The ancestral nucleotide sequence for subtype C was inferred to be the sequence at the basal node of this subtype using this phylogeny, the sequences from the databases (alignment II), and the TVM+I+G model above using marginal likelihood estimation (see below).

This inferred sequence does not include predicted ancestral sequence for portions of several variable regions (V1, V2, V4 and V5) and four additional short regions that could not be unambiguously aligned (these eight regions were removed from alignment I to produce alignment II). The following procedure was used to predict amino acid sequences for the complete gp160 including the highly variable regions. The inferred ancestral sequence was visually aligned to alignment I and translated using GDE (Smith et al., supra). Since the highly variable regions were deleted as complete codons, the translation was in the correct reading frame and codons were properly maintained. The ancestral amino acid sequence for the regions deleted from alignment II were predicted visually and refined using a parsimony-based sequence reconstruction for these sites using the computer program MacClade, version 3.08a (Maddison and Maddison. MacClade—Analysis of Phylogeny and Character Evolution—Version 3. Sinauer Associates, Inc. (1992)). This amino acid sequences was converted to DNA sequence optimized for expression in human cells using the BACKTRANSLATE program of the Wisconsin Sequence Analysis Package (GCG), version 10 and a human gene codon table from the Codon Usage Database (http://www.kazusa.orjp/codon/cgi-bin/showcodon.cgi?species=Homo+sapiens+[gbpri]).

### Example 2

Different methods are available to determine the maximum likelihood phylogeny for a given subtype. One such method is based on the coalescent theory, which is a mathematical description of the genealogy of a sample of gene sequences drawn from a large evolving population. Coalescence analysis takes into account the HIV population in vivo and in the larger epidemic and offers a way of understanding how sampled genealogies behave when different processes operate on the HIV population. This theory can be used to determine the sequence of the ancestral viral sequence, such as a founder, or MRCA. Exponentially growing populations have decreasing coalescent intervals going back in time, while the converse is true for a declining population.

Epidemics in the USA and Thailand are growing exponentially. The coalescent dates for subtype B epidemics in the USA and Thailand are in accordance with the epidemiologic data. The coalescent date for subtype E epidemic in Thailand is earlier than predicted from the epidemiologic data. Potential reasons that can account for this discrepancy include, for example, the existence of multiple introductions of HIV-1 (there is no evidence from phylogenetics on this point), the absence of HIV-1 detection in Thailand for about 7 years, and the difference in the mutation rates for env gene in the HIV-1 subtypes E and B.

The Unit of Reconstruction

This unit of reconstruction relates to the ancestral viral sequence (i.e., state) state that is reconstructed. There are three possible units of reconstruction: nucleotides, amino acids or codons. In one embodiment, the states of the individual nucleotides are reconstructed and the amino acid sequences are then determined on the basis of this reconstruction. In another embodiment, the amino acid ancestral states are directly reconstructed. In a typical embodiment, the codons are reconstructed using a likelihood-based procedure that uses a codon model of evolution. A codon model of evolution takes into account the frequencies of the codons and implicitly the probability of substituting one nucleotide for another—in other words, it incorporates both nucleotide and amino acid substitutions in a single model. Computer programs capable of doing this are available or can readily be developed, as will be appreciated by the skilled artisan.

Use of Marginal or Joint Likelihoods for Estimating the Ancestral States

The ancestral state can be estimated using either a marginal or a joint likelihood. The marginal and joint likelihoods differ on the basis of how ancestral states at other nodes in the phylogenetic tree estimated. For any particular tree, the probability that the ancestral state of a given site on a sequence alignment at the root is, for example, an A can be determined in different ways.

The likelihood that the nucleotide is an adenine (A) can be determined regardless of whether higher nodes (i.e., those nodes closer to the ancestral viral sequence, founder or MRCA) have an adenine, cytosine (C), guanine(G), or thymine (T). This is the marginal likelihood of the ancestral state being A.

Alternatively, the likelihood that the nucleotide is an A can be determined depending on whether the nodes above are A, C, G, or T. This estimation is the joint likelihood of A with all the other ancestral reconstructions for that site.

The joint likelihood is a preferred method when all the ancestral states along the entire tree need to be determined. To establish the most likely states at one given node, the marginal likelihood is preferably used. In case of uncertainty at a particular site, a likelihood estimate of the ancestral state allows testing whether one state is statistically better than another. If two possible ancestral states do not have statistically different likelihoods, or if one ends up with multiple states over a number of sites building all possible sequences is not desirable. The likelihoods of all combinations can however be computed and ranked, and only those above a certain critical value are used. For example, when two sites on a sequence, each with different likelihoods for A, C, G, T, are considered:

| | |
|---|---|
| L(A) L(C) L(G) L(T)* | * L represents the -lnL (the negative log- |
| | therefore, the smaller the more likely. |
| | likelihood); |
| Site 1 3 2 1.5 1 | |
| Site 2 10 7 5 1 | |

there are 16 possible sequence configurations, each with its own log-likelihood, that is simply the sum of the log-likelihoods for each base, which are:

| | | | |
|---|---|---|---|
| AA 13 | CA 12 | GA 11.5 | TA 11 |
| AC 10 | CC 9 | GC 8.5 | TC 8 |
| AG 8 | CG 7 | GG 6.5 | TG 6 |
| AT 4 | CT 3 | GT 2.5 | TT 2 |

In order of likelihood the ranking is:

TT, GT, CT, AT, TG, GG, CG, AG, TC, GC, CC, AC, TA, GA, CA, AA

The first four sequences have T at the second site. This results from the likelihood at that site being spread over a large range, resulting into a very low probability of having any nucleotide other than T at this site. At Site 1, however, any nucleotide tends to give quite similar likelihoods. This kind of ranking is one way of whittling down the number of possible sequences to look at if variation is to be taken into account.

The above variation in reconstructed ancestral states deals with variation that comes about because of the stochastic nature of the evolutionary process, and because of the probabilistic models of that process that are typically used. Another source of variation results from the sampling of sequences. One way of testing how sampling affects ancestral state reconstruction is to perform jackknife re-sampling on an existing data set. This involves deleting randomly without replacement of some portion (e.g., half) of the sequences, and reconstructing the ancestral state. Alternatively, the ancestral state can be estimated for each of a set of bootstrap trees, and the number of times a particular nucleotide was estimated can be reported as the ancestral state for a given site. The bootstrap trees are generated using bootstrapped data, but the ancestral state reconstructions use the bootstrap trees on the original data.

Different models of evolution can be used to reconstruct the ancestral states for the root node. Examples of models are known and can be chosen on a multitude of levels. For example, a model of evolution can be chosen by some heuristic means or by picking one that gives the highest likelihood for the ancestral sequence (obtained by summing the likelihoods over all sites). Alternatively the ancestral states are reconstructed at each site over all models of evolution, all of the likelihoods obtained summed, and the ancestral state chosen that has the maximum likelihood.

### Example 3

The conservation of HIV-1 subtype C CTL amino acid consensus epitopes was analyzed. The total number of epitopes was 395. The table below summarize the results of the similarly of each circulating viral sequence to the C subtype CTL consensus sequence. The determined ancestor viral sequence for the HIV-1 subtype C env protein (SEQ ID NO:4) has the highest score (98.48%). Note that the scores for several strains are below 65%, because truncated sequences were used.

| Sequence Name | Total AA number | Percentage CTL to Consensus |
|---|---|---|
| cCanc95-mod1 | 389 | 98.48% |
| cBR.92BR025 | 376 | 95.19% |
| cBI.BU910717 | 363 | 91.90% |
| cIN.21068 | 368 | 93.16% |
| cIN.301905 | 370 | 93.67% |
| cMW959.U08453 | 358 | 90.63% |
| cBW.96BW1210 | 365 | 92.41% |
| cBI.BU910316 | 367 | 92.91% |
| cZAM176.U86778 | 352 | 89.11% |
| cMW965.U08455 | 364 | 92.15% |
| cZAM174.16.U86768 | 351 | 88.86% |
| c84ZR085.U88822 | 322 | 81.52% |
| cSN.SE364A | 370 | 93.67% |
| cMW960.U08454 | 365 | 92.41% |
| cBI.BU910812 | 368 | 93.16% |
| cET.ETH2220 | 358 | 90.63% |
| cBI.BU910518 | 361 | 91.39% |
| cIN.94IN11246 | 361 | 91.39% |
| cBW.96BW15B03 | 359 | 90.89% |
| cDJ.DJ259A | 355 | 89.87% |
| cBI.BU910213 | 365 | 92.41% |
| cBW.96BW01B03 | 362 | 91.65% |
| cIND760.L07655 | 255 | 64.56% |
| cIN.301904 | 372 | 94.18% |
| cSO.SM145A | 354 | 89.62% |
| cCHN19.AF268277 | 356 | 90.13% |
| cIND747.L07653 | 255 | 64.56% |
| cBW.96BW0402 | 364 | 92.15% |
| cBI.BU910611 | 367 | 92.91% |
| cBI.BU910423 | 359 | 90.89% |
| cBW.96BW17B05 | 355 | 89.87% |
| cBW.96BW0502 | 367 | 92.91% |
| cUG.UG268A2 | 372 | 94.18% |
| cZAM18.L22954 | 365 | 92.41% |
| cIN.301999 | 368 | 93.16% |
| c91BR15.U39238 | 371 | 93.92% |
| cDJ.DJ373A | 361 | 91.39% |
| cBI.BU910112 | 369 | 93.42% |
| c93IN101.AB023804 | 365 | 92.41% |
| cBW.96BW16B01 | 361 | 91.39% |
| cBW.96BW11B01 | 361 | 91.39% |
| cINdiananc66 | 363 | 91.90% |

### Example 4

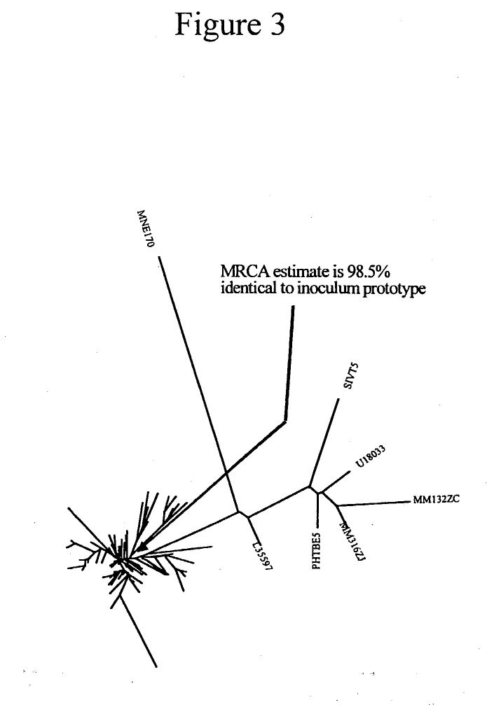

Ancestor sequence reconstruction was performed on simian immunodeficiency viruses grown in macaques. Macaques were infected and challenged with a relatively homogeneous SIV inoculum. Viral sequences were obtained up to three years following infection and were used to deduce an MRCA using maximum likelihood phylogeny analysis. The resulting sequence was compared to the consensus sequence of the inoculum. The MRCA sequence was found to be 97.4% identical to the virus inoculum. This figure improved to 98.2% when convergence at 5 glycosylation sites was removed—this convergence was due to readaptation of the virus from tissue culture to growth in the animal (Edmonson et al., J. Virol. 72:405-14 (1998)). The MRCA sequence and the consensus sequence were found to differ at 1.5% at the nucleotide level. FIG. 3 illustrates the determination of simian immunodeficiency virus MRCA phylogeny.

### Example 5

An experiment to test the biological activity of the HIV-1 subtype B ancestral viral env gene sequence was performed. A nucleic acid sequence encoding the HIV-1 subtype B ancestral viral env gene sequence was assembled from long (160-200 base) oligonucleotides; the assembled gene was designated ANC1. The biological activity of ANC1 HIV-1-B Env was evaluated in co-receptor binding and syncytium formation assays. The plasmid pANC1, harboring the determined and chemically synthesized HIV-1 subtype B Ancestor gp160 Env sequence, or a positive control plasmid containing the HIV-1 subtype B 89.6 gp160 Env, was transfected into COS7 cells. These cells are capable of taking up and expressing foreign DNA at high efficiencies and thus are routinely used to produce viral proteins for presentation to other cells. The transfected COS7 cells were then mixed with GHOST cells expressing either one of the two major HIV-1 co-receptor proteins, CCR5 or CXCR4. CCR5 is the predominant receptor used by HIV early in infection. CXCR4 is used later in infection, and use of the latter receptor is temporally associated with the development of disease. The COS7-GHOST-co-receptor+cells were then monitored for giant cell formation by light microscopy and for expression of viral Env protein by HIV-Env-specific antibody staining and fluorescence detection.

Cells expressing the ANC1 Env were shown to be expressed by virtue of binding to HIV-specific antibody and fluorescent detection, and to cause the formation of giant multinucleated cells in the presence of the CCR5 co-receptor, but not the CXCR4 co-receptor. The positive control 89.6 Env uses both CCR5 and CXCR4 and formed syncytia with cells expressing either co-receptor. Thus, the ANC1 Env protein was shown to be biologically active by co-receptor binding and syncytium formation.

### Example 6

Maximum likelihood phylogeny reconstruction differs from traditional consensus sequence determinations because a consensus sequence represents a sequence of the most common nucleotide or amino acid residue at each site in the sequence. Thus, a consensus sequence is subject to biased sampling. In particular, the determination of a consensus sequence can be biased if many samples have the same sequence. In addition, the consensus sequence is a real viral sequence.

In contrast, maximum likelihood phylogeny analysis is less likely to be affected by biased sample because it does not determine the sequence of a most recent common ancestor based solely on the frequencies of the each nucleotide at each position. The determined ancestral viral sequence is an estimate of a real virus, the virus that is the common ancestor of the sampled circulating viruses.

In the simplest of methods for determining an ancestral sequence, for a single site on a sequence alignment nucleotides are assigned to ancestral nodes such that the total number of changes between nodes is minimized; this approach is called a “most parsimonious reconstruction.” An alternative methodology, based on the principle of maximum likelihood, assigns nucleotides at the nodes such that the probability of obtaining the observed sequences, given a phylogeny, is maximized. The phylogeny is constructed by using a model of evolution that specifies the probabilities of nucleotide substitutions. The maximum likelihood phylogeny is the one that has the highest probability of giving the observed data.

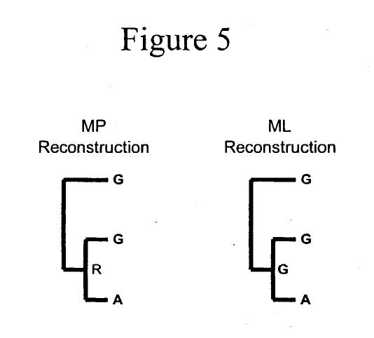

Referring to FIG. 5, a comparison is presented of parsimony methodology and maximum likelihood methodology of determining an ancestral viral sequence (e.g., a founder sequence or a most recent common ancestor sequence (MRCA)). The most parsimonious reconstruction (“MP”) can have the undesirable problem of creating an ambiguous state at the ancestral branch point (i.e., node). In this example, the two descendant sequences from this node have an adenine (A) or guanine (G) at a particular position in the sequence. The most parsimonious reconstruction (“MP Reconstruction”) for the ancestral sequence at this site is ambiguous, because there can be either an A or G (symbolized by “R”) at this position. In contrast, a maximum likelihood phylogeny analysis applies knowledge about sequence evolution. For example, likelihood analysis relies, in part, on the identity of nucleotides at the same position in other variants. Thus, in this example, a G to A mutation is more likely than an A to G change because variant at the adjacent node also has a G at the same position.

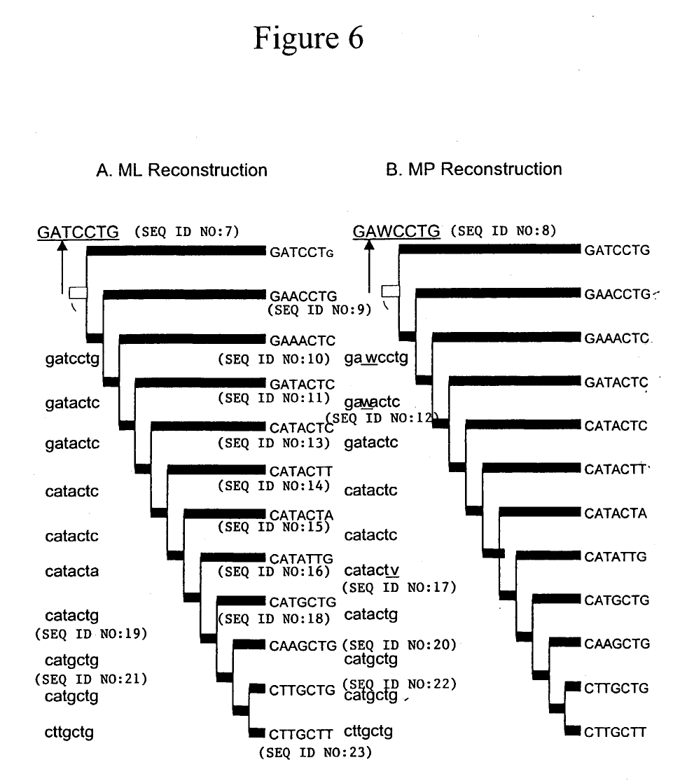

Referring to FIG. 6, another example illustrates the differences in these methodologies to determine a most recent common ancestor. In this example, twelve sequences of seven nucleotides are presented. These sequences share the illustrated evolutionary history. A consensus sequence calculated from these sequences is CATACTG. In panel A, the maximum likelihood reconstruction of the determined ancestral node is shown as GATCCTG. Other determined sequences are presented adjacent the other internal nodes. In panel B, the most parsimonious reconstruction at the same nodes is presented. As shown, the most parsimonious reconstruction predicts the consensus sequence GAWCCTG, where “W” symbolizes that either an A or T is equally possible to be at the third position. Similarly other most parsimonious reconstructions are shown at the various internal nodes. At the seventh internal node, the last nucleotide is indicated with the symbol “V” representing that an A, C or G might be present. Also note in this example, the consensus sequence differs in at least two sites (the 1^{st} and 4^{th} positions) from either the maximum likelihood- or parsimony-determined sequence for the MRCA.

### Example 8

### Reconstruction of Porcine Endogenous Retrovirus Ancestral Sequences

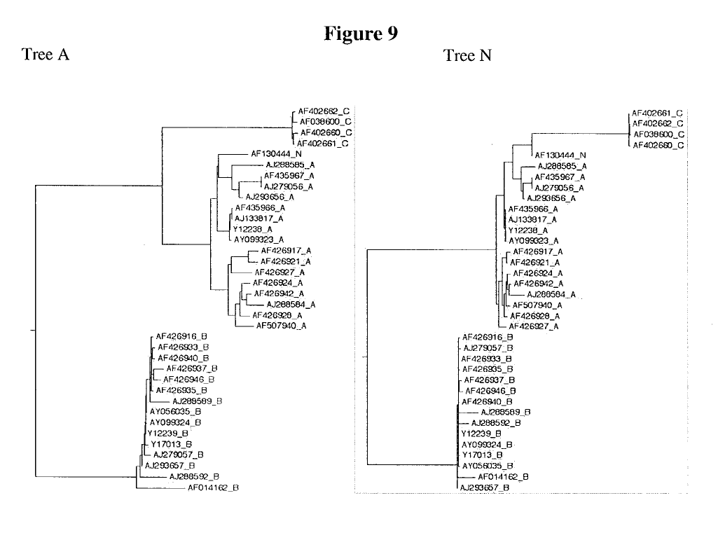

Sequences representing the env gene of Porcine Endogenous Retrovirus (PERV) were obtained from GenBank®. In selecting data for this reconstruction, putative recombinant forms were excluded (e.g., subtypes A/B (Lee, J.-H., et al. J Virol. 76:5548-5556, 2002) and A/C (Oldmixon, B. A., et al. J Virol. 76:3045-3048, 2002)). Some other sequences were excluded because they contained imbedded stop codons, and may have been pseudogenes rather than translationally competent open reading frames. A few of the sequences were derived from viruses obtained from human cell lines, and hence proven to be infectious. Although there is the possibility that such viruses had undergone selection for the ability to replicate within the host cell system, and so might differ from the naive, endogenous state, they were included to increase the number of sequences on which the reconstructions are based. The sequences (and subtypes) are AF130444 (A), AJ133817 (A), AY099323 (A), AY099324 (B), and AF402660 (C), none of which is an outlier in the phylogenetic trees (FIG. 9).

Seventeen subtype A sequences were used. Fifteen subtype B sequences were used. Four subtype C sequences were used. These original sequences were of several different lengths. The Genbank® accession numbers and bibliographic reference for the genomic sequence of each subtype of PERV env used in the ancestral sequence reconstruction is shown below. ds refers to a direct submission to GenBank®.

Subtype A

AF130444 (NIH = A) (Wilson, C. A., et al. J Virol. 74: 49-56,
2000)
AF426917 (Lee, J. -H., et al. J Virol. 76: 5548-5556, 2002)
AF426921 (Lee, J. -H., et al. J Virol. 76: 5548-5556, 2002)
AF426924 (Lee, J. -H., et al. J Virol. 76: 5548-5556, 2002)
AF426927 (Lee, J. -H., et al. J Virol. 76: 5548-5556, 2002)
AF426928 (Lee, J. -H., et al. J Virol. 76: 5548-5556, 2002)
AF426942 (Lee, J. -H., et al. J Virol. 76: 5548-5556, 2002)
AF435966 (Niebert, M., et al. J Virol. 76: 2714-2720, 2002.)
AF435967 (Niebert, M., et al. J Virol. 76: 2714-2720, 2002.)
AF507940 (Lu, M., et al ds)
AJ133817 (Czauderna, F., et al ds)
AJ279056 (Niebert, M., et al. J Virol. 76: 2714-2720, 2002.)
AJ288584 (Bosch, S., et al. J Virol. 74: 8575-8581, 2000)
AJ288585 (Bosch, S., et al. J Virol. 74: 8575-8581, 2000)
AJ293656 (Krach, U., et al. J Virol. 75: 5465-5472, 2001)
AY099323 (Bartosch, B., et al. J Gen Virol. 83: 2231-2240,
2002)
Y12238 (Le Tissier, P., et al. Nature 389: 681-682, 1997)

Subtype B

AF014162 (Haworth, C., et al ds)
AF426916 (Lee, J. -H., et al. J Virol. 76: 5548-5556, 2002)
AF426933 (Lee, J. -H., et al. J Virol. 76: 5548-5556, 2002)
AF426935 (Lee, J. -H., et al. J Virol. 76: 5548-5556, 2002)
AF426937 (Lee, J. -H., et al. J Virol. 76: 5548-5556, 2002)
AF426940 (Lee, J. -H., et al. J Virol. 76: 5548-5556, 2002)
AF426946 (Lee, J. -H., et al. J Virol. 76: 5548-5556, 2002)
AJ279057 (Niebert, M., et al. J Virol. 76: 2714-2720, 2002.)
AJ288589 (Bosch, S., et al. J Virol. 74: 8575-8581, 2000)
AJ288592 (Bosch, S., et al. J Virol. 74: 8575-8581, 2000)
AJ293657 (Krach, U., et al. J Virol. 75: 5465-5472, 2001)
AY056035 (Herring, C., G. et al. J Virol. 75: 12252-12265, 2001)
AY099324 (Bartosch, B., et al. J Gen Virol. 83: 2231-2240,
2002)
Y12239 (Le Tissier, P., et al. Nature 389: 681-682, 1997)
Y17013 (Czauderna, F., N. et al. J Virol. 74: 4028-4038, 2000)

Subtype C

AF038600 (Akiyoshi, D. E., et al. J Virol. 72: 4503-4507,
1998)
AF402660 (Blusch, J. H., et al ds)
AF402661 (Blusch, J. H., et al ds)
AF402662 (Blusch, J. H., et al ds)

Sequences were aligned with Clustal W using its default parameter settings (Thompson, J. D.,et al. Nucleic Acids Research 24:4876-4882, 1997), and then adjusted by hand to establish and preserve codon alignment across sequences (e.g., to manipulate gaps to insert them between codons). Next, phylogenetic trees for the sequences were inferred using Paup*v4b10 (Swofford, D. L. PAUP*: Phylogenetic analysis using parsimony (* and other methods). Sinauer, Sunderland, Mass., 2001). For Tree N, the aligned nucleotide sequences were used to estimate the tree. First a neighbor-joining (NJ) tree was estimated from maximum likelihood (ML) estimates of distance calculated under the GTR model with site variation in substitution rate (Γ-distributed in 4 bins and shape parameter α=0.5). Then the ML tree was estimated, using the estimated values of α and the R (substitution) matrix from the NJ tree, empirical nucleotide frequencies and using the NJ tree as starting point.

For Tree A, the aligned nucleotide sequences were translated to amino acids. The amino acid sequences were used to estimate the tree. First a neighbor-joining (NJ) tree was estimated from mean character difference. Then a heuristic search was made for the best tree using the distance optimality criterion of minimum evolution. Two trees of equal score were recovered, but since they differed only in the order of branching of two very short branches within the B clade, one was arbitrarily chosen for subsequent use.

A different phylogenetic tree was estimated when the input sequences were analyzed as amino acids (Tree A, FIG. 9) or nucleotides (Tree N, FIG. 9). In both trees, the three subtypes formed well defined clades. The major difference between the trees is the relationship of subtypes A and C. In Tree A, subtypes A and C are sister clades, whereas in Tree N subtype C is monophyletic within the subtype A clade.

Methods A, B, C, and N were used to reconstruct ancestral sequences either one or both of the trees as indicated below. The ancestral sequence was taken to be that for the basal node for each clade, when the tree was rooted using any of the other clades. In each case the sequences segregated into three distinct clades.

Method A. The sequences were analysed as amino acid sequences using the codeml module of PAML v3.0 running under Macintosh OS 9. The parameters were: input user tree, Tree A; no molecular clock; Jones matrix of transition probabilities; marginal reconstruction of sequences at internal nodes of tree. Otherwise, processes were assumed to be homogeneous across the tree and along the sequence.

Method B. The sequences were analysed as coding nucleotide sequences (ie codons) using the baseml module of PAML v3.0 running under Macintosh OS 9. The parameters were: input user tree, Tree A and Tree N; no molecular clock; HKY85 model of substitution; Mgene=4 and data file prefixed with GC in header line; κ (transition/transversion ratio) estimated with starting value=5; α (shape parameter for Γ distribution) set to 0.77 used with 4 bins; marginal reconstruction of sequences at internal nodes of tree. Otherwise, processes were assumed to be homogeneous across the tree and along the sequence.

Method C. The sequences were analysed as coding nucleotide sequences (ie codons) using the codeml module of PAML v3.0 running under Macintosh OS 9. The parameters were: input user tree, Tree A and Tree N; no molecular clock; data file prefixed with GC in header line; sequence interpreted as codons; nucleotide frequencies estimated in Codon position×base (3×4) table for each sequence; one dN/dS ratio; κ (transition/transversion ratio) estimated with starting value=2; α (shape parameter for Γ distribution) set to 0.77 used with 4 bins; marginal reconstruction of sequences at internal nodes of tree. Otherwise, processes were assumed to be homogeneous across the tree and along the sequence.

Method N. The sequences were analysed as non-coding nucleotide sequences using the baseml module of PAML v3.0 running under Macintosh OS 9. The parameters were: input user tree with branch lengths, Tree A and Tree N; GTR model of substitution; κ (transition/transversion ratio) estimated with starting value=5; α (shape parameter for Γ distribution) set to 0.946957 as obtained from tree estimation, and used with 4 bins; marginal reconstruction of sequences at internal nodes of tree. Otherwise processes were assumed to be homogeneous across the tree and along the sequence.

More than one ancestral sequence was reconstructed for each subtype. Where differences occurred, most commonly the reconstructions obtained under Method C differed from those obtained by other methods (Table 7). In each of these cases, the reconstruction placed an insertion in the sequences of both sister clades rather than in just one, even when the insertion did not occur in the sequences of one of the subtypes concerned. For example, Method C would reconstruct ancestral sequence . . . AAACCCAAA . . . for both subtypes even when all of the members of one subtype had sequence . . . AAA - - - AAA . . . The second most common, but much rarer, source of differences was the phylogenetic tree used, especially for subtype A. At a few sites, Methods B and N under the nucleotide tree, or Method B under the amino acid tree, reconstructed different nucleotides than did the other methods, or the same methods with the other tree. Lastly, there were a very few sites at which the reconstruction using amino acids differed from that using nucleotides.

The table below shows observed differences in reconstructed ancestral sequences, either as nucleotides or translated amino acids. Sites were classified according to the pattern of variation in reconstructed nucleotide (or amino acid) with respect to the phylogenetic tree or method used in the reconstruction. For example, 14 sites in the A ancestor showed two nucleotide reconstructions, one for Tree N and one for Tree A. The entries are the number of nucleotide (amino acid) sites where each pattern was found. The sequences reconstructed under Method A are included only in the comparison of amino acid sequences.

| | Site Patterns | | | | |
|---|---|---|---|---|---|
| | | | Method x | | |
| | | Method: | Tree: | Method x | Sequence: |
| Sub- | | C vs A, | BN or NN vs | Tree: | A vs B, |
| type | Tree: N vs A | B or N | others | BA vs others | C or N |
| A | 14 (5) | 41 (19) | 4 (3) | 5 | (1) |
| B | 3 (2) | 56 (20) | 0 (0) | 0 | (1) |
| C | 0 (0) | 117 (38) | 0 (0) | 0 | (0) |

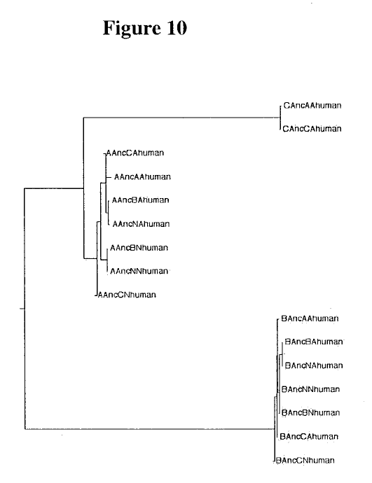

The nucleotide sequences were rewritten to use the most common codon in humans (http://www.kazusa.or.jp/codon/using GenBank Release 129.0 [Apr. 15, 2002]) (Nakamura, Y., et al. Nuc Acids Res. 26:334, 1998). These sequences are given in Table 9. The ancestral sequences reconstructed as amino acids using Method A were back-translated to nucleotides using the same table. The relative differences among the sequences are illustrated in FIG. 10. Subtype C is represented by only two unique ancestral sequences, one based on codons and one derived from all other methods.

Each combination of phylogenetic tree and method of reconstruction generated a different ancestral sequence for subtypes A and B. These reconstructed sequences differed primarily on whether a nucleotide or amino acid tree was used, or on whether a codon-based method of reconstruction was used. For both subtypes the reconstructed sequence generated using the nucleotide tree and the codon-based method was basal in the subtype clade of reconstructed sequences (FIG. 10). For subtype C the reconstructions differ according to whether the codon method was used or not. For each subtype, the differences in reconstructions are small relative to the differences among subtypes, indicating that each combination of tree and method generated similar results.

From the foregoing, it will be appreciated that, although specific embodiments of the invention have been described herein for the purpose of illustration, various modifications may be made without deviating from the spirit and scope of the invention. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

TABLE 1

(SEQ ID NO:1)

| | |
|---|---|
| 1 | ATGCGCGTGA AGGGCATCCG CAAGAACTAC CAGCACCTGT GGCGCTGGGG |
| 51 | CACCATGCTG CTGGCGATGC TGATGATCTG CTCCGCGGCC GAGAAGCTCT |
| 101 | GGGTGACCGT GTACTACGGC GTGCCCGTGT GGAAGGAGGC CACCACCACC |
| 151 | CTGTTCTGCG CCAGCCACCC CAAGGCTTAC GACACCGAGG TCCACAACGT |
| 201 | GTGGGCCACC CACGCCTGCG TGCCCACCGA CCCCAACCCC CAGGAGGTGG |
| 251 | TGCTGGAGAA CGTCACCGAG AACTTCAACA TGTGGAAGAA CAACATGGTG |
| 301 | GAGCAGATGC ACGAGGACAT CATCAGCCTG TGGGACCAGA GCCTGAAGCC |
| 351 | CTGCGTCAAG TTAACCCCCC TGTGCGTGAC CCTGAACTGC ACCGACGACC |
| 401 | TGCGCACCAA CGCCACCAAC ACCACCAACA GCAGCGCCAC CACCAACACC |
| 451 | ACCAGCAGCG GCGGCGCCAC GATGGAGGGC GAGAAGGGCG AGATCAAGAA |
| 501 | CTGCAGCTTC AACGTGACCA CCAGCATCCG CGACAAGATG CAGAAGGAGT |
| 551 | ACGCCCTGTT CTACAAGCTG GACGTGGTGC CCATCGACAA CGACAACAAC |
| 601 | AACACCAACA ACAACACCAG CTACCGCCTC ATCAACTGCA ACACCAGCGT |
| 651 | GATCACCCAG GCCTGCCCCA AGGTGAGCTT CGAGCCCATC CCCATCCACT |
| 701 | ACTGCACCCC CCCCGCCTTC GCCATCCTGA AGTGCAACGA CAAGAAGTTC |
| 751 | AACGGCACCG CCCCCTCCAC CAACGTGAGC ACCGTGCAGT GCACCCACCG |
| 801 | CATCCGCCCC CTGGTCAGCA CCCAGCTGCT GCTGAACGGC AGCCTGGCCG |
| 851 | AGGAGGAGGT CCTCATCCGC AGCGAGAACT TCACCGACAA CGCCAAGACC |
| 901 | ATCATCGTGC AGCTGAACGA GAGCGTGGAG ATCAACTGCA CGCGTCCCAA |
| 951 | CAACAACACC CGCAAGAGCA TCCCCATCGG CCCTGGCCGC GCCCTGTACC |
| 1001 | CCACCGGCAA GATCATCGGC GACATCCGCC AGGCCCACTG CAACCTGTCG |
| 1051 | CGAGCCAAGT CCAACAACAC CCTCAAGCAG ATCGTGACCA AGCTGCGCGA |
| 1101 | GCAGTTCGGC AACAACAAGA CCACCATCGT GTTCAACCAG AGCAGCGGCG |
| 1151 | GCGACCCCGA GATCGTGATG CACAGCTTCA ACTGCGGCGG CGAATTCTTC |
| 1201 | TACTGCAACA GCACCCAGCT GTTCAACAGC ACCTGGCACT TCAACGGCAC |
| 1251 | CTGGGGCAAC AACAACACCG AGCGCAGCAA CAACGCCGCC GACGACAACG |
| 1301 | ACACCATCAC CCTGCCCTGC CGCATCAAGC AGATCATCAA CATGTGGCAG |
| 1351 | GAGGTGGGCA AGCCCATGTA CGCCCCCCCC ATCACCGGCC AGATCCGCTG |
| 1401 | CAGCAGCAAC ATCACCGGCC TGCTGCTGAC TCGACACGGC GGCAACAACG |
| 1451 | AGAACACCAA CAACACCGAC ACCGAGATCT TCCCCCCCGG GGGCGGCGAC |
| 1501 | ATGCGCGACA ACTGGCGCAG CGAGCTGTAC AAGTACAAGC TGGTGAAGAT |
| 1551 | CGAGCCCCTG GGCGTGGCCC CCACCAAGGC CAACCGCCGC GTGGTGCAGC |
| 1601 | GCGAGAAGCG CGCCGTCCGC ATGCTGGGCG CCATGTTCCT GGGCTTCCTG |
| 1651 | GGCGCCGCCG GCAGCACCAT GGGCGCCGCC AGCATGACCC TGACCGTGCA |
| 1701 | GGCCCGCCAG CTGCTGAGCG GCATCGTGCA GCAGCAGAAC AACCTGCTGC |
| 1751 | GCGCCATCGA GGCCCAGCAG CACCTGCTGC AGCTGACCGT GTGGGGCATC |
| 1801 | AAGCAGCTGC AGGCCCGCGT GCTGGCCGTG GAGCGGTACC TGAAGGACCA |
| 1851 | GCAGCTGCTG GGCATCTGGG GCTGCAGCGG CAAGCTGATC TGCACCACCG |
| 1901 | CGGTGCCCTG GAACGCCAGC TGGAGCAACA AGAGCCTGGA CAAGATCTGG |
| 1951 | AACAACATGA CCTGGATGGA GTGGGAGCGC GAGATCGACA ACTACACCGG |
| 2001 | CCTGATCTAC ACCCTGATCG AGGAGAGCCA GAACCAGCAG GAGAAGAACG |
| 2051 | AGCAGGAGCT GCTGGAGCTG GACAAGTGGG CCAGCCTGTG GAACTGGTTC |
| 2101 | GATATCACCA ACTGGCTGTG GTACATCAAG ATCTTCATCA TGATCGTGGG |
| 2151 | CGCCCTGGTG GGCCTGCGCA TCGTGTTCGC CGTGCTGAGC ATCGTGAACC |
| 2201 | GCGTGCGCCA GGGCTACAGC CCCCTGAGCT TCCAGACCCG CCTGCCCGCC |
| 2251 | CCCCGCGGCC CCGACCGCCC CGAGGGCATC GAGGAGGAGG GCGGCGAGCG |
| 2301 | CGACCGCGAC CGCAGCGGGC GCCTGGTGAA CGGCTTCCTG GCCCTGATCT |
| 2351 | GGGACGACCT GCGCAGCCTG TGCCTGTTCA GCTACCACCG CCTGCGCGAC |
| 2401 | CTGCTGCTGA TCGTGGCCCG CATCGTGGAG CTGCTGGGCC GGCGCGGCTG |
| 2451 | GGAGGCCCTG AAGTATTGGT GGAACCTGCT GCAGTACTGG AGCCAGGAGC |
| 2501 | TGAAGAACAG CGCCGTGAGC CTGCTGAACG CCACCGCCAT CGCCGTGGCC |
| 2551 | GAGGGCACCG ACCGCGTGAT CGAGGTGGTG CAGCGCGCCT GCCGCGCCAT |
| 2601 | CCTGCACATC CCCCGCCGCA TCCGCCAGGG CCTGGAGCGC GCCCTGCTGT |
| 2651 | GA |

TABLE 2

(SEQ ID NO:2)

MRVKGIRKNY QHLWRWGTML LGMLMICSAA EKLWVTVYYG VPVWKEATTT
LFCASDAKAY DTEVHNVWAT HACVPTDPNP QEVVLENVTE NFNMWKNNMV EQMHEDIISL
WDQSLKPCVK LTPLCVTLNC TDDLRTNATN TTNSSATTNT TSSCGGTMEG EKGEIKNCSF
NVTTSIRDKN QKEYALFYKL DVVPIDNDNN NTNNNTSYRL INCNTSVITQ ACPKVSFEPI
PIHYCTPAGF AILKCNDKKF NGTGPCTNVS TVQCTHGIRP VVSTQLLLNG SLAEEEVVIR
SENFTDNAKT ITVQLNESVE INCTRPNNNT RKSIPIGPGR ALYATGKIIG DIRQAHCNLS
RAKWNNTLKQ TVTKLREQFG NNKTTIVFNQ SSGGDPEIVM HSFNCGGEFF YCNSTQLFNS
TWHFNGTWGN NNTERSNNAA DDNDTITLPC RIKQIINMWQ EVGKAMYAPP ISGQTRCSSN
ITGLLLTRDG GNNENTNNTD TEIFRPGGGD MRDNWRSELY KYKVVKIEPL GVAPTKAKRR
VVQREKRAVG MLGAMFLGFL GAAGSTMGAA SMTLTVQARQ LLSGTVQQQN NLLRAIEAQQ
HLLQLTVWGI KQLQARVLAV ERYLKDQQLL GIWGCSGKLI CTTAVPWNAS WSNKSLDKIW
NM4TWMEWER EIDNYTGLTY TLIEESQNQQ EKNEQELLEL DKWASLWNWF DTTNWLWYIK
IFIMTVGGLV GLRTVFAVLS IVNRVRQGYS PLSFQTRLPA PRGPDRPEGI EEEGGERDRD
RSGRLVNGFL ALTWDDLRSL CLFSYHRLRD LLLIVARIVE LLGRRGWEAL KYWWNLLQYW
SQELKNSAVS LLNATATAVA EGTDRVIEVV QRACRAILHT PRRIRQGLER ALL

TABLE 3

(SEQ ID NO:3)

ATGCGGGTGATGGGCATCCTGCGGAACTGCCAGCAGTGGTGGATCTGGGGCATCCTGGCC
TTCTGGATCCTGATGATCTGCAGCGTGATCGGCAACCTGTGGGTCACCGTGTACTACGGC
GTGCCCGTGTGGAAGGAGGCCAAGACCACCCTGTTCTCCGCCACCGACGCCAAGGCCTAC
GAGCGGGAGGTGCACAACGTGTGGGCCACCCACGCCTGCGTGCCCACCGACCCCAACCCC
CAGGAGATGGTGCTGGAGAACGTGACCGAGAACTTCAACATGTGGAAGAACGACATGGTG
GACCAGATGCACGAGGACATCATCAGCCTGTGGGACCAGAGCCTGAAGCCCTGCGTGAAG
CTGACCCCCCTGTGCGTGACCCTGAACTGCACCAACGTGACCAACACCAACAACAACAAC
AACACCAGCATGGGCGGCGAGATCAAGAACTGCAGCTTCAACATCACCACCGAGCTGCGG
GACAAGAAGCAGAAGGTGTACGCCCTGTTCTACCGGCTGGACATCGTGCCCCTGAACGAG
AACAGCAACAGCAACAGCAGCGAGTACCGGCTGATCAACTGCAACACCAGCGCCATCACC
CAGCCCTGCCCCAAGGTGAGCTTCGACCCCATCCCCATCCACTACTGCGCCCCCGCCGGC
TACCCCATCCTGAAGTGCAACAACAAGACCTTCAACGGCACCGGCCCCTGCAACAACGTG
AGCACCGTGCAGTGCACCCACGGCATCAAGCCCGTGGTGAGCACCCAGCTGCTGCTCAAC
GGCAGCCTGGCCGAGGAGGAGATCATCATCCGGAGCGAGAACCTGACCAACAACGCCAAG
ACCATCATCGTGCACCTGAACGAGAGCGTGGAGATCGTGTGCACCCGGCCCAACAACAAC
ACCCGGAAGAGCATCCGGATCGGCCCCGGCCAGACCTTCTACGCCACCGGCGACATCATC
GGCGACATCCGGCAGGCCCACTGCAACATCAGCGAGAAGGAGTGGAACAAGACCCTGCAG
CGGGTGGGCAACAAGCTGAAGGAGCACTTCCCCAACAAGACCATCAAGTTCGACCCCAGC
AGCGGCGGCGACCTGGAGATCACCACCCACAGCTTCAACTGCCGGGGCGAGTTCTTCTAC
TGCAACACCAGCAAGCTGTTCAACAGCACCTACAACAGCACCAACAACGGCACCACCAGC
AACAGCACCATCACCCTGCCCTGCCGGATCAAGCAGATCATCAACATGTGGCAGGGCGTG
GGCCGGGCCATGTACGCCCCCCCCATCGCCGGCAACATCACCTGCAAGAGCAACATCACC
GGCCTGCTGCTGACCCGGGACGGCGGCAACACCAACAACACCACCGAGACCTTCCGGCCC
CGCGGCGGCGACATGCCGGACAACTGGCGGAGCGAGCTGTACAACTACAAGGTGGTGGAG
ATCAAGCCCCTGGGCGTGGCCCCCACCGAGGCCAAGCCGCGGGTGGTGGAGCGGGAGAAG
CGGGCCGTCGGCATCGGCGCCGTCTTCCTGGCCTTCCTGGGCGCCGCCGGCAGCACCATG
GGCGCCGCCAGCATCACCCTGACCGTGCAGGCCCGGCAGCTGCTGAGCGGCATCGTGCAG
CAGCAGAGCAACCTGCTGCGGGCCATCGAGGCCCAGCAGCACATGCTGCAGCTGACCGTG
TGGGGCATCAAGCAGCTGCAGACCCGGGTGCTGGCCATCGAGCGGTACCTGAAGGACCAG
CAGCTGCTGGGCATCTGGGGCTGCAGCGGCAAGCTGATCTGCACCACCGCCGTGCCCTGG
AACAGCAGCTGGAGCAACAAGAGCCAGGACGACATCTCGGACAACATGACCTCGATGCAG
TGGGACCGGGAGATCAGCAACTACACCGACACCATCTACCGGCTGCTGGAGGACAGCCAG
AACCAGCAGGAGAAGAACGAGAAGGACCTGCTGGCCCTGGACAGCTGGAAGAACCTGTGG
AACTGGTTCGACATCACCAACTGGCTGTGGTACATCAAGATCTTCATCATGATCGTGGGC
GGCCTGATCGGCCTGCGGATCATCTTCGCCGTGCTGAGCATCGTCAACCGGGTGCGGCAG
GGCTACAGCCCCCTGAGCTTCCAGACCCTGACCCCCAACCCCCGGCCCCCCGACCGGCTG
GGCGGCATCGACCAGGAGCGCGGCCAGCACGACCGGGACCGGAGCATCCGGCTCCTGAGC
GGCTTCCTGGCCCTGGCCTGGGACGACCTGCGGAGCCTGTGCCTGTTCAGCTACCACCGG
CTGCGGGACTTCATCCTGATCCCCGCCCGGGGCGTGAACCTGCTCGGCCGGAGCACCCTG
CGGGGCCTGCAGCGGGGCTCGGAGGCCCTGAAGTACCTGGGCAGCCTGGTGCAGTACTGG
GGCCTGGAGCTGAAGAAGACCGCCATCAGCCTGCTGGACACCATCGCCATCGCCGTGGCC
GAGGGCACCGACCGGATCATCGAGCTGGTCCAGCGGATCTGCCGGGCCATCCGGAACATC
CCCCGGCGGATCCGGCAGGGCTTCGAGGCCGCCCTGCAGTGA

TABLE 4

(SEQ ID NO:4)

MRVMGILRNCQQWWIWGILGFWMLMICSVMGNLWVTVYYGVPVWKEAKTT
LFCASDAKAYEREVHNVWATHACVPTDPNPQEMVLENVTENFNMWKNDMV
DQMHEDIISLWDQSLKPCVKLTPLCVTLNCTNVTNTNNNNNTSMGGEIKN
CSFNITTELRDKKQKVYALFYRLDIVPLNENSNSNSSEYRLINCNTSAIT
QACPKVSFDPIPIHYCAPAGYAILKCNNKTFNGTGPCNNVSTVQCTHGIK
PVVSTQLLLNGSLAEEEIIIRSENLTNNAKTIIVHLNESVEIVCTRPNNN
TRKSIRIGPGQTFYATGDIIGDIRQAHCNISEKEWNKTLQRVGKKLKEHF
PNKTIKFEPSSGGDLEITTHSFNCRGEFFYCNTSKLFNSTYNSTNNGTTS
NSTITLPCRIKQIINMWQGVGRAMYAPPIAGNITCKSNITGLLLTRDGGN
TNNTTETFRPGGGDMRDNWRSELYKYKVVEIKPLGVAPTEAKRRVVEREK
RAVGIGAVFLGFLGAAGSTMGAASITLTVQARQLLSGIVQQQSNLLRAIE
AQQHMLQLTVWGIKQLQTRVLAIERYLKDQQLLGIWGCSGKLICTTAVPW
NSSWSNKSQDDIWDNNTWMQWDREISNYTDTIYRLLEDSQNQQEKNEKDL
LALDSWKNLWNWFDITNWLWYIKIFIMIVGGLIGLRIIFAVLSIVNRVRQ
GYSPLSFQTLTPNPRGPDRLGGIEEEGGEQDRDRSIRLVSGFLALAWDDL
RSLCLFSYHRLRDFILIAARGVNLLGRSSLRGLQRGWEALKYLGSLVQYW
GLELKKSAISLLDTIAIAVAEGTDRIIELVQRICRAIRNIPRRIRQGFEA
ALQ

TABLE 5

(SEQ ID NO:5)

ATGAGAGTGAAGGGGATCAGGAAGAACTATCAGCACTTCTGGAGATGGGG
CACCATGCTCCTTGGGATGTTGATCATCTGTAGCGCCCCCGAGAAGCTGT
GGGTGACCGTGTACTACCGCGTGCCCGTGTGGAAGGACGCCACCACCACC
CTGTTCTGCGCCAGCGACGCCAAGGCTTACGACACCCAGGTCCACAACGT
GTGGGCCACCCACGCCTGCGTGCCCACCGACCCCAACCCCCAGGAGGTGG
TGCTGGAGAACGTGACCGAGAACTTCAACATGTGGAAGAACAACATGGTG
GAGCACATGCACGACGACATCATCAGCCTGTGGGACCAGAGCCTGAAGCC
CTGCGTGAAGTTAACCCCCCTGTGCGTGACCCTGAACTGCACCGACGACC
TGCGCACCAACGCCACCAACACCACCAACAGCAGCGCCACCACCAACACC
ACCAGCAGCGGCGGCGGCACGATGGAGGGCGAGAAGGGCGAGATCAAGAA
CTGCAGCTTCAACGTGACCACCAGCATCCGCGACAAGATGCAGAAGGAGT
ACGCCCTGTTCTACAAGCTGGACGTGGTGCCCATCGACAACGACAACAAC
AACACCAACAACAACACCAGCTACCGCCTCATCAACTGCAACACCAGCGT
GATCACCCAGGCCTGCCCCAAGGTGAGCTTCGAGCCCATCCCCATCCACT
ACTGCACCCCCGCCGGCTTCGCCATCCTGAAGTGCAACGACAAGAAGTTC
AACGGCACCGGCCCCTGCACCAACGTGAGCACCCTGCAGTGCACCCACGG
CATCCGCCCCGTGGTGAGCACCCAGCTGCTGCTGAACGGCAGCCTGGCCG
AGGAGGAGGTGGTGATCCGCAGCGAGAACTTCACCGACAACGCCAAGACC
ATCATCGTGCAGCTGAACGAGAGCGTGGAGATCAACTGCACGCGTCCCAA
CAACAACACCCGCAAGAGCATCCCCATCGCCCCTGGCCGCGCCCTGTACG
CCACCGGCAAGATCATCGGCGACATCCGCCAGGCCCACTGCAACCTGTCG
CGAGCCAAGTGGAACAACACCCTGAAGCAGATCGTGACCAAGCTGCGCGA
GCAGTTCGGCAACAACAAGACCACCATCGTGTTCAACCAGACCAGCGGCG
GCGACCCCGAGATCGTGATGCACAGCTTCAACTGCGCCGGCGAATTCTTC
TACTGCAACACCACCCAGCTGTTCAACAGCACCTGGCACTTCAACGGCAC
CTGGGGCAACAACAACACCGAGCGCAGCAACAACGCCGCCGACGACAACG
ACACCATCACCCTGCCCTGCCGCATCAAGCAGATCATCAACATGTGGCAG
GAGGTGGGCAAGGCCATGTACGCCCCCCCCATCAGCGGCCAGATCCGCTG
CAGCAGCAACATCACCGGCCTGCTGCTGACTCGAGACGGCGGCAACAACG
AGAACACCAACAACACCGACACCGAGATCTTCCCCCCCGGGGGCGGCGAC
ATGCGCGACAACTGGCGCAGCGAGCTGTACAAGTACAAGGTGGTGAAGAT
CCAGCCCCTGGGCGTAGCACCCACCAAGGCAAAGAGAAGAGTGGTGCAGA
GAGAAAAAAGCGCAGTGGGAATGCTAGGAGCTATGTTCCTTGGGTTCTTG
GGAGCAGCAGGAAGCACTATGGGCGCAGCGTCAATGACGCTGACCGTACA
GGCCAGACAATTATTGTCTGGTATAGTGCAGCAGCAGAACAATCTGCTGA
GGGCTATTGAGGCGCAACAGCATCTGTTGCAACTCACAGTCTGGGGCATC
AAGCAGCTCCAGGCAAGAGTCCTGGCTGTGGAAAGATACCTAAAGGATCA
GCAGCTCCTGGGGATTTGGGGTTGCTCTGGAAAACTCATCTGCACCACTG
CTGTGCCTTGGAATGCTAGCTGGAGCAACAAGAGCCTGGACAAGATCTGG
AACAACATGACCTGGATGGAGTGGGAGCGCGAGATCGACAACTACACCGG
CCTGATCTACACCCTGATCGAGGAGAGCCAGAACCAGCAGGAGAAGAACG
AGCAGGAGCTGCTGGAGCTGGACAAGTGGGCCAGCCTGTGGAACTGGTTC
GATATCACCAACTGGCTGTGGTACATCAAGATCTTCATCATGATCGTGGG
CGGCCTGGTGGGCCTGCGCATCGTGTTCGCCGTGCTGAGCATCGTGAACC
GCGTGCGCCAGGGCTACAGCCCCCTGAGCTTCCAGACCCACCTGCCAGCC
CCGAGGGGACCCGACAGGCCCGAAGGAATCGAAGAAGAAGGTGGAGAGAG
AGACAGAGACAGATCCGGTCGATTAGTGAATGGATTCTTAGCACTTATCT
GGGACGACCTGCGGAGCCTGTGCCTCTTCAGCTACCACCGCTTGAGCGAC
TTACTCTTGATTGTAGCGAGGATTGTGGAACTTCTGGGACGCAGGGGGTG
GGAGGCCCTCAAATATTGGTGGAATCTCCTGCAGTACTGGAGTCAGGAAC
TAAAGAATAGCGCCGTGAGCCTGCTGAACGCCACCGCCATCGCCGTGGCC
GAGGGCACCGACCGCGTGATCGAGGTGGTGCAGCGCGCCTGCCGCGCCAT
CCTGCACATCCCCCGCCGCATCCGCCAGGGCCTGGAGCGCGCCCTGCTGT
GA

TABLE 6

(SEQ ID NO:6)

ATGAGAGTGATGGGGATACTGAGGAATTGTCAACAATGGTGCATATGGGG
CATCCTAGGCTTTTGGATGCTAATGATTTGTGACCTGATGGGCAACCTGT
GGGTGACCGTGTACTACGGCGTGCCCGTGTGCAAGGAGGCCAAGACCACC
CTGTTCTGCGCCAGCGACGCCAAGGCCTACGAGCGGGAGGTCCACAACCT
GTGGGCCACCCACGCCTCCGTGCCCACCGACCCCAACCCCCAGGAGATGG
TGCTGGAGAACGTGACCGAGAACTTCAACATGTGGAAGAACGACATGGTG
GACCAGATGCACGAGGACATCATCAGCCTGTGGGACCAGAGCCTGAACCC
CTGCGTGAAGCTGACCCCCCTGTGCGTGACCCTGAACTGCACCAACGTCA
CCAACACCAACAACAACAACAACACCAGCATGGGCGGCGAGATCAAGAAC
TGCAGCTTCAACATCACCACCGAGCTGCGGGACAAGAAGCAGAAGGTCTA
CGCCCTGTTCTACCGGCTGGACATCGTGCCCCTGAACGAGAACAGCAACA
GCAACAGCAGCGAGTACCGGCTGATCAACTGCAACACCAGCGCCATCACC
CAGGCCTGCCCCAAGGTGAGCTTCGACCCCATCCCCATCCACTACTGCGC
CCCCGCCGGCTACGCCATCCTGAAGTGCAACAACAAGACCTTCAACGGCA
CCGGCCCCTGCAACAACGTGAGCACCGTGCAGTGCACCCACGGCATCAAG
CCCGTGGTGAGCACCCAGCTGCTGCTGAACGGCAGCCTGGCCGAGGACGA
GATCATCATCCCGAGCGAGAACCTGACCAACAACCCCAAGACCATCATCG
TGCACCTGAACGAGAGCGTCGAGATCGTGTGCACCCGGCCCAACAACAAC
ACCCGGAAGAGCATCCGGATCCCCCCCGGCCAGACCTTCTACGCCACCGG
CGACATCATCGGCGACATCCGCCAGGCCCACTGCAACATCAGCGAGAAGG
AGTGGAACAAGACCCTGCAGCGGGTGGGCAAGAAGCTGAAGGAGCACTTC
CCCAACAAGACCATCAAGTTCGAGCCCAGCAGCGGCGGCGACCTGGAGAT
CACCACCCACAGCTTCAACTGCCGGGGCCAGTTCTTCTACTGCAACACCA
GCAAGCTGTTCAACAGCACCTACAACAGCACCAACAACGGCACCACCAGC
AACAGCACCATCACCCTGCCCTGCCGGATCAAGCAGATCATCAACATGTG
GCAGGGCGTGGGCCGGGCCATGTACGCCCCCCCCATCGCCGGCAACATCA
CCTGCAAGAGCAACATCACCGGCCTGCTGCTGACCCGGGACGGCGGCAAC
ACCAACAACACCACCGAGACCTTCCGGCCCGGCGGCGGCGACATGCGGCA
CAACTGGCGGAGCGAGCTGTACAAGTACAAGGTGGTGGAGATCAAGCCCC
TGGGCCTAGCACCCACTGAGGCAAAAAGGAGAGTGGTGCAGACAGAAAAA
AGAGCAGTGGGAATAGGAGCTGTGTTCCTTGGGTTCTTGGGACCAGCAGG
AAGCACTATGGGCGCGGCGTCAATAACGCTGACGGTACAGGCCAGACAAT
TATTGTCTGGTATAGTGCAACAGCAAAGCAATTTGCTGAGGGCTATAGAG
GCGCAACAGCATATGTTGCAACTCACGGTCTGGGGCATTAAGCAGCTCCA
GACAAGAGTCCTGGCTATAGAAAGATACCTAAAGGATCAGCACCTCCTGG
GCATTTGGGGCTGCTCTGGAAAACTCATCTGCACCACTGCTGTGCCTTGG
AACTCTACCTGGAGCAACAAGAGCCAGGACGACATCTGGGACAACATGAC
CTGGATGCAGTGGGACCGGGAGATCACCAACTACACCGACACCATCTACC
GGCTGCTGGAGGACAGCCAGAACCAGCAGGAGAAGAACGAGAAGGACCTG
CGACAGGCTCGGAGGAATCGAAGAAGAAGGTGGAGAGCAAGACAGAGACA
CTGGCTGTGGTACATCAAGATCTTCATCATGATCGTGGGCGGCCTGATCG
GCCTGCCCATCATCTTCGCCGTGCTGAGCATCGTGAACCGGGTGCGGCAG
GGCTACAGCCCCCTGAGCTTCCAGACCCTTACCCCAAACCCGAGGGGACC
CGACAGGCTCGGAGGAATCGAAGAAGAAGGTGGAGAGCAAGACACAGACA
GATCCATTCGATTAGTGAGCGGATTCTTAGCACTGGCCTGGGACGACCTG
CGGAGCCTGTGCCTCTTCAGCTACCACCGATTGAGAGACTTCATATTGAT
TGCAGCCAGAGGGTGGGAACTTCTGGCACGCAGCAGTCTCAGCGGACTGC
AGAGGGGGTGGGAACCCCTTAAGTATCTGGGAAGTCTTGTGCAGTATTGG
GGTCTGGAGCTAAAAAAGAGTGCTATTAGCCTGCTGGACACCATCGCCAT
CGCCGTCCCCGAGGGCACCGACCGGATCATCGAGCTGGTGCACCGGATCT
GCCGGGCCATCCGGAACATCCCCCGGCGGATCCGGCAGGGCTTCGAGGCC
GCCCTGCAGTGA

TABLE 7

| Recon- | | | | |
|---|---|---|---|---|
| struction | PERV | | | |
| Method | subtype | Tree | Nucleotide Sequence | SEQ ID NO: |
| B | A | A | ATGCATCCCACGTTAAGCCGGCGCCACCTCCCGATTCGCGGT | SEQ ID NO:7 |
| | | | GGAAAGCCGAAAAGACTGAAAATCCCCTTAAGCTTCGCCTCC | |
| | | | ATCGCGTGGTTCCTTACTCTGTCAATAACTCCTCAAGTTAAT | |
| | | | GGTAAACGCCTTGTGGACAGCCCCAACTCCCATAAACCCTTA | |
| | | | TCTCTCACCTGGTTACTTACTGACTCCGGTACAGGTATTAAT | |
| | | | ATTAACAGCACTCAAGGGGAGGCTCCCTTGGGCACCTGGTGG | |
| | | | CCTGAATTATATGTCTGCCTTCGATCAGTAATCCCTGGTCTC | |
| | | | AATCACCAGGCCACACCCCCCGATGTACTCCGTGCTTACGGG | |
| | | | TTTTACGTTTGCCCAGGACCCCCAAATAATGAAGAATATTGT | |
| | | | GGAAATCCTCAGGATTTCTTTTGCAAGCAATGGAGCTGCGTA | |
| | | | ACTTCTAATGATGGGAATTGCAAATGGCCAGTCTCTCAGCAA | |
| | | | GACAGAGTAAGTTACTCTTTTGTTAACAATCCTACCAGTTAT | |
| | | | AATCAATTTAATTATGGCCATGGGAGATGGAAAGATTGGCAA | |
| | | | CAGCCGGTACAAAAAGATGTACGAAATAAGCAAATAAGCTGT | |
| | | | CATTCGTTAGACCTAGATTACTTAAAAATAAGTTTCACTGAA | |
| | | | AAAGGAAAACAAGAAAATATTCAAAAGTGGGTAAATGGTATG | |
| | | | TCTTGGCGAATAGTGTACTATGGAGGCTCTGGGAGAAAGAAA | |
| | | | GGATCTGTTCTGACTATTCGCCTCAGAATAGAAACTCAGATG | |
| | | | GAACCTCCGGTTGCTATAGGACCAAATAAGGGTTTGGCCGAA | |
| | | | CAAGGACCTCCAATCCAAGAACAGAGGCCATCTCCTAACCCC | |
| | | | TCTCATTACAATACAACCTCTGGATCAGTCCCCACTGAGCCT | |
| | | | AACATCACTATTAAAACAGGGGCGAAACTTTTTAGCCTCATC | |
| | | | CAGCGAGCTTTTCAAGCTCTTAACTCCACGACTCCACAGCCT | |
| | | | ACCTCTTCTTGTTGGCTTTGCTTACCTTCGGCCCCACCTTAC | |
| | | | TATGAGGGAATGGCTAGAGCAGGGAAATTCAATGTGACAAAG | |
| | | | GAACATAGAGACCAATGTACATGGGGATCCCAAAATAAGCTT | |
| | | | ACCCTTACTGAGGTTTCTGGAAAAGGCACCTGCATAGGGATG | |
| | | | GTTCCCCCATCCCACCAACACCTTTGTAACCACACTGAAGCC | |
| | | | TTTAATCGAACCTCTGAGAGTCAATATCTGGTACCTGGTTAT | |
| | | | GACAGGTGGTGGGCATGTAATACTGGATTAACCCCTTGTGTT | |
| | | | TCCACCTTGGTTTTCAACCAAACTAAACACTTTTGCGTTATG | |
| | | | GTCCAAATTCTCCCCCGGGTGTACTACTATCCCGAAAAAGCA | |
| | | | GTCCTTGATGAATATGACTATACATATAATCGGCCAAAAAGA | |
| | | | GAGCCCATATCCCTGACACTAGCTGTAATGCTCGGATTGGGA | |
| | | | GTGGCTGCAGGCGTGGGAACAGGAACGGCTGCCCTAATCACA | |
| | | | GGACCGCAACAGCTGGAGAAAGGACTTAGTAACCTACATCGA | |
| | | | ATTGTAACGGAAGATCTCCAAGCCCTAGAAAAATCTGTCAGT | |
| | | | AACCTGGAGGAATCCCTAACCTCCTTATCTGAAGTGGTTCTA | |
| | | | CAGAACAGAAGGGGGTTAGATCTGTTATTTCTAAAAGAAGGA | |
| | | | GGGTTATGTGTAGCCTTAAAAGAGGAATGCTGCTTCTATGTA | |
| | | | GATCACTCACGACCCATCAGACACTCCATGAGCAAGCTTAGA | |
| | | | GAAAGCTTAGAGAGGCGTCGAAGGGAAAGAGAGGCTGACCAG | |
| | | | GGGTGGTTTGAAGOATGGTTCAACAGGTCTCCTTGGATGACC | |
| | | | ACCCTGCTTTCTGCTCTGACGGCACCCCTAGTAGTCCTGCTC | |
| | | | CTGTTACTTACAGTTGCGCCTTGCTTAATTAATAGGTTTGTT | |
| | | | GCCTTTGTTACAGAACCAGTGAGTGCAGTCCAGATCATGGTA | |
| | | | CTTAGGCAACAGTACCAAGGCCTTCTGAGCCAAGGAGAAACT | |
| | | | GACCTCTAGTAG | |
| B | A | N | ATGCATCCCACGTTAAGCCGGCGCCACCTCCCGATTCGGGGT | SEQ ID NO:9 |
| | | | GGAGAGCCGAAAAGACTGAAAATCCCCTTAAGCTTCGCCTCC | |
| | | | ATCGCTTGGTTCCTTACTCTGTCAATAACTCCTCAAGTTAAT | |
| | | | GGTAAACGCCTTGTGGACAGCCCGAACTCCCATAAACCCTTA | |
| | | | TCTCTCACCTGGTTACTTACTGACTCCGGTACAGGTATTACT | |
| | | | ATTAACAGCACTCAAGGGGAGGCTCCCTTAGGGACCTCGTGG | |
| | | | CCTGAGTTATATGTCTGCCTTCGATCGGTAATCCCTGGTCTC | |
| | | | AACGACCAGGCCACACCCCCCGATGTACTCCGTCCTTACAGG | |
| | | | TTTTATGTTTGCCCAGGACCCCCAAATAATGAAGAATATTGT | |
| | | | GGAAATCCTCAGGATTTCTTTTGCAAGCAATGGAGCTGCGTA | |
| | | | ACTTCTAATGATGGGAATTGGAAATGGCCAATCTCTCACCAA | |
| | | | GACAGAGTAAGTTACTCTTTTGTTAACAATCCTACCAGTTAT | |
| | | | AATCAATTTAATTATGGCCATGGGAGATGGAAAGATTGGCAA | |
| | | | CAGCGGGTACAAAAAGATGTACGAAATAAGCAAATAAGCTCT | |
| | | | AATTCGTTAGACCTAGATTACTTAAAAATAAGTTTCACTGAA | |
| | | | AAAGGAAAACAAGAAAATATTCAAAAGTGGGTAAATGGTATC | |
| | | | TCTTGGGGAATAATGTACTATGGAGGCTCTGGGAGAAGGAAA | |
| | | | GGATCTGTTCTGACTATTCGCCTCAGAATAGAAACTCAGATG | |
| | | | GAACCTCCGGTTGCTATAGGACCAAATAAGGGTTTGGCCGAA | |
| | | | CAAGGACCTCCAATCCAACAACAGAGGCCATCTCCTAACCCC | |
| | | | TCTGATTACAATACAACCTCTCGATCAGTCCCCACTGAGCCT | |
| | | | AACATCACTATTAAAACAGCCGCGAAACTTTTTACCCTCATC | |
| | | | CAGGGAGCTTTTCAAGCTCTTAACTCCACGACTCCAGAGGCT | |
| | | | ACCTCTTCTTGTTGGCTTTGCTTAGCTTCGGGCCCACCTTAC | |
| | | | TATGAGGGAATGGCTAGAGGAGGGAAATTCAATGTGACAAAG | |
| | | | GAACATAGAGACCAATGTACATGGGGATCCCAAAATAAGCTT | |
| | | | ACCCTTACTGAGGTTTCTGGAAAAGGCACCTGCATAGGGAGG | |
| | | | GTTCCCCCATCCCACCAACACCTTTGTAACCACACTGAAGCC | |
| | | | TTTAATCGAACCTCTGAGAGTCAGTATCTGGTACCTGGTTAT | |
| | | | GACAGGTGGTGCGCATGTAATACTGGATTAACCCCTTGTGTT | |
| | | | TCCACCTTGGTTTTCAACCAAACTAAAGACTTTTGTGTTATG | |
| | | | GTCCAAATTGTCCCCCGGGTGTACTACTATCCCCAAAAACCA | |
| | | | GTCCTTGATGAATATGACTATAGATATAATCGGCCAAAAAGA | |
| | | | GAACCCATATCCCTGACACTAGCTGTAATGCTCGGATTGCCA | |
| | | | GTGGCTGCAGGCGTGGGAACAGGAACGGCTGCCCTAATCACA | |
| | | | GGACCACAACAGCTGGAGAAAGGACTTAGTGACCTACATCCA | |
| | | | ATTGTAACGGAAGATCTCCAAGCCCTAGAAAAATCTGTCACT | |
| | | | AACCTAGAGGAATCCCTAACCTCCTTATCTGAAGTGGTTCTA | |
| | | | CAGAACAGAAGGGGGTTAGATCTGTTATTTCTAAAAGAAGGT | |
| | | | GGGTTATGTGTAGCCTTAAAAGAAGAATGTTGCTTCTATGTA | |
| | | | GATCACTCAOGAGCCATCAGAGACTCCATGAGCAAGCTTAGA | |
| | | | GAAACGTTAGAGAGGCGTCGAAGGGAAAGACAGGCTGACCAG | |
| | | | GGGTGGTTTGAAGGATGGTTCAACAGGTCTCCTTGGATGACC | |
| | | | ACCCTGCTTTCTGCTCTGACGGGACCCCTAGTAGTCCTGCTC | |
| | | | CTGTTACTTACAGTTGGGCCTTGCTTAATTAATAGGTTTGTT | |
| | | | GCCTTTGTTAGAGAACGAGTGAGTGCAGTCCAGATCATGGTA | |
| | | | CTTAGGCAACAGTACCAAGGCCTTCTGAGCCAAGGAGAAACT | |
| | | | GACCTCTAGTAG | |
| C | A | A | ATGCATCCCACGTTAAGCCGGCGCCACCTCCCGATTCGGGGT | SEQ ID NO:11 |
| | | | GGAAAGCCGAAAAGACTGAAAATCCCCTTAAGCTTCGCCTCC | |
| | | | ATCGCGTGGTTCCTTACTCTGTCAATAACTCCTCAAGTTAAT | |
| | | | GGTAAACGCCTTGTGGACAGCCCGAACTCCCATAAACCCTTA | |
| | | | TCTCTCACCTGGTTACTTACTCACTCCGGTACAGGTATTAAT | |
| | | | ATTAACAGCACTCAAGGGGAGCCTCCCTTAGGGACCTGGTGG | |
| | | | CCTGAATTATATGTCTCCCTTCGATCAGTAATCCCTGGTCTC | |
| | | | AATGACCAGGCCACACCCCCCGATGTACTCCGTGCTTACGGG | |
| | | | TTTTATGTTTGCCCAGGACCCCCAAATAATGAAGAATATTGT | |
| | | | GGAAATCCTCAGGATTTCTTTTGCAAGCAATGGAGCTGCGTA | |
| | | | ACTTCTAATGATGGGAATTGGAAATGGCCAGTCTCTCAGCAA | |
| | | | GACAGAGTAAGTTACTCTTTTGTTAACAATCCTACCAGTTAT | |
| | | | AATCAATTTAATTATGGCCATGGGAGATGGAAAGATTGGCAA | |
| | | | CAGCGGGTACAAAAAGATGTACCAAATAAGCAAATAAGCTGT | |
| | | | CATTCGTTAGACCTAGATTACTTAAAAATAAGTTTCACTGAA | |
| | | | AAAGGAAAACAAGAAAATATTCAAAAGTGGGTAAATGGTATG | |
| | | | TCTTGGGCAATAGTGTACTATGGAGGCTCTGCCAGAAAGAAA | |
| | | | GGATCTGTTCTOACTATTCGCCTCAGAATAGAAACTCAGATG | |
| | | | GAACCTCCGGTTGCTATAGCACCAAATAAGGGTTTGGCCGAA | |
| | | | CAAGGACCTCCAATCCAAGAACCACCGCATAACTTGCCGGTG | |
| | | | CCCCAGAGGCCATCTCCTAACCCCGACATAACACAGTCTGAT | |
| | | | TACAATACAACCTCTGGATCAGTCCCCACTAACACGCCTAGA | |
| | | | AACGAGCCTAACATCACTATTAAAACAGGGGCGAAACTTTTT | |
| | | | AGCCTCATCCAGGGAGCTTTTCAAGCTCTTAACTCCACGACT | |
| | | | CCAGAGGCTACCTCTTCTTGTTGGCTTTGCTTAGCTTCGGGC | |
| | | | CCACCTTACTATGAGGGAATGGCTAGAGGAGGGAAATTCAAT | |
| | | | GTGACAAAGGAACATAGAGACCAATGTACATGGGGATCCCAA | |
| | | | AATAAGCTTACCCTTACTGAGGTTTCTGGAAAAGGCACCTGC | |
| | | | ATAGGGAGGGTTCCCCCATCCCACCAACACCTTTGTAACCAC | |
| | | | ACTGAAGCCTTTAATCGAACCTCTGAGAGTCAATATCTCGTA | |
| | | | CCTGGTTATGACAGGTGGTGGGCATGTAATACTGCATTAACC | |
| | | | CCTTGTGTTTCCACCTTGGTTTTCAACCAAACTAAAGACTTT | |
| | | | TGCCTTATGGTCCAAATTGTCCCCCGGGTGTACTACTATCCC | |
| | | | GAAAAAGCAGTCCTTGATGAATATGACTATAGATATAATCGG | |
| | | | CCAAAAAGAGAACCCATATCCCTGACACTAGCTGTAATGCTC | |
| | | | GGATTGGGAGTGGCTGCAGGCGTGGGAACAGGAACGGCTGCC | |
| | | | CTAATCACAGGACCACAACAGCTGGAGAAAGGACTTAGTAAC | |
| | | | CTACATCGAATTGTAACGGAAGATCTCCAAGCCCTAGAAAAA | |
| | | | TCTGTCAGTAACCTGGAGGAATCCCTAACCTCCTTATCTGAA | |
| | | | GTGGTTCTACAGAACAGAAGGGGGTTAGATCTGTTATTTCTA | |
| | | | AAAGAAGGAGGGTTATGTGTAGCCTTAAAAGAGGAATGCTGC | |
| | | | TTCTATGTAGATCACTCAGGAGCCATCAGAGACTCCATGAGC | |
| | | | AAGCTTAGAGAAAGGTTAGAGAGGCGTCGAAGGCAAAGAGAG | |
| | | | GCTGACCAGGCGTCGTTTGAAGGATGGTTCAACAGGTCTCCT | |
| | | | TGGATGACCACCCTGCTTTCTGCTCTGACGGGACCCCTAGTA | |
| | | | GTCCTGCTCCTGTTACTTACAGTTGGGCCTTGCTTAATTAAT | |
| | | | AGGTTTGTTGCCTTTGTTAGAGAACGAGTGAGTGCAGTCCAG | |
| | | | ATCATGGTACTTAGGCAACAGTACCAAGGCCTTCTGAGCCAA | |
| | | | GGAGAAACTGACCTCTAC | |
| C | A | N | ATGCATCCCACGTTAAGCCGGCGCCACCTCCCGATTCGGGGT | SEQ ID NO:13 |
| | | | GGACAGCCGAAAAGACTGAAAATCCCCTTAAGCTTCGCCTCC | |
| | | | ATCGCTTCGTTCCTTACTCTGTCAATAACTCCTCAAGTTAAT | |
| | | | GGTAAACGCCTTGTGGACAGCCCGAACTCCCATAAACCCTTA | |
| | | | TCTCTCACCTGGTTACTTACTGACTCCGGTACAGGTATTACT | |
| | | | ATTAACAGCACTCAAGGGGAGGCTCCCTTAGGGACCTGGTGG | |
| | | | CCTGAATTATATGTCTGCCTTCGATCGGTAATCCCTGGTCTC | |
| | | | AACGACCACGCCACACCCCCCGATGTACTCCGTGCTTACGGG | |
| | | | TTTTATGTTTGCCCAGGACCCCCAAATAATGAAGAATATTGT | |
| | | | GGAAATCCTCAGGATTTCTTTTGCAAGCAATGGAGCTGCGTA | |
| | | | ACTTCTAATGATGGCAATTCGAAATGGCCAATCTCTCAGCAA | |
| | | | CACAGAGTAAGTTACTCTTTTGTTAACAATCCTACCAGTTAT | |
| | | | AATCAATTTAATTATGGCCATGGGAGATGGAAAGATTGGCAA | |
| | | | CAGCCCGTACAAAAAGATGTACGAAATAAGCAAATAAGCTGT | |
| | | | CATTCGTTAGACCTAGATTACTTAAAAATAAGTTTCACTGAA | |
| | | | AAAGCAAAACAAGAAAATATTCAAAAGTGGGTAAATCGTATG | |
| | | | TCTTGGGGAATACTGTACTATCGAGGCTCTGGGAGAAGGAAA | |
| | | | GGATCTGTTCTGACTATTCGCCTCAGAATAGAAACTCAGATG | |
| | | | GAACCTCCGGTTGCTATAGGACCAAATAAGGCTTTGCCCCAA | |
| | | | CAAGCACCTCCAATCCAAGAACCACCGCATAACTTGCCGGTG | |
| | | | CCCCAGAGGCCATCTCCTAACCCCGACATAACACAGTCTGAT | |
| | | | TACAATACAACCTCTGGATCAGTCCCCACTAACACGCCTACA | |
| | | | AACCACCCTAACATCACTATTAAAACAGGGGCCAAACTTTTT | |
| | | | AGCCTCATCCAGGCAGCTTTTCAAGCTCTTAACTCCACCACT | |
| | | | CCAGACGCTACCTCTTCTTGTTGGCTTTGCTTACCTTCGGGC | |
| | | | CCACCTTACTATGAGGGAATGGCTAGAGGAGGGAAATTCAAT | |
| | | | GTGACAAAGCAACATAGAGACCAATGTACATGGGGATCCCAA | |
| | | | AATAACCTTACCCTTACTGAGGTTTCTGGAAAAGGCACCTGC | |
| | | | ATAGGGAGGGTTCCCCCATCCCACCAACACCTTTGTAACCAC | |
| | | | ACTGAAGCCTTTAATCGAACCTCTGAGAGTCAGTATCTGGTA | |
| | | | CCTGGTTATCACAGGTGGTGGGCATGTAATACTGGATTAACC | |
| | | | CCTTGTGTTTCCACCTTGGTTTTCAACCAAACTAAAGACTTT | |
| | | | TGTGTTATGGTCCAAATTGTCCCCCGGGTGTACTACTATCCC | |
| | | | GAAAAACCAGTCCTTCATGAATATGACTATAGATATAATCGG | |
| | | | CCAAAAAGACAACCCATATCCCTGACACTAGCTGTAATGCTC | |
| | | | GGATTGGGAGTCGCTCCAGCCGTGGGAACAGGAACGGCTGCC | |
| | | | CTAATCACAGGACCACAACAGCTGCAGAAACGACTTAGTGAC | |
| | | | CTACATCGAATTGTAACGGAAGATCTCCAAGCCCTAGAAAAA | |
| | | | TCTGTCAGTAACCTAGAGGAATCCCTAACCTCCTTATCTGAA | |
| | | | GTGGTTCTACAGAACAGAAGGGGGTTAGATCTGTTATTTCTA | |
| | | | AAAGAAGGTGGGTTATGTGTAGCCTTAAAAGAAGAATGTTGC | |
| | | | TTCTATGTAGATCACTCAGGAGCCATCAGAGACTCCATGAGC | |
| | | | AAGCTTAGAGAAAGGTTAGAGAGGCGTCGAAGGGAAAGAGAG | |
| | | | GCTGACCAGGGGTGGTTTGAAGGATGGTTCAACAGGTCTCCT | |
| | | | TGGATGACCACCCTCCTTTCTGCTCTGACCGGACCCCTAGTA | |
| | | | GTCCTGCTCCTGTTACTTACAGTTGGGCCTTGCTTAATTAAT | |
| | | | AGGTTTGTTGCCTTTGTTAGAGAACGAGTGAGTGCAGTCCAG | |
| | | | ATCATGGTACTTAGGCAACAGTACCAAGGCCTTCTGAGCCAA | |
| | | | GGAGAAACTCACCTCTAC | |
| N | A | A | ATGCATCCCACGTTAAGCCGGCGCCACCTCCCGATTCGGGGT | SEQ ID NO:15 |
| | | | GGAAAGCCGAAAAGACTGAAAATCCCCTTAAGCTTCGCCTCC | |
| | | | ATCGCGTGGTTCCTTACTCTGTCAATAACTCCTCAAGTTAAT | |
| | | | GGTAAACGCCTTGTCGACAGCCCGAACTCCCATAAACCCTTA | |
| | | | TCTCTCACCTGGTTACTTACTGACTCCGGTACAGGTATTAAT | |
| | | | ATTAACAGCACTCAAGGGGAGCCTCCCTTAGGGACCTGGTGG | |
| | | | CCTGAATTATATGTCTGCCTTCGATCAGTAATCCCTGGTCTC | |
| | | | AATGACCAGGCCACACCCCCCGATGTACTCCGTGCTTACGGG | |
| | | | TTTTATGTTTGCCCAGGACCCCCAAATAATCAAGAATATTGT | |
| | | | GGAAATCCTCAGGATTTCTTTTGCAAGCAATGGAGCTGCGTA | |
| | | | ACTTCTAATGATGGGAATTGGAAATGGCCAGTCTCTCAGCAA | |
| | | | GACAGAGTAAGTTACTCTTTTGTTAACAATCCTACCAGTTAT | |
| | | | AATCAATTTAATTATGGCCATGGGAGATGGAAAGATTGGCAA | |
| | | | CAGCGGGTACAAAAAGATGTACGAAATAAGCAAATAAGCTGT | |
| | | | CATTCGTTAGACCTAGATTACTTAAAAATAAGTTTCACTGAA | |
| | | | AAAGGAAAACAAGAAAATATTCAAAAGTGCGTAAATGGTATG | |
| | | | TCTTGGGGAATAGTGTACTATGGAGGCTCTGGGAGAAAGAAA | |
| | | | GGATCTGTTCTGACTATTCGCCTCAGAATAGAAACTCAGATG | |
| | | | GAACCTCCGGTTGCTATAGGACCAAATAAGCGTTTGGCCGAA | |
| | | | CAAGGACCTCCAATCCAAGAACAGAGGCCATCTCCTAACCCC | |
| | | | TCTGATTACAATACAACCTCTGCATCAGTCCCCACTGAGCCT | |
| | | | AACATCACTATTAAAACAGGGGCGAAACTTTTTAGCCTCATC | |
| | | | CAGGGAGCTTTTCAAGCTCTTAACTCCACGACTCCAGAGGCT | |
| | | | ACCTCTTCTTGTTGGCTTTGCTTAGCTTCGGGCCCACCTTAC | |
| | | | TATGAGGGAATGGCTAGAGGAGGGAAATTCAATGTGACAAAG | |
| | | | GAACATAGAGACCAATGTACATGGGGATCCCAAAATAAGCTT | |
| | | | ACCCTTACTGAGGTTTCTGGAAAAGGCACCTGCATAGGGAGG | |
| | | | GTTCCCCCATCCCACCAACACCTTTGTAACCACACTGAAGCC | |
| | | | TTTAATCGAACCTCTGAGAGTCAATATCTGGTACCTGGTTAT | |
| | | | GACAGGTGGTGGGCATGTAATACTCGATTAACCCCTTGTGTT | |
| | | | TCCACCTTGGTTTTCAACCAAACTAAACACTTTTGCGTTATG | |
| | | | GTCCAAATTGTCCCCCGGGTGTACTACTATCCCGAAAAAGCA | |
| | | | GTCCTTGATGAATATGACTATAGATATAATCGGCCAAAAAGA | |
| | | | GAACCCATATCCCTGACACTAGCTGTAATGCTCGGATTGGGA | |
| | | | GTGGCTGCAGGCGTGGGAACAGGAACGGCTGCCCTAATCACA | |
| | | | GGACCACAACAGCTGCAGAAAGGACTTAGTAACCTACATCGA | |
| | | | ATTGTAACGGAAGATCTCCAAGCCCTAGAAAAATCTGTCAGT | |
| | | | AACCTGGAGCAATCCCTAACCTCCTTATCTGAAGTGGTTCTA | |
| | | | CAGAACAGAAGGGGGTTAGATCTGTTATTTCTAAAAGAAGGA | |
| | | | GGGTTATGTGTAGCCTTAAAAGAGGAATGCTGCTTCTATCTA | |
| | | | GATCACTCAGGAGCCATCAGAGACTCCATGAGCAAGCTTAGA | |
| | | | GAAAGGTTAGAGAGGCGTCGAAGGGAAAGACAGGCTGACCAG | |
| | | | CGGTGGTTTGAAGGATGGTTCAACAGGTCTCCTTGGATGACC | |
| | | | ACCCTGCTTTCTGCTCTCACGGCACCCCTACTAGTCCTGCTC | |
| | | | CTGTTACTTACAGTTGGGCCTTCCTTAATTAATAGGTTTGTT | |
| | | | GCCTTTGTTAGAGAACGAGTGAGTGCAGTCCAGATCATGGTA | |
| | | | CTTAGGCAACAGTACCAAGGCCTTCTGAGCCAAGGAGAAACT | |
| | | | GACCTCTAGTAG | |
| N | A | N | ATGCATCCCACGTTAAGCCGGCGCCACCTCCCGATTCGGGGT | SEQ ID NO:17 |
| | | | GGAGAGCCGAAAAGACTCAAAATCCCCTTAAGCTTCGCCTCC | |
| | | | ATCGCTTGGTTCCTTACTCTGTCAATAACTCCTCAAGTTAAT | |
| | | | GGTAAACGCCTTGTGGACAGCCCGAACTCCCATAAACCCTTA | |
| | | | TCTCTCACCTGGTTACTTACTGACTCCGGTACAGGTATTACT | |
| | | | ATTAACAGCACTCAAGGGGAGGCTCCCTTAGGGACCTGGTGG | |
| | | | CCTGAGTTATATGTCTGCCTTCGATCCGTAATCCCTGGTCTC | |
| | | | AACGACCAGGCCACACCCCCCGATGTACTCCGTGCTTACAGG | |
| | | | TTTTATGTTTGCCCAGGACCCCCAAATAATGAAGAATATTGT | |
| | | | GGAAATCCTCAGGATTTCTTTTGCAAGCAATGGACCTGCGTA | |
| | | | ACTTCTAATGATGGGAATTGCAAATGGCCAATCTCTCAGCAA | |
| | | | GACAGAGTAAGTTACTCTTTTGTTAACAATCCTACCAGTTAT | |
| | | | AATCAATTTAATTATGCCCATGGGAGATGGAAAGATTGGCAA | |
| | | | CAGCGGGTACAAAAAGATGTACGAAATAAGCAAATAAGCTGT | |
| | | | AATTCGTTAGACCTAGATTACTTAAAAATAAGTTTCACTGAA | |
| | | | AAAGGAAAACAAGAAAATATTCAAAAGTCGGTAAATGGTATG | |
| | | | TCTTGGGGAATAATGTACTATGGAGGCTCTGGGAGAAGGAAA | |
| | | | GGATCTGTTCTCACTATTCCCCTCAGAATAGAAACTCAGATG | |
| | | | GAACCTCCGGTTGCTATAGGACCAAATAAGGGTTTGGCCGAA | |
| | | | CAAGGACCTCCAATCCAAGAACAGAGGCCATCTCCTAACCCC | |
| | | | TCTGATTACAATACAACCTCTGGATCAGTCCCCACTGAGCCT | |
| | | | AACATCACTATTAAAACAGGGGCGAAACTTTTTAGCCTCATC | |
| | | | CACGGAGCTTTTCAAGCTCTTAACTCCACGACTCCAGAGGCT | |
| | | | ACCTCTTCTTGTTGGCTTTGCTTAGCTTCGGGCCCACCTTAC | |
| | | | TATGAGGGAATGGCTAGACGAGGGAAATTCAATCTGACAAAG | |
| | | | GAACATAGAGACCAATCTACATGGGCATCCCAAAATAACCTT | |
| | | | ACCCTTACTGAGGTTTCTGGAAAAGGCACCTGCATAGGGAGG | |
| | | | GTTCCCCCATCCCACCAACACCTTTGTAACCACACTGAAGCC | |
| | | | TTTAATCGAACCTCTGAGAGTCAGTATCTGCTACCTGGTTAT | |
| | | | GACAGGTGGTGGGCATGTAATACTGGATTAACCCCTTGTGTT | |
| | | | TCCACCTTGGTTTTCAACCAAACTAAAGACTTTTGTGTTATG | |
| | | | GTCCAAATTGTCCCCCGGGTGTACTACTATCCCGAAAAAGCA | |
| | | | GTCCTTGATGAATATGACTATAGATATAATCGGCCAAAAAGA | |
| | | | GAACCCATATCCCTGACACTAGCTGTAATGCTCGGATTGGGA | |
| | | | GTCGCTGCAGGCGTGGGAACAGGAACGGCTGCCCTAATCACA | |
| | | | GGACCACAACAGCTGGAGAAAGGACTTAGTGACCTACATCGA | |
| | | | ATTGTAACGGAAGATCTCCAAGCCCTAGAAAAATCTGTCAGT | |
| | | | AACCTAGAGGAATCCCTAACCTCCTTATCTGAAGTGGTTCTA | |
| | | | CACAACAGAAGGGGGTTAGATCTGTTATTTCTAAAAGAAGGT | |
| | | | GGGTTATGTGTAGCCTTAAAAGAAGAATGTTGCTTCTATGTA | |
| | | | GATCACTCAGGAGCCATCAGAGACTCCATGAGCAAGCTTAGA | |
| | | | GAAAGGTTAGAGAGGCGTCGAAGGGAAACAGACGCTGACCAG | |
| | | | GGGTGGTTTGAAGGATGGTTCAACAGGTCTCCTTGGATGACC | |
| | | | ACCCTGCTTTCTGCTCTGACGGGACCCCTAGTAGTCCTGCTC | |
| | | | CTGTTACTTACAGTTGGGCCTTGCTTAATTAATAGGTTTGTT | |
| | | | GCCTTTGTTAGAGAACGAGTGAGTGCAGTCCAGATCATGCTA | |
| | | | CTTAGGCAACAGTACCAAGGCCTTCTGAGCCAAGGAGAAACT | |
| | | | GACCTCTAGTAG | |
| B | B | A | ATGCATCCCACGTTAAGCTGGCGCCACCTCCCGACTCGGGGT | SEQ ID NO:19 |
| | | | GGAGAGCCGAAAAGACTGAGAATCCCCTTAAGCTTCGCCTCC | |
| | | | GGAGAGCCGAAAAGACTGAGAATCCCCTTAAGCTTCGCCTCC | |
| | | | AGTAAACGCCTTATAGACAGCTCGAACCCCCATAGACCTTTA | |
| | | | TCCCTTACCTGGCTGATTATTGACCCTGATACGGGTGTCACT | |
| | | | GTAAATAGCACTCGAGGTGTTGCTCCTACAGGCACCTGGTCG | |
| | | | CCTGAACTGCATTTCTGCCTCCGATTCATTAACCCCGCTGTT | |
| | | | AAAAGCACACCTCCCAACCTAGTCCGTAGTTATGGCTTCTAT | |
| | | | TGCTGCCCAGGCACAGAGAAAGAGAAATACTGTGGGGGTTCT | |
| | | | GGGGAATCCTTCTGTAGGAGATGGAGCTGCGTCACCTCCAAC | |
| | | | GATGGAGACTGGAAATGGCCGATCTCTCTCCAGGACCGGGTA | |
| | | | AAATTCTCCTTTGTCAATTCCGGCCCGGGCAAGTACAAAGTG | |
| | | | ATGAAACTATATAAAGATAAGACCTGCTCCCCATCAGACTTA | |
| | | | GATTATCTAAAGATAAGTTTCACTGAAAAAGGAAAACAGGAA | |
| | | | AATATTCAAAAGTGGATAAATGGTATGAGCTGGGGAATAGTT | |
| | | | TTTTATAAATATGGCGGGGGAGCAGGGTCCACTTTAACCATT | |
| | | | CGCCTTAGGATAGAGACGGGGACAGAACCCCCTGTGGCAGTG | |
| | | | GGACCCGATAAAGTACTGGCTGAACAGGGGCCCCCGGCCCTG | |
| | | | GAGCCACCGCATAACTTGCCGGTGCCCCAATTAACCTCGCTG | |
| | | | CGGCCTGACATAACACAGCCGCCTACCAACGGTACCACTGGA | |
| | | | TTGATTCCTACCAACACGCCTAGAAACTCCCCAGGTGTTCCT | |
| | | | GTTAAGACAGGACAGAGACTCTTCAGTCTCATCCAGGGAGCT | |
| | | | TTCCAAGCCATCAACTCCACCGACCCTGATGCCACTTCTTCT | |
| | | | TGTTGGCTTTGTCTATCCTCAGGGCCTCCTTATTATGAGGGG | |
| | | | ATGGCTAAAGAAGGAAAATTCAATGTGACCAAAGAGCATAGA | |
| | | | AATCAATGTACATGGGGGTCCCGAAATAAGCTTACCCTCACT | |
| | | | GAAGTTTCCGGGAAGGGGACATGCATAGGAAAAGCTCCCCCA | |
| | | | TCCCACCAACACCTTTGCTATAGTACTGTGGTTTATGAGCAG | |
| | | | GCCTCAGAAAATCAGTATTTAGTACCTGGTTATAACAGGTGG | |
| | | | TGGGCATGCAATACTGGGTTAACCCCCTGTGTTTCCACCTCA | |
| | | | GTCTTCAACCAATCCAAAGATTTCTGTGTCATGGTCCAAATC | |
| | | | GTCCCCCGAGTGTACTACCATCCTGAGGAAGTGGTCCTTGAT | |
| | | | GAATATGACTATCGGTATAACCGACCAAAAAGAGAACCCGTA | |
| | | | TCCCTTACCCTAGCTGTAATGCTCGGATTAGGGACGGCCGTT | |
| | | | GGCGTAGGAACAGGGACAGCTGCCCTGATCACAGGACCACAG | |
| | | | CAGCTAGAGAAAGGACTTGGTGAGCTACATGCGGCCATGACA | |
| | | | GAAGATCTCCGAGCCTTAGAGGAGTCTGTTAGCAACCTAGAA | |
| | | | GAGTCCCTGACTTCTTTGTCTGAAGTGGTTCTACAGAACCGG | |
| | | | AGGGGATTAGATCTGCTGTTTCTAAGAGAAGGTGGGTTATGT | |
| | | | GCAGCCTTAAAAGAAGAATGTTGCTTCTATGTAGATCACTCA | |
| | | | GGAGCCATCAGAGACTCCATGAGCAAGCTTAGAGAAAGGTTA | |
| | | | GAGAGGCGTCGAAGGGAAAGAGAGGCTGACCAGGGGTGGTTT | |
| | | | GAAGGATGGTTCAACAGGTCTCCTTGGATGACCACCCTGCTT | |
| | | | TCTGCTCTGACGGGACCCCTAGTAGTCCTGCTCCTGTTACTT | |
| | | | ACAGTTGGGCCTTGCTTAATTAATAGGTTTGTTGCCTTTGTT | |
| | | | AGAGAACGAGTGAGTGCAGTCCAGATCATGGTACTTAGGCAA | |
| | | | CAGTACCAAGGCCTTCTGAGCCAAGGAGAAACTGACCTCTAG | |
| | | | TAG | |
| B | B | N | ATGCATCCCACGTTAAGCTGGCGCCACCTCCCGACTCGGGGT | SEQ ID NO:21 |
| | | | ATGCATCCCACGTTAAGCTGGCGCCACCTCCCGACTCGGGGT | |
| | | | ATCGCCTGGTTCCTTACTCTAACAATAACTCCCCAGGCCAGT | |
| | | | AGTAAACGCCTTATAGACAGCTCGAACCCCCATAGACCTTTA | |
| | | | TCCCTTACCTGGCTGATTATTGACCCTGATACGGGTGTCACT | |
| | | | GTAAATAGCACTCGAGGTGTTGCTCCTAGAGGCACCTGGTGG | |
| | | | CCTGAACTGCATTTCTGCCTCCGATTGATTAACCCCGCTGTT | |
| | | | AAAAGCACACCTCCCAACCTAGTCCGTAGTTATGGGTTCTAT | |
| | | | TGCTGCCCAGGCACAGAGAAAGAGAAATACTGTGGGGGTTCT | |
| | | | GGGGAATCCTTCTGTAGGAGATGGAGCTGCGTCACCTCCAAC | |
| | | | GATGGAGACTGGAAATGGCCGATCTCTCTCCAGGACCGGGTA | |
| | | | AAATTCTCCTTTGTCAATTCCGGCCCGGGCAAGTACAAAGTG | |
| | | | ATGAAACTATATAAAGATAAGAGCTGCTCCCCATCAGACTTA | |
| | | | GATTATCTAAAGATAAGTTTCACTGAAAAAGGAAAACAGGAA | |
| | | | AATATTCAAAAGTGGATAAATGGTATGAGCTGGGGAATAGTT | |
| | | | TTTTATAAATATGGCGGGGGAGCAGGGTCCACTTTAACCATT | |
| | | | CGCCTTAGGATAGAGACGGGGACAGAACCCCCTGTGGCAGTG | |
| | | | GGACCCGATAAAGTACTGGCTGAACAGGGGCCCCCGGCCCTG | |
| | | | GAGCCACCGCATAACTTGCCGGTGCCCCAATTAACCTCGCTG | |
| | | | CGGCCTGACATAACACAGCCGCCTAGCAACAGTACCACTGGA | |
| | | | TTGATTCCTACCAACACGCCTAGAAACTCCCCAGGTGTTCCT | |
| | | | GTTAAGACAGGACAGAGACTCTTCAGTCTCATCCAGGGAGCT | |
| | | | TTCCAAGCCATCAACTCCACCGACCCTGATGCCACTTCTTCT | |
| | | | TGTTGGCTTTGTCTATCCTCAGGGCCTCCTTATTATGAGGGG | |
| | | | ATGGCTAAAGAAGGAAAATTCAATGTGACCAAAGAGCATAGA | |
| | | | AATCAATGTACATGGGGGTCCCGAAATAAGCTTACCCTCACT | |
| | | | GAAGTTTCCGGGAAGGGGACATGCATAGGAAAAGCTCCCCCA | |
| | | | TCCCACCAACACCTTTGCAATAGTACTGTGGTTTATGAGCAG | |
| | | | GCCTCAGAAAATCAGTATTTAGTACCTGGTTATAACAGGTCC | |
| | | | TGGGCATGCAATACTGGGTTAACCCCCTGTGTTTCCACCTCA | |
| | | | GTCTTCAACCAATCCAAAGATTTCTGTGTCATGGTCCAAATC | |
| | | | GTCCCCCGAGTGTACTACCATCCTGAGGAAGTGGTCCTTGAT | |
| | | | GAATATGACTATCGGTATAACCGACCAAAAAGAGAACCCGTA | |
| | | | TCCCTTACCCTAGCTGTAATGCTCGGATTAGGGACGGCCGTT | |
| | | | GGCGTAGGAACAGGGACAGCTGCCCTGATCACAGGACCACAG | |
| | | | CAGCTAGAGAAAGGACTTGGTGAGCTACATGCGGCCATGACA | |
| | | | GAAGATCTCCGAGCCTTAGAGGAGTCTGTTAGCAACCTAGAA | |
| | | | GAGTCCCTGACTTCTTTGTCTGAAGTGGTTCTACAGAACCGG | |
| | | | AGGGGATTAGATCTGCTGTTTCTAAGAGAACGTGGGTTATGT | |
| | | | GCAGCCTTAAAAGAAGAATGTTGCTTCTATGTAGATCACTCA | |
| | | | GGAGCCATCAGAGACTCCATGAGCAAGCTTAGAGAAAGGTTA | |
| | | | GAGAGGCGTCGAAGCCAAAGAGAGGCTGACCAGGGGTGGTTT | |
| | | | GAAGGATGGTTCAACAGGTCTCCTTGGATGACCACCCTGCTT | |
| | | | TCTGCTCTGACGGGGCCCCTAGTAGTCCTGCTCCTGTTACTT | |
| | | | ACAGTTGGGCCTTGCTTAATTAATAGGTTTGTTGCCTTTGTT | |
| | | | AGAGAACGAGTGAGTGCAGTCCAGATCATGGTACTTAGGCAA | |
| | | | CAGTACCAAGGCCTTCTGAGCCAAGGAGAAACTGACCTCTAC | |
| | | | TAG | |
| C | B | A | ATGCATCCCACGTTAAGCTGGCGCCACCTCCCGACTCGGGGT | SEQ ID NO:23 |
| | | | ATGCATCCCACGTTAAGCTGGCGCCACCTCCCGACTCGGGGT | |
| | | | ATCGCCTGGTTCCTTACTCTAACAATAACTCCCCAGGCCAGT | |
| | | | AGTAAACGCCTTATAGACAGCTCGAACCCCCATAGACCTTTA | |
| | | | TCCCTTACCTGGCTGATTATTGACCCTGATACGGGTGTCACT | |
| | | | GTAAATAGCACTCGAGGTGTTGCTCCTAGAGGCACCTGGTGG | |
| | | | CCTGAACTGCATTTCTGCCTCCGATTGATTAACCCCGCTGTT | |
| | | | AAAGACCAGAGCACACCTCCCAACCTAGTCCGTAGTTATGGG | |
| | | | TTCTATTGCTGCCCAGGCACACCAGAGAAAGAGAAATACTGT | |
| | | | GGCGGTTCTGGGGAATCCTTCTGTAGGAGATGGAGCTGCGTC | |
| | | | ACCTCCAACGATGGAGACTGGAAATGGCCGATCTCTCTCCAG | |
| | | | GACCGGGTAAAATTCTCCTTTGTCAATTCCGGCCCGGGCTAT | |
| | | | AATCAATTTAATTATGGCCATGGGAGATGGAAAGATTGGAAG | |
| | | | TACAAAGTGATGAAACTATATAAAGATAAGCAAATAAGCTGC | |
| | | | TCCCCATCAGACTTAGATTATCTAAAGATAAGTTTCACTGAA | |
| | | | AAAGGAAAACAGGAAAATATTCAAAAGTGGATAAATGGTATG | |
| | | | AGCTGGGGAATAGTTTTTTATAAATATGGCGGGGCAAAGGCA | |
| | | | GGGTCCACTTTAACCATTCGCCTTAGGATAGAGACGCGGACA | |
| | | | GAACCCCCTGTGGCAGTGGGACCCGATAAAGTACTGGCTGAA | |
| | | | CAGGGGCCCCCGGCCCTGGAGCCACCGCATAACTTGCCGGTG | |
| | | | CCCCAATTAACCTCGCTGCGGCCTGACATAACACAGCCGCCT | |
| | | | AGCAACGGTACCACTGGATTGATTCCTACCAACACGCCTAGA | |
| | | | AACTCCCCAGGTGTTCCTCTTAAGACAGGACAGAGACTCTTC | |
| | | | AGTCTCATCCAGGGAGCTTTCCAAGCCATCAACTCCACCGAC | |
| | | | CCTGATGCCACTTCTTCTTGTTGGCTTTGTCTATCCTCAGGG | |
| | | | GTGACCAAAGAGCATAGAAATCAATGTACATGGGGGTCCCGA | |
| | | | GTGACCAAAGAGCATAGAAATCAATGTACATGGGGGTCCCGA | |
| | | | AATAAGCTTACCCTCACTGAAGTTTCCGGGAAGGGGACATGC | |
| | | | ATAGGAAAAGCTCCCCCATCCCACCAACACCTTTGCTATAGT | |
| | | | ACTGTGGTTTATGAGCAGGCCTCAGAAAATCAGTATTTAGTA | |
| | | | CCTGGTTATAACAGGTGGTGGGCATGCAATACTGGGTTAACC | |
| | | | CCCTGTGTTTCCACCTCAGTCTTCAACCAATCCAAAGATTTC | |
| | | | TGTGTCATGGTCCAAATCCTCCCCCGAGTGTACTACCATCCT | |
| | | | GAGGAAGTGGTCCTTGATGAATATGACTATCGGTATAACCGA | |
| | | | CCAAAAAGAGAACCCGTATCCCTTACCCTAGCTGTAATGCTC | |
| | | | GGATTAGGGACGGCCGTTGGCGTAGGAACAGGGACAGCTGCC | |
| | | | CTGATCACAGGACCACAGCAGCTAGAGAAAGGACTTGGTGAG | |
| | | | CTACATGCGGCCATGACAGAAGATCTCCGAGCCTTAGAGGAG | |
| | | | TCTGTTAGCAACCTAGAAGAGTCCCTGACTTCTTTGTCTGAA | |
| | | | GTGGTTCTACAGAACCGGAGGGGATTAGATCTGCTGTTTCTA | |
| | | | AGAGAAGGTGGGTTATGTGCAGCCTTAAAAGAAGAATGTTGC | |
| | | | TTCTATGTAGATCACTCAGGAGCCATCAGAGACTCCATGAGC | |
| | | | AAGCTTAGAGAAAGGTTAGAGAGGCGTCGAAGGGAAAGAGAG | |
| | | | GCTGACCAGGGGTGGTTTGAAGGATGGTTCAACAGGTCTCCT | |
| | | | TGGATGACCACCCTGCTTTCTGCTCTGACGGGACCCCTAGTA | |
| | | | GTCCTGCTCCTGTTACTTACAGTTGGGCCTTGCTTAATTAAT | |
| | | | AGGTTTGTTGCCTTTGTTAGAGAACGAGTGAGTGCAGTCCAG | |
| | | | ATCATGGTACTTAGGCAACAGTACCAAGGCCTTCTGAGCCAA | |
| | | | GGAGAAACTGACCTCTAC | |
| C | B | N | ATGCATCCCACGTTAAGCTGGCGCCACCTCCCGACTCGGGGT | SEQ ID NO:25 |
| | | | GGAGAGCCGAAAAGACTGAGAATCCCCTTAAGCTTCGCCTCC | |
| | | | ATCGCCTGGTTCCTTACTCTAACAATAACTCCCCAGGCCAGT | |
| | | | AGTAAACGCCTTATAGACAGCTCGAACCCCCATAGACCTTTA | |
| | | | TCCCTTACCTGGCTGATTATTGACCCTGATACGGGTGTCACT | |
| | | | GTAAATAGCACTCGAGGTGTTGCTCCTAGAGGCACCTGGTGG | |
| | | | CCTGAACTGCATTTCTGCCTCCGATTGATTAACCCCGCTGTT | |
| | | | AAAGACCAGAGCACACCTCCCAACCTAGTCCGTAGTTATGCG | |
| | | | TTCTATTGCTGCCCAGGCACACCAGAGAAAGAGAAATACTGT | |
| | | | GGGGGTTCTGCGGAATCCTTCTGTAGGAGATGGAGCTGCGTC | |
| | | | ACCTCCAACGATGGAGACTGGAAATGGCCGATCTCTCTCCAG | |
| | | | GACCGGGTAAAATTCTCCTTTGTCAATTCCGGCCCGCGCTAT | |
| | | | AATCAATTTAATTATGGCCATGGGAGATGGAAAGATTGGAAG | |
| | | | TACAAACTGATGAAACTATATAAAGATAAGCAAATAAGCTGC | |
| | | | TCCCCATCAGACTTAGATTATCTAAAGATAAGTTTCACTGAA | |
| | | | AAAGGAAAACAGCAAAATATTCAAAAGTGGATAAATGGTATG | |
| | | | AGCTGGGGAATAGTTTTTTATAAATATGGCGGGGGAAGGGCA | |
| | | | GGGTCCACTTTAACCATTCGCCTTAGGATAGAGACGGGGACA | |
| | | | GAACCCCCTGTGGCAGTGGGACCCGATAAAGTACTGGCTGAA | |
| | | | CAGGGGCCCCCGGCCCTGGAGCCACCGCATAACTTGCCGGTG | |
| | | | CCCCAATTAACCTCGCTGCGGCCTGACATAACACAGCCGCCT | |
| | | | AGCAACAGTACCACTGGATTGATTCCTACCAACACGCCTAGA | |
| | | | AACTCCCCAGGTGTTCCTGTTAAGACAGGACAGAGACTCTTC | |
| | | | AGTCTCATCCAGGGAGCTTTCCAAGCCATCAACTCCACCGAC | |
| | | | CCTGATGCCACTTCTTCTTGTTGGCTTTGTCTATCCTCAGGG | |
| | | | CCTCCTTATTATGAGGGGATGGCTAAAGAAGGAAAATTCAAT | |
| | | | GTGACCAAAGAGCATAGAAATCAATGTACATGGGGGTCCCGA | |
| | | | AATAAGCTTACCCTCACTGAAGTTTCCGGGAAGGGGACATGC | |
| | | | ATAGGAAAAGCTCCCCCATCCCACCAACACCTTTGCAATAGT | |
| | | | ACTGTGGTTTATGAGCAGGCCTCAGAAAATCAGTATTTAGTA | |
| | | | CCTGGTTATAACAGGTGCTGGGCATGCAATACTGGGTTAACC | |
| | | | CCCTGTGTTTCCACCTCAGTCTTCAACCAATCCAAAGATTTC | |
| | | | TGTGTCATGGTCCAAATCGTCCCCCGAGTGTACTACCATCCT | |
| | | | GAGGAAGTGGTCCTTGATGAATATGACTATCGGTATAACCGA | |
| | | | CCAAAAAGAGAACCCGTATCCCTTACCCTAGCTGTAATGCTC | |
| | | | GGATTAGCGACGGCCGTTGGCGTAGGAACACGGACAGCTGCC | |
| | | | CTGATCACAGGACCACAGCAGCTAGAGAAAGCACTTGGTGAG | |
| | | | CTACATGCGGCCATGACAGAAGATCTCCGAGCCTTAGAGGAG | |
| | | | TCTGTTAGCAACCTAGAAGAGTCCCTGACTTCTTTGTCTGAA | |
| | | | GTGGTTCTACAGAACCGGAGGGGATTAGATCTGCTGTTTCTA | |
| | | | AGAGAAGGTGGGTTATGTGCAGCCTTAAAAGAAGAATGTTGC | |
| | | | TTCTATGTAGATCACTCAGGAGCCATCAGAGACTCCATGAGC | |
| | | | AAGCTTAGAGAAAGGTTAGAGAGCCGTCGAAGGGAAAGAGAG | |
| | | | GCTGACCAGGGGTGGTTTGAAGGATGGTTCAACAGGTCTCCT | |
| | | | TGGATGACCACCCTGCTTTCTGCTCTGACGGGGCCCCTAGTA | |
| | | | GTCCTGCTCCTGTTACTTACAGTTGGGCCTTGCTTAATTAAT | |
| | | | AGGTTTGTTGCCTTTGTTAGAGAACGAGTGAGTGCAGTCCAG | |
| | | | ATCATGGTACTTAGGCAACAGTACCAAGGCCTTCTGAGCCAA | |
| | | | GGAGAAACTGACCTCTAC | |
| N | B | A | ATGCATCCCACGTTAAGCTGGCGCCACCTCCCGACTCGGGGT | SEQ ID NO:27 |
| | | | GGAGAGCCGAAAAGACTGAGAATCCCCTTAAGCTTCGCCTCC | |
| | | | ATCGCCTGGTTCCTTACTCTAACAATAACTCCCCAGGCCAGT | |
| | | | AGTAAACGCCTTATAGACAGCTCGAACCCCCATAGACCTTTA | |
| | | | TCCCTTACCTGGCTGATTATTGACCCTGATACGGGTCTCACT | |
| | | | GTAAATAGCACTCGAGGTGTTGCTCCTAGAGGCACCTGGTGG | |
| | | | CCTGAACTGCATTTCTGCCTCCGATTGATTAACCCCGCTGTT | |
| | | | AAAAGCACACCTCCCAACCTACTCCGTAGTTATGGGTTCTAT | |
| | | | TGCTGCCCAGGCACAGAGAAAGAGAAATACTGTGGGGGTTCT | |
| | | | GGGCAATCCTTCTGTAGGAGATGGAGCTGCGTCACCTCCAAC | |
| | | | GATGGAGACTGGAAATGCCCGATCTCTCTCCAGGACCGGGTA | |
| | | | AAATTCTCCTTTGTCAATTCCGGCCCGGGCAAGTACAAAGTG | |
| | | | ATGAAACTATATAAAGATAACAGCTGCTCCCCATCAGACTTA | |
| | | | GATTATCTAAAGATAAGTTTCACTGAAAAAGGAAAACAGGAA | |
| | | | AATATTCAAAAGTGGATAAATGGTATGAGCTGGGGAATAGTT | |
| | | | TTTTATAAATATGGCGGGGGAGCAGGGTCCACTTTAACCATT | |
| | | | CGCCTTAGGATAGAGACGGGGACAGAACCCCCTGTGGCAGTG | |
| | | | GGACCCGATAAAGTACTGGCTCAACAGGGGCCCCCGGCCCTG | |
| | | | GAGCCACCGCATAACTTGCCGGTGCCCCAATTAACCTCGCTG | |
| | | | CGGCCTGACATAACACAGCCGCCTAGCAACGGTACCACTGGA | |
| | | | TTGATTCCTACCAACACGCCTAGAAACTCCCCAGGTGTTCCT | |
| | | | GTTAAGACAGGACAGACACTCTTCAGTCTCATCCAGCGAGCT | |
| | | | TTCCAAGCCATCAACTCCACCGACCCTGATGCCACTTCTTCT | |
| | | | TGTTGGCTTTGTCTATCCTCAGGGCCTCCTTATTATGAGGGG | |
| | | | ATGGCTAAAGAAGGAAAATTCAATGTGACCAAAGAGCATAGA | |
| | | | AATCAATGTACATGGGGGTCCCGAAATAAGCTTACCCTCACT | |
| | | | GAAGTTTCCGGGAAGGGGACATGCATAGGAAAAGCTCCCCCA | |
| | | | TCCCACCAACACCTTTGCTATAGTACTGTGGTTTATGAGCAG | |
| | | | GCCTCAGAAAATCAGTATTTAGTACCTGGTTATAACAGGTGG | |
| | | | TGGGCATGCAATACTGGGTTAACCCCCTGTGTTTCCACCTCA | |
| | | | GTCTTCAACCAATCCAAAGATTTCTGTGTCATGGTCCAAATC | |
| | | | GTCCCCCGAGTGTACTACCATCCTGAGGAAGTGGTCCTTGAT | |
| | | | GAATATGACTATCGGTATAACCGACCAAAAAGAGAACCCGTA | |
| | | | TCCCTTACCCTAGCTGTAATGCTCGGATTAGGGACGGCCGTT | |
| | | | GGCGTAGGAACAGGGACAGCTGCCCTGATCACAGGACCACAG | |
| | | | CAGCTAGAGAAAGGACTTGGTGAGCTACATGCGGCCATGACA | |
| | | | GAAGATCTCCGAGCCTTAGAGGAGTCTGTTAGCAACCTAGAA | |
| | | | GAGTCCCTGACTTCTTTGTCTGAAGTGGTTCTACACAAcCGG | |
| | | | AGGGGATTAGATCTGCTGTTTCTAAGAGAAGGTGGGTTATGT | |
| | | | GCAGCCTTAAAAGAAGAATGTTGCTTCTATGTAGATCACTCA | |
| | | | GGAGCCATCAGAGACTCCATGAGCAAGCTTAGAGAAAGGTTA | |
| | | | GAGAGGCGTCGAAGGGAAAGAGAGGCTGACCAGGGGTGGTTT | |
| | | | GAAGGATGGTTCAACAGGTCTCCTTGGATGACCACCCTGCTT | |
| | | | TCTGCTCTGACGGGACCCCTAGTAGTCCTGCTCCTGTTACTT | |
| | | | ACAGTTGGGCCTTGCTTAATTAATAGGTTTGTTGCCTTTGTT | |
| | | | AGAGAACGAGTGAGTGCAGTCCAGATCATGGTACTTAGGCAA | |
| | | | CAGTACCAAGGCCTTCTGAGCCAAGGAGAAACTGACCTCTAG | |
| | | | TAG | |
| N | B | N | ATGCATCCCACGTTAAGCTGGCGCCACCTCCCGACTCGGGGT | SEQ ID NO:29 |
| | | | GGAGAGCCGAAAAGACTGAGAATCCCCTTAAGCTTCGCCTCC | |
| | | | ATCGCCTGGTTCCTTACTCTAACAATAACTCCCCAGGCCAGT | |
| | | | AGTAAACGCCTTATAGACAGCTCGAACCCCCATAGACCTTTA | |
| | | | TCCCTTACCTGGCTGATTATTGACCCTGATACGGGTGTCACT | |
| | | | GTAAATAGCACTCGAGGTGTTGCTCCTAGAGGCACCTGGTGG | |
| | | | CCTGAACTGCATTTCTGCCTCCGATTGATTAACCCCGCTGTT | |
| | | | AAAAGCACACCTCCCAACCTAGTCCGTAGTTATGGGTTCTAT | |
| | | | TGCTGCCCAGGCACAGAGAAAGAGAAATACTGTGGGGGTTCT | |
| | | | GGGGAATCCTTCTGTAGGAGATGGAGCTGCGTCACCTCCAAC | |
| | | | GATGGAGACTGGAAATGGCCGATCTCTCTCCAGGACCGGGTA | |
| | | | AAATTCTCCTTTGTCAATTCCGGCCCGGGCAAGTACAAAGTG | |
| | | | ATGAAACTATATAAAGATAAGAGCTGCTCCCCATCAGACTTA | |
| | | | GATTATCTAAAGATAAGTTTCACTGAAAAAGGAAAACAGGAA | |
| | | | AATATTCAAAAGTGGATAAATGGTATGAGCTGGGGAATAGTT | |
| | | | TTTTATAAATATGGCGGGGGAGCAGGGTCCACTTTAACCATT | |
| | | | CGCCTTAGGATAGAGACGGGGACAGAACCCCCTGTGGCAGTG | |
| | | | GGACCCGATAAAGTACTGGCTGAACAGGGGCCCCCGGCCCTG | |
| | | | GAGCCACCGCATACTTGCCGGTGCCCCCAATTAACCTCGCTG | |
| | | | CGGCCTGACATAACACAGCCGCCTAGCAACAGTACCACTGGA | |
| | | | TTGATTCCTACCAACACGCCTAGAAACTCCCCAGGTGTTCCT | |
| | | | GTTAAGACAGGACAGAGACTCTTCAGTCTCATCCAGGGAGCT | |
| | | | TTCCAAGCCATCAACTCCACCGACCCTGATGCCACTTCTTCT | |
| | | | TGTTGGCTTTGTCTATCCTCAGGGCCTCCTTATTATGAGGGG | |
| | | | ATGGCTAAAGAAGGAAAATTCAATGTGACCAAAGAGCATAGA | |
| | | | AATCAATGTACATGGGGGTCCCGAAATAAGCTTACCCTCACT | |
| | | | GAAGTTTCCGGGAAGGGGACATGCATAGGAAAAGCTCCCCCA | |
| | | | TCCCACCAACACCTTTGCAATAGTACTGTGGTTTATGAGcAG | |
| | | | GCCTCAGAAAATCAGTATTTAGTACCTGGTTATAACAGGTGG | |
| | | | TGGGCATGCAATACTGGGTTAACCCCCTGTGTTTCCACCTCA | |
| | | | GTCTTCAACCAATCCAAAGATTTCTGTGTCATGGTCCAAATC | |
| | | | GTCCCCCGAGTGTACTACCATCCTGAGGAAGTGGTCCTTGAT | |
| | | | GAATATGACTATCGGTATAACCGACCAAAAAGAGAACCCGTA | |
| | | | TCCCTTACCCTAGCTGTAATGCTCGGATTAGGGACGGCCGTT | |
| | | | GGCGTAGGAACAGGGACAGCTGCCCTGATCACAGGACCACAG | |
| | | | CAGCTAGAGAAAGGACTTGGTGAGCTACATGCGGCCATGACA | |
| | | | GAAGATCTCCGAGCCTTAGAGGAGTCTGTTAGCAACCTAGAA | |
| | | | TCCCTTACCCTAGCTGTAATGCTCGGATTAGGGACGGCCGTT | |
| | | | AGGGGATTAGATCTGCTGTTTCTAAGAGAAGGTGGGTTATGT | |
| | | | GCAGCCTTAAAAGAAGAATGTTGCTTCTATGTAGATCACTCA | |
| | | | GGAGCCATCAGAGACTCCATGAGCAAGCTTAGAGAAAGGTTA | |
| | | | GAGAGGCGTCGAAGGGAAAGAGACGCTGAcCAGGGGTGGTTT | |
| | | | GAAGGATGGTTCAACAGGTCTCCTTCGATGACCACCCTGCTT | |
| | | | TCTGCTCTGACGGGGCCCCTAGTACTCCTCCTCCTGTTACTT | |
| | | | ACAGTTGGGCCTTGCTTAATTAATAGGTTTGTTGCCTTTGTT | |
| | | | AGAGAACGAGTGAGTGCAGTCCAGATCATGGTACTTAGGCAA | |
| | | | CACTACCAAGGCCTTCTGAGCCAAGGAGAAACTGACCTCTAG | |
| | | | TAG | |
| B | C | A | ATGCATCCCACGTTAAGCCGGCGCCACCTCCCGATTCGGGGT | SEQ ID NO:31 |
| | | | GGAAAGCCGAAAAGACTGAAAATCCCCTTAAGCTTCGCCTCC | |
| | | | ATCGCGTGGTTCCTTACTCTGTCAATAACCTCTCAGACTAAT | |
| | | | GGTATGCGCATAGGAGACAGCCTGAACTCCCATAAACCCTTA | |
| | | | TCTCTCACCTGGTTAATTACTGACTCCGGCACAGGTATTAAT | |
| | | | ATCAACAACACTCAAGGGGAGGCTCCTTTAGGAACCTGGTGG | |
| | | | CCTGATCTATACGTTTGCCTCAGATCAGTTATTCCTAGTCTG | |
| | | | ACCTCACCCCCAGATATCCTCCATGCTCACGGATTTTATGTT | |
| | | | TGCCCAGGACCACCAAATAATGGAAAACATTGCGGAAATCCC | |
| | | | AGAGATTTCTTTTGTAAACAATGGAACTGTGTAACCTCTAAT | |
| | | | GATGGATATTGGAAATGGCCAACCTCTCAGCAGGATAGGGTA | |
| | | | AGTTTTTCTTATGTCAACACCTATACCAGCTCTGGACAATTT | |
| | | | AATTACCTGACCTGGATTAGAACTGGAAGCCCCAAGTGCTCT | |
| | | | CCTTCAGACCTAGATTACCTAAAAATAAGTTTCACTGAGAAA | |
| | | | GGAAAACAAGAAAATATCCTAAAATGGGTAAATGGTATGTCT | |
| | | | TGGGGAATGGTATATTATGGAGGCTCGGGTAAACAACCAGGC | |
| | | | TCCATTCTAACTATTCGCCTCAAAATAAACCAGCTGGAGCCT | |
| | | | CCAATGGCTATAGGACCAAATACGGTCTTGACGGGTCAAAGA | |
| | | | CCCCCAACCCAAGGACCAGGACCATCCTCTAACATAACTTCT | |
| | | | GGATCAGACCCCACTGAGTCTAACAGCACGACTAAAATGGGG | |
| | | | GCAAAACTTTTTAGCCTCATCCAGGGAGCTTTTCAAGCTCTT | |
| | | | AACTCCACGACTCCAGAGGCTACCTCTTCTTGTTGGCTATGC | |
| | | | TTAGCTTTGGGCCCACCTTACTATGAAGGAATGGCTAGAAGA | |
| | | | GGGAAATTCAATGTGACAAAAGAACATAGAGACCAATGCACA | |
| | | | TGGGGATCCCAAAATAAGCTTACCCTTACTGAGGTTTCTGGA | |
| | | | AAAGGCACCTGCATAGGAAAGGTTCCCCCATCCCACCAACAC | |
| | | | CTTTGTAACCACACTGAAGCCTTTAATCAAACCTCTGAGAGT | |
| | | | CAATATCTGGTACCTGGTTATGACAGGTGGTGGGCATGTAAT | |
| | | | ACTGGATTAACCCCTTGTGTTTCCACCTTGGTTTTTAACCAA | |
| | | | ACTAAAGATTTTTGCATTATGGTCCAAATTGTTCCCCGAGTG | |
| | | | TATTACTATCCCGAAAAAGCAATCCTTGATGAATATGACTAC | |
| | | | AGAAATCATCGACAAAAGAGAGAACCCATATCTCTGACACTT | |
| | | | GCTGTGATGCTCGGACTTGGAGTGGCAGCAGGTGTAGGAACA | |
| | | | GGAACAGCTGCCCTGGTCACGGGACCACAGCAGCTAGAAACA | |
| | | | GGACTTAGTAACCTACATCGAATTGTAACAGAAGATCTCCAA | |
| | | | GCCCTAGAAAAATCTGTCAGTAACCTGGAGGAATCCCTAACC | |
| | | | TCCTTATCTGAAGTAGTCCTACAGAATAGAAGAGGGTTAGAT | |
| | | | TTATTATTTCTAAAAGAAGGAGGATTATGTGTAGCCTTGAAG | |
| | | | GAGGAATGCTGTTTTTATGTGGATCATTCAGGGGCCATCAGA | |
| | | | GACTCCATGAACAAACTTAGAGAAAGGTTGGAGAAGCGTCGA | |
| | | | AGGGAAAAGGAAACTACTCAAGGGTGGTTTGAGGGATGGTTC | |
| | | | AACAGGTCTCCTTGGTTGGCTACCCTACTTTCTGCTTTAACA | |
| | | | GGACCCTTAATAGTCCTCCTCCTGTTACTCACAGTTGGGCCA | |
| | | | TGTATTATTAACAAGTTAATTGCCTTCATTAGAGAACGAATA | |
| | | | AGTGCAGTCCAGATCATGGTACTTAGACAACAGTACCAAAGC | |
| | | | CCGTCTAGCAGGGAAGCTGGCCGCTAGTAGTAG | |
| B | C | N | ATGCATCCCACGTTAAGCCGGCGCCACCTCCCGATTCGGGGT | SEQ ID NO:33 |
| | | | GGAAAGCCGAAAAGACTGAAAATCCCCTTAAGCTTCGCCTCC | |
| | | | ATCGCGTGGTTCCTTACTCTGTCAATAACCTCTCAGACTAAT | |
| | | | GGTATGCGCATAGGAGACAGCCTGAACTCCCATAAACCCTTA | |
| | | | TCTCTCACCTGGTTAATTACTGACTCCGGCACAGGTATTAAT | |
| | | | ATCAACAACACTCAAGGGGAGGCTCCTTTAGGAACCTGGTGG | |
| | | | CCTGATCTATACGTTTGCCTCAGATCAGTTATTCCTAGTCTG | |
| | | | ACCTCACCCCCAGATATCCTCCATGCTCACGGATTTTATGTT | |
| | | | TGCCCAGGACCACCAAATAATGGAIAACATTGCGGAAATCCC | |
| | | | AGAGATTTCTTTTGTAAACAATGGAACTGTGTAACCTCTAAT | |
| | | | GATGGATATTGGAAATGGCCAACCTCTCAGCAGGATAGGGTA | |
| | | | AGTTTTTCTTATGTCAACACCTATACCAGCTCTGGACAATTT | |
| | | | AATTACCTGACCTGGATTAGAACTGGAAGCCCCAAGTGCTCT | |
| | | | CCTTCAGACCTAGATTACCTAAAAATAAGTTTCACTGAGAAA | |
| | | | GGAAAACAAGAAAATATCCTAAAATGGGTAAATCGTATGTCT | |
| | | | TGGGGAATGGTATATTATGGAGGCTCGGGTAAACAACCAGGC | |
| | | | TCCATTCTAACTATTCGCCTCAAAATAAACCAGCTGGAGCCT | |
| | | | CCAATGGCTATAGGACCAAATACGGTCTTGACGGGTCAAAGA | |
| | | | CCCCCAACCCAAGGACCAGGACCATCCTCTAACATAACTTCT | |
| | | | GGATCAGACCCCACTGAGTCTAACAGCACGACTAAAATGGGG | |
| | | | GCAAAACTTTTTAGCCTCATCCAGGGAGCTTTTCAAGCTCTT | |
| | | | AACTCCACGACTCCAGAGGCTACCTCTTCTTGTTGGCTATGC | |
| | | | TTAGCTTTGGGCCCACCTTACTATGAAGGAATGGCTAGAAGA | |
| | | | GGGAAATTCAATGTGACAAAAGAACATAGAGACCAATGCACA | |
| | | | TGGGGATCCCAAAATAAGCTTACCCTTACTGAGGTTTCTGGA | |
| | | | AAAGGCACCTGCATAGGAAAGCTTCCCCCATCCCACCAACAC | |
| | | | CTTTGTAACCACACTGAAGCCTTTAATCAAACCTCTGAGAGT | |
| | | | CAATATCTGGTACCTGGTTATGACAGGTGGTCGGCATGTAAT | |
| | | | ACTGGATTAACCCCTTGTGTTTCCACCTTGGTTTTTAACCAA | |
| | | | ACTAAAGATTTTTGCATTATGGTCCAAATTGTTCCCCGAGTG | |
| | | | TATTACTATCCCGAAAAAGCAATCCTTGATGAATATGACTAC | |
| | | | AGAAATCATCGACAAAAGAGAGAACCCATATCTCTGACACTT | |
| | | | GCTGTGATGCTCGGACTTGGACTGGCACCACGTGTAGGAACA | |
| | | | GGAACAGCTGCCCTGGTCACGGGACCACAGCAGCTAGAAACA | |
| | | | GGACTTAGTAACCTACATCGAATTGTAACAGAAGATCTCCAA | |
| | | | GCCCTAGAAAAATCTGTCAGTAACCTGGAGGAATCCCTAACC | |
| | | | TCCTTATCTGAAGTAGTCCTACAGAATAGAAGAGGGTTAGAT | |
| | | | TTATTATTTCTAAAAGAAGGAGGATTATGTGTAGCCTTGAAG | |
| | | | GAGGAATGCTGTTTTTATGTCCATCATTCAGGGGCCATCAGA | |
| | | | GACTCCATGAACAAACTTAGAGAAAGGTTGGAGAAGCGTCGA | |
| | | | AGGGAAAAGGAAACTACTCAAGGGTGGTTTGAGGGATGGTTC | |
| | | | AACACGTCTCCTTGGTTGGCTACCCTACTTTCTGCTTTAACA | |
| | | | GGACCCTTAATAGTCCTCCTCCTGTTACTCACAGTTGGGCCA | |
| | | | TGTATTATTAACAAGTTAATTGCCTTCATTAGAGAACGAATA | |
| | | | AGTGCAGTCCAGATCATGGTACTTAGACAACAGTACCAAAGC | |
| | | | CCGTCTAGCAGGGAAGCTGGCCGCTAGTAGTAG | |
| C | C | A | ATGCATCCCACGTTAAGCCGGCGCCACCTCCCGATTCCGGGT | SEQ ID NO:35 |
| | | | GGAAAGCCCAAAAGACTGAAAATCCCCTTAAGCTTCGCCTCC | |
| | | | ATCGCGTCGTTCCTTACTCTGTCAATAACCTCTCAGACTAAT | |
| | | | GGTATGCGCATAGGAGACAGCCTGAACTCCCATAAACCCTTA | |
| | | | TCTCTCACCTGGTTAATTACTGACTCCGGCACAGGTATTAAT | |
| | | | ATCAACAACACTCAAGGGGAGGCTCCTTTAGGAACCTGGTGG | |
| | | | CCTGATCTATACGTTTGCCTCAGATCAGTTATTCCTAGTCTG | |
| | | | AATGACCAGACCTCACCCCCAGATATCCTCCATGCTCACGGA | |
| | | | TTTTATGTTTGCCCAGGACCACCAAATAATGGAAAACATTGC | |
| | | | GGAAATCCCAGAGATTTCTTTTGTAAACAATGGAACTGTGTA | |
| | | | ACCTCTAATGATGGATATTGGAAATGCCCAACCTCTCAGCAG | |
| | | | GATAGGGTAAGTTTTTCTTATGTCAACACCTATACCAGCTCT | |
| | | | GGACAATTTAATTACGGCCATGGGAGATGGCTGACCTGGCAA | |
| | | | CACCGGGTACAAAAAGATATTAGAACTGCAACCCCCAAGTGC | |
| | | | TCTCCTTCACACCTAGATTACCTAAAAATAACTTTCACTGAG | |
| | | | AAAGGAAAACAACAAAATATCCTAAAATGCGTAAATGGTATG | |
| | | | TCTTGGGGAATGGTATATTATGGAGGCTCGGGTAAACAACCA | |
| | | | CGCTCCATTCTAACTATTCGCCTCAAAATAAACACTCAGCTG | |
| | | | GAGCCTCCAATGGCTATACGACCAAATACGGTCTTGACGGGT | |
| | | | CAAACACCCCCAACCCAAGCACCACCGCATAACTTGCCGGTG | |
| | | | CCCCAGGGACCATCCCCTAACCCCGACATAACACAGTCTGAT | |
| | | | TACAACATAACTTCTGGATCAGACCCCACTAACACGCCTAGA | |
| | | | AACGAGTCTAACAGCACGACTAAAATGGGGGCAAAACTTTTT | |
| | | | AGCCTCATCCAGGGAGCTTTTCAAGCTCTTAACTCCACCACT | |
| | | | CCAGACGCTACCTCTTCTTGTTGGCTATGCTTAGCTTTGGGC | |
| | | | CCACCTTACTATGAAGGAATGGCTAGAAGAGGGAAATTCAAT | |
| | | | GTGACAAAAGAACATAGAGACCAATGCACATGGGGATCCCAA | |
| | | | AATAAGCTTACCCTTACTGAGGTTTCTGGAAAAGGCACCTGC | |
| | | | ATAGGAAAGGTTCCCCCATCCCACCAACACCTTTGTAACCAC | |
| | | | ACTGAAGCCTTTAATCAAACCTCTGAGAGTCAATATCTGGTA | |
| | | | CCTGGTTATGACAGGTGGTGGGCATGTAATACTGGATTAACC | |
| | | | CCTTGTGTTTCCACCTTGGTTTTTAACCAAACTAAAGATTTT | |
| | | | TGCATTATGGTCCAAATTGTTCCCCCAGTGTATTACTATCCC | |
| | | | GAAAAAGCAATCCTTGATGAATATGACTACAGAAATCATCCA | |
| | | | CAAAAGAGAGAACCCATATCTCTGACACTTGCTGTGATGCTC | |
| | | | CGACTTGGAGTGGCAGCAGGTGTAGGAACAGGAACAGCTGCC | |
| | | | CTGGTCACGGGACCACAGCAGCTAGAAACACGACTTAGTAAC | |
| | | | CTACATCGAATTGTAACAGAAGATCTCCAAGCCCTAGAAAAA | |
| | | | TCTGTCAGTAACCTGGAGGAATCCCTAACCTCCTTATCTGAA | |
| | | | GTAGTCCTACAGAATAGAAGAGGGTTAGATTTATTATTTCTA | |
| | | | AAAGAAGGAGGATTATGTGTAGCCTTGAAGGAGGAATGCTGT | |
| | | | TTTTATGTGGATCATTCAGGGGCCATCAGAGACTCCATGAAC | |
| | | | AAACTTAGAGAAAGGTTGGAGAAGCGTCGAAGGGAAAAGGAA | |
| | | | ACTACTCAAGGGTGGTTTGAGCCATGGTTCAACAGGTCTCCT | |
| | | | TGGTTGGCTACCCTACTTTCTCCTTTAACAGCACCCTTAATA | |
| | | | GTCCTCCTCCTGTTACTCACAGTTGCGCCATGTATTATTAAC | |
| | | | AAGTTAATTGCCTTCATTAGAGAACGAATAAGTGCAGTCCAG | |
| | | | ATCATGGTACTTAGACAACAGTACCAAAGCCCGTCTAGCACG | |
| | | | GAAGCTGGCCGCCTCTAC | |
| C | C | N | ATGCATCCCACGTTAAGCCGGCGCCACCTCCCGATTCGGGGT | SEQ ID NO:37 |
| | | | GGAAAGCCGAAAAGACTGAAAATCCCCTTAAGCTTCGCCTCC | |
| | | | ATCGCGTGGTTCCTTACTCTGTCAATAACCTCTCAGACTAAT | |
| | | | GGTATGCGCATAGGAGACAGCCTGAACTCCCATAAACCCTTA | |
| | | | TCTCTCACCTGGTTAATTACTGACTCCGGCACAGGTATTAAT | |
| | | | ATCAACAACACTCAAGCGGAGGCTCCTTTAGGAACCTGGTGG | |
| | | | CCTGATCTATACGTTTGCCTCAGATCAGTTATTCCTAGTCTG | |
| | | | AATGACCAGACCTCACCCCCAGATATCCTCCATGCTCACGGA | |
| | | | TTTTATGTTTGCCCAGGACCACCAAATAATGGAAAACATTGC | |
| | | | GGAAATCCCAGAGATTTCTTTTGTAAACAATGGAACTGTGTA | |
| | | | ACCTCTAATGATGGATATTOGAAATGGCCAACCTCTCAGCAG | |
| | | | GATAGGGTAAGTTTTTCTTATGTCAACACCTATACCACCTCT | |
| | | | GGACAATTTAATTACGGCCATGGGAGATGGCTGACCTGGCAA | |
| | | | CAGCGGGTACAAAAAGATATTACAACTCGAAGCCCCAAGTGC | |
| | | | TCTCCTTCAGACCTAGATTACCTAAAAATAAGTTTCACTGAG | |
| | | | AAAGGAAAACAAGAAAATATCCTAAKATGGGTAAATGGTATG | |
| | | | TCTTGGGGAATGGTATATTATGGAGGCTCCGGTAAACAACCA | |
| | | | GGCTCCATTCTAACTATTCGCCTCAAAATAAACACTCAGCTG | |
| | | | GAGCCTCCAATGGCTATAGGACCAAATACGGTCTTGACGGGT | |
| | | | CAAAGACCCCCAACCCAACGACCACCGCATAACTTGCCGGTG | |
| | | | CCCCAGGGACCATCCCCTAACCCCGACATAACACAGTCTGAT | |
| | | | TACAACATAACTTCTGGATCAOACCCCACTAACACGCCTAGA | |
| | | | AACGAGTCTAACAGCACGACTAAAATGGGGGCAAAACTTTTT | |
| | | | AGCCTCATCCAGGGAGCTTTTCAAGCTCTTAACTCCACGACT | |
| | | | CCAGAGCCTACCTCTTCTTGTTCGCTATGCTTAGCTTTGGGC | |
| | | | CCACCTTACTATGAAGGAATGGCTAGAGAGAGGAAATTCAAT | |
| | | | GTGACAAAAGAACATAGAGACCAATGCACATGGGGATCCCAA | |
| | | | AATAAGCTTACCCTTACTOAGGTTTCTGGAAAAGGCACCTGC | |
| | | | ATAGGAAAGGTTCCCCCATCCCACCAACACCTTTGTAACCAC | |
| | | | ACTGAACCCTTTAATCAAACCTCTCAGAGTCAATATCTGGTA | |
| | | | CCTGGTTATGACAGGTGGTGGGCATGTAATACTGGATTAACC | |
| | | | CCTTGTGTTTCCACCTTGGTTTTTAACCAAACTAAAGATTTT | |
| | | | TGCATTATGGTCCAAATTGTTCCCCGAGTGTATTACTATCCC | |
| | | | GAAAAAGCAATCCTTGATGAATATGACTACAGAAATCATCGA | |
| | | | CAAAACAGAGAACCCATATCTCTGACACTTGCTGTGATGCTC | |
| | | | GGACTTGGAGTGGCAGCAGGTGTAGGAACAGGAACAGCTGCC | |
| | | | CTGGTCACGGGACCACAGCAGCTAGAAACAGGACTTAGTAAC | |
| | | | CTACATCGAATTGTAACAGAAGATCTCCAAGCCCTAGAAAAA | |
| | | | TCTGTCAGTAACCTGGAGGAATCCCTAACCTCCTTATCTGAA | |
| | | | GTAGTCCTACACAATAGAAGAGGCTTAGATTTATTATTTCTA | |
| | | | AAAGAAGGAGGATTATGTGTAGCCTTGAAGGAGGAATGCTGT | |
| | | | TTTTATGTGGATCATTCAGGGGCCATCAGAGACTCCATGAAC | |
| | | | AAACTTAGAGAAAGGTTGGAGAAGCGTCGAAGGGAAAAGGAA | |
| | | | ACTACTCAAGGGTGGTTTGAGGGATGGTTCAACAGGTCTCCT | |
| | | | TGGTTCGCTACCCTACTTTCTGCTTTAACAGGACCCTTAATA | |
| | | | GTCCTCCTCCTGTTACTCACAGTTGGGCCATGTATTATTAAC | |
| | | | AAGTTAATTGCCTTCATTAGAGAACCAATAAGTGCAGTCCAG | |
| | | | ATCATGGTACTTAGACAACAGTACCAAAGCCCGTCTAGCAGG | |
| | | | GAAGCTGGCCGCCTCTAC | |
| N | C | A | ATGCATCCCACGTTAAGCCGGCGCCACCTCCCGATTCGGGGT | SEQ ID NO:39 |
| | | | GGAAAGCCGAAAAGACTCAAAATCCCCTTAACCTTCGCCTCC | |
| | | | ATCGCGTGGTTCCTTACTCTGTCAATAACCTCTCAGACTAAT | |
| | | | GGTATGCGCATAGGAGACAGCCTGAACTCCCATAAACCCTTA | |
| | | | TCTCTCACCTGGTTAATTACTGACTCCGGCACACGTATTAAT | |
| | | | ATCAACAACACTCAAGGGGAGGCTCCTTTAGGAACCTGGTGG | |
| | | | CCTGATCTATACGTTTGCCTCAGATCAGTTATTCCTAGTCTG | |
| | | | ACCTCACCCCCAGATATCCTCCATGCTCACGGATTTTATGTT | |
| | | | TGCCCAGGACCACCAAATAATGGAAAACATTGCGGAAATCCC | |
| | | | AGAGATTTCTTTTGTAAACAATGGAACTGTGTAACCTCTAAT | |
| | | | GATGGATATTGGAAATGGCCAACCTCTCAGCAGGATAGGGTA | |
| | | | AGTTTTTCTTATGTCAACACCTATACCAGCTCTGGACAATTT | |
| | | | AATTACCTGACCTGGATTAGAACTGGAAGCCCCAAGTGCTCT | |
| | | | CCTTCAGACCTAGATTACCTAAAAATAAGTTTCACTGAGAAA | |
| | | | GGAAAACAAGAAAATATCCTAAAATGGGTAAATGGTATCTCT | |
| | | | TGGGGAATGGTATATTATGGAGGCTCGGGTAAACAACCAGGC | |
| | | | TCCATTCTAACTATTCGCCTCAAAATAAACCAGCTGGAGCCT | |
| | | | CCAATGGCTATAGGACCAAATACGGTCTTGACGGGTCAAAGA | |
| | | | CCCCCAACCCAACGACCAGGACCATCCTCTAACATAACTTCT | |
| | | | GGATCAGACCCCACTGAGTCTAACAGCACGACTAAAATGGGG | |
| | | | GCAAAACTTTTTAGCCTCATCCAGGGAGCTTTTCAAGCTCTT | |
| | | | AACTCCACGACTCCAGAGGCTACCTCTTCTTGTTGGCTATGC | |
| | | | TTAGCTTTGGGCCCACCTTACTATGAAGGAATGGCTAGAAGA | |
| | | | GGGAAATTCAATGTGACAAAAGAACATAGAGACCAATGCACA | |
| | | | TGGGGATCCCAAAATAAGCTTACCCTTACTGAGGTTTCTGGA | |
| | | | AAAGGCACCTGCATAGGAAACCTTCCCCCATCCCACCAACAC | |
| | | | CTTTCTAACCACACTGAAGCCTTTAATCAAACCTCTGAGAGT | |
| | | | CAATATCTGGTACCTCGTTATGACAGGTGGTGGGCATGTAAT | |
| | | | ACTGGATTAACCCCTTGTGTTTCCACCTTGGTTTTTAACCAA | |
| | | | ACTAAAGATTTTTGCATTATGGTCCAAATTGTTCCCCGAGTG | |
| | | | TATTACTATCCCGAAAAAGCAATCCTTGATGAATATGACTAC | |
| | | | AGAAATCATCGACAAAAGAGAGAACCCATATCTCTGACACTT | |
| | | | GCTGTGATCCTCGGACTTGGAGTGGCAGCAGGTGTAGGAACA | |
| | | | GGAACAGCTGCCCTGGTCACGGGACCACAGCAGCTAGAAACA | |
| | | | GGACTTAGTAACCTACATCGAATTGTAACAGAAGATCTCCAA | |
| | | | GCCCTAGAAAAATCTGTCAGTAACCTGGAGGAATCCCTAACC | |
| | | | TCCTTATCTGAAGTAGTCCTACAGAATAGAAGAGGGTTAGAT | |
| | | | TTATTATTTCTAAAAGAAGGAGGATTATGTGTAGCCTTGAAG | |
| | | | GAGGAATGCTGTTTTTATGTGGATCATTCAGGGGCCATCAGA | |
| | | | GACTCCATGAACAAACTTAGAGAAAGGTTGGAGAAGCGTCGA | |
| | | | AGGGAAAAGGAAACTACTCAAGGCTGGTTTCAGGGATGGTTC | |
| | | | AACAGGTCTCCTTGGTTGGCTACCCTACTTTCTGCTTTAACA | |
| | | | CGACCCTTAATAGTCCTCCTCCTGTTACTCACAGTTGGGCCA | |
| | | | TGTATTATTAACAAGTTAATTGCCTTCATTAGAGAACGAATA | |
| | | | AGTGCAGTCCAGATCATGGTACTTAGACAACAGTACCAAAGC | |
| | | | CCGTCTACCAGGGAAGCTGGCCCCTAGTAGTAG | |
| N | C | N | ATCCATCCCACGTTAAGCCGGCGCCACCTCCCGATTCGGGGT | SEQ ID NO:41 |
| | | | GGAAAGCCCAAAAGACTGAXAATCCCCTTAAGCTTCGCCTCC | |
| | | | ATCGCGTGGTTCCTTACTCTGTCAATAACCTCTCACACTAAT | |
| | | | GGTATGCGCATAGGAGACAGCCTGAACTCCCATAAACCCTTA | |
| | | | TCTCTCACCTGGTTAATTACTGACTCCGGCACAGGTATTAAT | |
| | | | ATCAACAACACTCAAGGGGACGCTCCTTTAGCAACCTGCTGG | |
| | | | CCTGATCTATACGTTTGCCTCAGATCAGTTATTCCTAGTCTG | |
| | | | ACCTCACCCCCAGATATCCTCCATGCTCACCGATTTTATGTT | |
| | | | TGCCCAGGACCACCAAATAATGGAAAACATTGCGGAAATCCC | |
| | | | AGAGATTTCTTTTGTAAACAATGGAACTGTGTAACCTCTAAT | |
| | | | GATGGATATTCGAAATCGCCAACCTCTCAGCACGATAGGGTA | |
| | | | AGTTTTTCTTATGTCAACACCTATACCAGCTCTGGACAATTT | |
| | | | AATTACCTGACCTGGATTAGAACTGGAAGCCCCAAGTGCTCT | |
| | | | CCTTCAGACCTAGATTACCTAAAAATAAGTTTCACTGAGAAA | |
| | | | GGAAAACAAGAAAATATCCTAAAATGGGTAAATGGTATGTCT | |
| | | | TGGGGAATGGTATATTATGGAGCCTCGGGTAAACAACCACGC | |
| | | | TCCATTCTAACTATTCGCCTCAAAATAAACCAGCTGGAGCCT | |
| | | | CCAATGGCTATAGGACCAAATACGGTCTTGACGGGTCAAAGA | |
| | | | CCCCCAACCCAAGGACCAGGACCATCCTCTAACATAACTTCT | |
| | | | GGATCAGACCCCACTGACTCTAACAGCACGACTAAAATGGGG | |
| | | | GCAAAACTTTTTAGCCTCATCCAGGGAGCTTTTCAAGCTCTT | |
| | | | AACTCCACCACTCCAGAGGCTACCTCTTCTTGTTGGCTATGC | |
| | | | TTAGCTTTGGGCCCACCTTACTATGAAGGAATGGCTAGAAGA | |
| | | | GGGAAATTCAATGTGACAAAAGAACATACACACCAATGCACA | |
| | | | TGGGGATCCCAAAATAAGCTTACCCTTACTGAGGTTTCTGGA | |
| | | | AAAGGCACCTGCATAGGAAAGGTTCCCCCATCCCACCAACAC | |
| | | | CTTTGTAACCACACTGAAGCCTTTAATCAAACCTCTGAGAGT | |
| | | | CAATATCTGGTACCTGGTTATGACAGGTGGTGGGCATGTAAT | |
| | | | ACTGGATTAACCCCTTGTGTTTCCACCTTGGTTTTTAACCAA | |
| | | | ACTAAAGATTTTTGCATTATGGTCCAAATTGTTCCCCGAGTG | |
| | | | TATTACTATCCCGAAAAAGCAATCCTTGATGAATATGACTAC | |
| | | | AGAAATCATCGACAAAAGAGAGAACCCATATCTCTGACACTT | |
| | | | GCTGTGATGCTCGGACTTGGAGTGGCACCAGGTGTAGGAACA | |
| | | | GGAACAGCTGCCCTGGTCACGGGACCACAGCAGCTAGAAACA | |
| | | | GGACTTAGTAACCTACATCGAATTGTAACAGAAGATCTCCAA | |
| | | | GCCCTAGAAAAATCTGTCAGTAACCTGGAGGAATCCCTAACC | |
| | | | TCCTTATCTGAAGTAGTCCTACAGAATAGAAGAGGGTTAGAT | |
| | | | TTATTATTTCTAAAAGAAGGAGGATTATGTGTAGCCTTGAAG | |
| | | | GACGAATGCTGTTTTTATGTGGATCATTCAGGGGCCATCAGA | |
| | | | GACTCCATGAACAAACTTAGAGAAAGGTTGGAGAAGCGTCGA | |
| | | | AGGGAAAAGGAAACTACTCAAGGGTGGTTTGAGGGATGGTTC | |
| | | | AACAGGTCTCCTTGGTTGGCTACCCTACTTTCTGCTTTAACA | |
| | | | GGACCCTTAATAGTCCTCCTCCTGTTACTCACAGTTGGGCCA | |
| | | | TGTATTATTAACAAGTTAATTGCCTTCATTAGAGAACGAATA | |
| | | | AGTGCAGTCCAGATCATGGTACTTAGACAACAGTACCAAAGC | |
| | | | CCGTCTACCAGGGAAGCTGGCCGCTAGTAGTAG | |

TABLE 8

| Recon- | | | | |
|---|---|---|---|---|
| struction | PERV | | | |
| Method | subtype | Tree | Amino Acid Sequence | SEQ ID NO: |
| A | A | A | MHPTLSRRHLPIRGGKPKRLKIPLSFASIAWFLTLSITPQ | SEQ ID NO:43 |
| | | | VNGKRLVDSPNSHKPLSLTWLLTDSGTGININSTQGEAPL | |
| | | | CTWWPELYVCLRSVIPGLNDQATPPDVLRAYGFYVCPGPP | |
| | | | NNEEYCGNPQDFFCKQWSCVTSNDGNWKWPVSQQDRVSYS | |
| | | | FVNNPTSYNQFNYGHGRWKDWQQRVQKDvRNKQISCNSLD | |
| | | | LDYLKISFTEKGKQENIQKWVNGMSWGIVYYGGSGRKKCS | |
| | | | VLTIRLRIETQMEPPVATGPNKGLAEQGPPIQEQRPSPNP | |
| | | | SDYNTTSGSVPTEPNITIKTGAKLFSLIQGAFQALNSTTP | |
| | | | EATSSCWLCLASGPPYYEGMARGGKFNVTKEHRDQCTWGS | |
| | | | QNKLTLTEVSGKGTCIGRVPPSHQHLCNHTEAFNRTSESQ | |
| | | | YLVPGYDRWWACNTGLTPCVSTLVFNQTKDFCVMVQIVPR | |
| | | | VYYYPEKAVLDEYDYRYNRPKREPISLTLAVMLGLGVAAG | |
| | | | VGTGTAALITGPQQLEKGLSNLHRIVTEDLQALEKSVSNL | |
| | | | EESLTSLSEVVLQNRRGLDLLFLKECGLCVALKEECCFYV | |
| | | | DHSGAIRDSMSKLRERLERRRREREADQGWFECWFNRSPW | |
| | | | MTTLLSALTCPLVVLLLLLTVGPCLINRFVAFVRERVSAV | |
| | | | QIMVLRQQYQGLLSQGECDLY | |
| B | A | A | MHPTLSRRHLPIRGGKPKRLKIPLSFASIAWFLTLSITPQ | SEQ ID NO:8 |
| | | | VNGKRLVDSPNSHKPLSLTWLLTDSGTGININSTQGEAPL | |
| | | | GTWWPELYVCLRSVIPCLNDQATPPDVLRAYGFYVCPGPP | |
| | | | NNEEYCCNPQDFFCKQWSCVTSNDGNWKWPVSQQDRVSYS | |
| | | | FVNNPTSYNQFNYGHGRWKDWQQRVQKDVRNKQISCHSLD | |
| | | | LDYLKISFTEKGKQENIQKWVNGMSWGIVYYGGSGRKKGS | |
| | | | VLTIRLRIETQMEPPVATGPNKGLAEQGPPIQEQRPSPNP | |
| | | | SDYNTTSGSVPTEPNITIKTGAKLFSLIQGAFQALNSTTP | |
| | | | EATSSCWLCLASGPPYYEGMARGGKFNVTKEHRDQCTWGS | |
| | | | QNKLTLTEVSGKGTCIGMVPPSHQHLCNHTEAFNRTSESQ | |
| | | | YLVPGYDRWWACNTGLTPCVSTLVFNQTKDFCVMVQIVPR | |
| | | | VYYYPEKAVLDEYDYRYNRPKREPISLTLAVMLGLGVAAG | |
| | | | VGTCTAALITGPQQLEKGLSNLHRIVTEDLQALEKSVSNL | |
| | | | EESLTSLSEVVLQNRRGLDLLFLKEGGLCVALKEECCFYV | |
| | | | DHSGAIRDSMSKLRERLERRRREREADQGWFEGWFNRSPW | |
| | | | MTTLLSALTGPLVVLLLLLTVGPCLINRFVAFVRERVSAV | |
| | | | QIMVLRQQYQGLLSQGETDL | |
| B | A | N | MHPTLSRRHLPIRGGEPKRLKIPLSFASIAWFLTLSITPQ | SEQ ID NO:10 |
| | | | VNGKRLVDSPNSHKPLSLTWLLTDSGTGITINSTQGEAPL | |
| | | | GTWWPELYVCLRSVIPGLNDQATPPDVLRAYRFYVCPGPP | |
| | | | NNEEYCCNPQDFFCKQWSCVTSNDGNWKWPISQQDRVSYS | |
| | | | FVNNPTSYNQFNYGHGRWKDWQQRVQKDVRNKQISCNSLD | |
| | | | LDYLKISFTEKGKQENIQKWVNGMSWGIMYYGGSGRRKGS | |
| | | | VLTIRLRIETQMEPPVATGPNKGLAEQGPPIQEQRPSPNP | |
| | | | SDYNTTSGSVPTEPNTTTKTGAKLFSLIQGAFQALNSTTP | |
| | | | EATSSCWLCLASGPPYYEGMARGGKFNVTKEHRDQCTWGS | |
| | | | QNKLTLTEVSGKGTCIGRVPPSHQHLCNHTEAFNRTSESQ | |
| | | | YLVPGYDRWWACNTGLTPCVSTLVFNQTKDFCVMVQIVPR | |
| | | | VYYYPEKAVLDEYDYRYNRPKREPISLTLAVMLGLGVAAG | |
| | | | VGTGTAALITGPQQLEKGLSDLHRIVTEDLQALEKSVSNL | |
| | | | EESLTSLSEVVLQNRRGLDLLFLKEGGLCVALKEECCFYV | |
| | | | DHSGAIRDSMSKLRERLERRRREREADQGWFEGWFNRSPW | |
| | | | MTTLLSALTGPLVVLLLLLTVGPCLINRFVAFVRERVSAV | |
| | | | QIMVLRQQYQGLLSQGETDL | |
| C | A | A | MHPTLSRRHLPIRGGKPKRLKIPLSFASIAWFLTLSITPQ | SEQ ID NO:12 |
| | | | VNGKRLVDSPNSHKPLSLTWLLTDSGTGININSTQGEAPL | |
| | | | GTWWPELYVCLRSVIPGLNDQATPPDVLRAYGFYVCPGPP | |
| | | | NNEEYCGNPQDFFCKQWSCVTSNDGNWKWPVSQQDRVSYS | |
| | | | FVNNPTSYNQFNYGHGRWKDWQQRVQKDVRNKQISCHSLD | |
| | | | LDYLKISFTEKGKQENTQKWVNGMSWGIVYYGGSGRKKGS | |
| | | | VLTIRLRIETQMEPPVAIGPNKGLAEQGPPIQEPPHNLPV | |
| | | | PQRPSPNPDITQSDYNTTSGSVPTNTPRNEPNITIKTGAK | |
| | | | LFSLIQGAFQALNSTTPEATSSCWLCLASCPPYYEGMARG | |
| | | | GKFNVTKEHRDQCTWGSQNKLTLTEVSGKCTCIGRVPPSH | |
| | | | QHLCNHTEAFNRTSESQYLVPCYDRWWACNTGLTPCVSTL | |
| | | | VFNQTKDFCVMVQIVPRVYYYPEKAVLDEYDYRYNRPKRE | |
| | | | PTSLTLAVMLGLGVAAGVGTGTAALITGPQQLEKGLSNLH | |
| | | | RIVTEDLQALEKSVSNLEESLTSLSEVVLQNRRGLDLLFL | |
| | | | KEGGLCVALKEECCFYVDHSGAIRDSMSKLRERLERRRRE | |
| | | | READQGWFECWFNRSPWMTTLLSALTGPLVVLLLLLTVGP | |
| | | | CLINRFVAFVRERVSAVQIMVLRQQYQGLLSQGETDLY | |
| C | A | N | MHPTLSRRHLPIRGGEPKRLKIPLSFASIAWFLTLSITPQ | SEQ ID NO:14 |
| | | | VNGKRLVDSPNSHKPLSLTWLLTDSGTGITINSTQCEAPL | |
| | | | GTWWPELYVCLRSVIPGLNDQATPPDVLHAYGFYVCPGPP | |
| | | | NNEEYCGNPQDFFCKQWSCVTSNDGNWKWPTSQQDRVSYS | |
| | | | FVNNPTSYNQFNYGHGRWKDWQQRVQKDVRNKQISCNSLD | |
| | | | LDYLKISFTEKGKQENIQKWVNGMSWGIVYYGGSGRRKGS | |
| | | | VLTIRLRIETQMEPPVAIGPNKGLAEQGPPIQEQRPSPNP | |
| | | | PQRPSPNPDITQSDYNTTSGSVPTNTPRNEPNITIKTCAK | |
| | | | LFSLIQGAFQALNSTTPEATSSCWLCLASGPPYYEGMARG | |
| | | | GKFNVTKEHRDQCTWGSQNKLTLTEVSGKGTCIGRVPPSH | |
| | | | QHLCNHTEAFNRTSESQYLVPGYDRWWACNTGLTPCVSTL | |
| | | | VFNQTKDFCVMVQIVPRVYYYPEKAVLDEYDYRYNRPKRE | |
| | | | PISLTLAVMLGLGVAAGVGTGTAALITGPQQLEKGLSDLH | |
| | | | RIVTEDLQALEKSVSNLEESLTSLSEVVLQNRRGLDLLFL | |
| | | | KEGGLCVALKEECCFYVDHSGAIRDSMSKLRERLERRRRE | |
| | | | READQGWFEGWFNRSPWMTTLLSALTGPLVVLLLLLTVGP | |
| | | | CLINRFVAFVRERVSAVQIMVLRQQYQGLLSQGETDLY | |
| N | A | A | MHPTLSRRHLPIRGGKPKRLKIPLSFASIAWFLTLSITPQ | SEQ ID NO:16 |
| | | | VNGKRLVDSPNSHKPLSLTWLLTDSCTGININSTQGEAPL | |
| | | | GTWWPELYVCLRSVIPGLNDQATPPDVLPAYGFYVCPGPP | |
| | | | NNEEYCGNPQDFFCKQWSCVTSNDGNWKWPVSQQDRVSYS | |
| | | | FVNNPTSYNQFNYGHCRWKDWQQRVQKDVRNKQISCHSLD | |
| | | | LDYLKISFTEKGKQENIQKWVNGMSWGIVYYGGSGRKKGS | |
| | | | VLTIRLRIETQMEPPVAIGPNKGLAEQCPPIQEQRPSPNP | |
| | | | SDYNTTSGSVPTEPNITIKTGAKLFSLIQGAFQALNSTTP | |
| | | | EATSSCWLCLASCPPYYEGMARGGKFNVTKEHRDQCTWGS | |
| | | | QNKLTLTEVSGKGTCIGRVPPSHQHLCNHTEAFNRTSESQ | |
| | | | YLVPGYDRWWACNTGLTPCVSTLVFNQTKDFCVMVQIVPR | |
| | | | VYYYPEKAVLDEYDYRYNRPKREPISLTLAVMLGLGVAAG | |
| | | | VGTGTAALITGPQQLEKGLSNLHRIVTEDLQALEKSVSNL | |
| | | | EESLTSLSEVVLQNRRGLDLLFLKECCLCVALKEECCFYV | |
| | | | DHSGAIRDSMSKLRERLERRRREREADQGWFEGWFNRSPW | |
| | | | MTTLLSALTGPLVVLLLLLTVGPCLINRFVAFVRERVSAV | |
| | | | QIMVLRQQYQGLLSQCETDL | |
| N | A | N | MHPTLSRRHLPIRGGEPKRLKIPLSFASIAWFLTLSITPQ | SEQ ID NO:18 |
| | | | VNGKRLVDSPNSHKPLSLTWLLTDSGTGITINSTQGEAPL | |
| | | | GTWWPELYVCLRSVIPGLNDQATPPDVLRAYRFYVCPGPP | |
| | | | NNEEYCGNPQDFFCKQWSCVTSNDGNWKWPISQQDRVSYS | |
| | | | EVNNPTSYNQFNYGHGRWKDWQQRVQKDVRNKQISCNSLD | |
| | | | LDYLKISFTEKGKQENIQKWVNGMSWGIMYYGGSGRRKGS | |
| | | | VLTIRLRIETQMEPPVAICPNKGLAEQGPPIQEQRPSPNP | |
| | | | SDYNTTSGSVPTEPNITIKTCAKLFSLIQGAFQALNSTTP | |
| | | | EATSSCWLCLASGPPYYEGMARGGKFNVTKEHRDQCTWCS | |
| | | | QNKLTLTEVSGKGTCIGRVPPSHQHLCNHTEAFNRTSESQ | |
| | | | YLVPGYDRWWACNTGLTPCVSTLVFNQTKDFCVMVQTVPR | |
| | | | VYYYPEKAVLDEYDYRYNRPKREPISLTLAVMLCLGVAAG | |
| | | | VGTCTAALITGPQQLEKGLSDLHRIVTEDLQALEKSVSNL | |
| | | | EESLTSLSEVVLQNRRGLDLLFLKEGGLCVALKEECCFYV | |
| | | | DHSGAIRDSMSKLRERLERRRREREADQGWFEGWFNRSPW | |
| | | | MTTLLSALTGPLVVLLLLLTVGPCLINRFVAFVRERVSAV | |
| | | | QIMXTLRQQYQGLLSQGETDL | |
| A | B | A | MHPTLSRRHLPTRGGEPKRLRIPLSFASILAWFLTLTITPQ | SEQ ID NO:44 |
| | | | ASSKRLIDSSNPHRPLSLTWLIIDPDTGVTVNSTRGVAPR | |
| | | | GTWWPELHFCLRLINPAVKSTPPNLVRSYGFYCCPGTEKE | |
| | | | KYCGGSGESFCRRWSCVTSNDGDWKWPISLQDRVKFSFVN | |
| | | | SGPGKYKVMKLYKDKSCSPSDLDYLKISFTEKGKQENIQK | |
| | | | WINGMSWGIVFYKYGGGAGSTLTIRLRTETGTEPPVAVGP | |
| | | | DKVLAEQGPPALEPPHNLPVPQLTSLRPDITQPPSNSTTG | |
| | | | LIPTNTPRNSPGVFVKTGQRLFSLIQGAFQAINSTDPDAT | |
| | | | SSCWLCLSSGPPYYEGHAKEGKFNVTKEHRNQCTWGSRNK | |
| | | | LTLTEVSCKGTCIGKAPPSHQHLCYSTVVYEQASENQYLV | |
| | | | PGYNRWWACNTGLTPCVSTSVFNQSKDFCVMVQIVPRVYY | |
| | | | HPEEVVLDEYDYRYNRPKREPVSLTLAVMLGLGTAVGVGT | |
| | | | GTAALITGPQQLEKGLGELHAAMTEDLRALEESVSNLEES | |
| | | | LTSLSEVVLQNRRCLDLLFLREGGLCAALKEECCFYVDHS | |
| | | | GAlRDSMSKLRERLERRRREREADQGWFEGWFNRSPWMTT | |
| | | | LLSALTGPLVVLLLLLTVGPCLTNRFVAFVRERVSAVQIM | |
| | | | VLRQQYQGLLSQGETDLY | |
| B | B | A | MHPTLSWRHLPTRGGEPKRLRIPLSFASIAWFLTLTITPQ | SEQ ID NO:20 |
| | | | ASSKRLIDSSNPHRPLSLTWLIIDPDTGVTVNSTRGVAPR | |
| | | | GTWWPELHFCLRLINPAVKSTPPNLVRSYGFYCCPGTEKE | |
| | | | KYCGGSGESFCRRWSCVTStWGDWKWPISLQDRVKFSFVN | |
| | | | SGPGKYKVMKLYKDKSCSPSDLDYLKISFTEKGKQENIQK | |
| | | | WINGMSWGIVFYKYGGGAGSTLTIRLRIETGTEPPVAVGP | |
| | | | DKVLAEQGPPALEPPHNLPVPQLTSLRPDITQPPSNGTTG | |
| | | | LIPTNTPRNSPGVPVKTGQRLFSLIQGAFQAINSTDPDAT | |
| | | | SSCWLCLSSGPPYYEGMAKEGKFNVTKEHRNQCTWGSRNK | |
| | | | LTLTEVSGKGTCIGKAPPSHQHLCYSTVVYEQASENQYLV | |
| | | | PGYNRWWACNTGLTPCVSTSVFNQSKDFCVMVQIVPRVYY | |
| | | | HPEEVVLDEYDYRYNRPKREPVSLTLAVMLGLGTAVGVGT | |
| | | | GTAALITGPQQLEKGLGELHAAMTEDLRALEESVSNLEES | |
| | | | LTSLSEVVLQNRRGLDLLFLREGGLCAALKEECCFYVDHS | |
| | | | GAlRDSMSKLRERLERRRREREADQGWFEGWFNRSPWMTT | |
| | | | LLSALTGPLVVLLLLLTVGPCLINRFVAFVRERVSAVQIM | |
| | | | VLRQQYQGLLSQGETDL | |
| B | B | N | MHPTLSWRHLPTRGGEPKRLRIPLSFASIAWFLTLTITPQ | SEQ ID NO:22 |
| | | | ASSKRLIDSSNPHRPLSLTWLIIDPDTGVTVNSTRGVAPR | |
| | | | GTWWPELHFCLRLINPAVKSTPPNLVRSYGFYCCPGTEKE | |
| | | | KYCGGSGESFCRRWSCVTSNDGDWKWPISLQDRVKFSFVN | |
| | | | SGPGKYKVMKLYKDKSCSPSDLDYLKISFTEKGKQENIQK | |
| | | | WINGMSWGIVFYKYGGGAGSTLTIRLRIETGTEPPVAVGP | |
| | | | DKVLAEQGPPALEPPHNLPVPQLTSLRPDITQPPSNSTTG | |
| | | | LIPTNTPRNSPGVPVKTGQRLFSLIQGAFQAINSTDPDAT | |
| | | | SSCWLCLSSGPPYYEGMAKEGKFNVTKEHRNQCTWGSRNK | |
| | | | LTLTEVSGKGTCIGKAPPSHQHLCNSTVVYEQASENQYLV | |
| | | | PGYNRWWACNTGLTPCVSTSVFNQSKDFCVMVQIVPRVYY | |
| | | | HPEEVVLDEYDYRYNRPKREPVSLTLAVMLGLGTAVGVGT | |
| | | | GTAALITGPQQLEKGLGELHAANTEDLRALEESVSNLEES | |
| | | | LTSLSEVVLQNRRGLDLLFLREGGLCAALKEECCFYVDHS | |
| | | | GAlRDSMSKLRERLERRRREREADQGWFEGWFNRSPWMTT | |
| | | | LLSALTGPLVVLLLLLTVGPCLINRFVAFVRERVSAVQIM | |
| | | | VLRQQYQGLLSQGETDL | |
| C | B | A | MHPTLSWRHLPTRGGEPKRLRIPLSFASIAWFLTLTITPQ | SEQ ID NO:24 |
| | | | ASSKRLIDSSNPHRPLSLTWLIIDPDTGVTVNSTRGVAPR | |
| | | | GTWWPELHFCLRLINPAVKDQSTPPNLVRSYGFYCCPGTP | |
| | | | EKEKYCGGSGESFCRRWSCVTSNDGDWKWPISLQDRVKFS | |
| | | | FVNSGPGYNQFNYGHGRWKDWKYKVMKLYKDKQISCSPSD | |
| | | | LDYLKISFTEKGKQENIQKWINGMSWGIVFYKYGGGKAGS | |
| | | | TLTIRLRIETGTEPPVAVGPDKVLAEQGPPALEPPHNLPV | |
| | | | PQLTSLRPDITQPPSNGTTGLIPTNTPRNSPGVPVKTGQR | |
| | | | LFSLIQGAFQAINSTDPDATSSCWLCLSSGPPYYEGMAKE | |
| | | | GKFNVTKEHRNQCTWGSRNKLTLTEVSGKGTCIGKAPPSH | |
| | | | QHLCYSTVVYEQASENQYLVPGYNRWWACNTGLTPCVSTS | |
| | | | VFNQSKDFCVMVQIVPRVYYHPEEVVLDEYDYRYNRPKRE | |
| | | | PVSLTLAVMLGLGTAVGVGTGTAALITGPQQLEKGLGELH | |
| | | | AAMTEDLRALEESVSNLEESLTSLSEVVLQNRRGLDLLFL | |
| | | | RECCLCAALKEECCFYVDHSGAIRDSMSKLRERLERRRRE | |
| | | | READQGWFEGWFNRSPWMTTLLSALTGPLVVLLLLLTVGP | |
| | | | CLINRFVAFVRERVSAVQIMVLRQQYQGLLSQGETDLY | |
| C | B | N | MHPTLSWRHLPTRGGEPKRLRIPLSFASIAWFLTLTITPQ | SEQ ID NO:26 |
| | | | ASSKRLIDSSNPHRPLSLTWLIIDPDTGVTVNSTRGVAPR | |
| | | | GTWWPELHFCLRLINPAVKDQSTPPNLVRSYGFYCCPGTP | |
| | | | EKEKYCGGSGESFCRRWSCVTSNDGDWKWPISLQDRVKFS | |
| | | | FVNSGPGYNQFNYGHCRWKDWKYKVMKLYKDKQISCSPSD | |
| | | | LDYLKISFTEKGKQENIQKWINGMSWGIVFYKYGGGRAGS | |
| | | | TLTIRLRIETGTEPPVAVGPDKVLAEQCPPALEPPHNLPV | |
| | | | PQLTSLRPDITQPPSNSTTGLIPTNTPRNSPGVPVKTGQR | |
| | | | LFSLIQCAFQAINSTDPDATSSCWLCLSSGPPYYEGMAKE | |
| | | | CKFNVTKEHRNQCTWGSRNKLTLTEVSGKGTCIGKAPPSH | |
| | | | QHLCNSTVVYEQASENQYLVPGYNRWWACNTGLTPCVSTS | |
| | | | VFNQSKDFCVMVQIVPRVYYHPEEVVLDEYDYRYNRPKRE | |
| | | | PVSLTLAVMLGLGTAVGVGTGTAALITGPQQLEKGLGELH | |
| | | | AAMTEDLRALEESVSNLEESLTSLSEVVLQNRRGLDLLFL | |
| | | | REGGLCAALKEECCFYVDHSGATRDSMSKLRERLERRRRE | |
| | | | READQGWFEGWFNRSPWMTTLLSALTGPLVVLLLLLTVGP | |
| | | | CLINRFVAFVRERVSAVQIMVLRQQYQGLLSQGETDLY | |
| N | B | A | MHPTLSWRHLPTRGGEPKRLRIPLSFASIAWFLTLTITPQ | SEQ ID NO:28 |
| | | | ASSKRLIDSSNPHRPLSLTWLIIDPDTGVTVNSTRGVAPR | |
| | | | GTWWPELHFCLRLTNPAVKSTPPNLVRSYGFYCCPGTEKE | |
| | | | KYCGGSGESFCRRWSCVTSNDCDWKWPISLQDRVKFSFVN | |
| | | | SGPGKYKVMKLYKDKSCSPSDLDYLKISFTEKCKQENIQK | |
| | | | WINGMSWGIVFYKYGGGAGSTLTIRLRIETGTEPPVAVGP | |
| | | | DKVLAEQGPPALEPPHNLPVPQLTSLRPDITQPPSNGTTG | |
| | | | LIPTNTPRNSPGVPVKTCQRLFSLIQGAFQAINSTDPDAT | |
| | | | SSCWLCLSSGPPYYECNAKEGKFNVTKEHRNQCTWGSRNK | |
| | | | LTLTEVSGKGTCIGKAPPSHQHLCYSTVVYEQASENQYLV | |
| | | | PCYNRWWACNTGLTPCVSTSVFNQSKDFCVMVQIVPRVYY | |
| | | | HPEEVVLDEYDYRYNRPKREPVSLTLAVMLGLCTAVGVGT | |
| | | | CTAALTTGPQQLEKGLCELHAAMTEDLRALEESvSNLEES | |
| | | | LTSLSEVVLQNRRGLDLLFLREGCLCAALKEECCFYVDHS | |
| | | | GAIRDSMSKLRERLERRRREREADQGwFEGwFNRSPWMTT | |
| | | | LLSALTGPLVVLLLLLTVGPCLINRFVAFVRERVSAvQIM | |
| | | | VLRQQYQGLLSQGETDL | |
| N | B | N | MHPTLSWRHLPTRGGEPKRLRIPLSFASIAWFLTLTITPQ | SEQ ID NO:30 |
| | | | ASSKRLIDSSNPHRPLSLTWLIIDPDTGVTVNSTRGVAPR | |
| | | | GTWWPELHFCLRLINPAVKSTPPNLVRSYCFYCCPGTEKE | |
| | | | KYCGCSGESFCRRWSCVTSNDCDWKWPISLQDRVKFSFVN | |
| | | | SGPGKYKVMKLYKDKSCSPSDLDYLKISFTEKGKQENIQK | |
| | | | WTNGMSWGIVFYKYCGGAGSTLTIRLRIETGTEPPVAVGP | |
| | | | DKVLAEQGPPALEPPHNLPVPQLTSLRPDITQPPSNSTTG | |
| | | | LIPTNTPRNSPGVPVKTGQRLFSLIQGAFQAINSTDPDAT | |
| | | | SSCWLCLSSGPPYYEGMAKEGKFNVTKEHRNQCTWGSRNK | |
| | | | LTLTEVSGKGTCIGKAPPSHQHLCNSTVVYEQASENQYLV | |
| | | | PGYNRWWACNTGLTPCVSTSVFNQSKDFCVMVQIVPRVYY | |
| | | | HPEEVVLDEYDYRYNRPKREPVSLTLAVNLCLGTAVCVGT | |
| | | | GTAALITGPQQLEKGLGELHAAMTEDLRALEESVSNLEES | |
| | | | LTSLSEVVLQNRRGLDLLFLREGGLCAALKEECCFYVDHS | |
| | | | GAlRDSMSKLRERLERRRREREADQCWFEGWFNRSPWNTT | |
| | | | LLSALTGPLVVLLLLLTVGPCLINRFVAFVRERVSAVQIM | |
| | | | VLRQQYQGLLSQGETDL | |
| A | C | A | MHPTLSRRHLPIRGGKPKRLKIPLSFASIAWFLTLSITSQ | SEQ ID NO:45 |
| | | | TNGMRIGDSLNSHKPLSLTWLITDSGTGININNTQGEAPL | |
| | | | GTWWPDLYVCLRSVIPSLTSPPDILHAHGFYVCPGPPNNG | |
| | | | KHCGNPRDFFCKQWNCVTSNDGYWKWPTSQQDRVSFSYVN | |
| | | | TYTSSGQFNYLTWIRTGSPKCSPSDLDYLKISFTEKGKQE | |
| | | | NILKWVNGMSWGMVYYGGSGKQPGSILTIRLKINQLEPPM | |
| | | | AIGPNTVLTGQRPPTQGPGPSSNITSGSDPTESNSTTKNG | |
| | | | AKLFSLIQGAFQALNSTTPEATSSCWLCLALGPPYYEGMA | |
| | | | RRGKFNVTKEHRDQCTWGSQNKLTLTEvSGKGTCIGKVPP | |
| | | | SHQHLCNHTEAFNQTSESQYLVPGYDRWWACNTGLTPCVS | |
| | | | TLVFNQTKDFCIMVQIVPRVYYYPEKAILDEYDYRNHRQK | |
| | | | REPISLTLAVMLCLCVAAGVGTGTAALvTGPQQLETGLSN | |
| | | | LHRIVTEDLQALEKSVSNLEESLTSLSEVVLQNRRGLDLL | |
| | | | FLKEGGLCVALKEECCFYVDHSGAIRDSMNKLRERLEKRR | |
| | | | REKETTQGWFEGWFNRSPWLATLLSALTGPLIVLLLLLTV | |
| | | | GPCIILNKLIAFIRERISAVQIMVLRQQYQSPSSREAGR | |
| B | C | A | MHPTLSRRHLPIRGGKPKRLKIPLSFASIAWFLTLSITSQ | SEQ ID NO:32 |
| | | | TNGMRIGDSLNSHKPLSLTWLITDSGTGININNTQGEAPL | |
| | | | GTWWPDLYVCLRSVIPSLTSPPDILHAHGFYVCPGPPNNG | |
| | | | KHCGNPRDFFCKQWNCVTSNDGYWKWPTSQQDRVSFSYVN | |
| | | | TYTSSGQFNYLTWIRTGSPKCSPSDLDYLKISFTEKGKQE | |
| | | | NILKWVNGMSWGMVYYGGSGKQPGSILTIRLKINQLEPPM | |
| | | | AIGPNTVLTGQRPPTQGPGPSSNITSGSDPTESNSTTKMG | |
| | | | AKLFSLIQGAFQALNSTTPEATSSCWLCLALGPPYYEGMA | |
| | | | RRGKFNVTKEHRDQCTWGSQNKLTLTEVSGKGTCIGKVPP | |
| | | | SHQHLCNHTEAFNQTSESQYLVPGYDRWWACNTGLTPCVS | |
| | | | TLVFNQTKDFCIMVQIVPRVYYYPEKAILDEYDYRNHRQK | |
| | | | REPISLTLAVMLGLGVAAGVGTGTAALVTGPQQLETGLSN | |
| | | | LHRIVTEDLQALEKSVSNLEESLTSLSEVVLQNRRGLDLL | |
| | | | FLKEGGLCVALKEECCFYVDHSGAIRDSMNKLRERLEKRR | |
| | | | REKETTQGWFEGWFNRSPWLATLLSALTGPLIVLLLLLTV | |
| | | | GPCIINKLIAFTRERISAVQIMVLRQQYQSPSSREAGR | |
| B | C | N | MHPTLSRRHLPIRGGKPKRLKIPLSFASIAWFLTLSITSQ | SEQ ID NO:34 |
| | | | TNGMRIGDSLNSHKPLSLTWLITDSGTGININNTQGEAPL | |
| | | | GTWWPDLYVCLRSVIPSLTSPPDILHAHGFYVCPGPPNNG | |
| | | | KHCGNPRDFFCKQWNCVTSNDGYWKWPTSQQDRVSFSYVN | |
| | | | TYTSSGQFNYLTWIRTCSPKCSPSDLDYLKISFTEKGKQE | |
| | | | NILKWVNGMSWCMVYYGGSGKQPGSILTIRLKINQLEPPM | |
| | | | AIGPNTVLTGQRPPTQGPGPSSNITSGSDPTESNSTTKMG | |
| | | | AKLFSLIQGAFQALNSTTPEATSSCWLCLALGPPYYEGMA | |
| | | | RRGKFNVTKEHRDQCTWGSQNKLTLTEVSGKGTCIGKVPP | |
| | | | SHQHLCNHTEAFNQTSESQYLVPGYDRWWACNTCLTPCVS | |
| | | | TLVFNQTKDFCIMVQIVPRVYYYPEKAILDEYDYRNHRQK | |
| | | | REPISLTLAVMLGLGVAAGVGTGTAALVTGPQQLETGLSN | |
| | | | LHRIVTEDLQALEKSVSNLEESLTSLSEVVLQNRRGLDLL | |
| | | | FLKEGGLCVALKEECCFYVDHSGAIRDSMNKLRERLEKRR | |
| | | | REKETTQGWFEGWFNRSPWLATLLSALTGPLIVLLLLLTV | |
| | | | GPCIINKLIAFIRERISAVQINXJLRQQYQSPSSREAGR | |
| C | C | A | MHPTLSRRHLPIRGGKPKRLKIPLSFASIAWFLTLSITSQ | SEQ ID NO:36 |
| | | | TNGMRIGDSLNSHKPLSLTWLITDSGTCININNTQGEAPL | |
| | | | GTWWPDLYVCLRSVIPSLNDQTSPPDILHAHGFYVCPGPP | |
| | | | NNGKHCGNPRDFFCKQWNCVTSNDGYWKWPTSQQDRVSFS | |
| | | | YVNTYTSSGQFNYCHGRWLTWQQRVQKDIRTGSPKCSPSD | |
| | | | LDYLKISFTEKGKQENILKWVNGMSWGMVYYGGSGKQPGS | |
| | | | ILTIRLKTNTQLEPPMAIGPNTVLTGQRPPTQGPPHNLPV | |
| | | | PQGPSPNPDITQSDYNITSCSDPTNTPRNESNSTTKMGAK | |
| | | | LFSLIQGAFQALNSTTPEATSSCWLCLALCPPYYEGMARR | |
| | | | GKFNVTKEHRDQCTWGSQNKLTLTEVSGKGTCICKVPPSH | |
| | | | QHLCNHTEAFNQTSESQYLVPGYDRWWACNTGLTPCvSTL | |
| | | | VFNQTKDFCIMVQIVPRVYYYPEKAILDEYDYRNHRQKRE | |
| | | | PISLTLAVMLGLGVAAGVGTGTAALVTGPQQLETGLSNLH | |
| | | | RIVTEDLQALEKSVSNLEESLTSLSEVVLQNRRGLDLLFL | |
| | | | KEGGLCVALKEECCFYVDHSGAIRDSMNKLRERLEKRRRE | |
| | | | KETTQGWFEGWFNRSPWLATLLSALTGPLIVLLLLLTVGP | |
| | | | CIINKLIAFIRERISAVQIMVLRQQYQSPSSREAGRLY | |
| C | C | N | MHPTLSRRHLPTRGGKPKRLKIPLSFASIAWFLTLSITSQ | SEQ ID NO:38 |
| | | | TNGMRIGDSLNSHKPLSLTWLITDSGTGININNTQGEAPL | |
| | | | GTWWPDLYVCLRSVIPSLNDQTSPPDILHAHGFYVCPGPP | |
| | | | NNGKHCGNPRDFFCKQWNCVTSNDGYWKWPTSQQDRVSFS | |
| | | | YVNTYTSSGQFNYGHGRWLTWQQRvQKDIRTGSPKCSPSD | |
| | | | LDYLKISFTEKGKQENILKWVNGMSWGMVYYGGSGKQPGS | |
| | | | ILTIRLKINTQLEPPMAIGPNTVLTGQRPPTQGPPHNLFV | |
| | | | PQGPSPNPDITQSDYNITSGSDPTNTPRNESNSTTKMGAK | |
| | | | LFSLIQGAFQALNSTTPEATSSCWLCLALGPPYYEGMARR | |
| | | | GKFNVTKEHRDQCTWGSQNKLTLTEVSGKGTCIGKVPPSH | |
| | | | QHLCNHTEAFNQTSESQYLVPGYDRWWACNTGLTPCVSTL | |
| | | | VFNQTKDFCIMVQIVPRVYYYPEKATLDEYDYRNHRQKRE | |
| | | | PISLTLAVMLGLGVAAGVGTGTAALVTGPQQLETGLSNLH | |
| | | | RIVTEDLQALEKSVSNLEESLTSLSEVVLQNRRGLDLLFL | |
| | | | KEGGLCVALKEECCFYVDHSGAIRDSMNKLRERLEKRRRE | |
| | | | KETTQGWFEGWFNRSPWLATLLSALTCPLIVLLLLLTVGP | |
| | | | CTINKLIAFIRERTSAVQIMVLRQQYQSPSSREAGRLY | |
| N | C | A | MHPTLSRRHLPIRGGKPKRLKIPLSFASILAWFLTLSITSQ | SEQ ID NO:40 |
| | | | TNGMRIGDSLNSHKPLSLTWLTTDSGTGININNTQCEAPL | |
| | | | GTWWPDLYVCLRSVIPSLTSPPDILHAHGFYVCPGPPNNG | |
| | | | KHCGNPRDFFCKQWNCVTSNDGYWKWPTSQQDRVSFSYVN | |
| | | | TYTSSGQFNYLTWIRTGSPKCSPSDLDYLKISFTEKCKQE | |
| | | | NILKWVNGMSWGMVYYGGSGKQPGSTLTIRLKINQLEPPM | |
| | | | AIGPNTVLTGQRPPTQGPGPSSNITSGSDPTESNSTTKNG | |
| | | | AKLFSLIQGAFQALNSTTPEATSSCWLCLALGPPYYEGMA | |
| | | | RRGKFNVTKEHRDQCTWCSQNKLTLTEvSCKGTCICKVPP | |
| | | | SHQHLCNHTEAFNQTSESQYLVPGYDRWWACNTGLTPCVS | |
| | | | TLVFNQTKDFCIMVQIVPRVYYYPEKAILDEYDYRNHRQK | |
| | | | REPISLTLAVMLGLGVAAGVGTGTAALVTGPQQLETGLSN | |
| | | | LHRIVTEDLQALEKSVSNLEESLTSLSEVVLQNRRCLDLL | |
| | | | FLKEGGLCVALKEECCFYVDHSCAIRDSNNKLRERLEKRR | |
| | | | REKETTQGWFECWFNRSPWLATLLSALTGPLIVLLLLLTV | |
| | | | GPCIINKLIAFIRERISAVQIMVLRQQYQSPSSREACR | |
| N | C | N | MHPTLSRRHLPIRGGKPKRLKIPLSFASIAWFLTLSITSQ | SEQ ID NO:42 |
| | | | TNGMRTCDSLNSHKPLSLTWLITDSGTGININNTQGEAPL | |
| | | | GTWWPDLYVCLRSVIPSLTSPPDILHAHGFYVCPCPPNNG | |
| | | | KHCGNPRDFFCKQWNCVTSNDGYWKWPTSQQDRVSFSYVN | |
| | | | TYTSSGQPNYLTWTRTGSPKCSPSDLDYLKISFTEKGKQE | |
| | | | NILKWVNGMSWGMVYYGCSGKQPGSILTIRLKINQLEPPM | |
| | | | AIGPNTVLTGQRPPTQGPCPSSNITSGSDPTESNSTTKMC | |
| | | | AKLFSLIQGAFQALNSTTPEATSSCWLCLALGPPYYECMA | |
| | | | RRGKFNVTKEHRDQCTWCSQNKLTLTEVSGKGTCIGKVPP | |
| | | | SHQHLCMITEAFNQTSESQYLVPGYDRWWACNTGLTPCVS | |
| | | | TLVFNQTKDFCIMVQIVPRVYYYPEKAILDEYDYRNHRQK | |
| | | | REPISLTLAVMLGLCVAAGVGTGTAALVTGPQQLETGLSN | |
| | | | LHRIVTEDLQALEKSVSNLEESLTSLSEVVLQNRRGLDLL | |
| | | | FLKECGLCVALKEECCFYVDHSGAIRDSNNKLRERLEKRR | |
| | | | REKETTQGWFEGWFNRSPWLATLLSALTGPLIVLLLLLTV | |
| | | | GPCIINKLIAFIRERISAVQIMVLRQQYQSPSSREAGR | |

TABLE 9

| Recon- | | | | |
|---|---|---|---|---|
| struction | PERV | | | |
| Method | subtype | Tree | Nucleotide Sequence (human codon usage) | SEQ ID NO: |
| A | A | A | ATGCACCCCACCCTGACCCCGCGGCACCTGCCCATCCGGGG | SEQ ID NO:46 |
| | | | CGCCAACCCCAAGCGGCTGAAGATCCCCCTGAGCTTCCCCA | |
| | | | GCATCGCCTGGTTCCTGACCCTGAGCATCACCCCCCAGCTG | |
| | | | AACGGCAAGCGGCTGGTGGACAGCCCCAACACCCACAAGCC | |
| | | | CCTGAGCCTGACCTGGCTGCTGACCGACAGCGGCACCGGCA | |
| | | | TCAACATCAACAGCACCCAGGGCGAGGCCCCCCTGGGCACC | |
| | | | TGGTGGCCCGAGCTGTACGTGTGCCTCCGGAGCGTGATCCC | |
| | | | CGGCCTGAACGACCACGCCACCCCCCCCGACGTGCTGCGGG | |
| | | | CCTACGGCTTCTACGTGTGCCCCGGCCCCCCCAACAACGAG | |
| | | | GAGTACTGCGGCAACCCCCAGGACTTCTTCTGCAAGCAGTG | |
| | | | GAGCTGCGTGACCAGCAACGACGGCAACTGCAAGTGGCCCG | |
| | | | TGAGCCAGCAGGACCGGGTGAGCTACAGCTTCGTGAACAAC | |
| | | | CCCACCAGCTACAACCAGTTCAACTACGGCCACGGCCGGTG | |
| | | | GAAGGACTGGCAGCAGCGGGTGCAGAAGGACGTGCGGAACA | |
| | | | AGCAGATCAGCTCCAACAGCCTGGACCTGGACTACCTGAAG | |
| | | | ATCAGCTTCACCGAGAAGGGCAAGCAGGAGAACATCCAGAA | |
| | | | GTGGGTGAACGGCATGAGCTGGGGCATCGTGTACTACGGCG | |
| | | | GCAGCGGCCGGAAGAACGGCAGCGTGCTGACCATCCGGCTG | |
| | | | CGGATCGAGACCCAGATGGAGCCCCCCGTGGCCATCGGCCC | |
| | | | CAACAAGGGCCTGCCCGAGCAGGGCCCCCCCATCCAGGAGC | |
| | | | AGCGGCCCAGCCCCAACCCCAGCGACTACAACACCACCAGC | |
| | | | GGCAGCGTGCCCACCGAGCCCAACATCACCATCAAGACCGG | |
| | | | CGCCAAGCTGTTCAGCCTGATCCAGGGCGCCTTCCAGGCCC | |
| | | | TCAACACCACCACCCCCGAGGCCACCAGCAGCTGCTGGCTG | |
| | | | TGCCTGGCCAGCGGCCCCCCCTACTACGAGGGCATGGCCCG | |
| | | | GGGCGGCAAGTTCAACGTGACCAAGGAGCACCGGGACCAGT | |
| | | | GCACCTGGGGCAGCCAGAACAAGCTGACCCTGACCGAGGTG | |
| | | | AGCGGCAAGGGCACCTGCATCGGCCGGGTGCCCCCCAGCCA | |
| | | | CCAGCACCTGTGCAACCACACCGAGGCCTTCAACCGGACCA | |
| | | | GCGAGAGCCAGTACCTGGTGCCCGGCTACGACCGGTGGTGC | |
| | | | GCCTGCAACACCGCCCTGACCCCCTGCGTGAGCACCCTGGT | |
| | | | GTTCAACCAGACCAAGGACTTCTGCGTGATGGTCCAGATCG | |
| | | | TGCCCCGGGTGTACTACTACCCCGAGAAGGCCGTGCTGGAC | |
| | | | GAGTACGACTACCGGTACAACCGGCCCAAGCGGGAGCCCAT | |
| | | | CAGCCTGACCCTGGCCGTGATGCTGGGCCTGGGCGTGGCCC | |
| | | | CCGGCGTGGGCACCGGCACCGCCGCCCTGATCACCGGCCCC | |
| | | | CAGCAGCTGGAGAAGGGCCTGAGCAACCTGCACCGGATCGT | |
| | | | GACCGAGGACCTGCAGGCCCTGGACAAGAGCGTGAGCAACC | |
| | | | TGGAGGAGAGCCTGACCAGCCTGAGCGAGGTGGTGCTGCAG | |
| | | | AACCGGCGGGGCCTGGACCTGCTGTTCCTGAAGGAGGGCGG | |
| | | | CCTGTGCGTGGCCCTGAAGGAGGAGTGCTGCTTCTACGTGG | |
| | | | ACCACAGCGGCGCCATCCCGGACAGCATGACCAAGCTGCGG | |
| | | | GAGCGGCTGGAGCGCCCGCGGCGGGAGCGGGACCCCGACCA | |
| | | | CGCCTGGTTCGAGGCCTGGTTCAACCGGAGCCCCTGGATGA | |
| | | | CCACCCTGCTGAGCGCCCTGACCGGCCCCCTGGTGGTGCTG | |
| | | | CTGCTGCTGCTGACCGTGGGCCCCTGCCTGATCAACCCGTT | |
| | | | CGTGGCCTTCGTGCCGGAGCGGGTGAGCGCCGTGCAGATCA | |
| | | | TGGTGCTGCGGCAGCAGTACCAGGGCCTGCTGACCCAGGGC | |
| | | | GACGCCGACCTGTAC | |
| B | A | A | ATGCACCCCACCCTGAGCCGGCGCCACCTGCCCATCCGGGG | SEQ ID NO:47 |
| | | | CGGCAAGCCCAACCGGCTGAAGATCCCCCTGAGCTTCGCCA | |
| | | | GCATCGCCTGGTTCCTGACCCTGAGCATCACCCCCCAGGTG | |
| | | | AACGGCAAGCGGCTCGTGGACAGCCCCAACAGCCACAAGCC | |
| | | | CCTGAGCCTGACCTGGCTGCTGACCGACAGCGGCACCGGCA | |
| | | | TCAACATCAACAGCACCCAGGGCGAGGCCCCCCTGGGCACC | |
| | | | TGGTGGCCCGAGCTGTACGTGTGCCTGCGGAGCGTGATCCC | |
| | | | CGCCCTGAACGACCAGGCCACCCCCCCCGACGTGCTGCGGG | |
| | | | CCTACGGCTTCTACGTGTGCCCCGGCCCCCCCAACAACGAG | |
| | | | GAGTACTGCGGCAACCCCCAGGACTTCTTCTGCAAGCAGTG | |
| | | | GAGCTGCGTGACCAGCAACGACGGCAACTGGAAGTGGCCCG | |
| | | | TGAGCCAGCAGGACCGGGTGACCTACAGCTTCGTGAACAAC | |
| | | | CCCACCAGCTACAACCAGTTCAACTACGGCCACGGCCGGTG | |
| | | | GAAGGACTGGCAGCAGCGGGTGCAGAAGGACGTGCGGAACA | |
| | | | AGCAGATCAGCTGCCACAGCCTGGACCTGGACTACCTGAAG | |
| | | | ATCAGCTTCACCGAGAAGGGCAAGCAGGAGAACATCCAGAA | |
| | | | GTGGGTGAACGGCATGAGCTGGGGCATCGTGTACTACGGCG | |
| | | | GCAGCGGCCGGAAGAAGGGCAGCGTGCTGACCATCCGGCTG | |
| | | | CGGATCGAGACCCAGATGGAGCCCCCCGTGGCCATCCGCCC | |
| | | | CAACAAGGGCCTGGCCGAGCAGGGCCCCCCCATCCAGGAGC | |
| | | | ACCGGCCCAGCCCCAACCCCAGCGACTACAACACCACCAGC | |
| | | | GGCAGCGTGCCCACCGAGCCCAACATCACCATCAAGACCGG | |
| | | | CGCCAAGCTGTTCAGCCTGATCCAGGGCGCCTTCCAGGCCC | |
| | | | TGAACAGCACCACCCCCGAGGCCACCAGCAGCTGCTGGCTG | |
| | | | TGCCTGGCCAGCGGCCCCCCCTACTACGAGGGCATGGCCCG | |
| | | | GGGCGGCAAGTTCAACGTGACCAAGGAGCACCGGGACCAGT | |
| | | | GCACCTGGGGCAGCCAGAACAAGCTGACCCTGACCGAGGTG | |
| | | | AGCGGCAAGGGCACCTGCATCGGCATGGTGCCCCCCAGCCA | |
| | | | CCAGCACCTGTGCAACCACACCGAGGCCTTCAACCGGACCA | |
| | | | GCGAGAGCCAGTACCTGGTGCCCCGCTACGACCGGTGGTGG | |
| | | | GCCTGCAACACCGGCCTGACCCCCTGCGTGAGCACCCTGGT | |
| | | | GTTCAACCAGACCAAGGACTTCTGCGTGATGGTGCAGATCG | |
| | | | TGCCCCGGGTGTACTACTACCCCGAGAAGGCCGTGCTGGAC | |
| | | | GAGTACGACTACCGGTACAACCGGCCCAAGCCGGAGCCCAT | |
| | | | CAGCCTGACCCTGGCCGTGATGCTGGGCCTGGGCGTGGCCC | |
| | | | CCGGCGTGGGCACCGGCACCGCCGCCCTGATCACCGGCCCC | |
| | | | CAGCAGCTGGAGAAGGGCCTGAGCAACCTGCACCGCATCGT | |
| | | | GACCGAGCACCTGCAGGCCCTCGAGAACAGCGTGAGCAACC | |
| | | | TGGAGGAGAGCCTGACCAGCCTGAGCGAGGTGGTGCTGCAG | |
| | | | AACCGGCGGGGCCTGGACCTGCTGTTCCTGAAGGAGGGCGG | |
| | | | CCTGTCCGTGGCCCTGAAGGAGGAGTCCTGCTTCTACGTGG | |
| | | | ACCACAGCGGCGCCATCCGGGACAGCATGAGCAAGCTGCGG | |
| | | | GAGCGCCTGGAGCGGCGGCGGCGGGAGCGGGAGGCCGACCA | |
| | | | GGGCTGGTTCCAGCGCTGGTTCAACCGGAGCCCCTGGATCA | |
| | | | CCACCCTGCTGACCCCCCTGACCGGCCCCCTGCTGGTGCTG | |
| | | | CTGCTGCTCCTGACCCTGGCCCCCTGCCTGATCAACCGGTT | |
| | | | CGTGGCCTTCGTGCGCGAGCCGGTGAGCGCCGTGCAGATCA | |
| | | | TGGTGCTGCGGCAGCAGTACCAGGGCCTGCTCAGCCAGGGC | |
| | | | GAGACCGACCTGTGATGA | |
| B | A | N | ATGCACCCCACCCTGAGCCGGCGGCACCTCCCCATCCGGGG | SEQ ID NO:48 |
| | | | CGGCGAGCCCAAGCGGCTGAAGATCCCCCTGAGCTTCGCCA | |
| | | | GCATCGCCTGGTTCCTGACCCTGAGCATCACCCCCCAGGTG | |
| | | | AACGGCAAGCGGCTGGTGGACAGCCCCAACAGCCACAAGCC | |
| | | | CCTGAGCCTGACCTGGCTGCTGACCGACAGCGGCACCGGCA | |
| | | | TCACCATCAACAGCACCCAGGCCGAGGCCCCCCTGGGCACC | |
| | | | TGGTGGCCCGAGCTGTACGTGTGCCTGCCGAGCGTGATCCC | |
| | | | CGGCCTCAACGACCAGGCCACCCCCCCCGACGTGCTGCGGG | |
| | | | CCTACCGCTTCTACGTGTGCCCCGGCCCCCCCAACAACGAG | |
| | | | GAGTACTGCGCCAACCCCCAGGACTTCTTCTGCAAGCAGTG | |
| | | | GAGCTGCGTGACCAGCAACGACGGCAACTGGAAGTGGCCCA | |
| | | | TCAGCCAGCAGGACCGGGTGAGCTACAGCTTCGTGAACAAC | |
| | | | CCCACCACCTACAACCAGTTCAACTACGGCCACGGCCGGTG | |
| | | | CAAGGACTGGCAGCAGCGGGTGCAGAAGGACGTGCGGAACA | |
| | | | AGCAGATCACCTGCAACAGCCTGGACCTGGACTACCTGAAG | |
| | | | ATCAGCTTCACCGAGAAGGGCAAGCAGGAGAACATCCAGAA | |
| | | | GTGGGTGAACGGCATGAGCTGGGGCATCATGTACTACGGCG | |
| | | | GCAGCGGCCGGCGGAAGGGCAGCGTGCTGACCATCCCGCTG | |
| | | | CGGATCGAGACCCAGATGGAGCCCCCCGTGGCCATCGGCCC | |
| | | | CAACAAGGGCCTGGCCCAGCAGGGCCCCCCCATCCAGGAGC | |
| | | | AGCGGCCCAGCCCCAACCCCAGCGACTACAACACCACCAGC | |
| | | | GGCAGCGTGCCCACCGAGCCCAACATCACCATCAAGACCGG | |
| | | | CGCCAAGCTGTTCAGCCTGATCCAGGGCGCCTTCCAGGCCC | |
| | | | TGAACAGCACCACCCCCGACCCCACCAGCAGCTGCTGGCTG | |
| | | | TGCCTGGCCAGCGGCCCCCCCTACTACGAGGGCATGGCCCG | |
| | | | GGGCGGCAAGTTCAACGTGACCAAGGAGCACCGGGACCAGT | |
| | | | GCACCTGCGGCACCCAGAACAAGCTGACCCTGACCGAGGTG | |
| | | | AGCGGCAAGCGCACCTGCATCGGCCGGGTGCCCCCCAGCCA | |
| | | | CCAGCACCTGTGCAACCACACCGAGGCCTTCAACCGGACCA | |
| | | | GCGAGAGCCAGTACCTGGTGCCCGGCTACGACCCCTCGTGG | |
| | | | GCCTGCAACACCGGCCTGACCCCCTGCGTGAGCACCCTGGT | |
| | | | GTTCAACCAGACCAAGGACTTCTGCGTGATGGTGCAGATCG | |
| | | | TGCCCCGGGTGTACTACTACCCCGAGAAGGCCGTGCTGGAC | |
| | | | GAGTACGACTACCGGTACAACCCGCCCAAGCGGGAGCCCAT | |
| | | | CAGCCTGACCCTGGCCGTGATGCTGGGCCTGGGCGTGGCCG | |
| | | | CCGGCGTGGGCACCGGCACCCCCGCCCTGATCACCGGCCCC | |
| | | | CAGCAGCTGGAGAAGGGCCTGAGCGACCTGCACCGGATCGT | |
| | | | GACCGAGGACCTGCAGGCCCTGGAGAAGAGCGTGAGCAACC | |
| | | | TGGAGGAGAGCCTGACCACCCTGAGCGAGGTGGTGCTGCAG | |
| | | | AACCCGCGGGGCCTGGACCTGCTGTTCCTGAAGGAGGGCGG | |
| | | | CCTGTGCGTGGCCCTGAAGGAGGAGTGCTGCTTCTACGTGG | |
| | | | ACCACAGCCCCGCCATCCGGGACAGCATGACCAAGCTGCGG | |
| | | | GAGCGGCTGGAGCGGCGGCGGCGGGAGCGGGAGGCCGACCA | |
| | | | GGGCTGGTTCGAGGGCTGGTTCAACCGGAGCCCCTGGATCA | |
| | | | CCACCCTGCTGAGCGCCCTGACCGGCCCCCTGGTGGTGCTG | |
| | | | CTGCTGCTGCTGACCGTGGGCCCCTGCCTGATCAACCGGTT | |
| | | | CGTGGCCTTCGTCCGGGAGCCGGTGAGCCCCGTGCAGATCA | |
| | | | TCGTCCTGCGGCAGCAGTACCAGGGCCTGCTGAGCCAGGGC | |
| | | | GACACCGACCTGTGATGA | |
| C | A | A | ATGCACCCCACCCTGAGCCGGCGGCACCTGCCCATCCGGGG | SEQ ID NO:49 |
| | | | CGGCAAGCCCAAGCGGCTGAAGATCCCCCTGAGCTTCGCCA | |
| | | | GCATCGCCTGGTTCCTGACCCTGAGCATCACCCCCCAGGTG | |
| | | | AACGGCAAGCGGCTGGTGGACAGCCCCAACAGCCACAAGCC | |
| | | | CCTGAGCCTGACCTGGCTGCTGACCGACAGCGGCACCGGCA | |
| | | | TCAACATCAACAGCACCCAGGGCGAGGCCCCCCTGGGCACC | |
| | | | TGGTGGCCCGAGCTGTACGTGTGCCTGCGGAGCGTGATCCC | |
| | | | CGGCCTGAACGACCAGGCCACCCCCCCCGACGTCCTGCGGG | |
| | | | CCTACGGCTTCTACGTGTGCCCCGGCCCCCCCAACAACGAG | |
| | | | GAGTACTGCGGCAACCCCCAGGACTTCTTCTGCAAGCAGTG | |
| | | | GAGCTGCGTGACCAGCAACGACGGCAACTGGAAGTGGCCCG | |
| | | | TGAGCCAGCAGGACCGGGTGAGCTACAGCTTCGTGAACAAC | |
| | | | CCCACCAGCTACAACCAGTTCAACTACGGCCACGGCCGGTG | |
| | | | GAAGGACTGGCAGCAGCGGGTGCAGAAGGACGTGCGGAACA | |
| | | | AGCAGATCAGCTGCCACAGCCTGGACCTGGACTACCTGAAG | |
| | | | ATCAGCTTCACCGAGAAGGGCAAGCAGGAGAACATCCAGAA | |
| | | | GTGGGTGAACGGCATGACCTGGGGCATCGTGTACTACGCCG | |
| | | | GCACCGGCCGGAAGAAGCGCAGCGTGCTGACCATCCCCCTG | |
| | | | CGGATCGAGACCCAGATGGAGCCCCCCGTGGCCATCGGCCC | |
| | | | CAACAAGGGCCTGGCCGAGCAGGGCCCCCCCATCCAGGAGC | |
| | | | CCCCCCACAACCTGCCCGTGCCCCAGCGGCCCAGCCCCAAC | |
| | | | CCCGACATCACCCAGAGCGACTACAACACCACCAGCGGCAG | |
| | | | CGTGCCCACCAACACCCCCCCGAACGAGCCCAACATCACCA | |
| | | | TCAAGACCGGCGCCAAGCTGTTCAGCCTGATCCAGGGCCCC | |
| | | | TTCCAGGCCCTGAACACCACCACCCCCGAGGCCACCAGCAG | |
| | | | CTGCTGGCTGTGCCTCGCCAGCGGCCCCCCCTACTACGAGG | |
| | | | GCATGGCCCGGGGCGGCAAGTTCAACGTGACCAAGGAGCAC | |
| | | | CGGGACCAGTGCACCTGGGGCAGCCAGAACAAGCTGACCCT | |
| | | | GACCGAGGTGAGCCGCAAGGGCACCTGCATCGGCCGGGTGC | |
| | | | CCCCCAGCCACCACCACCTGTGCAACCACACCGAGGCCTTC | |
| | | | AACCGGACCAGCGAGAGCCAGTACCTGGTGCCCGGCTACGA | |
| | | | CCGGTGGTGGGCCTCCAACACCGGCCTGACCCCCTGCGTGA | |
| | | | GCACCCTGGTGTTCAACCAGACCAAGCACTTCTGCGTGATG | |
| | | | GTGCAGATCGTGCCCCGCGTGTACTACTACCCCGAGAAGGC | |
| | | | CGTGCTGGACGAGTACGACTACCGGTACAACCGGCCCAAGC | |
| | | | GGGAGCCCATCAGCCTGACCCTGGCCGTGATGCTGGGCCTG | |
| | | | GCCGTGGCCGCCGGCGTGGCCACCCGCACCGCCGCCCTGAT | |
| | | | CACCGGCCCCCAGCAGCTGGAGAACGGCCTGAGCAACCTGC | |
| | | | ACCGGATCGTGACCGAGGACCTGCAGGCCCTGCAGAAGAGC | |
| | | | GTGAGCAACCTGGAGGAGAGCCTGACCAGCCTGACCGAGGT | |
| | | | GGTGCTGCAGAACCGGCGGGGCCTGGACCTGCTGTTCCTGA | |
| | | | AGGAGGGCGGCCTGTGCGTGGCCCTGAAGGAGGAGTGCTGC | |
| | | | TTCTACGTGGACCACAGCGGCGCCATCCGGGACAGCATGAG | |
| | | | CAAGCTGCGGGAGCGGCTGGAGCGGCGGCGGCGGGAGCGGG | |
| | | | AGGCCGACCAGGGCTCGTTCGAGGGCTGGTTCAACCGGAGC | |
| | | | CCCTGGATGACCACCCTGCTGAGCGCCCTGACCGGCCCCCT | |
| | | | GGTGGTGCTGCTGCTGCTGCTGACCCTGGGCCCCTGCCTGA | |
| | | | TCAACCGGTTCGTGGCCTTCGTGCGGGAGCGGGTGAGCGCC | |
| | | | GTGCAGATCATGGTGCTGCGGCAGCAGTACCAGGGCCTGCT | |
| | | | GAGCCAGGGCCAGACCGACCTGTAC | |
| C | A | N | ATGCACCCCACCCTGAGCCGGCGGCACCTGCCCATCCGGGG | SEQ ID NO:50 |
| | | | CGGCGAGCCCAAGCCGCTGAAGATCCCCCTCAGCTTCGCCA | |
| | | | GCATCGCCTGGTTCCTGACCCTGAGCATCACCCCCCAGGTG | |
| | | | AACGGCAACCGGCTGGTGGACAGCCCCAACACCCACAAGCC | |
| | | | CCTGAGCCTGACCTGGCTGCTGACCGACAGCGGCACCGGCA | |
| | | | TCACCATCAACACCACCCAGGGCGAGGCCCCCCTGGGCACC | |
| | | | TGGTGGCCCGAGCTGTACGTGTGCCTGCGGAGCGTGATCCC | |
| | | | CGGCCTGAACGACCAGCCCACCCCCCCCGACGTGCTGCGGG | |
| | | | CCTACGGCTTCTACGTGTGCCCCGGCCCCCCCAACAACGAG | |
| | | | CAGTACTCCGGCAACCCCCAGGACTTCTTCTGCAAGCAGTG | |
| | | | GAGCTGCGTGACCAGCAACGACGGCAACTGGAAGTGGCCCA | |
| | | | TCAGCCAGCAGGACCGGGTGAGCTACAGCTTCGTGAACAAC | |
| | | | CCCACCAGCTACAACCAGTTCAACTACGGCCACGGCCGGTG | |
| | | | GAAGGACTGGCAGCAGCGGGTGCAGAAGGACGTGCGGAACA | |
| | | | AGCAGATCAGCTGCCACAGCCTGGACCTCGACTACCTGAAG | |
| | | | ATCAGCTTCACCGAGAAGGGCAAGCAGGAGAACATCCAGAA | |
| | | | GTGGGTGAACCGCATGAGCTGGGGCATCGTGTACTACGGCG | |
| | | | GCAGCGGCCGGCGGAAGGGCAGCGTGCTGACCATCCGGCTG | |
| | | | CGGATCGAGACCCAGATGGAGCCCCCCGTGGCCATCGGCCC | |
| | | | CAACAAGGGCCTGGCCGAGCACGGCCCCCCCATCCAGGAGC | |
| | | | CCCCCCACAACCTGCCCGTGCCCCAGCGGCCCAGCCCCAAC | |
| | | | CCCGACATCACCCAGAGCGACTACAACACCACCAGCGGCAG | |
| | | | CGTGCCCACCAACACCCCCCGGAACGAGCCCAACATCACCA | |
| | | | TCAAGACCGGCGCCAAGCTGTTCAGCCTCATCCAGGGCGCC | |
| | | | TTCCACGCCCTGAACAGCACCACCCCCGAGGCCACCAGCAG | |
| | | | CTGCTCCCTGTGCCTGGCCAGCGGCCCCCCCTACTACGAGG | |
| | | | GCATGGCCCGGGGCGGCAAGTTCAACCTGACCAAGGAGCAC | |
| | | | CGGGACCAGTGCACCTGGGGCAGCCAGAACAAGCTGACCCT | |
| | | | GACCGAGGTCAGCGGCAAGGGCACCTGCATCGGCCGGGTGC | |
| | | | CCCCCAGCCACCAGCACCTGTGCAACCACACCGAGGCCTTC | |
| | | | AACCGGACCAGCGAGAGCCAGTACCTGGTGCCCGGCTACGA | |
| | | | CCGGTGGTGGGCCTGCAACACCGGCCTGACCCCCTGCGTGA | |
| | | | GCACCCTGGTGTTCAACCAGACCAAGGACTTCTGCGTGATG | |
| | | | GTGCAGATCGTGCCCCGGGTGTACTACTACCCCGAGAAGGC | |
| | | | CGTGCTGGACGACTACGACTACCGGTACAACCGGCCCAAGC | |
| | | | GGGAGCCCATCAGCCTGACCCTGGCCGTGATGCTGGGCCTG | |
| | | | CGCGTGGCCGCCGGCGTGGGCACCGGCACCGCCGCCCTGAT | |
| | | | CACCGGCCCCCAGCAGCTGGAGAAGGGCCTGAGCGACCTGC | |
| | | | ACCGGATCGTGACCCAGGACCTGCAGGCCCTGGAGAAGACC | |
| | | | CTGAGCAACCTGGAGGACAGCCTGACCAGCCTGAGCGACGT | |
| | | | GGTGCTGCAGAACCGGCGGGCCCTGGACCTGCTGTTCCTGA | |
| | | | AGGAGGGCGGCCTGTGCGTGCCCCTGAAGGAGGAGTGCTGC | |
| | | | TTCTACGTGGACCACAGCGGCGCCATCCGGGACAGCATGAG | |
| | | | CAAGCTGCGGGAGCGGCTGGAGCGGCGGCGGCGGGAGCGGG | |
| | | | AGGCCGACCAGGGCTGGTTCGAGGGCTGGTTCAACCGGAGC | |
| | | | CCCTGGATGACCACCCTGCTGAGCGCCCTGACCGGCCCCCT | |
| | | | GGTGGTGCTGCTGCTGCTCCTGACCGTGGGCCCCTGCCTGA | |
| | | | TCAACCCGTTCGTGGCCTTCGTGCGGCAGCGGGTGAGCGCC | |
| | | | GTGCAGATCATGGTGCTGCGGCAGCAGTACCAGGGCCTGCT | |
| | | | GAGCCAGGGCGAGACCCACCTGTAC | |
| N | A | A | ATGCACCCCACCCTGACCCGGCGGCACCTGCCCATCCGGGG | SEQ ID NO:51 |
| | | | CGGCAAGCCCAAGCGGCTGAAGATCCCCCTGAGCTTCGCCA | |
| | | | GCATCGCCTGGTTCCTGACCCTGAGCATCACCCCCCAGGTG | |
| | | | AACGGCAAGCGGCTGGTGGACAGCCCCAACAGCCACAAGCC | |
| | | | CCTGAGCCTGACCTGCCTGCTGACCGACAGCGGCACCGGCA | |
| | | | TCAACATCAACAGCACCCAGCGCGAGGCCCCCCTGGGCACC | |
| | | | TGGTGGCCCGAGCTGTACGTGTGCCTGCGGAGCGTGATCCC | |
| | | | CGGCCTGAACGACCAGGCCACCCCCCCCGACGTGCTGCGGG | |
| | | | CCTACGGCTTCTACGTGTGCCCCGGCCCCCCCAACAACGAG | |
| | | | GAGTACTGCGGCAACCCCCAGGACTTCTTCTGCAAGCAGTG | |
| | | | GAGCTGCGTGACCAGCAACGACGGCAACTGGAAGTGGCCCG | |
| | | | TGAGCCAGCAGCACCGGGTGAGCTACAGCTTCCTGAACAAC | |
| | | | CCCACCAGCTACAACCAGTTCAACTACGCCCACGGCCGGTG | |
| | | | GAAGGACTGGCAGCAGCGGGTGCAGAAGGACGTGCGGAACA | |
| | | | AGCAGATCAGCTGCCACAGCCTCGACCTGGACTACCTGAAG | |
| | | | ATCAGCTTCACCGAGAAGGGCAAGCAGGAGAACATCCAGAA | |
| | | | GTGGGTGAACGGCATGAGCTCGGGCATCGTGTACTACCGCG | |
| | | | GCAGCGGCCCGAAGAAGGCCAGCGTGCTGACCATCCGCCTG | |
| | | | CGGATCGAGACCCAGATGGAGCCCCCCGTGGCCATCGGCCC | |
| | | | CAACAAGGGCCTGGCCGAGCAGGGCCCCCCCATCCAGGAGC | |
| | | | AGCGGCCCAGCCCCAACCCCAGCGACTACAACACCACCAGC | |
| | | | GGCAGCGTGCCCACCGAGCCCAACATCACCATCAAGACCGG | |
| | | | CGCCAAGCTGTTCAGCCTGATCCAGGGCGCCTTCCAGGCCC | |
| | | | TGAACAGCACCACCCCCGAGGCCACCAGCAGCTGCTGGCTG | |
| | | | TGCCTGGCCAGCGGCCCCCCCTACTACGAGGGCATGGCCCG | |
| | | | GGGCGGCAAGTTCAACGTGACCAAGGAGCACCGCCACCAGT | |
| | | | GCACCTGGGGCAGCCAGAACAAGCTGACCCTGACCGAGGTG | |
| | | | AGCGGCAAGGGCACCTGCATCGGCCGGGTGCCCCCCAGCCA | |
| | | | CCAGCACCTGTGCAACCACACCGAGGCCTTCAACCGGACCA | |
| | | | GCGAGAGCCAGTACCTGGTCCCCGGCTACGACCGGTGGTGG | |
| | | | GCCTGCAACACCGGCCTGACCCCCTCCGTGAGCACCCTGGT | |
| | | | GTTCAACCAGACCAAGGACTTCTGCGTGATGGTGCAGATCG | |
| | | | TGCCCCGGGTCTACTACTACCCCGAGAAGGCCGTGCTGGAC | |
| | | | GAGTACGACTACCGGTACAACCGGCCCAAGCGCGAGCCCAT | |
| | | | CAGCCTGACCCTGGCCGTGATGCTGGGCCTGGGCGTGGCCG | |
| | | | CCGGCGTGGGCACCGGCACCGCCGCCCTGATCACCGGCCCC | |
| | | | CAGCAGCTGGAGAACGGCCTCAGCAACCTGCACCGCATCGT | |
| | | | GACCGAGGACCTGCAGGCCCTGGAGAAGAGCGTGAGCAACC | |
| | | | TCGAGGAGAGCCTGACCAGCCTGAGCGAGGTGGTGCTGCAG | |
| | | | AACCGGCGGGGCCTGGACCTGCTGTTCCTGAAGGAGGGCGC | |
| | | | CCTGTCCGTGGCCCTGAAGGAGGAGTGCTGCTTCTACGTGG | |
| | | | ACCACAGCGCCGCCATCCGGGACAGCATGAGCAAGCTGCGG | |
| | | | GAGCGGCTGGAGCGGCGGCGGCGGGAGCGGGAGGCCGACCA | |
| | | | GGGCTGGTTCGAGGGCTGGTTCAACCGGAGCCCCTGGATGA | |
| | | | CCACCCTGCTGAGCGCCCTGACCGGCCCCCTGGTGGTGCTG | |
| | | | CTGCTGCTGCTGACCGTGGGCCCCTGCCTGATCAACCGGTT | |
| | | | CCTGGCCTTCGTGCGGGAGCGGGTGAGCGCCGTGCAGATCA | |
| | | | TGGTGCTGCGGCAGCAGTACCAGGGCCTGCTGAGCCAGGGC | |
| | | | GAGACCGACCTGTGATGA | |
| N | A | N | ATGCACCCCACCCTGAGCCGGCGGCACCTGCCCATCCGGGG | SEQ ID NO:52 |
| | | | CGGCGAGCCCAAGCGGCTGAAGATCCCCCTGAGCTTCGCCA | |
| | | | GCATCGCCTGGTTCCTGACCCTGAGCATCACCCCCCAGGTG | |
| | | | AACGGCAAGCGGCTGGTGGACAGCCCCAACACCCACAAGCC | |
| | | | CCTGAGCCTGACCTGGCTGCTGACCGACAGCGGCACCGGCA | |
| | | | TCACCATCAACAGCACCCAGGGCGAGGCCCCCCTGGGCACC | |
| | | | TGGTGGCCCGAGCTGTACGTGTCCCTGCGGAGCGTGATCCC | |
| | | | CGGCCTGAACGACCAGGCCACCCCCCCCGACGTGCTCCCGG | |
| | | | CCTACCGGTTCTACGTGTGCCCCGCCCCCCCCAACAACGAG | |
| | | | GAGTACTGCGGCAACCCCCAGGACTTCTTCTCCAAGCAGTG | |
| | | | GAGCTCCGTGACCAGCAACCACGGCAACTGGAAGTGGCCCA | |
| | | | TCAGCCAGCAGGACCGGGTGAGCTACAGCTTCGTGAACAAC | |
| | | | CCCACCACCTACAACCAGTTCAACTACGGCCACGGCCGGTG | |
| | | | GAAGGACTGGCAGCAGCGGCTGCAGAAGGACGTGCGGAACA | |
| | | | AGCAGATCAGCTGCAACAGCCTGGACCTGGACTACCTGAAG | |
| | | | ATCAGCTTCACCGAGAAGGGCAAGCACGAGAACATCCAGAA | |
| | | | GTGGGTGAACGGCATGAGCTGGGGCATCATGTACTACGGCG | |
| | | | GCAGCGGCCGGCGGAAGGGCAGCGTGCTGACCATCCGGCTG | |
| | | | CGGATCGAGACCCAGATGGAGCCCCCCGTGGCCATCGGCCC | |
| | | | CAACAAGGGCCTGGCCGAGCAGGGCCCCCCCATCCAGGAGC | |
| | | | AGCGGCCCAGCCCCAACCCCAGCGACTACAACACCACCACC | |
| | | | GGCAGCGTGCCCACCGACCCCAACATCACCATCAAGACCGG | |
| | | | CGCCAAGCTGTTCAGCCTGATCCAGGGCGCCTTCCAGGCCC | |
| | | | TGAACACCACCACCCCCGAGGCCACCAGCAGCTGCTGGCTG | |
| | | | TGCCTGGCCACCGGCCCCCCCTACTACGAGGGCATGGCCCG | |
| | | | GGGCGGCAAGTTCAACGTGACCAAGGAGCACCGGGACCAGT | |
| | | | GCACCTGGGGCAGCCAGAACAAGCTGACCCTGACCGAGGTG | |
| | | | AGCGCCAAGGGCACCTGCATCGGCCGGGTGCCCCCCACCCA | |
| | | | CCAGCACCTGTGCAACCACACCGAGGCCTTCAACCGGACCA | |
| | | | GCGAGAGCCAGTACCTGGTGCCCGGCTACGACCGGTGGTGG | |
| | | | GCCTGCAACACCGGCCTGACCCCCTGCGTGAGCACCCTGGT | |
| | | | GTTCAACCAGACCAAGGACTTCTGCGTGATGGTGCAGATCG | |
| | | | TGCCCCGGGTGTACTACTACCCCGAGAAGGCCGTGCTGGAC | |
| | | | GACTACGACTACCGCTACAACCGGCCCAAGCGGGAGCCCAT | |
| | | | CAGCCTGACCCTGGCCGTGATGCTGGGCCTGGCCGTGGCCG | |
| | | | CCGGCGTGGGCACCGGCACCGCCGCCCTGATCACCGGCCCC | |
| | | | CAGCAGCTGGAGAAGGGCCTGAGCGACCTGCACCGGATCGT | |
| | | | GACCGAGGACCTGCAGGCCCTGGAGAAGAGCGTGAGCAACC | |
| | | | TGGAGGACAGCCTGACCAGCCTGACCGAGGTGGTGCTGCAG | |
| | | | AACCGGCGGGGCCTGGACCTGCTGTTCCTGAAGGAGGCCGG | |
| | | | CCTGTGCGTGGCCCTGAACGAGCAGTGCTGCTTCTACGTGG | |
| | | | ACCACAGCGGCGCCATCCCGGACAGCATGAGCAAGCTGCGG | |
| | | | GAGCGGCTGGACCGGCGGCGCCGGGAGCGGGAGGCCGACCA | |
| | | | GGGCTGGTTCGAGGGCTGGTTCAACCGGAGCCCCTGGATGA | |
| | | | CCACCCTGCTGAGCGCCCTGACCGGCCCCCTGGTGGTGCTG | |
| | | | CTCCTGCTGCTGACCGTGCGCCCCTGCCTGATCAACCGGTT | |
| | | | CGTGGCCTTCGTGCGGGAGCGGGTGAGCGCCGTGCAGATCA | |
| | | | TGGTGCTGCGGCAGCAGTACCAGGGCCTGCTGAGCCAGCCC | |
| | | | GAGACCGACCTGTGATGA | |
| A | B | A | ATGCACCCCACCCTGAGCCGGCGGCACCTGCCCACCCGGGG | SEQ ID NO:53 |
| | | | CGGCGAGCCCAAGCGGCTGCGGATCCCCCTGAGCTTCGCCA | |
| | | | GCATCGCCTGGTTCCTGACCCTGACCATCACCCCCCAGGCC | |
| | | | AGCAGCAAGCGGCTGATCGACAGCAGCAACCCCCACCGGCC | |
| | | | CCTGAGCCTGACCTGGCTGATCATCGACCCCGACACCGGCG | |
| | | | TCACCGTGAACACCACCCGGGGCGTGGCCCCCCGGGGCACC | |
| | | | TGCTGGCCCGAGCTCCACTTCTGCCTGCGGCTGATCAACCC | |
| | | | CGCCGTGAAGAGCACCCCCCCCAACCTGGTGCGGAGCTACG | |
| | | | GCTTCTACTGCTGCCCCGGCACCGAGAAGGAGAAGTACTGC | |
| | | | GGCGGCAGCGGCGAGAGCTTCTGCCGGCGGTGGAGCTGCGT | |
| | | | GACCAGCAACGACGGCGACTGGAAGTGGCCCATCAGCCTGC | |
| | | | ACGACCGCGTGAAGTTCAGCTTCGTGAACAGCGGCCCCGGC | |
| | | | AAGTACAAGGTGATGAAGCTGTACAAGGACAAGAGCTGCAG | |
| | | | CCCCAGCGACCTGGACTACCTGAAGATCAGCTTCACCGAGA | |
| | | | AGGGCAAGCAGGAGAACATCCAGAAGTGGATCAACCGCATG | |
| | | | AGCTGGGGCATCGTGTTCTACAAGTACGGCGGCGGCGCCGG | |
| | | | CAGCACCCTGACCATCCGGCTGCGGATCGAGACCGGCACCG | |
| | | | AGCCCCCCGTGGCCGTGGGCCCCGACAAGGTGCTGGCCGAG | |
| | | | CACGGCCCCCCCGCCCTCGAGCCCCCCCACAACCTGCCCGT | |
| | | | GCCCCAGCTGACCAGCCTGCGGCCCGACATCACCCAGCCCC | |
| | | | CCAGCAACAGCACCACCGGCCTGATCCCCACCAACACCCCC | |
| | | | CGGAACAGCCCCGGCGTGCCCGTGAAGACCGGCCAGCGGCT | |
| | | | GTTCAGCCTGATCCACGGCGCCTTCCAGGCCATCAACAGCA | |
| | | | CCCACCCCGACGCCACCAGCAGCTGCTGGCTGTGCCTGAGC | |
| | | | AGCGGCCCCCCCTACTACGAGGGCATGGCCAAGGAGGGCAA | |
| | | | GTTCAACGTGACCAAGGAGCACCGGAACCAGTGCACCTGGG | |
| | | | GCAGCCGGAACAAGCTGACCCTGACCGAGGTGAGCGGCAAG | |
| | | | GGCACCTGCATCGGCAAGGCCCCCCCCAGCCACCAGCACCT | |
| | | | GTGCTACAGCACCGTGGTGTACGAGCAGGCCAGCCAGAACC | |
| | | | AGTACCTGGTGCCCGGCTACAACCGGTGGTGGGCCTGCAAC | |
| | | | ACCGGCCTGACCCCCTGCCTGAGCACCAGCGTGTTCAACCA | |
| | | | GAGCAAGGACTTCTGCGTGATGGTGCAGATCGTGCCCCGGG | |
| | | | TGTACTACCACCCCGAGGAGGTGGTGCTGGACGAGTACGAC | |
| | | | TACCGGTACAACCGGCCCAAGCGGCAGCCCGTGAGCCTGAC | |
| | | | CCTCCCCGTGATGCTGGGCCTGGGCACCGCCGTGGGCGTGG | |
| | | | GCACCGGCACCCCCGCCCTGATCACCGGCCCCCAGCACCTG | |
| | | | GAGAAGGGCCTGGGCGAGCTGCACGCCGCCATGACCGAGGA | |
| | | | CCTGCGGGCCCTGGAGGAGAGCGTGAGCAACCTGGAGGAGA | |
| | | | GCCTGACCAGCCTGAGCGAGGTGGTGCTGCAGAACCGGCGG | |
| | | | GGCCTGGACCTGCTGTTCCTGCGGGACGGCGGCCTGTGCGC | |
| | | | CGCCCTGAAGCAGGAGTGCTGCTTCTACGTGGACCACAGCG | |
| | | | GCGCCATCCGGGACAGCATGAGCAAGCTGCGGGAGCCGCTG | |
| | | | CAGCGCCGGCCGCGGGAGCGGGAGGCCGACCAGGGCTGGTT | |
| | | | CGAGGGCTGGTTCAACCGGAGCCCCTGGATGACCACCCTGC | |
| | | | TGAGCGCCCTGACCGGCCCCCTGGTGGTGCTGCTGCTGCTG | |
| | | | CTGACCGTGGGCCCCTGCCTGATCAACCGGTTCGTGGCCTT | |
| | | | CGTGCGGGAGCGGGTGAGCGCCGTGCAGATCATGGTGCTGC | |
| | | | GGCAGCAGTACCAGGGCCTCCTGAGCCAGGGCGAGACCGAC | |
| | | | CTGTAC | |
| B | B | A | ATCCACCCCACCCTGAGCTCGCGGCACCTGCCCACCCCGGG | SEQ ID NO:54 |
| | | | CGGCGAGCCCAAGCGGCTGCGGATCCCCCTGAGCTTCGCCA | |
| | | | GCATCGCCTGGTTCCTGACCCTGACCATCACCCCCCAGGCC | |
| | | | AGCAGCAAGCGGCTGATCGACAGCAGCAACCCCCACCGGCC | |
| | | | CCTGAGCCTGACCTCGCTGATCATCGACCCCGACACCGGCG | |
| | | | TGACCGTGAACAGCACCCGGGGCGTGGCCCCCCGGGGCACC | |
| | | | TGGTGGCCCGAGCTGCACTTCTGCCTGCGGCTGATCAACCC | |
| | | | CGCCGTGAAGAGCACCCCCCCCAACCTGGTGCGGAGCTACG | |
| | | | GCTTCTACTGCTGCCCCGGCACCGAGAAGGAGAAGTACTGC | |
| | | | GGCGGCAGCGGCGAGAGCTTCTGCCGGCGGTGGAGCTGCGT | |
| | | | GACCAGCAACGACGGCGACTGGAAGTGGCCCATCAGCCTGC | |
| | | | AGGACCGGGTGAAGTTCAGCTTCGTGAACAGCGGCCCCGGC | |
| | | | AAGTACAAGGTGATGAAGCTGTACAAGGACAAGAGCTCCAG | |
| | | | CCCCAGCGACCTGGACTACCTGAAGATCAGCTTCACCGAGA | |
| | | | AGCGCAAGCAGGAGAACATCCAGAAGTGGATCAACGGCATG | |
| | | | AGCTGGCGCATCGTGTTCTACAAGTACGGCGGCGGCGCCGG | |
| | | | CAGCACCCTGACCATCCGGCTGCGGATCGAGACCGGCACCG | |
| | | | AGCCCCCCGTGGCCGTGGGCCCCGACAAGGTGCTGGCCGAG | |
| | | | CACCGCCCCCCCGCCCTGGAGCCCCCCCACAACCTGCCCGT | |
| | | | GCCCCAGCTGACCAGCCTGCGGCCCGACATCACCCAGCCCC | |
| | | | CCAGCAACGGCACCACCGGCCTGATCCCCACCAACACCCCC | |
| | | | CGGAACAGCCCCGGCGTGCCCGTGAAGACCGGCCAGCGGCT | |
| | | | GTTCAGCCTGATCCAGGGCGCCTTCCAGGCCATCAACAGCA | |
| | | | CCGACCCCGACGCCACCAGCAGCTGCTGGCTGTGCCTGAGC | |
| | | | AGCGGCCCCCCCTACTACGAGGGCATGGCCAAGCAGGGCAA | |
| | | | GTTCAACGTGACCAAGGAGCACCGGAACCAGTGCACCTGGG | |
| | | | GCAGCCGGAACAAGCTGACCCTGACCGAGGTGAGCGGCAAG | |
| | | | GGCACCTGCATCGGCAAGGCCCCCCCCAGCCACCAGCACCT | |
| | | | GTGCTACAGCACCGTGGTGTACGAGCAGGCCAGCGAGAACC | |
| | | | AGTACCTGGTGCCCGGCTACAACCGGTGGTGGGCCTGCAAC | |
| | | | ACCGGCCTGACCCCCTGCGTCAGCACCAGCGTGTTCAACCA | |
| | | | GAGCAAGGACTTCTGCGTGATGGTGCAGATCGTGCCCCCGG | |
| | | | TGTACTACCACCCCGAGGAGGTGGTGCTGGACCAGTACCAC | |
| | | | TACCGCTACAACCGGCCCAAGCGGGAGCCCGTGAGCCTGAC | |
| | | | CCTCCCCGTGATCCTGGGCCTGGGCACCGCCGTGGGCGTGG | |
| | | | GCACCCGCACCCCCGCCCTGATCACCGGCCCCCAGCAGCTG | |
| | | | GAGAACGGCCTGGGCGAGCTGCACGCCGCCATGACCGAGGA | |
| | | | CCTGCCGGCCCTGGAGGAGAGCGTGAGCAACCTGGAGGAGA | |
| | | | GCCTGACCAGCCTGAGCCAGGTGGTGCTGCAGAACCGGCGG | |
| | | | GGCCTCGACCTGCTGTTCCTGCGGGAGGGCGGCCTGTGCGC | |
| | | | CCCCCTGAAGGAGGAGTGCTGCTTCTACGTGGACCACAGCG | |
| | | | GCGCCATCCGGGACAGCATGAGCAAGCTGCGGGAGCGGCTG | |
| | | | GAGCGGCCGCGGCGGGAGCGCCAGGCCGACCAGGGCTGGTT | |
| | | | CGAGGGCTGGTTCAACCGGAGCCCCTGCATGACCACCCTGC | |
| | | | TGAGCGCCCTGACCCGCCCCCTGGTGGTGCTGCTGCTGCTG | |
| | | | CTGACCGTGGGCCCCTGCCTGATCAACCGGTTCGTGGCCTT | |
| | | | CGTGCGGGAGCGGGTGACCGCCGTGCACATCATGGTGCTGC | |
| | | | GGCAGCAGTACCAGGGCCTGCTGAGCCAGGGCGAGACCGAC | |
| | | | CTGTGATGA | |
| B | B | N | ATGCACCCCACCCTGAGCTGGCGGCACCTGCCCACCCGGGG | SEQ ID NO:55 |
| | | | CGGCGAGCCCAAGCGGCTGCGGATCCCCCTGAGCTTCGCCA | |
| | | | GCATCGCCTGGTTCCTGACCCTGACCATCACCCCCCAGGCC | |
| | | | AGCACCAAGCGGCTGATCGACAGCAGCAACCCCCACCGGCC | |
| | | | CCTCAGCCTGACCTGGCTGATCATCGACCCCGACACCGGCG | |
| | | | TGACCGTGAACAGCACCCGCGGCGTGGCCCCCCGGGGCACC | |
| | | | TGGTGGCCCGAGCTGCACTTCTGCCTGCGGCTGATCAACCC | |
| | | | CGCCGTGAACAGCACCCCCCCCAACCTGGTGCGGAGCTACG | |
| | | | GCTTCTACTGCTGCCCCGGCACCGAGAAGGAGAAGTACTCC | |
| | | | GGCGGCAGCGGCGAGAGCTTCTGCCGCCGGTGGAGCTGCCT | |
| | | | GACCAGCAACGACGGCGACTGGAAGTGGCCCATCAGCCTGC | |
| | | | AGGACCGGCTGAAGTTCAGCTTCGTGAACAGCGGCCCCGGC | |
| | | | AAGTACAACGTGATGAAGCTGTACAAGGACAAGAGCTGCAG | |
| | | | CCCCAGCGACCTGGACTACCTGAAGATCAGCTTCACCGAGA | |
| | | | AGGGCAAGCAGGAGAACATCCAGAAGTGGATCAACGGCATG | |
| | | | AGCTGGGGCATCGTGTTCTACAAGTACGGCGGCGGCGCCGG | |
| | | | CAGCACCCTGACCATCCGGCTGCGGATCGAGACCGGCACCG | |
| | | | AGCCCCCCGTGGCCGTGCGCCCCGACAAGGTGCTGGCCGAG | |
| | | | CAGGGCCCCCCCGCCCTGGAGCCCCCCCACAACCTGCCCGT | |
| | | | GCCCCAGCTGACCAGCCTGCGGCCCGACATCACCCAGCCCC | |
| | | | CCAGCAACAGCACCACCGGCCTGATCCCCACCAACACCCCC | |
| | | | CGGAACAGCCCCGGCGTGCCCGTGAAGACCGGCCAGCGCCT | |
| | | | GTTCAGCCTGATCCAGGGCGCCTTCCAGGCCATCAACACCA | |
| | | | CCGACCCCGACGCCACCAGCAGCTGCTGGCTGTCCCTGAGC | |
| | | | AGCGGCCCCCCCTACTACGAGGGCATGGCCAAGCAGGGCAA | |
| | | | GTTCAACGTGACCAAGGAGCACCGGAACCAGTGCACCTGGG | |
| | | | GCAGCCGGAACAAGCTGACCCTGACCGAGGTGAGCGGCAAG | |
| | | | GGCACCTGCATCGGCAAGGCCCCCCCCAGCCACCAGCACCT | |
| | | | GTGCAACAGCACCGTGGTGTACGACCAGGCCAGCGAGAACC | |
| | | | AGTACCTGGTGCCCGGCTACAACCCGTGGTGCGCCTGCAAC | |
| | | | ACCGGCCTGACCCCCTGCGTCAGCACCAGCGTGTTCAACCA | |
| | | | GAGCAAGGACTTCTGCGTGATGGTGCAGATCGTGCCCCGGG | |
| | | | TGTACTACCACCCCGAGGAGGTGGTGCTGGACGAGTACGAC | |
| | | | TACCGGTACAACCGGCCCAAGCGGGAGCCCGTGAGCCTGAC | |
| | | | CCTGGCCGTGATGCTGGGCCTGGGCACCGCCGTGGGCGTGG | |
| | | | GCACCGGCACCGCCGCCCTGATCACCGGCCCCCAGCAGCTG | |
| | | | GAGAAGGGCCTGGGCGAGCTGCACGCCGCCATGACCGAGGA | |
| | | | CCTGCGGGCCCTGGAGGAGAGCGTGAGCAACCTGGAGGAGA | |
| | | | CCCTCACCAGCCTGAGCGAGGTGGTCCTGCAGAACCGGCGG | |
| | | | GGCCTGGACCTGCTGTTCCTGCGGGAGGGCGGCCTGTGCGC | |
| | | | CGCCCTGAAGGAGGAGTGCTGCTTCTACGTGGACCACAGCG | |
| | | | CCCCCATCCGGGACAGCATGACCAAGCTGCGCGAGCGGCTG | |
| | | | GAGCGGCGGCGGCGGGAGCGGGAGGCCGACCAGGGCTGGTT | |
| | | | CGAGGGCTGGTTCAACCGGAGCCCCTGGATGACCACCCTGC | |
| | | | TGAGCGCCCTGACCGGCCCCCTGGTGGTGCTGCTGCTGCTG | |
| | | | CTGACCGTGGGCCCCTGCCTGATCAACCGGTTCGTGGCCTT | |
| | | | CGTGCGGGACCGGGTGAGCGCCGTGCAGATCATGGTGCTGC | |
| | | | GGCAGCAGTACCAGGGCCTGCTGAGCCAGGGCGAGACCGAC | |
| | | | CTGTGATGA | |
| C | B | A | ATGCACCCCACCCTGAGCTGGCGGCACCTCCCCACCCGGGG | SEQ ID NO:56 |
| | | | CGGCGAGCCCAAGCGGCTGCGGATCCCCCTGAGCTTCCCCA | |
| | | | GCATCGCCTGGTTCCTGACCCTGACCATCACCCCCCAGCCC | |
| | | | AGCAGCAAGCGGCTGATCGACAGCAGCAACCCCCACCGGCC | |
| | | | CCTGAGCCTGACCTGGCTGATCATCGACCCCGACACCGGCG | |
| | | | TGACCGTGAACAGCACCCGGGCCGTGGCCCCCCGGGGCACC | |
| | | | TGGTGGCCCGAGCTGCACTTCTGCCTGCGGCTGATCAACCC | |
| | | | CGCCGTGAAGCACCAGAGCACCCCCCCCAACCTGGTGCGGA | |
| | | | GCTACGGCTTCTACTGCTGCCCCGGCACCCCCGAGAAGGAG | |
| | | | AAGTACTGCGGCGGCAGCGGCGAGAGCTTCTGCCGGCGGTG | |
| | | | GACCTGCGTGACCACCAACGACGGCGACTGGAAGTGGCCCA | |
| | | | TCAGCCTGCAGGACCGGGTGAAGTTCAGCTTCGTGAACAGC | |
| | | | GGCCCCGGCTACAACCAGTTCAACTACGGCCACGGCCGGTG | |
| | | | GAAGGACTCGAAGTACAAGGTGATGAACCTGTACAAGGACA | |
| | | | AGCAGATCAGCTGCAGCCCCAGCGACCTGGACTACCTGAAG | |
| | | | ATCAGCTTCACCGAGAACGGCAAGCACCAGAACATCCAGAA | |
| | | | GTGGATCAACGGCATGAGCTGGGGCATCGTCTTCTACAACT | |
| | | | ACGGCGGCGGCAAGGCCGGCAGCACCCTGACCATCCGGCTG | |
| | | | CGGATCGAGACCGGCACCGAGCCCCCCGTGGCCGTGCGCCC | |
| | | | CGACAAGGTGCTGGCCGAGCAGGGCCCCCCCGCCCTGGAGC | |
| | | | CCCCCCACAACCTGCCCGTGCCCCAGCTGACCAGCCTGCCG | |
| | | | CCCGACATCACCCAGCCCCCCAGCAACGGCACCACCGGCCT | |
| | | | GATCCCCACCAACACCCCCCGGAACAGCCCCGCCGTGCCCG | |
| | | | TGAAGACCGGCCAGCGGCTGTTCAGCCTGATCCAGCGCGCC | |
| | | | TTCCAGGCCATCAACAGCACCCACCCCGACGCCACCAGCAG | |
| | | | CTCCTGGCTGTGCCTGAGCAGCGGCCCCCCCTACTACGAGG | |
| | | | CCATGGCCAAGGAGGGCAAGTTCAACGTGACCAAGGAGCAC | |
| | | | CGGAACCAGTGCACCTCGGGCAGCCGGAACAAGCTGACCCT | |
| | | | GACCGAGGTGAGCGGCAAGGGCACCTGCATCGGCAAGGCCC | |
| | | | CCCCCAGCCACCAGCACCTGTGCTACAGCACCGTGGTGTAC | |
| | | | GAGCAGGCCAGCGAGAACCAGTACCTGGTGCCCGGCTACAA | |
| | | | CCGGTGGTGGGCCTGCAACACCGGCCTGACCCCCTGCGTGA | |
| | | | GCACCAGCGTGTTCAACCAGAGCAAGGACTTCTGCGTGATG | |
| | | | GTGCAGATCGTGCCCCGGGTGTACTACCACCCCCAGGAGGT | |
| | | | GGTGCTGGACGAGTACGACTACCGGTACAACCGCCCCAAGC | |
| | | | GGGAGCCCGTGAGCCTGACCCTGGCCGTGATGCTGGGCCTG | |
| | | | GGCACCGCCGTGGGCGTGGGCACCGGCACCGCCGCCCTGAT | |
| | | | CACCGGCCCCCAGCAGCTGGAGAAGGGCCTGGGCGAGCTGC | |
| | | | ACGCCGCCATGACCGAGGACCTGCGGGCCCTGGAGGAGAGC | |
| | | | GTGAGCAACCTGGAGGAGAGCCTGACCAGCCTGAGCGAGGT | |
| | | | GGTGCTGCAGAACCGGCGGGGCCTGGACCTGCTGTTCCTGC | |
| | | | GGGAGGGCGGCCTGTGCCCCGCCCTGAACGAGGAGTGCTGC | |
| | | | TTCTACGTGGACCACAGCGGCGCCATCCCGGACAGCATGAG | |
| | | | CAAGCTGCGGGAGCGGCTGGAGCGGCGGCGGCGGGAGCGCG | |
| | | | AGGCCGACCAGGGCTGGTTCGAGGGCTGGTTCAACCGGACC | |
| | | | CCCTCCATGACCACCCTGCTGAGCGCCCTGACCGGCCCCCT | |
| | | | GGTGGTCCTGCTGCTGCTGCTGACCGTGGGCCCCTGCCTGA | |
| | | | TCAACCCCTTCCTGGCCTTCGTGCGGGAGCGGGTGAGCGCC | |
| | | | GTCCAGATCATGGTCCTGCGGCAGCAGTACCAGGGCCTGCT | |
| | | | GAGCCACGGCCAGACCGACCTGTAC | |
| C | B | N | ATGCACCCCACCCTGAGCTGGCGGCACCTGCCCACCCGGGG | SEQ ID NO:57 |
| | | | CGGCGAGCCCAAGCGGCTGCGGATCCCCCTGAGCTTCGCCA | |
| | | | GCATCGCCTGGTTCCTGACCCTGACCATCACCCCCCAGGCC | |
| | | | AGCAGCAAGCGGCTGATCGACAGCAGCAACCCCCACCGGCC | |
| | | | CCTGAGCCTGACCTGGCTGATCATCGACCCCGACACCGGCG | |
| | | | TGACCGTGAACAGCACCCGGGGCGTGGCCCCCCGGGGCACC | |
| | | | TGGTGGCCCGAGCTGCACTTCTGCCTGCGCCTGATCAACCC | |
| | | | CGCCGTGAAGGACCAGAGCACCCCCCCCAACCTGGTGCGGA | |
| | | | GCTACCGCTTCTACTGCTGCCCCGGCACCCCCGACAAGGAG | |
| | | | AAGTACTGCGGCGCCAGCGGCGAGACCTTCTGCCCGCGGTG | |
| | | | GAGCTGCGTGACCAGCAACGACGGCGACTCCAAGTGGCCCA | |
| | | | TCAGCCTGCAGGACCGGGTGAAGTTCAGCTTCGTGAACAGC | |
| | | | GGCCCCGGCTACAACCAGTTCAACTACGGCCACGGCCGGTG | |
| | | | CAAGGACTGGAAGTACAAGGTGATCAAGCTGTACAAGGACA | |
| | | | AGCAGATCAGCTCCAGCCCCAGCGACCTGGACTACCTGAAG | |
| | | | ATCAGCTTCACCGACAAGGGCAAGCACGAGAACATCCAGAA | |
| | | | GTGGATCAACGCCATGAGCTGGGGCATCGTGTTCTACAAGT | |
| | | | ACGGCGGCGCCCGGGCCGGCAGCACCCTCACCATCCGGCTG | |
| | | | CGGATCGAGACCGGCACCGACCCCCCCGTGGCCCTGCGCCC | |
| | | | CGACAAGGTGCTGGCCGAGCAGCGCCCCCCCGCCCTGGAGC | |
| | | | CCCCCCACAACCTGCCCGTGCCCCAGCTGACCAGCCTGCGG | |
| | | | CCCGACATCACCCAGCCCCCCAGCAACACCACCACCGGCCT | |
| | | | GATCCCCACCAACACCCCCCGGAACACCCCCCGCGTGCCCC | |
| | | | TGAAGACCGGCCACCGCCTCTTCAGCCTGATCCACGGCGCC | |
| | | | TTCCAGGCCATCAACAGCACCGACCCCGACGCCACCAGCAG | |
| | | | CTGCTGGCTGTGCCTGAGCAGCGGCCCCCCCTACTACGAGG | |
| | | | GCATGGCCAAGGAGGGCAACTTCAACGTGACCAAGGAGCAC | |
| | | | CGGAACCAGTGCACCTGGGGCAGCCGGAACAAGCTGACCCT | |
| | | | GACCGAGGTGAGCGGCAAGGGCACCTGCATCGGCAAGGCCC | |
| | | | CCCCCAGCCACCAGCACCTGTGCAACAGCACCGTGGTGTAC | |
| | | | GAGCAGCCCAGCGAGAACCAGTACCTGGTGCCCGGCTACAA | |
| | | | CCCGTGGTGGGCCTGCAACACCGGCCTGACCCCCTGCGTGA | |
| | | | GCACCAGCCTGTTCAACCAGAGCAAGGACTTCTGCGTGATG | |
| | | | GTGCAGATCGTGCCCCGGGTGTACTACCACCCCCAGGAGGT | |
| | | | GGTGCTGGACGAGTACGACTACCGGTACAACCCGCCCAAGC | |
| | | | GGGAGCCCGTGAGCCTGACCCTGGCCGTGATGCTGGGCCTG | |
| | | | GGCACCGCCGTGGGCGTGGGCACCGGCACCGCCGCCCTGAT | |
| | | | CACCGGCCCCCAGCAGCTGGAGAAGGGCCTGGGCGAGCTGC | |
| | | | ACGCCGCCATGACCGAGGACCTGCCGGCCCTGGAGGAGAGC | |
| | | | GTGAGCAACCTCGAGGAGAGCCTGACCAGCCTGAGCGAGGT | |
| | | | GGTGCTGCAGAACCGGCGGGGCCTGGACCTGCTGTTCCTGC | |
| | | | GGGAGGGCGGCCTGTGCGCCGCCCTGAAGGAGGAGTGCTGC | |
| | | | TTCTACGTGGACCACAGCGGCGCCATCCGGGACAGCATGAG | |
| | | | CAAGCTGCGGGAGCGGCTGGAGCGGCGGCGGCGGGAGCGGG | |
| | | | ACGCCGACCAGGGCTGGTTCGAGGGCTGGTTCAACCGAAGC | |
| | | | CCCTGGATGACCACCCTGCTGAGCCCCCTGACCGGCCCCCT | |
| | | | GGTGGTGCTGCTGCTGCTGCTGACCGTGGGCCCCTGCCTGA | |
| | | | TCAACCGGTTCGTGGCCTTCGTGCGGGAGCGGGTGAGCGCC | |
| | | | GTGCAGATCATGGTGCTGCGGCAGCAGTACCAGGGCCTGCT | |
| | | | GAGCCAGGGCGAGACCGACCTGTAC | |
| N | B | A | ATGCACCCCACCCTGAGCTGGCGGCACCTGCCCACCCGGGG | SEQ ID NO:58 |
| | | | CGGCGAGCCCAAGCGGCTCCGGATCCCCCTGAGCTTCGCCA | |
| | | | GCATCGCCTGGTTCCTGACCCTCACCATCACCCCCCAGGCC | |
| | | | AGCAGCAAGCGGCTGATCGACAGCACCAACCCCCACCGGCC | |
| | | | CCTGAGCCTGACCTGGCTGATCATCGACCCCGACACCGGCG | |
| | | | TGACCGTGAACAGCACCCGGGGCGTGGCCCCCCGGGGCACC | |
| | | | TGGTGGCCCCAGCTGCACTTCTGCCTGCGGCTGATCAACCC | |
| | | | CGCCGTGAAGAGCACCCCCCCCAACCTGGTGCGGAGCTACG | |
| | | | GCTTCTACTGCTGCCCCGGCACCGACAACGAGAAGTACTGC | |
| | | | GGCGGCAGCGGCGAGAGCTTCTCCCGGCGGTGGAGCTGCGT | |
| | | | GACCAGCAACGACGCCCACTGGAAGTCGCCCATCAGCCTGC | |
| | | | AGGACCGGGTGAAGTTCAGCTTCCTGAACAGCGGCCCCGGC | |
| | | | AAGTACAAGGTGATGAAGCTCTACAACGACAAGAGCTGCAG | |
| | | | CCCCAGCGACCTGGACTACCTGAAGATCAGCTTCACCGAGA | |
| | | | AGGGCAAGCAGGAGAACATCCAGAAGTGGATCAACGGCATG | |
| | | | ACCTCGGGCATCGTGTTCTACAAGTACGGCGGCGGCGCCGG | |
| | | | CAGCACCCTGACCATCCGGCTGCGGATCGAGACCGGCACCG | |
| | | | AGCCCCCCGTGGCCGTGGGCCCCGACAAGGTGCTGGCCGAG | |
| | | | CACCGCCCCCCCGCCCTGGAGCCCCCCCACAACCTGCCCGT | |
| | | | GCCCCAGCTGACCAGCCTGCGGCCCGACATCACCCAGCCCC | |
| | | | CCAGCAACGGCACCACCGGCCTGATCCCCACCAACACCCCC | |
| | | | CGGAACACCCCCGGCGTGCCCGTGAAGACCGGCCAGCGGCT | |
| | | | GTTCAGCCTGATCCAGGGCGCCTTCCAGGCCATCAACAGCA | |
| | | | CCGACCCCGACGCCACCACCAGCTGCTGGCTGTGCCTGAGC | |
| | | | ACCGGCCCCCCCTACTACGAGGGCATGCCCAAGGAGGGCAA | |
| | | | GTTCAACGTGACCAAGGAGCACCGGAACCAGTGCACCTGGG | |
| | | | GCAGCCGGAACAAGCTGACCCTGACCGAGGTGAGCGCCAAG | |
| | | | GGCACCTGCATCGGCAAGGCCCCCCCCAGCCACCAGCACCT | |
| | | | GTGCTACACCACCGTGGTGTACGAGCAGGCCAGCGAGAACC | |
| | | | AGTACCTGGTGCCCGGCTACAACCGGTGGTGCGCCTCCAAC | |
| | | | ACCGGCCTGACCCCCTGCCTGAGCACCAGCGTGTTCAACCA | |
| | | | GAGCAAGGACTTCTGCGTGATGGTGCAGATCGTGCCCCGGG | |
| | | | TGTACTACCACCCCGAGGAGGTCGTGCTGGACGAGTACGAC | |
| | | | TACCGGTACAACCGGCCCAAGCCGGACCCCGTGAGCCTGAC | |
| | | | CCTGGCCGTGATGCTGGGCCTGCCCACCGCCGTGGGCGTGG | |
| | | | GCACCGGCACCGCCGCCCTGATCACCCGCCCCCAGCAGCTG | |
| | | | GAGAAGGGCCTGGGCGAGCTGCACGCCGCCATGACCGAGGA | |
| | | | CCTGCGCGCCCTCGAGGAGAGCGTGACCAACCTGGAGGAGA | |
| | | | GCCTGACCAGCCTGAGCGAGGTGGTGCTGCAGAACCGGCGG | |
| | | | GGCCTGGACCTGCTGTTCCTGCCCGAGGGCGGCCTGTGCGC | |
| | | | CGCCCTGAAGGAGGAGTGCTGCTTCTACGTGGACCACAGCG | |
| | | | GCGCCATCCGGGACAGCATGAGCAAGCTGCGGGAGCGGCTG | |
| | | | GAGCGGCGGCGGCGGGAGCCGGAGGCCCACCAGGGCTGGTT | |
| | | | CGAGGGCTGGTTCAACCGGAGCCCCTGGATGACCACCCTGC | |
| | | | TGAGCGCCCTGACCGGCCCCCTGGTGGTGCTGCTGCTGCTG | |
| | | | CTGACCGTGGGCCCCTGCCTGATCAACCGGTTCGTGGCCTT | |
| | | | CGTGCGGGAGCCGGTGAGCGCCGTGCAGATCATGGTGCTGC | |
| | | | GGCAGCAGTACCAGGGCCTGCTGAGCCAGGGCGAGACCGAC | |
| | | | CTGTGATGA | |
| N | B | N | ATGCACCCCACCCTGAGCTGGCGGCACCTGCCCACCCGGGG | SEQ ID NO:59 |
| | | | CGGCGACCCCAAGCGGCTGCGGATCCCCCTGAGCTTCGCCA | |
| | | | GCATCGCCTGGTTCCTGACCCTGACCATCACCCCCCAGGCC | |
| | | | AGCAGCAAGCGGCTGATCCACAGCAGCAACCCCCACCGGCC | |
| | | | CCTGAGCCTGACCTGGCTGATCATCGACCCCGACACCGGCG | |
| | | | TGACCGTGAACAGCACCCCGCGCGTGGCCCCCCGGGGCACC | |
| | | | TGGTGGCCCGAGCTGCACTTCTGCCTGCGGCTGATCAACCC | |
| | | | CGCCGTGAAGAGCACCCCCCCCAACCTGGTCCGGAGCTACC | |
| | | | GCTTCTACTGCTCCCCCGGCACCGAGAAGGAGAAGTACTGC | |
| | | | GGCGGCAGCGGCGAGAGCTTCTGCCGGCGGTGGAGCTGCGT | |
| | | | GACCAGCAACGACGGCGACTGGAAGTGGCCCATCAGCCTGC | |
| | | | AGGACCGGGTGAAGTTCAGCTTCGTGAACACCGGCCCCGGC | |
| | | | AAGTACAACGTGATGAAGCTGTACAAGGACAAGAGCTGCAG | |
| | | | CCCCAGCGACCTGGACTACCTGAAGATCAGCTTCACCGAGA | |
| | | | AGGGCAAGCAGGAGAACATCCAGAAGTGGATCAACGGCATG | |
| | | | AGCTGGGGCATCCTGTTCTACAAGTACGGCGGCGGCGCCGC | |
| | | | CAGCACCCTGACCATCCGGCTGCCCATCGAGACCGGCACCG | |
| | | | AGCCCCCCGTGGCCGTGGGCCCCGACAAGGTGCTGGCCGAG | |
| | | | CAGGCCCCCCCCGCCCTGGAGCCCCCCCACAACCTGCCCGT | |
| | | | GCCCCAGCTGACCAGCCTGCGGCCCGACATCACCCACCCCC | |
| | | | CCAGCAACAGCACCACCGGCCTGATCCCCACCAACACCCCC | |
| | | | CGGAACAGCCCCGGCGTGCCCGTGAAGACCGGCCAGCGGCT | |
| | | | GTTCAGCCTGATCCAGGGCGCCTTCCAGGCCATCAACAGCA | |
| | | | CCGACCCCGACGCCACCAGCAGCTGCTGGCTGTGCCTGAGC | |
| | | | AGCGGCCCCCCCTACTACGAGGGCATGGCCAAGGAGGCCAA | |
| | | | GTTCAACGTGACCAAGGAGCACCGGAACCAGTGCACCTGGG | |
| | | | GCAGCCGGAACAAGCTGACCCTGACCGAGGTGAGCGGCAAG | |
| | | | GGCACCTGCATCGGCAAGGCCCCCCCCAGCCACCAGCACCT | |
| | | | GTGCAACAGCACCGTGGTGTACGACCAGGCCAGCGAGAACC | |
| | | | AGTACCTGGTGCCCGGCTACAACCGGTGGTCGGCCTGCAAC | |
| | | | ACCGGCCTGACCCCCTGCGTGAGCACCACCGTGTTCAACCA | |
| | | | GAGCAAGGACTTCTGCGTGATGGTCCAGATCGTGCCCCGGG | |
| | | | TGTACTACCACCCCGAGGAGGTGGTGCTGGACGAGTACGAC | |
| | | | TACCGGTACAACCGGCCCAAGCGGGAGCCCGTGAGCCTGAC | |
| | | | CCTCGCCGTGATGCTGGGCCTGGGCACCCCCGTGGGCGTGG | |
| | | | GCACCGGCACCGCCGCCCTGATCACCGGCCCCCAGCACCTG | |
| | | | GAGAAGGGCCTGGGCGAGCTGCACGCCGCCATGACCCAGGA | |
| | | | CCTGCGGGCCCTCGAGGAGAGCCTGAGCAACCTGGACGACA | |
| | | | GCCTGACCAGCCTGAGCGAGGTGGTGCTGCAGAACCGGCGG | |
| | | | GGCCTGGACCTGCTGTTCCTCCGGGAGGGCGGCCTGTGCGC | |
| | | | CGCCCTGAAGGAGGAGTGCTGCTTCTACCTGGACCACAGCG | |
| | | | GCGCCATCCGGGACAGCATGAGCAAGCTGCGGGAGCCGCTG | |
| | | | GACCGGCGGCGGCGGGAGCGGGAGGCCGACCAGGGCTGGTT | |
| | | | CGAGGGCTGGTTCAACCGGAGCCCCTGGATGACCACCCTGC | |
| | | | TGAGCGCCCTCACCGGCCCCCTGGTGGTGCTGCTGCTGCTG | |
| | | | CTGACCGTGGGCCCCTGCCTGATCAACCGGTTCGTGCCCTT | |
| | | | CGTGCGGGAGCGGGTGAGCGCCGTGCAGATCATGGTGCTGC | |
| | | | GGCAGCAGTACCAGGCCCTGCTGAGCCAGGGCGAGACCGAC | |
| | | | CTGTGATGA | |
| A | C | A | ATGCACCCCACCCTGAGCCGGCGGCACCTGCCCATCCGGGG | SEQ ID NO:60 |
| | | | CGGCAAGCCCAAGCGGCTGAAGATCCCCCTGAGCTTCGCCA | |
| | | | GCATCGCCTGGTTCCTGACCCTGAGCATCACCAGCCAGACC | |
| | | | AACGGCATGCGGATCGGCGACAGCCTGAACAGCCACAAGCC | |
| | | | CCTGAGCCTGACCTGGCTCATCACCGACAGCGGCACCGGCA | |
| | | | TCAACATCAACAACACCCAGGGCGAGGCCCCCCTGCCCACC | |
| | | | TGGTGGCCCGACCTGTACGTGTGCCTGCGGAGCGTGATCCC | |
| | | | CAGCCTGACCAGCCCCCCCGACATCCTGCACGCCCACGGCT | |
| | | | TCTACGTGTGCCCCGGCCCCCCCAACAACGGCAAGCACTGC | |
| | | | CGCAACCCCCGGGACTTCTTCTCCAAGCAGTGGAACTCCGT | |
| | | | GACCAGCAACGACGGCTACTGGAAGTGGCCCACCAGCCAGC | |
| | | | AGGACCGGGTGAGCTTCAGCTACGTGAACACCTACACCAGC | |
| | | | AGCGGCCAGTTCAACTACCTGACCTGGATCCGGACCGGCAG | |
| | | | CCCCAAGTGCAGCCCCAGCGACCTGGACTACCTGAAGATCA | |
| | | | GCTTCACCGAGAAGCGCAAGCAGGAGAACATCCTGAAGTGG | |
| | | | GTGAACGGCATGAGCTGCGGCATGGTGTACTACGGCGGCAG | |
| | | | CGGCAAGCAGCCCGGCAGCATCCTGACCATCCGGCTGAAGA | |
| | | | TCAACCAGCTGGAGCCCCCCATGGCCATCGGCCCCAACACC | |
| | | | GTGCTGACCGGCCAGCGGCCCCCCACCCAGGGCCCCGGCCC | |
| | | | CAGCAGCAACATCACCAGCGGCAGCGACCCCACCGAGAGCA | |
| | | | ACACCACCACCAAGATGGGCGCCAAGCTGTTCAGCCTGATC | |
| | | | CAGGGCGCCTTCCAGGCCCTGAACAGCACCACCCCCGACGC | |
| | | | CACCAGCAGCTGCTGGCTGTGCCTGGCCCTGGGCCCCCCCT | |
| | | | ACTACGAGGGCATGGCCCGGCGGGGCAAGTTCAACGTGACC | |
| | | | AAGCAGCACCGGGACCAGTGCACCTGGGGCAGCCAGAACAA | |
| | | | GCTGACCCTGACCGAGGTGAGCGGCAAGGGCACCTGCATCG | |
| | | | GCAAGGTGCCCCCCAGCCACCAGCACCTGTGCAACCACACC | |
| | | | CAGGCCTTCAACCAGACCAGCGAGAGCCAGTACCTGGTGCC | |
| | | | CGGCTACGACCGGTGGTGGGCCTGCAACACCGGCCTGACCC | |
| | | | CCTCCGTGAGCACCCTGGTGTTCAACCACACCAAGGACTTC | |
| | | | TCCATCATGGTGCAGATCGTGCCCCGGGTGTACTACTACCC | |
| | | | CGACAAGGCCATCCTGGACGAGTACGACTACCGGAACCACC | |
| | | | GGCAGAAGCGGGAGCCCATCAGCCTGACCCTGGCCGTGATG | |
| | | | CTGGGCCTGGGCGTGGCCGCCGGCGTGGGCACCGGCACCGC | |
| | | | CGCCCTGGTGACCGGCCCCCAGCAGCTGGAGACCGGCCTGA | |
| | | | GCAACCTGCACCGGATCGTGACCGAGGACCTGCAGGCCCTG | |
| | | | CAGAAGAGCGTGAGCAACCTGGAGGAGAGCCTGACCAGCCT | |
| | | | GAGCGAGGTGGTGCTGCAGAACCGGCGGGGCCTGGACCTGC | |
| | | | TGTTCCTGAAGGAGGGCGGCCTGTGCGTGGCCCTGAAGGAG | |
| | | | GAGTGCTGCTTCTACGTGGACCACAGCCGCGCCATCCGGGA | |
| | | | CAGCATGAACAAGCTGCGGGAGCGGCTGGAGAAGCGGCGGC | |
| | | | GGGAGAAGGACACCACCCAGGGCTGGTTCGAGGGCTGGTTC | |
| | | | AACCGGAGCCCCTCGCTGGCCACCCTGCTGAGCGCCCTCAC | |
| | | | CGGCCCCCTGATCGTGCTGCTGCTGCTGCTGACCGTGGGCC | |
| | | | CCTGCATCATCAACAAGCTCATCGCCTTCATCCGGGAGCGG | |
| | | | ATCAGCGCCGTGCAGATCATGGTCCTGCGCCAGCAGTACCA | |
| | | | GAGCCCCAGCACCCGGGAGCCCGGCCCG | |
| B | C | A | ATCCACCCCACCCTGAGCCGGCGGCACCTGCCCATCCGGGG | SEQ ID NO:61 |
| | | | CGGCAAGCCCAAGCGGCTGAAGATCCCCCTGAGCTTCCCCA | |
| | | | GCATCGCCTGGTTCCTGACCCTGAGCATCACCAGCCAGACC | |
| | | | AACGGCATGCGGATCGGCGACAGCCTGAACAGCCACAAGCC | |
| | | | CCTGAGCCTCACCTGGCTGATCACCGACAGCGGCACCGGCA | |
| | | | TCAACATCAACAACACCCAGGGCGAGGCCCCCCTGGGCACC | |
| | | | TGGTGGCCCCACCTGTACCTGTGCCTGCGGAGCGTGATCCC | |
| | | | CAGCCTGACCAGCCCCCCCGACATCCTGCACGCCCACGGCT | |
| | | | TCTACGTGTGCCCCGGCCCCCCCAACAACGGCAAGCACTGC | |
| | | | GGCAACCCCCGGGACTTCTTCTGCAAGCAGTGCAACTGCGT | |
| | | | GACCAGCAACGACGGCTACTGGAAGTGGCCCACCAGCCAGC | |
| | | | AGGACCGGGTGAGCTTCAGCTACGTGAACACCTACACCAGC | |
| | | | AGCGGCCAGTTCAACTACCTGACCTGGATCCGGACCCGCAC | |
| | | | CCCCAAGTGCAGCCCCAGCGACCTGGACTACCTGAAGATCA | |
| | | | GCTTCACCGAGAAGGGCAAGCAGGAGAACATCCTGAAGTGG | |
| | | | GTGAACGGCATGAGCTGGGGCATGGTCTACTACGGCGGCAG | |
| | | | CGGCAAGCAGCCCGGCAGCATCCTGACCATCCGGCTGAAGA | |
| | | | TCAACCAGCTGGAGCCCCCCATGGCCATCGGCCCCACAACC | |
| | | | CTGCTGACCGGCCAGCGGCCCCCCACCCACGGCCCCCGCCC | |
| | | | CAGCAGCAACATCACCAGCGGCAGCGACCCCACCGAGAGCA | |
| | | | ACAGCACCACCAAGATGGGCGCCAAGCTGTTCAGCCTGATC | |
| | | | CAGGGCGCCTTCCAGGCCCTGAACAGCACCACCCCCGAGGC | |
| | | | CACCAGCAGCTGCTGGCTGTGCCTGGCCCTGGGCCCCCCCT | |
| | | | ACTACGAGGGCATGGCCCGGCGCGGCAAGTTCAACGTGACC | |
| | | | AACGAGCACCGGGACCAGTGCACCTGGCGCAGCCAGAACAA | |
| | | | GCTGACCCTGACCGAGGTGAGCGGCAAGGCCACCTGCATCG | |
| | | | GCAAGGTGCCCCCCAGCCACCAGCACCTGTGCAACCACACC | |
| | | | GAGGCCTTCAACCAGACCAGCGAGAGCCAGTACCTGGTGCC | |
| | | | CGGCTACGACCCGTGGTGGGCCTGCAACACCGGCCTGACCC | |
| | | | CCTGCGTGAGCACCCTGGTGTTCAACCAGACCAAGGACTTC | |
| | | | TGCATCATGGTGCAGATCGTGCCCCGGGTGTACTACTACCC | |
| | | | CGAGAAGGCCATCCTGGACGAGTACGACTACCGCAACCACC | |
| | | | GGCAGAAGCGGGAGCCCATCAGCCTGACCCTGGCCGTGATG | |
| | | | CTGGCCCTCGGCGTGGCCGCCGGCGTGGGCACCGGCACCGC | |
| | | | CGCCCTGGTGACCCGCCCCCAGCACCTGGAGACCGGCCTGA | |
| | | | GCAACCTGCACCGGATCGTGACCGAGGACCTGCAGGCCCTG | |
| | | | GAGAAGAGCGTGAGCAACCTGGAGCAGAGCCTGACCAGCCT | |
| | | | GAGCGAGGTGGTGCTGCAGAACCGGCGGGGCCTGGACCTGC | |
| | | | TGTTCCTGAAGGAGCGCGGCCTGTGCGTGGCCCTGAAGGAC | |
| | | | GAGTGCTGCTTCTACGTGGACCACAGCGGCGCCATCCGGGA | |
| | | | CAGCATGAACAAGCTGCGGGAGCGGCTGGAGAAGCGGCGGC | |
| | | | GGGAGAAGGAGACCACCCAGCCCTGGTTCGAGGGCTGGTTC | |
| | | | AACCCGAGCCCCTGGCTGGCCACCCTGCTGAGCGCCCTGAC | |
| | | | CGGCCCCCTGATCGTGCTGCTGCTGCTGCTGACCGTGGGCC | |
| | | | CCTGCATCATCAACAAGCTGATCGCCTTCATCCGGGAGCGG | |
| | | | ATCAGCGCCGTGCAGATCATGGTGCTGCCGCAGCAGTACCA | |
| | | | GAGCCCCAGCAGCCGGGAGGCCGGCCGGTGATGATGA | |
| B | C | N | ATGCACCCCACCCTGAGCCGGCGGCACCTGCCCATCCGGGG | SEQ ID NO:62 |
| | | | CGGCAAGCCCAAGCGGCTGAAGATCCCCCTGAGCTTCGCCA | |
| | | | GCATCGCCTGGTTCCTGACCCTGAGCATCACCAGCCAGACC | |
| | | | AACGGCATGCGGATCGGCGACAGCCTGAACAGCCACAAGCC | |
| | | | CCTGAGCCTGACCTGGCTGATCACCGACAGCGGCACCGGCA | |
| | | | TCAACATCAACAACACCCAGGGCGAGGCCCCCCTGGGCACC | |
| | | | TGGTGGCCCGACCTGTACGTGTGCCTGCGGAGCGTGATCCC | |
| | | | CAGCCTGACCAGCCCCCCCCACATCCTGCACGCCCACGGCT | |
| | | | TCTACGTGTGCCCCGGCCCCCCCAACAACGGCAAGCACTGC | |
| | | | GGCAACCCCCGGGACTTCTTCTGCAAGCAGTGGAACTGCGT | |
| | | | GACCAGCAACGACGGCTACTGGAACTGGCCCACCAGCCAGC | |
| | | | AGGACCGGGTGAGCTTCAGCTACGTGAACACCTACACCAGC | |
| | | | AGCGGCCAGTTCAACTACCTGACCTGGATCCGGACCGGCAG | |
| | | | CCCCAAGTGCAGCCCCAGCGACCTGGACTACCTGAAGATCA | |
| | | | GCTTCACCGAGAAGGGCAAGCAGGAGAACATCCTGAAGTGG | |
| | | | GTGAACGGCATGAGCTGGGGCATGGTGTACTACGCCGGCAC | |
| | | | CGGCAAGCAGCCCGGCAGCATCCTGACCATCCGGCTCAAGA | |
| | | | TCAACCAGCTGGAGCCCCCCATGGCCATCGGCCCCAACACC | |
| | | | GTGCTGACCGGCCACCGGCCCCCCACCCAGGGCCCCGGCCC | |
| | | | CAGCAGCAACATCACCAGCGGCACCGACCCCACCGAGAGCA | |
| | | | ACAGCACCACCAAGATGGGCGCCAAGCTGTTCAGCCTGATC | |
| | | | CAGGGCGCCTTCCAGGCCCTGAACAGCACCACCCCCGAGGC | |
| | | | CACCAGCAGCTGCTGGCTGTGCCTGGCCCTGGGCCCCCCCT | |
| | | | ACTACGAGGGCATGGCCCGGCGGGGCAAGTTCAACGTGACC | |
| | | | AAGGAGCACCGGGACCAGTGCACCTGCGGCAGCCAGAACAA | |
| | | | GCTGACCCTGACCGAGGTGAGCGGCAAGGGCACCTGCATCG | |
| | | | GCAACGTGCCCCCCAGCCACCAGCACCTGTGCAACCACACC | |
| | | | GAGGCCTTCAACCAGACCAGCGAGAGCCAGTACCTGGTGCC | |
| | | | CGGCTACGACCGGTGGTGGGCCTGCAACACCGGCCTGACCC | |
| | | | CCTGCGTGACCACCCTGGTGTTCAACCAGACCAAGGACTTC | |
| | | | TGCATCATGGTGCAGATCGTGCCCCGGGTGTACTACTACCC | |
| | | | CGAGAAGGCCATCCTGGACGAGTACGACTACCGGAACCACC | |
| | | | GGCAGAAGCGGGAGCCCATCAGCCTGACCCTGGCCGTGATG | |
| | | | CTGGGCCTGGGCGTGGCCGCCGGCGTGGGCACCGGCACCGC | |
| | | | CGCCCTGGTGACCGGCCCCCAGCAGCTGGAGACCGGCCTCA | |
| | | | GCAACCTGCACCGGATCGTGACCGACGACCTGCAGGCCCTG | |
| | | | GAGAACAGCGTGAGCAACCTGGAGGAGAGCCTGACCAGCCT | |
| | | | GAGCGAGGTGGTGCTGCAGAACCGGCGGGGCCTGGACCTGC | |
| | | | TGTTCCTGAACCAGGGCGCCCTGTGCGTGGCCCTGAAGGAG | |
| | | | GAGTCCTGCTTCTACGTGGACCACAGCGGCGCCATCCCGGA | |
| | | | CAGCATGAACAAGCTGCGGGAGCGGCTGGAGAAGCGCCCGC | |
| | | | GGGAGAAGGAGACCACCCAGGGCTGGTTCGAGGGCTGGTTC | |
| | | | AACCGGAGCCCCTGCCTGGCCACCCTGCTGAGCGCCCTGAC | |
| | | | CGGCCCCCTGATCGTGCTGCTGCTGCTGCTGACCGTGGGCC | |
| | | | CCTGCATCATCAACAAGCTGATCGCCTTCATCCGGGAGCGG | |
| | | | ATCAGCGCCGTGCAGATCATGGTGCTGCGGCAGCAGTACCA | |
| | | | GAGCCCCAGCAGCCGGGAGGCCGGCCGGTGATGATGA | |
| C | C | A | ATGCACCCCACCCTGAGCCGCCGGCACCTCCCCATCCCGGG | SEQ ID NO:63 |
| | | | CGGCAAGCCCAAGCGGCTGAAGATCCCCCTGAGCTTCCCCA | |
| | | | GCATCGCCTGGTTCCTGACCCTGAGCATCACCAGCCAGACC | |
| | | | AACGGCATGCGCATCGGCGACAGCCTGAACAGCCACAAGCC | |
| | | | CCTGAGCCTGACCTGGCTGATCACCGACAGCGGCACCGGCA | |
| | | | TCAACATCAACAACACCCAGGGCGACGCCCCCCTGGGCACC | |
| | | | TGGTGGCCCGACCTGTACGTGTGCCTGCGGAGCGTGATCCC | |
| | | | CAGCCTGAACGACCAGACCAGCCCCCCCGACATCCTGCACG | |
| | | | CCCACGGCTTCTACGTGTGCCCCGGCCCCCCCAACAACCGC | |
| | | | AAGCACTGCGGCAACCCCCGGGACTTCTTCTGCAACCAGTG | |
| | | | GAACTGCGTGACCAGCAACCACGGCTACTGGAAGTGGCCCA | |
| | | | CCAGCCAGCAGGACCGGGTGAGCTTCAGCTACGTGAACACC | |
| | | | TACACCAGCAGCGGCCAGTTCAACTACGGCCACGGCCGGTG | |
| | | | CCTGACCTGGCAGCAGCGGGTGCAGAAGGACATCCGGACCG | |
| | | | GCAGCCCCAAGTGCAGCCCCACCGACCTGGACTACCTGAAG | |
| | | | ATCAGCTTCACCGAGAAGGGCAAGCAGGAGAACATCCTGAA | |
| | | | GTGGGTGAACGGCATGAGCTGGGGCATGGTGTACTACGGCG | |
| | | | GCAGCGGCAAGCAGCCCGGCAGCATCCTGACCATCCGGCTG | |
| | | | AAGATCAACACCCAGCTGGAGCCCCCCATGGCCATCGGCCC | |
| | | | CAACACCGTGCTGACCGGCCAGCGGCCCCCCACCCAGGCCC | |
| | | | CCCCCCACAACCTGCCCGTGCCCCAGGGCCCCAGCCCCAAC | |
| | | | CCCGACATCACCCAGAGCGACTACAACATCACCAGCGGCAG | |
| | | | CGACCCCACCAACACCCCCCGGAACGAGAGCAACAGCACCA | |
| | | | CCAAGATGGGCGCCAAGCTGTTCAGCCTGATCCAGGGCGCC | |
| | | | TTCCAGGCCCTGAACAGCACCACCCCCGAGGCCACCAGCAG | |
| | | | CTGCTGCCTGTGCCTGGCCCTCGGCCCCCCCTACTACGAGG | |
| | | | GCATGGCCCGGCGGGGCAAGTTCAACGTGACCAAGCAGCAC | |
| | | | CGGGACCAGTGCACCTGGGGCAGCCAGAACAAGCTGACCCT | |
| | | | GACCGAGGTGACCGGCAAGGGCACCTGCATCGGCAAGGTGC | |
| | | | CCCCCAGCCACCAGCACCTGTGCAACCACACCGAGGCCTTC | |
| | | | AACCAGACCAGCGAGAGCCAGTACCTGGTGCCCGGCTACGA | |
| | | | CCGGTGGTGGGCCTCCAACACCGGCCTGACCCCCTGCGTGA | |
| | | | GCACCCTGGTGTTCAACCAGACCAAGGACTTCTGCATCATG | |
| | | | GTGCAGATCGTGCCCCGGGTGTACTACTACCCCGAGAAGGC | |
| | | | CATCCTGGACGAGTACGACTACCGGAACCACCGGCAGAAGC | |
| | | | GCGAGCCCATCAGCCTGACCCTGGCCGTGATGCTGGGCCTG | |
| | | | GGCGTGGCCGCCGGCGTGGGCACCGGCACCGCCGCCCTGGT | |
| | | | GACCGGCCACCAGCAGCTGGAGACCGGCCTGAGCAACCTGC | |
| | | | ACCGGATCGTGACCGAGGACCTGCAGGCCCTGGAGAAGAGC | |
| | | | GTGAGCAACCTGGAGGAGAGCCTGACCAGCCTGAGCGAGGT | |
| | | | GGTGCTGCAGAACCGGCGGGGCCTGCACCTGCTGTTCCTGA | |
| | | | AGGAGGGCCGCCTGTGCGTGGCCCTGAAGGAGGAGTGCTGC | |
| | | | TTCTACGTGGACCACAGCGGCGCCATCCGGGACAGCATGAA | |
| | | | CAAGCTGCGGGAGCGGCTGGAGAAGCGGCGCCGGGAGAAGC | |
| | | | AGACCACCCAGGGCTGGTTCGAGGGCTGCTTCAACCGGAGC | |
| | | | CCCTGGCTGGCCACCCTGCTGAGCGCCCTGACCGGCCCCCT | |
| | | | GATCGTGCTGCTGCTGCTGCTCACCGTGGGCCCCTGCATCA | |
| | | | TCAACAAGCTGATCGCCTTCATCCGGGAGCGGATCAGCGCC | |
| | | | GTGCAGATCATGGTGCTGCCCCAGCAGTACCAGAGCCCCAC | |
| | | | CAGCCGGGAGGCCGGCCGGCTGTAC | |
| C | C | N | ATGCACCCCACCCTGAGCCGGCGGCACCTGCCCATCCGGGG | SEQ ID NO:64 |
| | | | CGGCAAGCCCAAGCGGCTGAAGATCCCCCTGAGCTTCGCCA | |
| | | | GCATCGCCTGGTTCCTGACCCTGAGCATCACCAGCCAGACC | |
| | | | AACGGCATGCGGATCGGCGACAGCCTGAACAGCCACAAGCC | |
| | | | CCTGAGCCTGACCTGGCTGATCACCGACAGCGGCACCGGCA | |
| | | | TCAACATCAACAACACCCAGGCCGAGGCCCCCCTGGGCACC | |
| | | | TGGTGGCCCGACCTGTACGTGTGCCTGCGGAGCGTGATCCC | |
| | | | CAGCCTCAACCACCAGACCAGCCCCCCCGACATCCTGCACG | |
| | | | CCCACGGCTTCTACGTGTGCCCCGGCCCCCCCAACAACGGC | |
| | | | AAGCACTGCGCCAACCCCCGGGACTTCTTCTGCAAGCAGTG | |
| | | | GAACTGCGTGACCAGCAACGACGGCTACTGGAAGTGGCCCA | |
| | | | CCAGCCACCAGGACCGGGTGAGCTTCAGCTACGTGAACACC | |
| | | | TACACCACCACCGGCCAGTTCAACTACGGCCACGGCCGGTG | |
| | | | GCTGACCTGGCAGCAGCGGGTGCAGAACGACATCCGGACCG | |
| | | | GCAGCCCCAAGTGCAGCCCCAGCGACCTGGACTACCTGAAG | |
| | | | ATCAGCTTCACCGAAGAAGGGCAGCAGGAGAACATCCTGAA | |
| | | | GTGGGTGAACCGCATGAGCTGGGCCATGGTGTACTACGGCG | |
| | | | GCAGCGGCAAGCAGCCCGGCAGCATCCTGACCATCCGGCTG | |
| | | | AAGATCAACACCCAGCTGGAGCCCCCCATGGCCATCGGCCC | |
| | | | CAACACCGTGCTGACCGGCCAGCGGCCCCCCACCCAGGGCC | |
| | | | CCCCCCACAACCTGCCCGTCCCCCAGGGCCCCAGCCCCAAC | |
| | | | CCCGACATCACCCAGAGCGACTACAACATCACCAGCGGCAG | |
| | | | CGACCCCACCAACACCCCCCGGAACGAGAGCAACAGCACCA | |
| | | | CCAAGATGGGCGCCAAGCTGTTCAGCCTGATCCAGGGCCCC | |
| | | | TTCCAGGCCCTGAACAGCACCACCCCCGAGGCCACCAGCAG | |
| | | | CTGCTGGCTGTCCCTGGCCCTGGGCCCCCCCTACTACGAGG | |
| | | | GCATGGCCCGGCGGGGCAAGTTCAACGTGACCAAGGAGCAC | |
| | | | CGGGACCAGTGCACCTGGGGCAGCCAGAACAACCTGACCCT | |
| | | | GACCGACGTCACCGGCAAGGCCACCTGCATCGGCAAGGTGC | |
| | | | CCCCCAGCCACCAGCACCTGTGCAACCACACCGAGGCCTTC | |
| | | | AACCAGACCAGCGAGAGCCAGTACCTGGTGCCCGGCTACGA | |
| | | | CCGGTGGTCGGCCTGCAACACCGGCCTGACCCCCTGCGTGA | |
| | | | GCACCCTGGTGTTCAACCAGACCAAGGACTTCTGCATCATG | |
| | | | GTGCAGATCGTGCCCCGGGTGTACTACTACCCCGAGAAGGC | |
| | | | CATCCTGGACGAGTACGACTACCGGAACCACCGCCAGAAGC | |
| | | | GGGACCCCATCAGCCTGACCCTCGCCGTGATGCTGGGCCTG | |
| | | | GGCGTGGCCGCCGGCGTGGGCACCGGCACCGCCGCCCTGGT | |
| | | | GACCGGCCCCCAGCAGCTGGAGACCCGCCTGAGCAACCTGC | |
| | | | ACCGGATCGTGACCGAGGACCTGCAGGCCCTGGAGAAGACC | |
| | | | GTGAGCAACCTGGACGAGAGCCTGACCAGCCTGAGCGAGGT | |
| | | | GGTGCTGCAGAACCGGCGGCGCCTGGACCTGCTGTTCCTGA | |
| | | | AGGAGCGCGGCCTGTGCGTGGCCCTGAAGGAGGAGTGCTGC | |
| | | | TTCTACGTGGACCACAGCGGCGCCATCCGGGACACCATGAA | |
| | | | CAAGCTGCGGGACCGGCTGGAGAAGCGGCGGCGGGAGAAGG | |
| | | | AGACCACCCAGGGCTGGTTCGAGGGCTGGTTCAACCGGAGC | |
| | | | CCCTGGCTGGCCACCCTGCTGAGCGCCCTGACCGGCCCCCT | |
| | | | GATCGTGCTGCTGCTGCTGCTGACCGTGGGCCCCTGCATCA | |
| | | | TCAACAAGCTGATCGCCTTCATCCGCGAGCGGATCAGCGCC | |
| | | | CTGCAGATCATGGTGCTGCGGCAGCAGTACCAGAGCCCCAG | |
| | | | CAGCCGGGAGGCCGGCCGCCTGTAC | |
| N | C | A | ATGCACCCCACCCTGAGCCGGCGGCACCTGCCCATCCGGGG | SEQ ID NO:65 |
| | | | CGGCAAGCCCAAGCGGCTGAAGATCCCCCTGAGCTTCGCCA | |
| | | | GCATCGCCTGGTTCCTGACCCTGAGCATCACCAGCCAGACC | |
| | | | AACGGCATGCGGATCGGCGACAGCCTGAACACCCACAAGCC | |
| | | | CCTGAGCCTGACCTGGCTGATCACCGACAGCGGCACCGGCA | |
| | | | TCAACATCAACAACACCCAGGGCGAGGCCCCCCTGGGCACC | |
| | | | TGGTGGCCCGACCTGTACGTGTGCCTGCGGAGCGTGATCCC | |
| | | | CAGCCTGACCAGCCCCCCCGACATCCTGCACGCCCACCGCT | |
| | | | TCTACGTGTGCCCCGGCCCCCCCAACAACGGCAAGCACTGC | |
| | | | GGCAACCCCCGGGACTTCTTCTGCAAGCAGTGGAACTGCGT | |
| | | | GACCAGCAACGACGGCTACTGGAAGTGGCCCACCAGCCAGC | |
| | | | AGGACCGGGTCAGCTTCAGCTACGTGAACACCTACACCAGC | |
| | | | AGCGGCCAGTTCAACTACCTGACCTGGATCCGGACCGGCAG | |
| | | | CCCCAAGTGCAGCCCCAGCGACCTCGACTACCTGAAGATCA | |
| | | | GCTTCACCGAGAAGGGCAACCAGGAGAACATCCTGAAGTGG | |
| | | | GTGAACGGCATGAGCTCGGGCATGGTGTACTACGGCGGCAG | |
| | | | CGGCAAGCAGCCCGGCAGCATCCTGACCATCCGGCTGAAGA | |
| | | | TCAACCAGCTGCAGCCCCCCATCGCCATCGGCCCCAACACC | |
| | | | GTGCTGACCCGCCAGCGGCCCCCCACCCAGGGCCCCGGCCC | |
| | | | CAGCAGCAACATCACCAGCGGCAGCGACCCCACCGAGAGCA | |
| | | | ACAGCACCACCAAGATGGGCGCCAAGCTGTTCACCCTGATC | |
| | | | CAGGGCGCCTTCCAGGCCCTGAACAGCACCACCCCCGAGGC | |
| | | | CACCAGCAGCTGCTGGCTGTGCCTGGCCCTGGCCCCCCCCT | |
| | | | ACTACGAGGGCATGGCCCGGCGGGGCAAGTTCAACGTCACC | |
| | | | AACCAGCACCCGGACCAGTGCACCTGGGGCAGCCAGAACAA | |
| | | | GCTGACCCTGACCGAGGTGAGCGGCAAGGGCACCTGCATCG | |
| | | | GCAAGGTGCCCCCCAGCCACCAGCACCTGTGCAACCACACC | |
| | | | GAGGCCTTCAACCAGACCAGCGAGAGCCAGTACCTGGTGCC | |
| | | | CGGCTACGACCCGTGGTGGGCCTGCAACACCGGCCTGACCC | |
| | | | CCTGCGTGAGCACCCTGGTGTTCAACCAGACCAAGGACTTC | |
| | | | TGCATCATGGTGCAGATCGTGCCCCCGGTGTACTACTACCC | |
| | | | CGAGAAGGCCATCCTGGACGAGTACGACTACCGGAACCACC | |
| | | | GGCAGAAGCGGGAGCCCATCAGCCTCACCCTGGCCGTGATG | |
| | | | CTGGGCCTGGGCGTGGCCGCCGGCGTGGGCACCGGCACCGC | |
| | | | CGCCCTGGTGACCGGCCCCCAGCAGCTGGAGACCGGCCTGA | |
| | | | GCAACCTGCACCGGATCGTGACCGAGGACCTGCAGGCCCTG | |
| | | | GAGAAGACCGTGAGCAACCTGGAGGAGAGCCTGACCAGCCT | |
| | | | GAGCGAGGTGGTGCTGCAGAACCGGCGGGGCCTGGACCTGC | |
| | | | TGTTCCTGAAGGAGGCCGGCCTGTGCGTGGCCCTGAAGGAG | |
| | | | GAGTGCTGCTTCTACGTGGACCACAGCGGCGCCATCCGGGA | |
| | | | CAGCATGAACAAGCTGCGGGAGCGGCTGGAGAAGCGGCGGC | |
| | | | GGGAGAAGGAGACCACCCAGGGCTGGTTCGAGGGCTGGTTC | |
| | | | AACCGGAGCCCCTGGCTGGCCACCCTGCTGAGCGCCCTGAC | |
| | | | CGGCCCCCTGATCGTGCTGCTGCTGCTGCTGACCGTGGGCC | |
| | | | CCTGCATCATCAACAAGCTGATCGCCTTCATCCGGGACCGG | |
| | | | ATCAGCGCCGTGCAGATCATGGTCCTGCGGCAGCAGTACCA | |
| | | | GAGCCCCAGCAGCCGGGAGGCCGGCCGGTGATGATGA | |
| N | C | N | ATGCACCCCACCCTGAGCCGGCCGCACCTGCCCATCCGGGG | SEQ ID NO:66 |
| | | | CGGCAAGCCCAAGCGGCTGAAGATCCCCCTGAGCTTCGCCA | |
| | | | GCATCGCCTGGTTCCTGACCCTGAGCATCACCAGCCAGACC | |
| | | | AACGGCATGCGGATCGGCGACAGCCTGAACAGCCACAAGCC | |
| | | | CCTGAGCCTGACCTGGCTGATCACCGACAGCGGCACCGGCA | |
| | | | TCAACATCAACAACACCCAGGGCGAGGCCCCCCTGGGCACC | |
| | | | TGGTGGCCCGACCTGTACGTGTGCCTGCGGAGCGTGATCCC | |
| | | | CAGCCTGACCAGCCCCCCCGACATCCTGCACGCCCACGGCT | |
| | | | TCTACGTGTCCCCCGGCCCCCCCAACAACGGCAAGCACTGC | |
| | | | GGCAACCCCCGGGACTTCTTCTGCAAGCAGTGGAACTGCGT | |
| | | | GACCAGCAACGACGGCTACTGCAAGTGGCCCACCAGCCAGC | |
| | | | AGGACCGGGTGAGCTTCACCTACGTGAACACCTACACCAGC | |
| | | | AGCGGCCAGTTCAACTACCTGACCTGGATCCGGACCGGCAG | |
| | | | CCCCAAGTGCAGCCCCAGCGACCTGGACTACCTGAAGATCA | |
| | | | GCTTCACCGAGAAGGGCAAGCAGGAGAACATCCTGAACTGG | |
| | | | GTGAACGGCATGAGCTGGGGCATGGTCTACTACGGCGGCAG | |
| | | | CGGCAAGCAGCCCGGCAGCATCCTGACCATCCGGCTGAAGA | |
| | | | TCAACCAGCTGGAGCCCCCCATGGCCATCGGCCCCAACACC | |
| | | | GTGCTGACCGGCCAGCGGCCCCCCACCCAGGGCCCCGGCCC | |
| | | | CAGCAGCAACATCACCAGCGGCAGCGACCCCACCGACAGCA | |
| | | | ACAGCACCACCAACATGGGCGCCAAGCTGTTCAGCCTGATC | |
| | | | CAGGGCCCCTTCCAGGCCCTGAACAGCACCACCCCCGAGGC | |
| | | | CACCAGCAGCTGCTGGCTGTGCCTGGCCCTGGGCCCCCCCT | |
| | | | ACTACGAGGGCATGGCCCGGCGGGGCAAGTTCAACGTGACC | |
| | | | AAGGAGCACCGGGACCAGTGCACCTGGGGCAGCCAGAACAA | |
| | | | GCTGACCCTGACCGAGGTGAGCGGCAAGGGCACCTGCATCG | |
| | | | GCAAGGTGCCCCCCAGCCACCAGCACCTGTGCAACCACACC | |
| | | | GAGGCCTTCAACCAGACCAGCGAGAGCCAGTACCTCGTCCC | |
| | | | CGGCTACGACCGGTGGTGGGCCTGCAACACCGGCCTGACCC | |
| | | | CCTGCGTGAGCACCCTGGTCTTCAACCAGACCAAGGACTTC | |
| | | | TGCATCATGGTGCAGATCGTGCCCCGGGTGTACTACTACCC | |
| | | | CGAGAAGGCCATCCTGGACGAGTACGACTACCGGAACCACC | |
| | | | GGCACAAGCGGGAGCCCATCAGCCTGACCCTGGCCGTGATG | |
| | | | CTGGGCCTGGGCGTGGCCGCCGGCGTGGGCACCGGCACCGC | |
| | | | CGCCCTGGTGACCGGCCCCCAGCACCTGGAGACCGGCCTGA | |
| | | | GCAACCTGCACCGGATCGTGACCGAGGACCTGCAGGCCCTG | |
| | | | GAGAAGAGCGTGAGCAACCTGGAGGAGAGCCTGACCACCCT | |
| | | | GAGCGAGGTGGTGCTGCAGAACCGGCGGGGCCTGGACCTGC | |
| | | | TGTTCCTGAAGGAGGGCGGCCTGTGCGTGGCCCTGAAGGAG | |
| | | | GAGTGCTGCTTCTACGTGGACCACAGCGCCGCCATCCGGGA | |
| | | | CAGCATGAACAAGCTGCGGGAGCGCCTGGAGAAGCGGCGGC | |
| | | | GGGAGAAGGAGACCACCCAGGGCTGGTTCGAGGGCTCGTTC | |
| | | | AACCGGAGCCCCTGGCTGGCCACCCTGCTCAGCGCCCTGAC | |
| | | | CGGCCCCCTGATCGTGCTGCTGCTGCTGCTGACCGTGGGCC | |
| | | | CCTGCATCATCAACAAGCTGATCGCCTTCATCCGGGAGCGG | |
| | | | ATCAGCGCCGTGCAGATCATGGTGCTCCCGCAGCAGTACCA | |
| | | | GAGCCCCAGCAGCCGGGAGGCCGGCCCGTGATGATGA | |

What is claimed is:

1. An isolated ancestral viral nucleic acid sequence, and fragments thereof, wherein the sequence is a determined founder sequence of a highly diverse viral strain, subtype or group of an endogenous retrovirus

2. The sequence of claim 1, wherein the ancestral viral nucleic acid sequence is of Porcine Endogenous Retrovirus (PERV) subtype A, B, or C.

3. The sequence of claim 1, wherein the ancestral viral nucleic acid sequence is an env nucleic acid sequence or a fragment thereof.

4. The sequence of claim 1, wherein the sequence has at least 70% identity with the sequence set forth in SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, or SEQ ID NO:41 and wherein the sequence does not have 100% identity with any circulating variant.

5. The sequence of claim 1, which encodes an ancestor protein of SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:43, SEQ ID NO:44, or SEQ ID NO:45.

6. The sequence of claim 1, wherein the sequence is optimized for expression in a human host.

7. The sequence of claim 6, wherein the sequence has at least 70% identity with the sequence set forth in SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, or SEQ ID NO:66 and wherein the sequence does not have 100% identity with any circulating variant.

8. An isolated ancestor protein or fragment thereof from an endogenous retrovirus.

9. The ancestor protein of claim 8, wherein the endogenous retrovirus is PERV of subtype A, B, or C.

10. The isolated ancestor protein of claim 9, which comprises the contiguous sequence of SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

11. The isolated ancestor protein of claim 9, which is at least 10 contiguous amino acids of a PERV subtype A env ancestor protein, a PERV subtype B env ancestor protein, or a PERV subtype C env ancestor protein.

12. An isolated expression construct comprising the following operably linked elements:
a transcriptional promoter;
a nucleic acid encoding an endogenous retrovirus ancestor protein; and
a transcriptional terminator.

13. The expression construct of claim 12, wherein the nucleic acid encodes SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

14. A cultured prokaryotic or eukaryotic cell transformed or transfected with the expression construct of claim 12.

15. An isolated host cell comprising the expression construct of claim 12.

16. A composition for inducing an immune response in a recipient mammal comprising a viral ancestor protein or an antigenic fragment of a viral ancestor protein, wherein the viral ancestor protein is from a virus of a donor species.

17. The composition of claim 16, wherein the ancestor protein of fragment of the ancestor protein is derived from the sequence set forth in SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:43, SEQ ID NO:44, or SEQ ID NO:45.

18. An isolated antibody that binds specifically to an endogenous retrovirus ancestor protein and that binds specifically to a plurality of circulating descendant endogenous retrovirus ancestor proteins.

19. A method of preparing an ancestral endogenous retroviral amino acid sequence, the method comprising:
(a) selecting replication-competent sequences of an endogenous retrovirus;
(b) determining an ancestral endogenous retroviral sequence by maximum likelihood phylogeny analysis that is a most recent common ancestor of the given endogenous retroviral sequences, the ancestral endogenous retroviral sequence representative of the evolutionary center of an evolutionary tree of the given endogenous retroviral sequences; and
(c) synthesizing a viral sequence that is not 100% identical to any of the given endogenous retroviral sequences but whose deduced amino acid sequence is at least 70% identical to any of them.

20. A method for inducing an immune response to a donor virus in a transplant recipient or a potential transplant recipient, the method comprising:
administering to the transplant recipient or potential transplant recipient an immunologically effective amount of a composition comprising a donor virus ancestor protein or an antigenic fragment thereof.

21. A method for inducing an immune response to a donor virus in a transplant recipient or a potential transplant recipient, the method comprising:
administering to the transplant recipient or potential transplant recipient a composition comprising a nucleic acid encoding a donor virus ancestor protein or an antigenic fragment thereof.

22. A method for making a vaccine, the method comprising:
expressing a nucleic acid encoding an endogenous retrovirus ancestor protein in a host cell; and
isolating a preparation comprising the endogenous retrovirus ancestor protein from the host cell.

23. A method for detecting infection with an endogenous retrovirus, the method comprising:
providing a sample comprising nucleic acid molecules present in a biological sample obtained from a subject;
contacting a sample with a probe, wherein the probe is a nucleic acid according to claim 1, and
determining if the sample comprises a nucleic acid molecule that hybridizes to the probe.

24. A method for performing xenotransplantation in a subject, the method comprising:
administering to a subject a composition according to claim 16, and
transplanting in the subject an organ from a different species than the species of the subject.
